SEQUENCE LISTING (135 sequences)

SEQ ID NO: 1 - DNA, 1305 bp, Homo sapiens
  source 1..1305: mol_type = unassigned DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgac

SEQ ID NO: 2 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 3 - protein, 1161 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 4 - DNA, 3486 bp, synthetic construct
  source 1..3486: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 5 - protein, 995 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 6 - DNA, 2988 bp, Synthetic construct
  source 1..2988: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagttga

SEQ ID NO: 7 - protein, 1165 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 8 - DNA, 3498 bp, synthetic construct
  source 1..3498: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 9 - protein, 999 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 10 - DNA, 3000 bp, synthetic construct
  source 1..3000: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagttga

SEQ ID NO: 11 - protein, 892 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 12 - DNA, 2679 bp, synthetic construct
  source 1..2679: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgaccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 13 - protein, 892 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 14 - DNA, 2679 bp, synthetic construct
  source 1..2679: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatccaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgaccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 15 - protein, 892 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 16 - DNA, 2679 bp, synthetic construct
  source 1..2679: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 17 - protein, 896 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 18 - DNA, 2691 bp, synthetic construct
  source 1..2691: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgaccgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 19 - protein, 896 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 20 - DNA, 2691 bp, synthetic construct
  source 1..2691: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 21 - protein, 845 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 22 - DNA, 2538 bp, synthetic construct
  source 1..2538: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagttga

SEQ ID NO: 23 - protein, 1011 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 24 - DNA, 3036 bp, synthetic construct
  source 1..3036: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 25 - protein, 957 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 26 - DNA, 2874 bp, synthetic construct
  source 1..2874: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagttga

SEQ ID NO: 27 - protein, 1123 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 28 - DNA, 3372 bp, synthetic construct
  source 1..3372: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 29 - protein, 666 aa, synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 30 - DNA, 2001 bp, synthetic construct
  source 1..2001: mol_type = other DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 31 - protein, 681 aa, synthetic construct
MYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 32 - DNA, 2046 bp, synthetic construct
  source 1..2046: mol_type = other DNA
atgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 33 - protein, 620 aa, synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 34 - DNA, 1863 bp, synthetic construct
  source 1..1863: mol_type = other DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 35 - protein, 636 aa, synthetic construct
MYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 36 - DNA, 1911 bp, synthetic construct
  source 1..1911: mol_type = other DNA
atgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 37 - protein, 670 aa, synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 38 - DNA, 2013 bp, synthetic construct
  source 1..2013: mol_type = other DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 39 - protein, 685 aa, synthetic construct
MYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 40 - DNA, 2058 bp, synthetic construct
  source 1..2058: mol_type = other DNA
atgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 41 - protein, 742 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 42 - DNA, 2229 bp, synthetic construct
  source 1..2229: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 43 - protein, 739 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 44 - DNA, 2220 bp, synthetic construct
  source 1..2220: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 45 - protein, 739 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 46 - DNA, 2220 bp, synthetic construct
  source 1..2220: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 47 - protein, 733 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 48 - DNA, 2202 bp, synthetic construct
  source 1..2202: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 49 - protein, 1155 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 50 - DNA, 3468 bp, synthetic construct
  source 1..3468: mol_type = other DNA
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 51 - protein, 1159 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 52 - DNA, 3480 bp, synthetic construct
  source 1..3480: mol_type = other DNA
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 53 - protein, 615 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQPGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 54 - DNA, 1845 bp, synthetic construct
  source 1..1845: mol_type = other DNA
atggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctaccagcggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagcggctcccgggccaagagaagcggatccggcgccaccaacttcagcctgctgaagcagcccggagacgtggaagaaaaccctggccctaggatgagcatcggcctgctgtgctgcgccgccctgagcctgctgtgggcaggtccagtgaatgctggtgtcactcagaccccaaaattccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggc

SEQ ID NO: 55 - DNA, 19 bp, synthetic construct
  source 1..19: mol_type = other DNA
gccttccatctgttgctgc

SEQ ID NO: 56 - DNA, 19 bp, synthetic construct
  source 1..19: mol_type = other DNA
acaccttgccgatgtcgag

SEQ ID NO: 57 - DNA, 49 bp, synthetic construct
  source 1..49: mol_type = other DNA
aatgatacggcgaccaccgagatctacactctttccctacacgacgctc

SEQ ID NO: 58 - DNA, 27 bp, synthetic construct
  source 1..27: mol_type = other DNA
gatctacactctttccctacacgacgc

SEQ ID NO: 59 - DNA, 21 bp, synthetic construct
  source 1..21: mol_type = other DNA
aggaagagttcttgcagctcg

SEQ ID NO: 60 - DNA, 25 bp, synthetic construct
  source 1..25: mol_type = other DNA
aatgatacggcgaccaccgagatct

SEQ ID NO: 61 - DNA, 27 bp, synthetic construct
  source 1..27: mol_type = other DNA
gatctacactctttccctacacgacgc

SEQ ID NO: 62 - DNA, 17 bp, synthetic construct
  source 1..17: mol_type = other DNA
accgtgggcttgtactc

SEQ ID NO: 63 - DNA, 357 bp, synthetic construct
  source 1..357: mol_type = other DNA
caggtccagctggtgcagtctggggctgaggtgaagaggcctggggcctcagtgcaggtatcctgcagagcatctggctatagtatcaatacttactatatgcagtgggtgcggcaggcccctggagcaggccttgagtggatgggcgttatcaaccccagtggtgtcacaagttacgcacagaagttccagggcagagtcactttgaccaacgacacgtccacaaacacagtctacatgcagttgaacagtctgacatctgccgacacggccgtctactactgtgcgagatgggccttatggggggacttcggtatggacgtctggggcaagggaaccctggtcaccgtctcgagt

SEQ ID NO: 64 - protein, 119 aa, synthetic construct
QVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSS

SEQ ID NO: 65 - DNA, 321 bp, synthetic construct
  source 1..321: mol_type = other DNA
gacatccagatgacccagtctccttccaccctgtctgcatctattggagacagagtcaccatcacctgccgggccagtgagggtatttatcactggttggcctggtatcagcagaagccagggaaagcccctaaactcctgatctataaggcctctagtttagccagtggggctccatcaaggttcagcggcagtggatctgggacagatttcactctcaccatcagcagcctgcagcctgatgattttgcaacttattactgccaacaatatagtaattatccgctcactttcggcggagggaccaagctggagatcaaa

SEQ ID NO: 66 - protein, 107 aa, synthetic construct
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 67 - DNA, 351 bp, synthetic construct
  source 1..351: mol_type = other DNA
gaggtgcagctgttggagtctgggggaggcttggtacagcctggggggtccctgagaccctcctgtgcagcctctggattcacctttagcagctatgccatgagctgggtccgccaggctccagggaaggggctggagtgggtctcagctattagtggtagtggtggtagcacacactacgcagactccgtgaagggccggttcaccatctccagagacaattccaagaacacgctgtatctgcaaatgaacagcctgagagccgaggacacggccgtgtattactgtgtgagaggaggggctttgggctttgacaactggggccgagggacaatggtcaccgtctcgagt

SEQ ID NO: 68 - protein, 117 aa, synthetic construct
EVQLLESGGGLVQPGGSLRPSCAASGFTFSSYAMSWVRQAPGKGLEWVSAISGSGGSTHYADSVKGRFTISRDNSKNTLYLQMNSLRAEDTAVYYCVRGGALGFDNWGRGTMVTVSS

SEQ ID NO: 69 - DNA, 321 bp, synthetic construct
  source 1..321: mol_type = other DNA
gtgctgactcagccaccctcggtgtctgaagcccccaggcagagggtcaccatttcctgttctggaagcacctccaacatcggaagtaatgttgttaactggtaccagcagttcccaggaaagcctcccaaagtcgtcatctattatgatgatctggtggcctcaggggtctctgaccgattttccggctccaggtctggcacttcagcctccctggccatcagtgggctccagtctgaagatgaagctgattattattgttcagcatgggatgacagcctgaatgcctgggtgttcggtggagggaccaaggtcaccgtc

SEQ ID NO: 70 - protein, 107 aa, synthetic construct
VLTQPPSVSEAPRQRVTISCSGSTSNIGSNVVNWYQQFPGKPPKVVIYYDDLVASGVSDRFSGSRSGTSASLAISGLQSEDEADYYCSAWDDSLNAWVFGGGTKVTV

SEQ ID NO: 71 - protein, 124 aa, synthetic construct
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSS

SEQ ID NO: 72 - protein, 112 aa, synthetic construct
DIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 73 - protein, 112 aa, synthetic construct
DIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 74 - DNA, 750 bp, synthetic construct
  source 1..750: mol_type = other DNA
gaagtgcaattggtgcagagcggtggaggacttgtgaaacctggtggatccctgagactttcctgtgccgcttcgggcttcaccttctcggactactacatgtcctggattcgccaggcccctggaaagggactggaatgggtgtcatacatcagctcctccggttccaccatctactatgccgattccgtgaagggcagattcaccatctcgcgcgacaacgccaagaacactctctatctgcaaatgaactcactgcgggctgaggacaccgcggtctactactgcgcccgggacctcagcggaaagtccagcggatggtcccattacttcgattactggggacagggaaccctggtcaccgtgtccagcggcggggggggctcgggtggcggcggctccggcggcggcgggagcaacttcatgctgactcagccccactccgtgtccgagagcccgggaaagaccgtgactatttcgtgcacacggtcctccgggagcattgcgaacaactacgtgcagtggtaccagcagcggcccgatagggccccaaccactgtgatctacgaagatgaccagcggccgtctggagtcccggaccgcttctcggggtccatcgactcatcatccaattccgcatcgctgacgatcagcggactgaagatcgaggacgaagccgattactactgccagtcctacgacggcaccaactgggtctttgggggtggaaccaagctgactgtgctcgga

SEQ ID NO: 75 - protein, 64 aa, synthetic construct
EVQLVESGGGLVQPGRSLRLSCAASGFTFSSYAMSWVRQAPGKGLEWVAVISYDGSNKYYADSV

SEQ ID NO: 76 - protein, 245 aa, synthetic construct
QVQLVQSGAEVKKPGSSVKVSCKASGGTFSSYAISWVRQAPGQGLEWMGIINPSGGSTSYAQKFQGRVTMTRDTSTSTVYMELSSLRSEDTAVYYCAREYSSSRVDAFDIWGQGTMVTVSSGGGGSGGGGSGGGGSSYELTQPPSVSVSPGQTATITCSGDDLGSKYVCWYQQKPGHSPVLIIYDDSDRPSGIPERFSGSNSGNTATLTISRVEAGDEADYYCQVWDSSSDHAVFGGGTQLTVLG

SEQ ID NO: 77 - DNA, 318 bp, synthetic construct
  source 1..318: mol_type = other DNA
gatatccagctgactcagtccccgtcattcctgtccgcctccgtgggagacagagtgaccatcacctgtcgggccacttcctccgtgtcaagcatgaactggtatcagcagaagcccgggaaggccccaaagccgctgattcacgcgacgtccaacctggcttccggcgtgccgagccggttctccggctcggggagcgggactgagtacaccctgactatttcctcgcttcaacccgaggactttgctacctactactgccaacagtggaccttcaatcctccgacattcggacagggtaccaagttggaaatcaag

SEQ ID NO: 78 - DNA, 366 bp, synthetic construct
  source 1..366: mol_type = other DNA
caagtccaactcgtccagtccggtgcagaagtcaagaaacctggagcatccgtgaaagtgtcttgcaaagcctccggctacaccttcaccaactacaacctccattgggtcagacaggcccccggacaaggactcgaatggatgggagcgatctacccgggaaactacgacaccagctacaaccagaagttcaagggccgcgtgactatgaccgccgataagagcacctccaccgcctacatggaactgtcctcgctgaggtccgaggacactgcggtgtactactgcgcccgcgtggacttcggacactcacggtattggtacttcgacgtctggggacagggcactaccgtgaccgtgtcgagc

SEQ ID NO: 79 - protein, 111 aa, synthetic construct
QSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 80 - protein, 127 aa, synthetic construct
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSS

SEQ ID NO: 81 - protein, 111 aa, synthetic construct
EIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPTFGPGTKVDIK

SEQ ID NO: 82 - protein, 120 aa, synthetic construct
QVQLVQSGGGVVQPGKSLRLSCAASGFTFSIFAMHWVRQAPGKGLEWVATISYDGSNAFYADSVEGRFTISRDNSKDSLYLQMDSLRPEDTAVYYCVKAGDGGYDVFDSWGQGTLVTVSS

SEQ ID NO: 83 - protein, 269 aa, synthetic construct
MALPVTALLLPLALLLHAARPQVQLVQSGGGVVQPGKSLRLSCAASGFTFSIFAMHWVRQAPGKGLEWVATISYDGSNAFYADSVEGRFTISRDNSKDSLYLQMDSLRPEDTAVYYCVKAGDGGYDVFDSWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPTFGPGTKVDIK

SEQ ID NO: 84 - DNA, 2679 bp, synthetic construct; identical to SEQ ID NO: 16
  source 1..2679: mol_type = other DNA

SEQ ID NO: 85 - DNA, 2679 bp, synthetic construct; identical to SEQ ID NO: 16
  source 1..2679: mol_type = other DNA

SEQ ID NO: 86 - DNA, 5124 bp, synthetic construct
  source 1..5124: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttcccatggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 87 - protein, 853 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 88 - DNA, 2553 bp, synthetic construct
  source 1..2553: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 89 - protein, 850 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 90 - DNA, 2535 bp, synthetic construct
  source 1..2535: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 91 - protein, 844 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 92 - DNA, 2505 bp, synthetic construct
  source 1..2505: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 93 - protein, 834 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 94 - DNA, 2640 bp, synthetic construct
  source 1..2640: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 95 - protein, 879 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 96 - DNA, 2535 bp, synthetic construct
  source 1..2535: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 97 - protein, 844 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 98 - DNA, 2454 bp, synthetic construct
  source 1..2454: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgccctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 99 - protein, 817 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 100 - DNA, 2391 bp, synthetic construct
  source 1..2391: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 101 - protein, 796 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 102 - DNA, 2352 bp, synthetic construct
  source 1..2352: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 103 - protein, 783 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 104 - DNA, 2661 bp, synthetic construct
  source 1..2661: mol_type = other DNA
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 105 - protein, 886 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 106 - DNA, 2673 bp, synthetic construct
  source 1..2673: mol_type = other DNA
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 107 - protein, 890 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 108 - DNA, 2538 bp, synthetic construct
  source 1..2538: mol_type = other DNA
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgaccgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 109 - protein, 845 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIIEVMYPPPYLDNEKSNGTIIHVKGKHLCPSPLFPGPSKPFWVLVVVGGVLACYSLLVTVAFIIFWVKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 110 - DNA, 2547 bp, synthetic construct
  source 1..2547: mol_type = other DNA
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgaccgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 111 - protein, 848 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 112 - DNA, 2538 bp, synthetic construct
  source 1..2538: mol_type = other DNA
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 113 - protein, 845 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 114 - DNA, 2547 bp, synthetic construct
  source 1..2547: mol_type = other DNA
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 115 - protein, 848 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 116 - DNA, 2409 bp, synthetic construct
  source 1..2409: mol_type = other DNA
atggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctaccagcggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggctcccgggccaagagaagcggatccggcgccaccaacttcagcctgctgaagcagcccggagacgtggaagaaaaccctggccctaggatgagcatcggcctgctgtgctgcgccgccctgagcctgctgtgggcaggtccagtgaatgctggtgtcactcagaccccaaaattccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggctga

SEQ ID NO: 117 - protein, 802 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSRAKRSGSGATNFSLLKQPGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 118 - DNA, 2409 bp, synthetic construct
  source 1..2409: mol_type = other DNA
atggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctaccagcggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagcggctcccgggccaagagaagcggatccggcgccaccaacttcagcctgctgaagcagcccggagacgtggaagaaaaccctggccctaggatgagcatcggcctgctgtgctgcgccgccctgagcctgctgtgggcaggtccagtgaatgctggtgtcactcagaccccaaaattccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 119 - protein, 802 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQPGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 120 - DNA, 1737 bp, synthetic construct
  source 1..1737: mol_type = other DNA
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 121 - protein, 578 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 122 - DNA, 1845 bp, synthetic construct
  source 1..1845: mol_type = other DNA
atgcagtcgggcactcactggagagttctgggcctctgcctcttatcagttggtgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 123 - protein, 614 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 124 - DNA, 1770 bp, synthetic construct
  source 1..1770: mol_type = other DNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaataagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 125 - protein, 589 aa, synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 126 - DNA, 1716 bp, synthetic construct
  source 1..1716: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 127 - protein, 571 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 128 - DNA, 1929 bp, synthetic construct
  source 1..1929: mol_type = other DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctgctatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtcaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 129 - protein, 642 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 130 - DNA, 1854 bp, synthetic construct
  source 1..1854: mol_type = other DNA
atgcggccgcggctgtggctcctcttggccgcgcagctgacagttctccatggcaactcagtcctccagcagacccctgcatacataaaggtgcaaaccaacaagatggtgatgctgtcctgcgaggctaaaatctccctcagtaacatgcgcatctactggctgagacagcgccaggcaccgagcagtgacagtcaccacgagttcctggccctctgggattccgcaaaagggactatccacggtgaagaggtggaacaggagaagatagctgtgtttcgggatgcaagccggttcattctcaatctcacaagcgtgaagccggaagacagtggcatctacttctgcatgatcgtcgggagccccgagctgaccttcgggaagggaactcagctgagtgtggttgatttccttcccaccactgcccagcccaccaagaagtccaccctcaagaagagagtgtgccggttacccaggccagagacccagaagggcccactttgtagccccatcacccttggcctgctggtggctggcgtcctggttctgctggtttccctgggagtggccatccacctgtgctgccggcggaggagagcccggcttcgtttcatgaaacaattttataaaaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 131 - protein, 617 aa, synthetic construct
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 132 - DNA, 9813 bp, synthetic construct
  source 1..9813: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaatctatgtcgggtgcggagaaagaggtaatgaaatggatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcatttggctaccttaagagagtcagggcccatatatttcgaaacaagtttgtacaaaaagcaggcaccagcgctgtgtaagcggaactagagttacgctagggataacagggtaatatagcctgcaggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattcgatatcaagcttatcggtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 133 - skipped

SEQ ID NO: 134 - DNA, 1455 bp, synthetic construct
  source 1..1455: mol_type = other DNA
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgc

SEQ ID NO: 135 - protein, 485 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0195572 A1
(43) Pub. Date: Jun. 19, 2025

(54) METHODS AND COMPOSITIONS COMPRISING FUSION PROTEINS FOR IMPROVED IMMUNOTHERAPIES

(71) Applicants: New York Genome Center, Inc., New York, NY (US); New York University, New York, NY (US)

(72) Inventors: Neville E. Sanjana, New York, NY (US); Mateusz Legut, Jersey City, NJ (US)

(21) Appl. No.: 18/843,949

(22) PCT Filed: Mar. 15, 2023
(86) PCT No.: PCT/US2023/064452

§ 371 Date: Sep. 4, 2024

Related U.S. Application Data
(60) Provisional application No. 63/320,100, filed on Mar. 15, 2022.

(51) Int. Cl. A61K 35/17; A61K 40/11; A61K 40/31; A61K 40/32; A61K 40/42; A61P 35/00; C07K 14/705; C07K 14/725; C07K 14/73; C07K 16/28; C07K 16/30; C12N 5/0783; C12N 15/86

(52) U.S. Cl. CPC A61K 35/17; A61K 40/11; A61K 40/31; A61K 40/32; A61K 40/4211; A61K 40/4255; A61K 40/4269; A61P 35/00; C07K 14/7051; C07K 14/70514; C07K 14/70517; C07K 14/70521; C07K 14/70578; C07K 16/2827; C07K 16/2896; C07K 16/30; C12N 5/0636; C12N 15/86; A61K 2239/13; A61K 2239/21; A61K 2239/57; C07K 2317/622; C07K 2319/02; C07K 2319/03; C12N 2510/00; C12N 2740/15043

(57) ABSTRACT

Provided herein are nucleic acids, expression cassettes, modified lymphocytes and compositions comprising the same which include a sequence encoding a fusion protein, TCR or CAR that includes a domain of LTBR. In certain embodiments, the cell is a T cell. Methods of treatment using the provided compositions are also described.

### STATEMENT OF GOVERNMENT SUPPORT

This invention was made with government support under R00HG008171, DP2HG010099, and R01CA218668 awarded by the National Institutes of Health and D18AP00053 awarded by the Defense Advanced Research Projects Agency. The government has certain rights in the invention.

### REFERENCE TO AN ELECTRONIC SEQUENCE LISTING

The contents of the electronic sequence listing (NYG-LIPP-158PCT.xml; Size: 304,622 bytes; and Date of Creation: Mar. 15, 2023) is herein incorporated by reference in its entirety.

### BACKGROUND

Cellular immunotherapies with engineered autologous patient T cells redirected against a chosen tumor antigen have yielded great efficacy against blood cancers, resulting in five approvals for chimeric antigen receptors (CARs) by the US Food and Drug Administration (FDA) so far⁶. By contrast, CAR therapy for solid tumors has shown a much lower efficacy overall, owing to the suppression of T cell effector function in the tumor microenvironment. Even for blood malignancies, with the exception of B acute lymphoblastic leukemia, most patients do not experience a durable response, with resistance being primarily due to T cell dysfunction rather than antigen loss⁷. Considerable efforts have been devoted to identifying genes and pathways that contribute to T cell dysfunction^{8,9}. However, comprehensive, genome-wide screens for modulators of T cell function thus far have been limited to loss-of-function screens²⁻⁴.

The advances in CRISPR genome engineering have made it possible to readily knock out every gene in the genome in a scalable and customizable manner. Although its large size makes it challenging (albeit not impossible¹⁰) to deliver Cas9 via lentivirus to primary T cells, alternative approaches have been developed, which rely on transient delivery of Cas9 protein² or mRNA¹¹, or on constitutive Cas9 expression in engineered isogenic mouse strains³. These approaches, however, are not amenable to gain-of-function screens in human cells, which require continuous expression of the transcriptional activator that drives target gene expression.

What is needed is improved compositions and methods for more effective immunotherapies.

### SUMMARY OF THE INVENTION

Provided herein, in a first aspect, is a lymphocyte genetically modified to express a chimeric antigen receptor (CAR). The CAR includes an antigen binding domain; a transmembrane domain; and a signaling domain. In certain embodiments, at least one domain comprises an LTBR domain. In other embodiments, at least one domain comprises a domain from a gene of Table 3. In certain embodiments, the LTBR domain is an LTBR intracellular domain, or fragment or variant thereof. In certain embodiments, the LTBR intracellular domain comprises amino acids 249 to 435 of SEQ ID NO: 2, or a fragment, deletion, or variant thereof. In certain embodiments, the LTBR intracellular domain has a deletion in at least amino acids 393 to 435.

In another embodiment, the CAR includes an antigen binding domain; a transmembrane domain; a co-stimulatory signaling domain; and a signaling domain. In certain embodiments, at least one domain comprises an LTBR domain. In other embodiments, at least one domain comprises a domain from a gene of Table 3. In certain embodiments, the LTBR domain is an LTBR intracellular domain, or fragment or variant thereof. In certain embodiments, the LTBR intracellular domain comprises amino acids 249 to 435 of SEQ ID NO: 2, or a fragment, deletion, or variant thereof. In certain embodiments, the LTBR intracellular domain has a deletion in at least amino acids 393 to 435.

In another aspect, a nucleic acid molecule is provided. The molecule includes a sequence that encodes a chimeric antigen receptor (CAR). The CAR includes an antigen binding domain; a transmembrane domain; and a signaling domain. In certain embodiments, at least one domain comprises an LTBR domain. In other embodiments, at least one domain comprises a domain from a gene of Table 3. In certain embodiments, the LTBR domain is an LTBR intracellular domain, or fragment or variant thereof. In certain embodiments, the LTBR intracellular domain comprises amino acids 249 to 435 of SEQ ID NO: 2, or a fragment, deletion, or variant thereof. In certain embodiments, the LTBR intracellular domain has a deletion in at least amino acids 393 to 435. In another embodiment, the CAR includes an antigen binding domain; a transmembrane domain; a co-stimulatory signaling domain; and a signaling domain. In certain embodiments, at least one domain comprises an LTBR domain In other embodiments, at least one domain comprises a domain from a gene of Table 3. In certain embodiments, the LTBR domain is an LTBR intracellular domain, or fragment or variant thereof. In certain embodiments, the LTBR intracellular domain comprises amino acids 249 to 435 of SEQ ID NO: 2, or a fragment, deletion, or variant thereof. In certain embodiments, the LTBR intracellular domain has a deletion in at least amino acids 393 to 435.

In another aspect, an expression cassette is provided that includes a nucleic acid molecule that includes a sequence that encodes a chimeric antigen receptor (CAR).

In another aspect, a method treating cancer in a subject in need thereof is provided. The method includes administering a composition comprising a modified lymphocyte as described herein.

In another aspect, a method of treating a viral disease in a subject in need thereof is provided. The method includes administering a composition comprising a modified lymphocyte In another aspect, a method of treating an autoimmune in a subject in need thereof is provided. The method includes administering a composition comprising a modified lymphocyte In another aspect, a fusion protein comprising an LTBR domain and at least one domain from a second protein, that is not LTBR is provided.

In another aspect, a host cell comprising a nucleic acid molecule or expression cassette as described herein, is provided.

Other aspects and advantages of the invention will be readily apparent from the following detailed description of the invention.

### BRIEF DESCRIPTION OF THE DRAWINGS

FIG. 1A-FIG. 1D show a genome-scale overexpression screen to identify genes that boost the proliferation of primary human T cells. (FIG. 1A) Overview of the pooled ORF screen. CD4⁺ and CD8⁺ T cells were separately isolated from peripheral blood from three healthy donors. The barcoded genome-scale ORF library was then introduced into CD3/CD28-stimulated T cells, followed by selection of transduced cells. After 14 days of culture, T cells were labelled with carboxy fluorescein succinimidyl ester (CFSE) and restimulated to induce proliferation. By comparing counts of specific ORF barcodes before and after cell sorting, we identified ORFs enriched in the CFSElow population. (FIG. 1B) Normalized enrichment of individual barcodes for the indicated genes in the CD4⁺ screen. (FIG. 1C) Robust rank aggregation of genes in both CFSER^{low}CD4⁺ and CFSE^{low}CD8⁺ T cells, based on consistent enrichment of individual barcodes for each gene. (FIG. 1D) Enrichment in individual donors and T cell populations of top-ranked genes (grouped by function and relevance to T cell proliferation) selected for further study. Neutral genes (MHC-I complex and cell-type specific differentiation markers) are included for comparison. Gene names are colored on the basis of the differential expression in CD3/CD28-stimulated and resting T cells (green, upregulated: red, downregulated: grey, no change; black, no expression)⁴¹.

FIG. 2A-FIG. 2F show overexpression of top-ranked ORFs increases the proliferation, activation and cytokine secretion of CD4⁺ and CD8⁺ T cells. (FIG. 2A) CD4+ and CD8⁺ T cells from screen-independent donors were separately isolated and then transduced with lentiviruses encoding top-ranked ORFs together with a selection marker. After transduction and selection. T cells were restimulated before measurement of proliferation, expression of activation markers and cytokine secretion. (FIG. 2B) Proliferation of T cells transduced with top-ranked genes as the relative proliferation, which is defined as the ratio of stimulated cells to the corresponding unstimulated control, normalized to tNGFR. A minimum of two donors was tested per overexpressed gene, in biological triplicate. Boxes show 25th-75th percentiles with a line at the mean: whiskers extend to maximum and minimum values DUPD1 is also known as DUSP29. (FIG. 2C) Mean relative proliferation of ORF-transduced T cells in CD4⁺ and CD8⁺ T cells, normalized to tNGFR. Significant genes in both T cell subsets or either of them are marked (Student's two-sided? test P<0.05 and false discovery rate <0.1). (FIG. 2D) Representative expression of CD25 or CD154 after restimulation. The numbers on the histograms correspond to the percentage of gated cells (CD8⁺CD154⁺) or the mean fluorescence intensity (MFI). Dashed lines indicate the gate used to enumerate CD154⁺ cells (CD8⁺) or MFI for control (tNGFR) cells. (FIG. 2E) Secretion of IL-2 and IFNγ after restimulation, normalized to tNGFR. Only genes that significantly increase T cell proliferation in CD4⁺, CD8⁺ or both T cell subsets are shown. A minimum of two donors was tested in triplicate per gene. Boxes show 25th-75th percentiles with a line at the mean; whiskers extend to maximum and minimum values. (FIG. 2F) Intersection between different T cell activation phenotypes that are significantly (P<0.05) improved by a given ORF in CD8⁺ or CD4⁺ T cells.

FIG. 3A-FIG. 3E show single-cell OverCITE-seq identifies shared and distinct transcriptional programs that are induced by gene overexpression in T cells. (FIG. 3A) OverCITE-seq captures overexpression (ORF) constructs, transcriptomes, TCR clonotypes, cell-surface proteins and treatment hashtags in single cells. (FIG. 3B) ORF assignment rate in resting and CD3/CD28-stimulated T cells. (FIG. 3C) Antibody-derived tag sequencing (ADTs; right) yields similar NGFR expression in tNGFR-transduced T cells to flow cytometry (left) with tNGFR-transduced T cells. Untransduced cells (left) or cells assigned a non-tNGFR ORF (right) are shown in grey. (FIG. 3D) Uniform manifold approximation and projection (UMAP) representation of single-cell transcriptomes after unsupervised clustering of OverCITE-seq-captured ORF singlets. The inset in the top left identifies stimulated and resting T cells as given by treatment hashtags. For each cluster, a subset of the top 20 differentially expressed genes is shown. HIST1H1B is also known as H1-5 and HIST1H3C is also known as H3C3. (FIG. 3E) ORF prevalence in two representative clusters. Standardized residual values are from a chi-squared test. ORFs of interest are shown.

FIG. 4A-FIG. 4K show LTBR overexpression improves T cell function through activation of the canonical NF-κB pathway. (FIG. 4A) Differential expression of genes in resting LTBR and tNGFR (negative control) T cells. Genes highlighted in red are those with a twofold or greater change in expression and an adjusted P<0.05. (FIG. 4B) Significantly enriched GO biological processes in LTBR-overexpressing T cells (p<0.05). (FIG. 4C) Cell viability of CD8+T cells transduced with LTBR or tNGFR lentivirus, either restimulated with CD3/CD28 for four days or left unstimulated (n=2 donors with 3 biological replicates each). (FIG. 4D) PD-1 expression on resting LTBR or tNGFR T cells stimulated with a 3:1 excess of CD3/CD28 beads every three days, for up to three rounds of consecutive stimulation. (FIG. 4E) ICAM-1 expression (resting) and IL-2 secretion (activated) by T cells transduced with Flag-tagged LTBR mutants, normalized to wild-type LTBR (n=6 replicates across two experiments). (FIG. 4F) Enrichment of transcription factor motifs in differentially accessible chromatin (top 10 motifs from each comparison). (FIG. 4G) Quantification of phosphorylated RELA (phospho-RELA) in LTBR or tNGFR T cells stimulated with CD3/CD28 antibodies for the indicated periods of time. (FIG. 4H. FIG. 4I) Quantification of phosphorylated IκBα (FIG. 4H) or mature NF-κB2 (FIG. 4I) in resting or CD3/CD28-stimulated (15 min) LTBR or tNGFR cells. (FIG. 4J) IFNγ secretion by stimulated LTBR or tNGFR cells after CRISPR knockout of the indicated genes (n=18, 3 sgRNAs in 2 donors in 3 biological replicates). IFNγ quantities are normalized to corresponding non-targeting (NT) controls (either LTBR or tNGFR) to allow comparisons of the relative effects of gene knockout on T cell activation. (FIG. 4K) Expression levels of core LTBR genes (n=274 genes) in LTBR and tNGFR cells after CRISPR knockout of RELA or RELB (normalized to non-targeting control in LTBR cells). Boxes show 25th-75th percentiles with a line at the median: whiskers extend to 1.5 times the interquartile range. Unpaired two-sided t test P values (FIG. 4C. FIG. 4G-FIG. 4K): not significant (NS) P>0.05, *P<0.05, ** P<0.01, *** P<0 001. **** P<0.0001. Error bars, s.e.m.; n=3 biological replicates, unless stated otherwise.

FIG. 5A-FIG. 5I show top-ranked genes improve antigen-specific T cell responses and tumor killing. (FIG. 5A-FIG. 5G) Co-delivery of anti-CD19 CARs and ORFs to T cells from healthy donors. (FIG. 5A) Schematic of tricistronic vector and CAR T cell experiments. (FIG. 5B, FIG. 5C) Secretion of IFNγ (FIG. 5B) and IL-2 (FIG. 5C) after overnight co-incubation of CD8⁺ T cells with Nalm6 cells at a 1.1 ratio (n=3 biological replicates, representative of 2 donors). (FIG. 5D) Representative images of Nalm6 GFP⁺ cells co-incubated for 48 h with CAR T cells or untransduced control T cells. Scale bar. 200 μm. (FIG. 5E) Nalm6 GFP⁺ cell proliferation (normalized total GFP per well) after co-incubation with T cells co-expressing 19-28z CAR and LTBR or tNGFR (negative control) at the indicated effector-to-target ratios. (FIG. 5F) Quantification of Nalm6 GFP⁺ clearance for T cells co-expressing 19-28z or 18-BBz CARs and top-ranked genes (n=3 biological replicates, representative of 2 donors), normalized to tNGFR at an effector-to-target ratio of 0.25 and after 48 h of co-incubation. (FIG. 5G) 19-BBz CAR T cells co-expressing LTBR or tNGFR were co-incubated at a 1:1 ratio with Nalm6 cells every 3 days for up to 3 rounds of stimulation (n=3 biological replicates). Seven days after repeated antigen stimulation. CAR T cells were re-exposed to Nalm6 cells. IFNγ secretion was measured after overnight incubation. (FIG. 5H) Co-delivery of anti-CD19 CARs and ORFs to total PBMCs from a patient with diffuse large B cell lymphoma. Transduced T cells were incubated alone, or co-incubated with CD19⁺ Nalm6 or CD19⁻ Jurkat cell lines at a 1:1 ratio (n=3 biological replicates, representative of 2 patients). Secretion of IFNγ and IL2 was measured after overnight incubation. For the Nalm6 condition, numbers above indicated column pairs are the fold increase in cytokine secretion by LTBR cells over tNGFR (negative control) cells. (FIG. 5I) Delivery of ORFs to Vγ9Vδ2 T cells. Secretion of IFNγ and IL-2 after overnight co-incubation with the pancreatic ductal adenocarcinoma (PDAC) line Capan-2, pre-treated with zoledronate to boost phosphoantigen accumulation (n=3 biological replicates). Data are mean #s.e.m. where appropriate.

FIG. 6A-FIG. 6Q show design of the human ORF library screen in primary T cells. (FIG. 6A) Barcoded vector design for ORF overexpression. (FIG. 6B) Distribution of the number of barcodes per ORF in the library. (FIG. 6C) Vector design for quantifying the effect of different promoters and ORF insert sizes on lentiviral transduction efficiency. EFS—elongation factor-1α short promoter, CMV—cytomegalovirus promoter, PGK-phosphoglycerate kinase-1 promoter. (FIG. 6D) Sequential gating strategy and representative histograms of cells transduced with marker gene rat CD2 under different promoters. (FIG. 6E) Percentage of positive cells and (FIG. 6F) mean fluorescence intensity (MFI) of rat CD2 (rCD2) expressed from the EFS and CMV promoters, following puromycin selection of transduced primary CD4⁺ T cells. Each data point indicates individual transduction (n=3 biological replicates). Error bars are SEM. (FIG. 6G) Distribution of ORF sizes in the genome-scale library. The size of TCR-rCD2 construct tested in panels FIG. 6E and FIG. 6F is marked. (FIG. 6H) Titration of CD3/CD28 antibodies. T cells were labelled with CFSE, stimulated and incubated for 4 days. Gate for proliferating T cells was set to include cells that proliferated at least twice (third CFSE peak) (FIG. 6I) Expansion of T cells from three healthy donors transduced with the ORF library. (FIG. 6J) Representative CFSE profile of restimulated CD8⁺ and CD4⁺ T cells before the sort. The CFSElow sort gate is marked. (FIG. 6K) Recovery of individual barcodes or corresponding ORFs in transduced T cells and plasmid used for lentivirus production. Respective samples from three donors were computationally pooled together at equal number of reads prior to counting how many barcodes or ORFs were present with a minimum of one read. (FIG. 6L) Distribution of reads corresponding to ORFs of different sizes. ORFs were assigned to ten quantiles based on their size, with Q1 being smallest size and Q10 being the largest size (n=1.161 ORFs per quantile). Box shows 25-75 percentile with a line at the median; whiskers extend to 1.5× interquartile range. (FIG. 6M) Enrichment of genes in both CFSE^{low} CD4⁺ and CD8⁺ T cells, calculated by collapsing individual barcodes into corresponding genes. Significantly enriched genes (log, fold change higher than 0.5 and adjusted p-value lower than 0.05) are marked in red. Immune response genes of interest are marked. (FIG. 6N) Overlap of significantly enriched genes from FIG. 6M in individual screen populations (CD4⁺, CD8⁺) analyzed separately. (FIG. 6O) Normalized enrichment of individual barcodes for indicated genes in the CD8⁺ screen. (FIG. 6P) GO biological processes for significantly enriched genes in FIG. 6M. (FIG. 6Q) Overlap of significantly enriched genes with differentially expressed genes between CD3/CD28 stimulated and naive T cells⁴¹.

FIG. 7A-FIG. 7J show overexpression of select ORFs in screen independent donors. (FIG. 7A) Histograms of selected ORF expression in T cells after puromycin selection. (FIG. 7B) Quantification of tNGFR expression in transduced CD4⁺ and CD8⁺ T cells. Puromycin selection was complete after 7 days post transduction. To maintain T cells in culture, they were restimulated with CD3/CD28 on days 21 and 42. (FIG. 7C) Correlation between ORF sizes and changes in proliferation relative to tNGFR. Mean log, fold-changes are shown. (FIG. 7D) Proliferation of restimulated CD8⁺ or (FIG. 7E) CD4⁺ T cells relative to tNGFR in individual donors (n=3 biological replicates). Mean and SEM are shown. (FIG. 7F, FIG. 7G) Proliferation of T cells transduced with ORFs that significantly improved T cell proliferation (see FIG. 2C) measured by dilution of CellTrace Yellow. Representative CellTrace Yellow histograms and fitted distributions (FIG. 7F) as well as quantifications of the proliferation index (FIG. 7G) are shown (n=3 biological replicates). P values: <0.0001, 0.0008, <0.0001, 0.011, 0.0031, 0.0007, <0.0001, 0.28, 0.004, <0.0001, 0.58, 0.01, 0.0003, <0.0001, 0.036, 0.0049 (left to right). (FIG. 7H) Viability of ORF-transduced T cells 4 days after CD3/CD28 restimulation Representative data from one donor (out of 4 donors tested) are shown (n=3 biological replicates). (FIG. 7I, FIG. 7J) Cell cycle analysis of T cells stimulated with CD3/CD28 for 24 h. Gating was performed based on isotype and fluorescence minus one controls. Representative gating (FIG. 7I) as well as (FIG. 7J) quantification of cells in the S-G2-M phases (for stimulated T cells) are shown (n=6 biological replicates from two donors). P values: 1, 0.29, 0.0065, 0.17, 0.0051, 1, 0.13, 0.55, 0.0004, 0.98, 0.0088, 0.68, 0.91, 0.7, 1 (left to right). Statistical significance for panels FIG. 7G and FIG. 7I: one way ANOVA with Dunnett's multiple comparisons test * p<0.05, ** p<0.01, *** p<0.001, **** p<0.0001. Error bars indicate SEM.

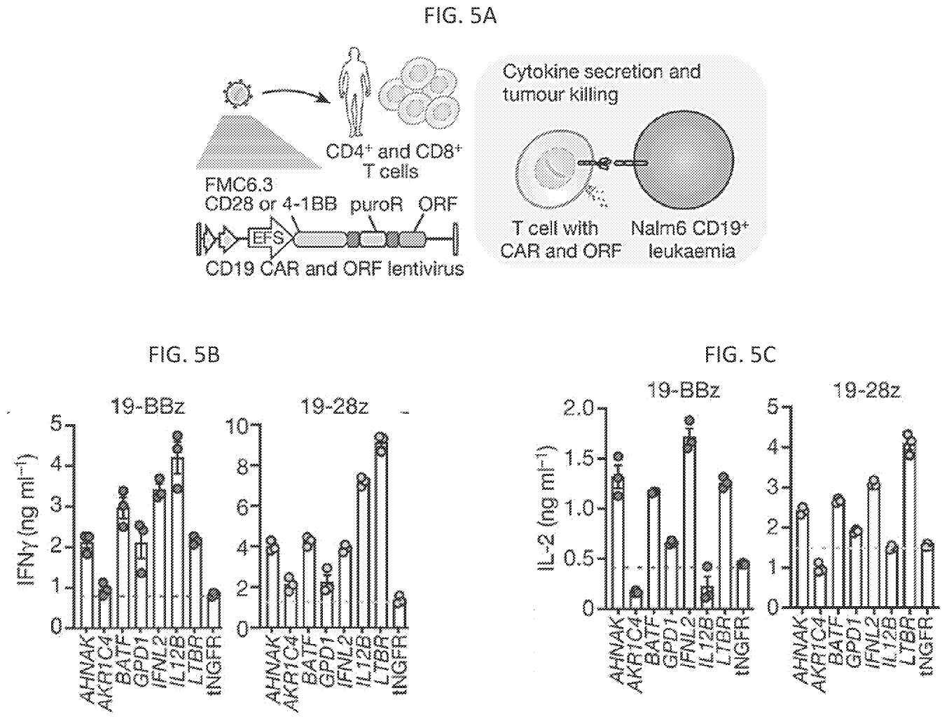

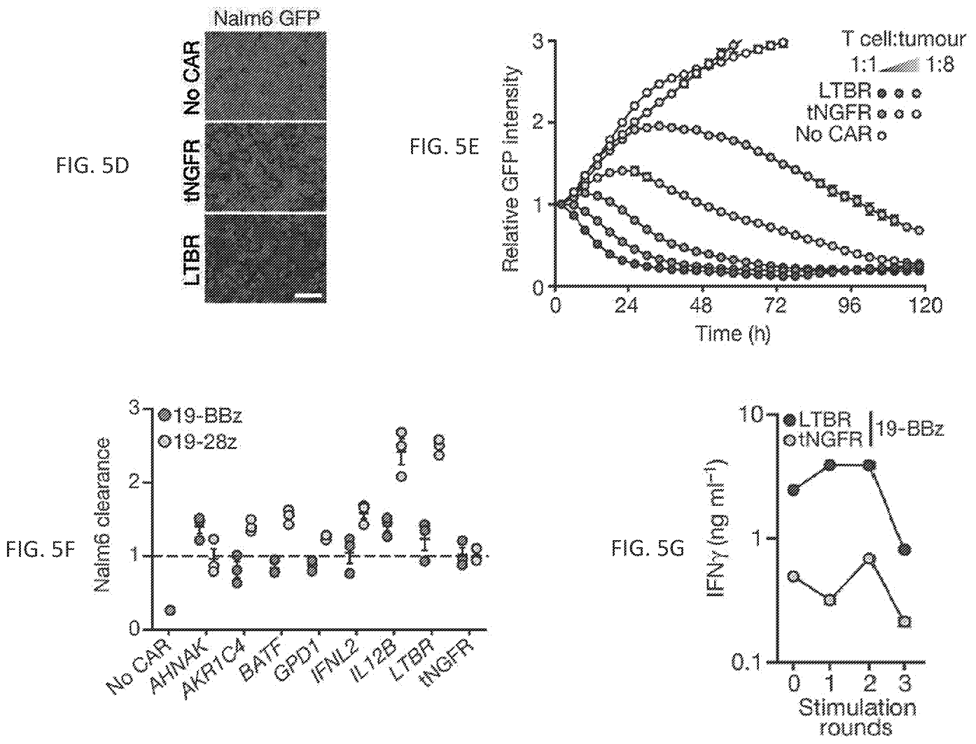

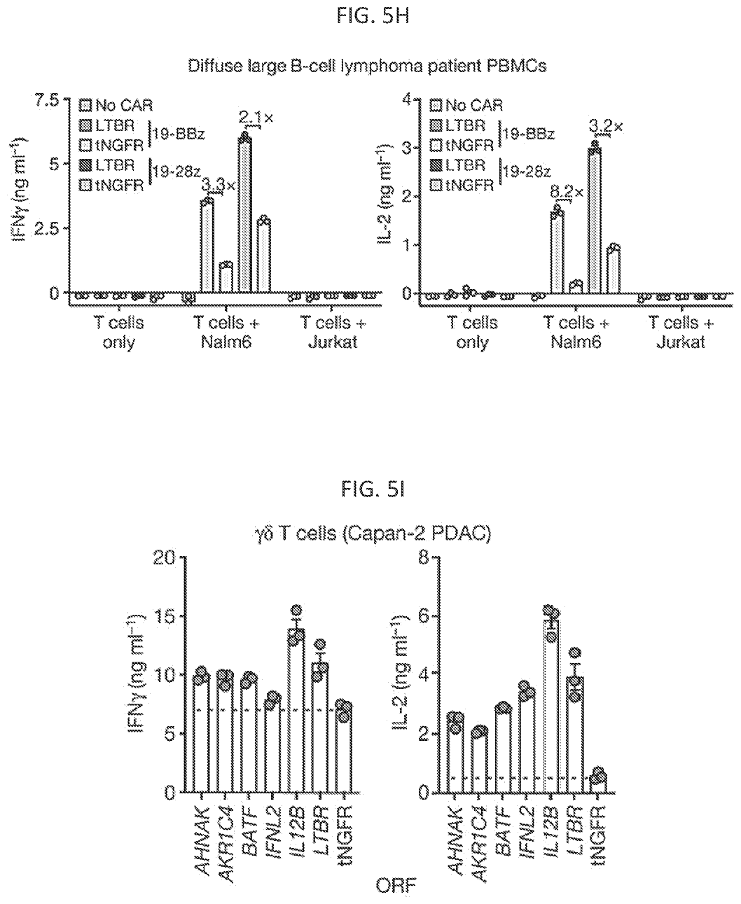

FIG. 5A-FIG. 8E show functional response of ORF-overexpressing T cells. (FIG. 8A) Quantitative expression of CD25 or CD154 following restimulation. A minimum of two donors was tested in triplicate per gene. Only genes that significant increase T cell proliferation in CD4″, CD8⁺ or both T cell subsets are shown. Mean and SEM are shown. (FIG. 8B. FIG. 8C) Sensitivity to antigen dose. T cells were incubated with indicated anti-CD3 antibody concentrations for 24 h and the amount of secreted IFNγ was quantified. Representative dose-response curve fitting (FIG. 8B) and IC₅₀ quantifications (FIG. 8C) are shown (n=2 biological replicates) (FIG. 8D) Quantification of secreted IL-2 and IFNγ in T cells incubated alone or with CD3/CD28 antibodies for 24 h. Representative data from one out of four donors (n=3 biological replicates) are shown. (FIG. 8E) Multiplexed quantification of selected secreted cytokines and chemokines by ORF-transduced T cells after 24 h of CD3/CD28 stimulation. Means of duplicate measurements (from independent samples) z-score normalized to tNGFR are shown.

FIG. 9A-FIG. 9J show OverCITE-seq identifies ORFs and their transcriptional effects. (FIG. 9A) Quality parameters of cells as identified by gel bead barcodes. Negative, singlets and doublets are assigned based on cell hashing. (FIG. 9B) Proportion of stimulated and resting T cells among cells assigned to each ORF. Chi-squared test p-values are shown for ORFs with significantly shifted (uneven) distributions of stimulated and rested cells. (FIG. 9C) Cell-cycle corrected scaled expression of the overexpressed gene in the cells transduced with the respective ORF and negative control (tNGFR). Two-sided Wilcoxon test p-values shown above the violin plots indicate the statistical significance of gene expression level between specific ORF and tNGFR-transduced T cells. Box shows 25-75 percentile with a line at the median, whiskers extend to maximum and minimum values. N=71 (ADA), 147 (AHCY), 190 (AHNAK), 119 (AKR1C4), 124 (ATF6B), 179 (BATF), 137 (CALML3), 189 (CDK1), 129 (CDK2), 236 (CLIC1), 84 (CRLF2), 91 (CXCL12), 88 (CYP27A1), 129 (DBI), 26 (DCLRE1B), 261 (DUPD1), 25 (FOSB), 119 (GPD1), 124 (GPN3), 199 (IFNL2), 60 (IL12B), 70 (IL1RN), 156 (ITM2A), 74 (LTBR), 88 (MRPL18), 167 (MRPL51), 107 (MS4A3), 69 (NFYB), 355 (NGFR), 261 (RAN), 182 (SLC10A7), and 56 (ZNF830) single cells. (FIG. 9D) Expression of all ORF genes by cells assigned each ORF. Each row is z-score normalized. (FIG. 9E) Distribution of individual ORF frequencies in clusters. Numbers of ORF cells and the chi-squared test residuals are displayed. Chi-squared test p-values indicating whether ORF distribution in each cluster significantly differs from overall ORF distribution are shown on top of the plot. Proportions of stimulated and resting T cells in each cluster are shown underneath the cluster label. (FIG. 9F, FIG. 9G) Spearman correlations between transcriptional profiles of selected ORF cells in resting (FIG. 9F) and stimulated (FIG. 9G) populations. (FIG. 9H) Fold change of top differentially expressed genes between cells with the indicated ORFs in resting and stimulated T cells. For each condition, the ORFs with the strongest transcriptional changes (compared to tNGFR cells) are shown. (FIG. 9I) Differential gene expression in stimulated ORF T cells compared to resting T cells. Genes with significant expression changes in at least one ORF are shown (DESeq2 adjusted p<0.05). For all genes, we display log₂ fold-change of each ORF (stimulated) to tNGFR (resting), normalized to log: fold-change of tNGFR (stimulated) to tNGFR (resting). Genes of interest in each cluster arc labelled. (FIG. 9J) Mean TCR clonotype diversity in ORF cells

FIG. 10A-FIG. 10M show functional analysis of LTBR overexpression in T cells. (FIG. 10A) LTBR expression in the indicated human primary tissues from the Genotype-Tissue Expression (GTEx) project v8⁷⁵ (n=948 donors). Box shows 25-75 percentile with a line at the median. (FIG. 10B) LTBR expression in peripheral blood mononuclear cells (PBMCs) from 31,021 cells from 2 donors⁷⁶. Cell types indicated are derived from Harmony tSNE clustering of single-cell transcriptomes. (FIG. 10C) Overlap between significantly upregulated genes in LTBR cells compared to tNGFR cells identified in single-cell or bulk RNA-seq. (FIG. 10D. FIG. 10E) TCF1 expression in LTBR or tNGFR transduced T cells. (FIG. 10D) Representative histograms of TCF1 expression and the gate for TCF1+ cells (dashed line) are shown, as well as (FIG. 10E) quantification of TCF1⁺ cells (n=3 biological replicates). (FIG. 10F-FIG. 10H) ICAM-1. CD70, CD74, and MHC-11 expression in LTBR and tNGFR T cells. Representative histograms (FIG. 10F), quantification (FIG. 10G) in n=3 donors (CD8⁺) or n=4 donors (CD4⁺) and time course (FIG. 10H) of expression in LTBR and tNGFR cells after CD3/CD28 stimulation (n=3 biological replicates). (FIG. 10I) Differentiation phenotype of NGFR and LTBR transduced T cells (n=4 donors, CD4⁺ and CD8⁺ separately). CM: Central memory. EM: Effector memory. Differentiation was defined based on CD45RO and CCR7 expression (naive: CD45RO^{neg} CCR7⁺, CM: CD45RO⁺ CCR7⁺, EM: CD45RO⁺ CCR7^{neg}, effector CD45RO^{neg} CCR7⁺). (FIG. 10J) Representative dot plots of T cell viability after CD3/CD28 stimulation. Viable cells are in the lower left quadrant. (FIG. 10K) Cell viability of CD4⁺ T cells transduced with LTBR or tNGFR lentivirus, either restimulated with CD3/CD28 for four days or left unstimulated (n=2 donors with 3 biological replicates each). (FIG. 10L, FIG. 10M) LTBR and tNGFR cells were stimulated with a 3:1 excess of CD3/CD28 beads every three days for up to three rounds of stimulation. Following repeated stimulation, expression of TIM-3 and LAG-3 (FIG. 10L) was measured in resting cells, and secretion of IFNγ and IL2 (FIG. 10M) was measured in restimulated cells (n=3 biological replicates). Statistical significance for panels FIG. 10E, FIG. 10I, and FIG. 10K: two-sided unpaired/-test: for panel FIG. 10G: two-sided paired t-test. Error bars indicate SEM.

FIG. 11A-FIG. 11K show LTBR ligands and expression of LTBR via mRNA or with deletion and point mutants. (FIG. 11A) IL2 secretion after 24 h stimulation with CD3/CD28 antibodies. Where indicated, recombinant soluble LTA (1 ng/mL) or LIGHT (10 ng/mL) were added together with CD3/CD28 antibodies. CD4⁺ T cells from one donor were tested in triplicate. (FIG. 11B, FIG. 11C) CD4⁺ and CD8⁺ T cells from two donors were co-incubated for 24 h with CD3/CD28 antibodies or recombinant soluble LTA or LIGHT and then IL2 (FIG. 11B) and IFNγ (FIG. 11C) were measured (n=3 biological replicates). (FIG. 10D, FIG. 10E) Differentiation phenotype (FIG. 10D) or proliferation (FIG. 10E) after restimulation of tNGFR and LTBR transduced T cells (n=3 biological replicates) incubated cither with IL2 alone or with LTA (1 ng/mL) or LIGHT (10 ng/mL) for the duration of culture. CM: Central memory. EM: Effector memory. Unpaired two-sided t-test p values are shown. (FIG. 11F, FIG. 11I) Transient LTBR or tNGFR expression via mRNA nucleofection (FIG. 101F). T cells were either nucleofected with LTBR or tNGFR mRNA (n=3 biological replicates), and the surface expression of LTBR (FIG. 11G), tNGFR (FIG. 11H) or four genes upregulated in LTBR cells (FIG. 11I) was monitored over 21 days. At each timepoint the expression of target genes was normalized to matched tNGFR control (FIG. 11J) Schematic representation of FLAG-tagged LTBR mutants. (FIG. 11K) LTBR and FLAG expression in T cells transduced with LTBR mutants. Error bars indicate SEM.

FIG. 12A-FIG. 12I show chromatin accessibility in LTBR T cells. (FIG. 12A) Principal component (PC) analysis of global accessible chromatin regions of LTBR and tNGFR T cells. either resting or stimulated with CD3/CD28 for 24 h. (FIG. 12B) Differentially accessible chromatin regions between stimulated and resting tNGFR, stimulated and resting LTBR, resting LTBR and resting tNGFR, and stimulated LTBR and stimulated tNGFR. Numbers of peaks gained/lost are shown (using absolute log₂ fold change of 1 and adjusted p value <0.1 as cut-off). (FIG. 12C, FIG. 12D) Changes in chromatin accessibility (FIG. 12C) for differentially expressed (adjusted p<0.05) genes or in gene expression (FIG. 12D) for differentially accessible (adjusted p<0.05) regions. Two-sided t-test p values are shown. Box shows 25-75 percentile with a line at the median; whiskers extend to 1.5×interquartile range. N=614 genes (FIG. 12C) or genomic regions (FIG. 12D). (FIG. 12E, FIG. 12F) Chromatin accessibility profiles at loci more (FIG. 12E) or less open (FIG. 12F) in LTBR compared to tNGFR cells, resting or stimulated for 24 h. The y-axis represents normalized reads (scale: 0-860 for BATF3, 0-1950 for IL13, 0-1230 for TRAF1, 0-1000 for TNFSF4, 0-300 for PDCD1, 0-2350 for LAG3). (FIG. 12G) Chromatin accessibility in resting or stimulated LTBR and tNGFR cells. Each row represents a peak significantly enriched in LTBR over matched tNGFR control (log 2 fold change >1. DESeq2 adjusted p value <0.05). Peaks were clustered using k-means clustering and selected genes at/near peaks from each cluster are indicated. (FIG. 12H) Correlations for each ATAC sample (biological replicate) based on the bias-corrected deviations. (FIG. 12I) Top transcription factor (TF) motifs enriched in the differentially accessible chromatin regions in resting LTBR cells compared to resting tNGFR cells.

FIG. 13A-FIG. 13P show proteomic and functional genomic assays of NF-κB activation. (FIG. 13A) Phospho-RELA staining by intracellular flow cytometry in LTBR and tNGFR cells. Gating for identification of phospho-RELA+ cells is shown. (FIG. 13B, FIG. 13C) Western blot quantification of key proteins in the NF-κB pathway in LTBR and tNGFR cells. resting or stimulated with CD3/CD28 for 15 min. Representative gels (FIG. 13B) or quantification of band intensity relative to GAPHD (FIG. 13C) are shown (n=3 biological replicates). Unpaired two-sided: test p values are shown. (FIG. 13D) Representation of the LTBR signaling pathway. Each gene is colored based on the differential expression in LTBR over matched tNGFR cells (CD4⁺ and CD8⁺ T cells, resting or stimulated for 24 h). (FIG. 13E-FIG. 13G) Simultaneous gene knockout via CRISPR and ORF overexpression. T cells were transduced with a lentiviral vector co-expressing a single guide RNA (sgRNA) and the LTBR ORF. After transduction, Cas9 protein was delivered via nucleofection. (FIG. 13F) Representative expression of target genes in LTBR cells co-expressing an sgRNA targeting B2M, an essential component of the MHC-I complex, or TRBC1/2, an essential component of the αβ TCR. (FIG. 13G) Quantification of IFNγ after restimulation (n=3 sgRNAs). (FIG. 13H-FIG. 13O) Representative protein-level based quantification of gene knockout efficiency. Representative histograms (FIG. 13H, FIG. 13J, FIG. 13L) and quantification of relative expression levels of LTA, LIGHT, and RELA (FIG. 13I, FIG. 13K, FIG. 13M) are shown (n=3 sgRNAs). Dashed lines represent gates used to enumerate cells expressing a given protein. Representative gel (FIG. 13N) and quantification of RELB expression (FIG. 13O) are shown (n=3 sgRNAs for RELB and 2 non-targeting control sgRNAs). (FIG. 13P) Identification of 274 genes identified as enriched in both CD4⁺ and CD8⁺ T cells transduced with LTBR over matched tNGFR controls (“core LTBR” genes). Error bars indicate SEM.

FIG. 14A-FIG. 14P show co-delivery of ORFs with CD19-targeting CARs. (FIG. 14A) Transduction efficiency of CAR+ORF lentiviral vectors or ORF alone (n=4 biological replicates) (FIG. 14B, FIG. 14C) CAR expression level as determined by staining with anti-mouse Fab F (ab) 2. Representative histograms (FIG. 14B) and quantification of CAR expression relative to tNGFR (FIG. 14C) is shown for two healthy donors and two patients with diffuse large B cell lymphoma (DLBCL). (FIG. 14D) Expansion curves of CAR+ORF transduced T cells (n=4 biological replicates). (FIG. 14E) LTBR expression in autologous CD14⁺ monocytes and T cells transduced with LTBR alone or CAR+LTBR. (FIG. 14F-FIG. 14I) Expression of ICAM-1 (FIG. 14F). CD70 (FIG. 14G), CD74 (FIG. 14H) and MHC-II (FIG. 14I) by T cells transduced with LTBR ORF only, CAR+LTBR or CAR+tNGFR. All data are normalized to tNGFR only (no CAR). Unpaired two-sided t test p values are shown. (FIG. 14J-FIG. 14M) Expression of exhaustion markers PD-1 (FIG. 14J), TIM-3 (FIG. 14K), LAG-3 (FIG. 14L) and CD39 (FIG. 14M) in CAR+ORF T cells. (FIG. 14N) Differentiation phenotype of CAR+ORF T cells. CM: Central memory. EM: Effector memory. Differentiation was defined based on CD45RO and CCR7 expression (naive: CD45RO^{neg} CCR7⁺, CM: CD45RO⁺CCR7⁺, EM: CD45RO⁺CCR7^{neg}, effector CD45RO^{neg} CCR7^{neg}). (FIG. 14O, FIG. 14P) Expression of activation markers CD25 (FIG. 14O) and CD69 (FIG. 14P) in CAR+ORF T cells incubated alone or with Nalm6 cells for 24 h. Error bars indicate SEM. N=3 biological replicates, unless indicated otherwise.

FIG. 15A-FIG. 15P show top-ranked genes from the ORF screen boost antigen-specific T cell responses. (FIG. 15A, FIG. 15B) Co-delivery of anti-CD19 CARs and ORFs to T cells from healthy donors. (FIG. 15A) IFNγ and (FIG. 15B) IL2 secretion after overnight co-incubation of CD4⁺ T cells with Nalm6 cells at 1:1 ratio (n=3 biological replicates, representative of two donors). (FIG. 15C, FIG. 15D) IFNγ (FIG. 15C) or IL-2 (FIG. 15D) secretion by CAR+ORF or ORF only T cells co-incubated for 24 h either alone or with Nalm6 cells. (FIG. 15E) Cytotoxicity of 19-BBz CAR T cells expressing tNGFR or LTBR ORF after co-incubation with Nalm6 GFP cells. (FIG. 15F) Quantification of Nalm6 clearance (relative to Nalm6 co-incubated with untransduced T cells) for CAR+ORF or ORF alone T cells at different effector: target ratios. Unpaired two-sided t-test p values: 0.011, 1.3×10-4, 0.072, 0.02, 0.021, 0.52, 0.087, 1, 0.51 (left to right). (FIG. 15G) Representative images of T cells transduced with 19-28z CAR and NGFR or LTBR, co-incubated with CD19+Nalm6 GFP cells for 48 h at 1:1 ratio. Scale bar: 200 μm. (FIG. 15H-FIG. 15J) Repeated stimulation of CAR+ORF T cells with Nalm6 cells. IL-2 secretion (FIG. 15I), or Nalm6 survival (FIG. 15J), by 19-BBz CAR LTBR or tNGFR T cells re-challenged with Nalm6 after repeated stimulation with Nalm6 cells every three days, for up to three rounds of stimulation. (FIG. 15K) Secretion of cytokines IL2 and IFNγ by CAR/LTBR or CAR/tNGFR T cells from two patients with DLBCL after overnight incubation with Nalm6 target cells. Two-sided paired t-test p value is shown. (FIG. 15L) Representative staining of ORF-transduced T cells endogenously expressing Vγ9Vδ2 TCR. (FIG. 15M) Quantification of ORF-transduced T cells expressing Vγ9V82 TCR. (FIG. 15N, FIG. 15O) IL2 (FIG. 15N) or IFNγ (FIG. 15O) secretion after 24 h co-incubation of ORF transduced Vγ9V82 T cells with leukemia cell lines. (FIG. 15P) IL2 or IFNγ secretion after 24 h co-incubation of ORF transduced Vγ9Vδ2 T cells with BxPC3, a pancreatic ductal adenocarcinoma cell line. Cell lines in panels (FIG. 15N-FIG. 15P) were pre-treated with zoledronic acid prior to co-incubation. Error bars indicate SEM. N=3 biological replicates are shown, unless indicated otherwise.

FIG. 16A-FIG. 16F show Top-ranked genes improve antigen-specific CAR T cell responses in solid tumor. (FIG. 16A) Codelivery of anti-mesothelin CARs and ORFs to T cells from healthy donors. (FIG. 16B-FIG. 16D) Secretion of cytokines IFNγ and IL2 by CD4+ and CD8+ T cells co-transduced with anti-mesothelin CARs and ORF, after an overnight co-incubation with a mesothelin-high cell line Capan-2 (FIG. 16B, FIG. 16C) or mesothelin-low cell line BxPC3 (FIG. 16D). No specific cytokine secretion was observed in T cells incubated alone. N=3 biological replicates. Dashed line indicated the level of cytokine secretion in regular CAR T cells (i.e., co-expressing tNGFR). (FIG. 16E, FIG. 16F) Killing of GFP+ mesothelin-high Capan-2 or mesothelin-low BxPC3 after co-incubation with engineered CAR T cells at 1:2 T cell to cancer cell ratio for 48 b. Cancer cell killing was normalized by dividing the integrated GFP signal in wells containing regular CAR T cells (i.e. co-expressing tNGFR) by the integrated GFP signal in specified samples. Ratio above one indicates higher killing, i.e. lower GFP signal in specified samples (and thus lower number of cancer cells) than in matched CAR control (CAR+tNGFR). WT or no CAR=untransduced T cells.

FIG. 17A-FIG. 17D show Top-ranked genes improve antigen-specific TCR T cell responses in solid tumor. (FIG. 17A) Codelivery of anti-NY-ESO-1 TCR and ORFs to T cells from healthy donors. (FIG. 17B, FIG. 17C) Secretion of cytokines IFNγ and IL-2 by CD8+ T cells co-transduced with anti-NY-ESO-1 TCR and ORF, after an overnight coincubation with a melanoma cell line A375. No specific cytokine secretion was observed in T cells incubated alone. N=3 biological replicates. Dashed line indicated the level of cytokine secretion in regular TCR T cells (i.e., co-expressing tNGFR). (FIG. 17E, FIG. 17F) Killing of GFP⁺A375 cells co-incubation with engineered TCR T cells at 1:1 T cell to cancer cell ratio for 48 h. Cancer cell killing was normalized by dividing the integrated GFP signal in wells containing only A375 cells but no T cells by the integrated GFP signal in specified samples. No TCR=untransduced T cells.

FIG. 18A-FIG. 18G provide an overview of OverCITE-seq.

FIG. 19 is a listing of the clinical trials relating to chimeric antigen receptors available on clinicaltrials.gov.

FIG. 20 is a listing of the clinical trials relating to T cell receptors available on clinicaltrials.gov.

FIG. 21A-FIG. 21N show insertion of the intracellular domain of LTBR into the CAR signaling domains results in superior efficacy. (FIG. 21A) Schematic representation of anti-CD19 CARs utilizing the CD28 costimulatory domain (FIG. 21B) Schematic representation of anti-CD19 CARs utilizing the 41-BB costimulatory domain. (FIG. 21C, FIG. 21D) Surface expression of the CAR in T cells transduced with the constructs shown in FIG. 21A and FIG. 21B. respectively. No CAR indicates untransduced T cells. (FIG. 21E, FIG. 21F) Expression of LTBR-induced genes CD54 (also known as ICAM-1) and CD74 in resting T cells transduced with constructs shown in FIG. 21A and FIG. 21B, respectively. Surface expression was normalized to the matched control (CAR T cells co-expressing tNGFR). N=2 individually transduced samples (CD4+ and CD8+ T cells). (FIG. 21G, FIG. 21H) Differentiation of T cells transduced with constructs shown in FIG. 21A and FIG. 21B, respectively (naïve: CD45RO− CCR7+, central memory [CM]: CD45RO+ CCR7+, effector memory [EM]: CD45RO+ CCR7−, effector: CD45RO− CCR7−). (FIG. 21I-FIG. 21L) Secretion of cytokines IFNγ and IL2 by CD4+ and CD8+ T cells transduced with constructs shown in FIG. 21A and FIG. 21B, respectively, after an overnight co-incubation with a CD19+ leukemia cell line Nalm6. No specific cytokine secretion was observed in T cells incubated alone. N=3 biological replicates. Dashed line indicated the level of cytokine secretion in regular CAR T cells (i.e. co-expressing tNGFR). (FIG. 21M, FIG. 21N) Killing of GFP+ CD19+ leukemia cell line Nalm6 after co-incubation with engineered CAR T cells at high (1:2 T cell to cancer cell ratio) or low (1:8 T cell to cancer cell ratio) for 48 h or 96 h. Cancer cell killing was normalized by dividing the integrated GFP signal in wells containing regular CAR T cells (i.e. coexpressing tNGFR) by the integrated GFP signal in specified samples. Ratio above one indicates higher killing, i.e. lower GFP signal in specified samples (and thus lower number of Nalm6 cancer cells) than in matched CAR control (CAR+tNGFR). WT or no CAR=untransduced T cells.

FIG. 22A-FIG. 22C show LTBR potentiates the activity of a 1st generation CAR. (FIG. 22A) Schematic representation of anti-CD19 CARs (generation 0-no signaling domains, 1st generation—only CD3z. 2nd generation—4-1BB and CD3z) co-expressed together with tNGFR (negative control) or LTBR. (FIG. 22B, FIG. 22C) Secretion of cytokines IFNγ and IL2 by CD4+ and CD8+ T cells transduced constructs shown in FIG. 22A after an overnight co-incubation with a CD19+ leukemia cell line Nalm6. No specific cytokine secretion was observed in T cells incubated alone. N=3 biological replicates. Dashed line indicated the level of cytokine secretion in regular 2^{nd} generation CAR T cells (i.e., co-expressing tNGFR). WT—untransduced T cells.

FIG. 23A-FIG. 23B show extracellular LTBR potentiates T cell function when fused to the stalk, transmembrane and signaling domains of a 2nd generation CAR. (FIG. 23A) Schematic representation of constructs tested. (FIG. 23B) Secretion of cytokines IFNγ and IL2 by CD4+ and CD8+ T cells transduced constructs shown in FIG. 23A after an overnight co-incubation with CD3 and CD28 antibodies. No specific cytokine secretion was observed in T cells incubated alone. N=2 biological replicates. Dashed line (where applicable) shows cytokine secretion in untransduced (WT—wild type) T cells.

FIG. 24A-FIG. 24E CARs with only the intracellular LTBR signaling domain boost T cell response to TCR stimulation and activate the NFκB pathway. (FIG. 24A) Schematic representation of CAR constructs composed of the antigen targeting moiety, a stalk (CD8 or CD28), a transmembrane domain derived either from the same protein as the stalk or from LTBR, and the intracellular domain of LTBR. (FIG. 24B) Schematic representation of 2nd generation CARs or CARs lacking any signaling domain, co-expressed with tNGFR (negative control) or LTBR. (FIG. 24C) Secretion of cytokines IFNγ and IL2 by CD4+ and CD8+ T cells transduced constructs shown in FIG. 24A and FIG. 24B after an overnight co-incubation with CD3 and CD28 antibodies. No specific cytokine secretion was observed in T cells incubated alone. N=3 biological replicates. Dashed line (where applicable) shows cytokine secretion in T cells expressing only the anti-CD19 targeting moiety but no signaling domains (19-CD8+tNGFR). WT-untransduced (wild-type) cells. (FIG. 24D) Expression of CD74 either on resting T cells or T cells co-incubated with CD19+ target cells overnight. Dashed lines show CD74 expression in T cells expressing only the anti-CD19 targeting moiety but no signaling domains (19-CD8+tNGFR)-incubated alone or with target cells. WT—untransduced (wild-type) cells.

FIG. 25A-FIG. 25C show schematic representations of incorporating LTBR into the TCR complex. (FIG. 25A) αβTCR designs: (FIG. 25A) γδTCR designs; (FIG. 25A) αβT cell co-receptor designs.

FIG. 26A-FIG. 26K show LTBR co-delivery improves antitumor activity of B7-H3 CAR T cells. FIG. 26A shows differentiation of transduced and untransduced T cells (naïve: CD45RO− CCR7+, central memory: CD45RO+ CCR7+, effector memory: CD45RO+ CCR7−, effector: CD45RO− CCR7−). Expression of surface markers CD54 (FIG. 26B) and CD74 (FIG. 26C), n=3. Secretion of IFNγ (FIG. 26D) or IL2 (FIG. 26E) after overnight co-incubation of engineered T cells with B7-H3+ cell lines BT12, BT16 and A375, and a B7-H3^{negative} cell line Nalm6, n=3. Killing of GFP⁺B7-H3+ cell lines BT12, BT16 and A375 (FIG. 26F) or GFP+ B7-H3^{negative} cell line Nalm6 (FIG. 26G) after co-incubation with engineered CAR T cells at 2:1 T cell to cancer cell ratio. FIG. 26H-FIG. 26K show in vivo testing of B7-H3 CAR T cells in NSG mice implanted with A375 cells in the left flank. Due to the aggressive tumor growth in the majority of animals, the experiment was terminated on day 25. (FIG. 26H) Experimental design. On day 13 post tumor inoculation, tumor volume was measured using calipers. Based on the tumor burden, mice were staged into five groups (n=4-5 mice per group) so that there was no difference in median burden between groups. After staging, mice were injected with 1:1 mix of human CD4 and CD8 T cells, either untransduced or transduced with B7-H3 CAR and control gene tNGFR or LTBR. High dose=5 million T cells per mouse: low dose=1 million T cells per mouse. (FIG. 26I) Body weight changes after tumor implantation. (FIG. 26J) Mean tumor size per group. Mice were removed from the study and euthanized once the tumor exceeded 200 mm³ volume or at veterinarian's, blinded to the study design, discretion. (FIG. 26K) Tumor size in individual mice.

FIG. 27A-FIG. 27G show CAR fusions with truncated LTBR intracellular tail. (FIG. 27A) Literature-based annotation of the functional elements within the LTBR signaling tail. Numbers indicate amino acid numbering as per https://www.uniprot.org/uniprotkb/P36941. (FIG. 27B) Schematic depicting fusion of LTBR intracellular tail to the C-terminus of a 19-28-z CAR. For V1-V9 variants of the intracellular tail, dashed lines indicate which part of LTBR protein was included in the fusion with a CAR. All sequences (amino acid and protein) are listed in the sequence listing. (FIG. 27C) Surface expression of 19-28-z CAR in transduced T cells (FIG. 27D) Differentiation of transduced and untransduced T cells (naïve: CD45RO− CCR74, central memory: CD45RO+CCR7+, effector memory: CD45RO+ CCR7−, effector: CD45RO− CCR7−) (FIG. 27E) Intracellular/nuclear expression of TCF1. The relative levels of TCF1 were normalized to the signal in 19-28-z+tNGFR CAR T cells. (FIG. 27F) Surface expression of CD54 and CD74, normalized to the signal in 19-28-z+tNGFR CAR T cells. (FIG. 27G) Secretion of IL2 and IFNγ cytokines upon overnight co-incubation of anti-CD19 CAR T cells with CD19+ leukemia cells Nalm6. Absolute quantities of secreted cytokines were normalized to the quantity secreted by 19-28-z+tNGFR CAR T cells.

FIG. 28A-FIG. 28N show LTBR signaling tail fusion to a mesothelin targeting CAR. (FIG. 28A) Construct designs. (FIG. 28B) CAR surface expression on transduced T cells. (FIG. 28C) CAR T cell proliferation in response to antigenic stimulation. T cells were isolated. transduced with lentivirus and selected. 14 days after isolation CAR T cells were co-incubated with mesothelin+ PDAC cell line Capan-2 at 1:1 ratio (n=3). T cells were periodically counted and split to adjust to a 0.5×10⁶ cell/mL density. (FIG. 28D) Killing of GFP+ mesothelin+ Capan-2 cell line after co-incubation with engineered CAR T cells at 1:4 T cell to cancer cell ratio. (FIG. 28E) Cytokine secretion after 24 h co-incubation of engineered CAR T cells with mesothelin+Capan-2 cell line. (FIG. 28F) Surface expression of CD54 and CD74 on engineered CD4 and CD8 T cells. Median fluorescence intensity of staining for each sample was z-score normalized by subtraction to the median fluorescence intensity of the corresponding control (cither Meso-BB-z or Meso-28-z) and divided by the standard deviation of the sample group. (FIG. 28G) Intracellular expression of TCF1 in engineered T cells. Median fluorescence intensity of staining for each sample was z-score normalized by subtraction to the median fluorescence intensity of the corresponding control (either Meso-BB-z or Meso-28-z) and divided by the standard deviation of the sample group. (FIG. 28H) Cytokine secretion after 24 h co-incubation of engineered CAR T cells pan-T cell stimulation using CD3/CD28. (FIG. 28I) Schematic depicting sequential co-transduction of T cells with two lentiviral particles, one encoding a mesothelin targeting CAR and the other a natural full length LTBR or a fusion of LTBR intracellular domain with a CAR. (FIG. 28J) CAR surface expression on sequentially transduced T cells. (FIG. 28K) LTBR surface expression on sequentially transduced T cells. (FIG. 28L) Surface expression of CD54 and CD74 on sequentially transduced CD4 and CD8 T cells, normalized to corresponding untransduced T cells. (FIG. 28M, FIG. 28N) IFNγ (FIG. 28M) or IL2 (FIG. 28N) secretion after 24 h co-incubation of sequentially transduced CAR T cells with mesothelin+Capan-2 cell line.

FIG. 29A-FIG. 29I show LTBR signaling tail fusion to the members of the TCR-CD3 complex. (FIG. 29A) Construct designs for TCR fusions. (FIG. 29B) TCR surface expression on transduced T cells. (FIG. 29C) Surface expression of CD54 and CD74 on transduced T cells. (FIG. 29D) Cytokine secretion upon 24 h co-incubation of engineered T cells with NY-ESO-1+ melanoma line A375. (FIG. 29E) Construct designs for CD3 fusions. (FIG. 29F) Schematic depiction of sequential transduction of T cells with lentiviral particles encoding an NY-ESO-1 TCR and CD3 genes, natural or fused on the C-terminus with the LTBR signaling tail. (FIG. 29G) TCR surface expression on sequentially transduced T cells. (FIG. 29H) Surface expression of CD54 and CD74 on sequentially transduced T cells. (FIG. 29I) Cytokine secretion upon 24 h co-incubation of sequentially transduced T cells with NY-ESO-1+ melanoma line A375.

FIG. 30A-FIG. 30G show LTBR signaling tail fusion to the members of the CD8 complex. (FIG. 30A) Construct designs for CD8 fusions. (FIG. 30B) Schematic depiction of sequential transduction of T cells with an NY-ESO-1 TCR and CD8 genes, natural or fused with the LTBR intracellular tail. (FIG. 30C, FIG. 30D) Surface expression of CD54 (FIG. 30C) and CD74 (FIG. 30D) on transduced T cells. (FIG. 30E) Intracellular expression of TCF1 in transduced T cells. (FIG. 30F) Cytokine secretion upon 24 h co-incubation of engineered T cells with HLA-A2+ cell line HEK293T pulsed with indicated concentration of the NY-ESO-1 derived peptide epitope. (FIG. 30G) Antigen sensitivity of engineered T cells calculated based on the sigmoidal fitting of the levels of the indicated cytokine secretion as a function of peptide concentration. MFI-median fluorescence intensity.

FIG. 31 shows ORF library vector engineering. Lentivirus encoding puromycin resistance and a barcoded ORF library (as indicated) was produced in HEK 293T cells, concentrated and used to transduce activated T cells. Two days after transduction, engineered T cells from each condition were split and one set was selected with puromycin while the other was treated with vehicle. After a two-day selection, T cells were counted and transduction efficiency was determined by dividing the live cell number in the condition treated with puromycin to the matched, untreated control. Dashed line indicates survival of untransduced T cells treated with puromycin.

### DETAILED DESCRIPTION OF THE INVENTION

The engineering of autologous patient T cells for adoptive cell therapies has revolutionized the treatment of several types of cancer¹. However, further improvements are needed to increase response and cure rates. Provided herein are modified T cells that include nucleic acids encoding fusion proteins that include LTBR, or domains thereof. When overexpressed in T cells. LTBR induced profound transcriptional and epigenomic remodeling. leading to increased T cell effector functions and resistance to exhaustion in chronic stimulation settings through constitutive activation of the canonical NF-κB pathway LTBR and other highly ranked genes improved the antigen-specific responses of chimeric antigen receptor T cells and γδ T cells, highlighting their potential for future cancer-agnostic therapies⁵. We provide improved CAR. TCR and related T cell therapies for treatment of cancer and other diseases.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

As used throughout this specification and the claims, the terms “comprising”, “containing”. “including”, and its variants are inclusive of other components, elements, integers, steps and the like. Conversely, the term “consisting” and its variants are exclusive of other components, elements, integers, steps and the like.

It is to be noted that the term “a” or “an”, refers to one or more, for example, “T cell”, is understood to represent one or more T cell(s). As such, the terms “a” (or “an”), “one or more.” and “at least one” is used interchangeably herein.

As used herein, the term “about” means a variability of plus or minus 10% from the reference given, unless otherwise specified.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone). and “B” (alone). Likewise, the term “and/or” as used in a phrase such as “A, B, and/or C” is intended to encompass each of the following aspects: A, B, and C: A, B, or C; A or C; A or B: B or C; A and C; A and B: B and C; A (alone): B (alone); and C (alone)

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991): Qhtsuka et al. J. Biol. Chem. 260:2605-2608(1985); and Rossolim et af., Mol. Cell. Probes 8:91-98(1994))

The terms “nucleic acid sequence,” “nucleotide sequence,” or “polynucleotide sequence” are used interchangeably and refer to a contiguous nucleic acid sequence. The sequence can be cither single stranded or double stranded DNA or RNA, e.g., an mRNA.

Nucleic acids described herein can be cloned using routine molecular biology techniques, or generated de novo by DNA synthesis, which can be performed using routine procedures by service companies having business in the field of DNA synthesis and/or molecular cloning (e.g. GeneArt, GenScript, Life Technologies, Eurofins). The nucleic acid sequences encoding aspects of a CRISPR-Cas editing system described herein are assembled and placed into any suitable genetic element, e.g., naked DNA, phage, transposon, cosmid, episome, etc., which transfers the sequences carried thereon to a host cell, e.g., for generating non-viral delivery systems (e.g., RNA-based systems, naked DNA, or the like), or for generating viral vectors in a packaging host cell, and/or for delivery to a host cells in a subject. In one embodiment, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

“Variants” of proteins or peptides as defined in the context of the present invention may be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g., its specific inhibitory property. “Variants” of proteins or peptides as defined in the context of the present invention may comprise conservative amino acid substitution(s) compared to their native, i.e., non-mutated physiological, sequence. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bonds, e.g., side chains which have a hydroxyl function. This means that e.g., an amino acid having a polar side chain is replaced by another amino acid having a likewise polar side chain, or, for example, an amino acid characterized by a hydrophobic side chain is substituted by another amino acid having a likewise hydrophobic side chain (e.g., serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g., using CD spectra (circular dichroism spectra) (Urry, 1985, Absorption, Circular Dichroism and ORD of Polypeptides, in: Modern Physical Methods in Biochemistry, Neuberger et al. (cd.), Elsevier, Amsterdam). A variant may also include a non-natural amino acid.

A “variant” of a protein or peptide may have at least 70%, 75%, 80%, 85%, 90%, 95%, 98% or 99% amino acid identity over a stretch of 10, 20, 30, 50, 75, 100 or more amino acids of such protein or peptide, or over the full length of the protein or peptide.

The term “gene” can refer to a segment of DNA involved in producing or encoding a polypeptide chain. It may include regions preceding and following the coding region (leader and trailer) as well as intervening sequences (introns) between individual coding segments (exons).

As used herein, the terms “coding region” and “region encoding” and grammatical variants thereof, refer to an open reading frame (ORF) in a polynucleotide that upon expression yields a polypeptide or protein.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The term “domain” refers to a region of the protein's polypeptide chain that is self-stabilizing and that folds independently from the rest of the protein. The protein domain need not be identical to the native protein from which it is derived, but may be a variant thereof, including a variant that has a deletion, truncation, etc.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA, and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleic acid sequence encoding an amino acid sequence” includes all nucleic acid sequences that are degenerate versions of each other and that encode the same amino acid sequence. A nucleic acid sequence that encodes a protein or an RNA may also include introns to the extent that the nucleotide sequence encoding the protein may in some version contain an intron(s).

The term “expression” is used herein in its broadest meaning and comprises the production of RNA, of protein, or of both RNA and protein. Expression may be transient or may be stable.

The terms “expressing” and “overexpression” refer to increasing the expression of a gene or protein. The terms refer to an increase in expression, for example, in increase in the amount of mRNA or protein expressed in a T cell, other lymphocyte or host cell, of at least 10%, as compared to a reference control level, or an increase of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%. Various methods for expression and/or overexpression are known to those of skill in the art, and include, but are not limited to, stably or transiently introducing a exogenous polynucleotide encoding a fusion protein. TCR, or CAR to be expressed and/or overexpressed in the cell or inducing expression or overexpression of an endogenous gene encoding the protein in the cell.

The term “autologous” refers to any material derived from the same subject to whom it is later to be re-introduced.

The term “exogenous” refers to any material introduced from or produced outside an organism, cell, tissue or system.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, an “expression cassette” refers to a nucleic acid molecule which encodes one or more ORFs or genes, e.g., an effector-enhancing gene, or a CAR or TCR or component thereof. An expression cassette also contains a promoter and may contain additional regulatory elements that control expression of one or more elements of a gene editing system in a host cell. In one embodiment, the expression cassette may be packaged into the capsid of a viral vector (e.g., a viral particle). In one embodiment, such an expression cassette for generating a viral vector as described herein is flanked by packaging signals of the viral genome and other expression control sequences such as those described herein.

The term “regulatory element” or “regulatory sequence” refers to expression control sequences which are contiguous with the nucleic acid sequence of interest and expression control sequences that act in trans or at a distance to control the nucleic acid sequence of interest. As described herein, regulatory elements comprise but are not limited to, promoter: enhancer. transcription factor: transcription terminator: efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE): sequences that enhance translation efficiency (i.e., Kozak consensus sequence): sequences that enhance protein stability; and when desired, sequences that enhance secretion of the encoded product. Also, see Goeddel; Gene Expression Technology: Methods in Enzymology 185, Academic Press, San Diego, CA (1990). Regulatory sequences include those which direct constitutive expression of a nucleic acid sequence in many types of target cell and those which direct expression of the nucleic acid sequence only in certain target cells (e.g., tissue-specific regulatory sequences).

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The term “constitutive” when referring to a promoter specifies a nucleotide sequence which, when operably linked with a polynucleotide which encodes or specifies a gene product, causes the gene product to be produced in a cell under most or all physiological conditions of the cell. The term “inducible” or “regulatable” when referring to a promoter specifies a nucleotide sequence which, when operably linked with a polynucleotide which encodes or specifies a gene product, causes the gene product to be produced in a cell substantially only when an inducer which corresponds to the promoter is present in the cell. In certain embodiments, the inducible promoter is activated in response to T cell stimulation In certain embodiments, the promoter is an NFAT, AP1, NFκB, or IRF4 promoter. The term “tissue-specific” when referring to a promoter specifies a nucleotide sequence which, when operably linked with a polynucleotide encodes or specified by a gene, causes the gene product to be produced in a cell substantially only if the cell is a cell of the tissue type corresponding to the promoter. Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription Exemplary promoters include the CMV IE gene, EF-1α, ubiquitin C, or phosphoglycerokinase (PGK) promoters.

The term “operably linked” refers to functional linkage between one or more regulatory sequences and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, where necessary to join two protein coding regions, are in the same reading frame.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

In certain embodiments, one or more genes are encoded by a nucleic acid sequence that is delivered to a host cell by a vector or a viral vector, of which many are known and available in the art. In one embodiment, provided is a vector comprising an expression cassette as described herein. In one embodiment, a vector is a non-viral vector. In another embodiment, a vector is a viral vector. A “viral vector” refers to a synthetic or artificial viral particle in which an expression cassette containing a nucleic acid sequence of interest is packaged in a viral capsid or envelope. Examples of viral vectors include but are not limited to lentivirus, adenoviruses, retroviruses (y-retroviruses and lentiviruses), poxviruses, adeno-associated viruses (AAVs), baculoviruses, herpes simplex viruses. In one embodiment, the viral vector is replication defective. A “replication-defective virus” refers to a viral vector, wherein any viral genomic sequences also packaged within the viral capsid or envelope are replication-deficient, i.e., they cannot generate progeny virions but retain the ability to infect cells.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR& gene delivery technology from Oxford BioMedica, the LENTIMAX™ vector system from Lentigen and the like. Nonclinical types of lentiviral vectors are also available and would be known to one skilled in the art.

Provided herein, in one aspect, is an engineered lentiviral vector comprising the sequence of SEQ ID NO: 132, or a sequence sharing at least 90% identity with SEQ ID NO: 132. In certain embodiments, the sequence shares at least 90%, 91%, 92%, 93%, 94%, 95%, 96%, 97%, 98%, or 99% identity with SEQ ID NO: 132. The engineered vector is useful for, inter alia, the ORF screens described herein. In certain embodiments, an open reading frame (ORF) for a gene of interest is inserted within the vector sequence. In certain embodiments, a barcode is inserted within the vector sequence SEQ ID NO: 133 provides an exemplary embodiment in which various primer binding sites, meganuclease recognition sites for restriction digests, and cloning recombination sites have been inserted in the sequence at the site that the ORF and/or barcode can be inserted. Primer binding sites, restriction sites, cloning sites and the like can be included in addition to the ORF and/or barcode. In certain embodiments, the ORF and/or barcode is/are inserted after nucleotide 3291 of SEQ ID NO: 132.

In certain embodiments, the vector is a non-viral plasmid that comprises an expression cassette described herein, e.g., naked DNA, naked plasmid DNA, RNA, and mRNA: coupled with various compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

Plasmids, other cloning and expression vectors, properties thereof, and constructing/manipulating methods thereof that can be used in accordance with the present invention are readily apparent to those of skill in the art. In one embodiment, an expression cassette as described herein is engineered into a suitable genetic element (a vector) useful for generating viral vectors and/or for introduction to a host cell, e.g., naked DNA, phage, transposon, cosmid, episome, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY.

The term “transfected” refers to a process by which exogenous nucleic acid is transferred or introduced into the host cell. A “transfected” cell is one which has been transfected with exogenous nucleic acid. The cell includes the primary subject cell and its progeny.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

RNA or DNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

As used herein, the term “subject” means a mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, non-human primate and others. As used herein, the term “subject” is used interchangeably with “patient”.

### Compositions

Provided herein are compositions which include nucleic acids, expression cassettes, and/or lymphocytes which include coding sequences for certain genes (or fragments thereof). which have been shown to enhance T cell survival, proliferation and/or effector function (collectively referred to herein as an “effector-enhancing gene”). In certain embodiments, the effector-enhancing gene comprises any of the genes identified in Table 1, 2, or 3.

TABLE 1
gene_symbol

| | | | | | | | |
|---|---|---|---|---|---|---|---|
| RPLP1 | EMC10 | SFTPC | PSMB1 | SEC61G | PRAP1 | KLK11 | TSPAN1 |
| HDHD2 | CCR4 | PAFAH1B3 | THRA | SNAPIN | CBX8 | TMEM43 | CELA1 |
| DBI | RIMS3 | RPL29 | SLC37A4 | IGHV7-81 | FGL1 | PRSS8 | HEMK1 |
| UBE2A | IFNL2 | CZIB | HNRNPK | OXA1L | KIAA0930 | SLC39A1 | METTL7B |
| WDR70 | GLYCTK | C11orf16 | ASB6 | F2RL1 | AHNAK | KRTAP19-5 | TPI1 |
| AHSA1 | POLG2 | PRTN3 | RRAGB | PFKFB4 | PDLIM3 | GPN3 | PRSS48 |
| ARMC7 | OR1D5 | UBE2D1 | MAPK3 | LRRC29 | CD59 | GJB7 | MEIS3 |
| PKLR | SZRD1 | GTPBP8 | HLA-DOA | SPPL2C | LTBR | VSTM1 | EXOSC5 |
| TIMMDC1 | DYRK4 | CRADD | SPIC | DHX40 | SLC10A7 | TIMP4 | HSFX3 |
| TRH | CUTA | CDK17 | HADH | RAN | ACTL8 | LIG3 | OSM |
| NFYB | RNF186 | RAB24 | RPL14 | KDSR | MEI1 | C1orf210 | GSTK1 |
| SPATS1 | ACSMS | TAZ | IFT81 | IL27 | GTF3C6 | CEP43 | RUVBL2 |
| PRND | TAFA2 | PDK4 | TMEM44 | NRBP2 | RTBDN | FKBP1A | EDDM3A |
| TRMT12 | TFF1 | FNDC8 | TLE1 | UHMK1 | RAD51C | ETFBKMT | TARBP2 |
| LENG1 | CLN6 | CCDC137 | GNPDA1 | QTRT1 | ZCCHC12 | FBXO6 | PGAP2 |
| SMOC1 | FBXO30 | CST8 | BAX | S100A8 | VWC2L | RHEX | MAGEA12 |
| BOLL | CLEC19A | NLRP5 | LGALS12 | PDXK | PRR19 | TRAF2 | PEX11A |
| VASH1 | TMEM263 | LRP5L | DAND5 | SLC52A1 | COMMD8 | NOL6 | CA12 |
| CTPS2 | CALML3 | FHIT | LTA4H | UBL4B | OR10G3 | SNURF | GPR37L1 |
| TSHB | F2RL2 | SNAPC5 | TXN | MID2 | ADAT2 | RAB8A | EID3 |
| BATF | DCT | DTNA | ELN | CNOT2 | CALCB | MAB21L2 | GRIK2 |
| ACP2 | PLPPR2 | BDKRB2 | IFITM3 | IDH3B | SYS1 | IFNA21 | ADPRS |
| SULT1C4 | ASMTL | TANK | RPL13A | IL10 | PSG1 | TMIGD1 | SLC23A3 |
| CDC42 | FBXL2 | SEMA4G | ARSJ | WAS | DPH1 | H2AC6 | ADH4 |
| ZNF830 | RNF11 | PIEZO1 | APTX | ALKBH3 | LRRC71 | IGSF10 | H3C10 |
| MMP2 | ZMYM3 | CD1B | RIT2 | GOSR2 | CDK2 | HMHB1 | TRPV2 |
| YPEL4 | CAMKMT | SLC39A4 | CPSF4 | RNF114 | DCLRE1B | PLA2G1B | HOXA6 |
| EXOSC1 | GPHB5 | VRK2 | ADAM2 | HLF | UBE2D4 | KCNJ12 | NCR3 |
| SPRR2B | NMS | ENHO | APEX2 | SRP9 | RORA | KLK2 | KLK15 |
| HOGA1 | HAAO | SWSAP1 | TMEM205 | OR10W1 | STK25 | PPP1R8 | WDFY1 |
| LARS2 | WDTC1 | RALB | CAPNS2 | AKAP7 | SLC4A4 | CSRP1 | ZCRB1 |
| EDA | RASGEF1C | POU2F2 | NELFE | TMEM107 | TRIM36 | TIMM8B | EMP3 |
| CCNE1 | NFC | NNAT | S100A9 | TMEM134 | CENPW | POU4F3 | S1PR3 |
| SPAG11A | CHMP2A | ECT2 | IFRD2 | PWP1 | MS4A3 | FANCC | TOR1AIP2 |
| MRFAP1L1 | HCFC2 | C1D | CSF3 | CLIC1 | CDA | PCP2 | MRPS12 |
| SKAP1 | GPATCH11 | MORN4 | GPNMB | C5orf24 | PGS1 | TRIM74 | SSX3 |
| ANXA9 | DUPD1 | DCUN1D3 | XAGE3 | RBIS | SH3RF3 | PPP1R2B | HEPACAM2 |
| CYP27A1 | FCF1 | TESC | RPP30 | EVA1C | GLT1D1 | KCNA4 | CTRB1 |
| MRPL50 | HSD17B7 | IL1RN | APBB1IP | USP24 | LHFPL5 | HSD11B2 | SLC7A11 |
| TRIM51 | ORIN2 | GPD1 | CRHBP | SYNGR2 | SERPINB4 | RSKR | PEX12 |
| SLC1A7 | HHLA3 | DOK6 | AFTPH | DDIT4 | STAU2 | TBCCD1 | TMED9 |
| RPL30 | MC3R | FAM216A | ZNF232 | GGCX | EIF1AY | SYT10 | C16orf70 |
| PLSCR1 | RASGEF1A | GALNT3 | H2AC14 | ITM2A | GPR3 | ATP5MPL | SLC25A48 |
| LIN37 | PDP2 | XIAP | SELENOH | SUN5 | YIF1A | TBRG4 | TGIF1 |
| MASP2 | MIF4GD | PATE1 | S100A11 | EDC3 | OR6B2 | ALDH1A3 | MRPL17 |
| ENPP1 | GTF2B | ANGPT4 | CDCP2 | P2RY6 | KLRD1 | POFUT1 | PCMTD1 |
| MAPRE3 | CTRB2 | HAVCR2 | BCL7A | SLC25A39 | PRDX5 | TEX35 | SOSTDC1 |
| REEP4 | ANAPC16 | MYL4 | CERS4 | RAP1A | ZNF689 | DECR2 | TNFAIP8 |
| COPS8 | DHRSX | H2BC21 | OR3A2 | DRAP1 | RASSF1 | S100A7 | CABLES1 |
| TP53 | CCL13 | TUBB | IL31 | GLYAT | TAAR5 | LRRC25 | GSK3B |
| ITIH1 | CTSE | ASCL3 | CSH1 | IL23A | VGLL3 | LAMTOR1 | PODXL2 |
| PPIC | LSM1 | PTPN7 | STPG2 | ASB1 | BRWD1 | TPPP3 | ACSM3 |
| EXOSC4 | PAGE5 | MRPL18 | HSPBAP1 | TSPAN31 | FUT10 | DKC1 | CGB2 |
| TM2D2 | AHCY | FAM71F2 | RHBDD2 | RRAGA | DPEP2 | BANF2 | FMO4 |
| LYPD4 | MGAT4D | NMU | CCBE1 | SRGN | SDC4 | CD244 | TBC1D20 |
| LDHC | HPCAL1 | SNCB | BNIPL | OR52W1 | MRPL51 | DMAP1 | RPL18 |
| CISH | TRIM5 | LRTM1 | LAMP2 | TMEM37 | FAM180A | RSPO1 | TWIST2 |
| CGGBP1 | PHF23 | DEFB134 | CHST4 | DCK | MRPS21 | THNSL1 | RTL8C |
| GABRB1 | IFNE | CD14 | ESM1 | TIRAP | ING3 | PELI1 | GDAP1 |
| PACC1 | CXCL12 | SUMO3 | PRG3 | DAB1 | SEC31A | CEP112 | SAMSN1 |
| PKIB | IQCD | PHPT1 | LRRC8D | OR4K17 | PSMB6 | ENPP3 | SNRK |
| NBR1 | ETFA | UHRF2 | OLFM1 | RAB27B | TNFSF14 | HEATR9 | RNF5 |
| MKNK1 | SMAP1 | BIN3 | TAC1 | SELENOW | NACA | PTCHD1 | STX8 |
| CPXM2 | PTRH2 | AGAP1 | CA2 | MATN1 | PRR15L | WDR1 | PELI2 |
| IFTAP | PSMD4 | CRIP1 | C3AR1 | DEFB132 | VAMP2 | GFAP | CRYBA1 |
| HAUS1 | SERAC1 | SLC17A4 | GUCA2A | ORM2 | NAGK | SRSF12 | OCEL1 |
| WDR4 | B3GAT3 | TRAPPC6A | MRPL53 | KRT14 | OTX1 | NEIL2 | AK3 |
| TMPRSS6 | LAPTM5 | GRPEL1 | HMCES | GRID1 | PRSS37 | INSL5 | EME1 |
| SPINK9 | WDR20 | FOSB | GAK | NPY | SLC25A4 | F11R | ABHD14B |
| AKR1C4 | CKMT1A | SLC31A2 | PPP2R5E | DEFB135 | PAX9 | CLEC3A | MOB3C |
| MLLT11 | SLC14A1 | WFDC3 | DOK1 | RNASEH2B | ROGDI | ATF6B | WDR62 |
| TCEAL7 | CRLF2 | PLA2G12A | CDK1 | GHITM | ARL11 | POLR2C | KIF26B |
| UBQLN3 | ZNF706 | TMEM143 | CTSW | CDC25C | C2orf73 | FSCB | THEM4 |
| PSMA2 | LAD1 | SPANXN5 | FAM78A | TNIP1 | MAPRE2 | MMP16 | HUWE1 |
| IL12B | SIRT5 | GALP | SMIM14 | MTG1 | NELFCD | LRAT | LCN10 |
| COX7A2L | TTC9C | BOD1L1 | KRTAP3-3 | TMEM126A | WDR77 | RPL21 | ELF2 |
| PYGB | HCST | NIT1 | RALGPS2 | HAPLN3 | AMIGO3 | DYNAP | HPRT1 |
| OR10X1 | YARS2 | REEP3 | ZDHHC5 | DDIT3 | TMEM189 | UBE2M | PPP2R2B |
| ACBD4 | BEX3 | SLC35C2 | MPDU1 | TRIM47 | CLDN14 | UTP4 | MRPL49 |
| MIER2 | PSMD3 | PIFO | SLC39A11 | OR13G1 | INSIG2 | OR2H1 | BMPER |
| HP1BP3 | QPRT | SGCA | CASTOR1 | AGPAT4 | ZDHHC6 | C8orf37 | C6orf141 |
| CD3E | CA5B | TRMT44 | PRB3 | AK2 | LMBRD1 | EIF4E3 | ADAP2 |
| CNR2 | PLD3 | RNF182 | STMN2 | PCSK4 | IRX6 | ADA | SMCP |
| UPP2 | LMAN2L | PDCD1 | RACK1 | PGLYRP1 | GNB1L | DDX49 | TMEM68 |
| TRIP6 | CA11 | DBF4B | PFN4 | BBS1 | H2BC15 | H6PD | R3HDM4 |
| SNRPA | TRIM22 | SUPT4H1 | DIMT1 | TEX38 | CCDC127 | SSMEM1 | IRF4 |
| OR2M4 | MYL6 | SHFL | SCG5 | CORO2B | PPIG | RNF185 | CEP20 |
| SPINT4 | MLX | BRD2 | DERL1 | TEX12 | SLFN5 | TNFSF18 | PHKG1 |
| PYCR2 | CALML5 | MDFI | CHGB | SPIB | C2CD4B | ARHGAP28 | APCDD1 |
| MRPL15 | WNT78 | JAGN1 | GPR17 | LETM1 | SLC26A5 | REXO2 | IL18RAP |
| EOLA2 | DUS1L | TMEM182 | SMU1 | FXYD1 | CCL17 | STX1A | UBXN1 |
| POLD3 | TRPV5 | KRT20 | FAM32A | HOMER1 | | | |

Provided herein are expression cassettes that include nucleic acid sequences that encode fusion proteins that include at least a fragment of one or more effector-enhancing genes. In certain embodiments, the gene comprises any of the genes identified in Table 1, above, or a fragment or variant thereof. In another embodiments, the gene comprises any of the genes identified in Table 2, below, or a fragment or variant thereof. In another embodiments, the gene comprises any of the genes identified in Table 3, below, or a fragment or variant thereof. Desirable fragments include protein domains, such as an intracellular signaling domain, a transmembrane domain, or extracellular domain. In certain embodiment, the expression cassette includes more than one effector-enhancing gene fragment.

As used herein, where reference to a specific gene of Table 1, Table 2, or Table 3 is mentioned, it is intended that the use of the coding sequence for the full-length protein, a fragment having a deletion or truncation, a domain, or a variant having one or more substitutions in the amino acid, is intended. For example, in certain embodiments, the nucleic acid encodes a protein sequence having a deletion in the N terminus. In certain embodiments, the nucleic acid encodes a protein sequence having a deletion in the C terminus. In one embodiment, the nucleic acid encodes a protein having of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, or at least 125 amino acids.

In one embodiment, the effector-enhancing gene is LTBR, LTBR, a receptor endogenously expressed by professional antigen presenting cells but not lymphocytes, was identified as a strong synthetic driver of both T-cell proliferation and secretion of key cytokines: IL-2 and IFNγ. Using a multimodal single-cell sequencing approach, it was shown that LTBR induces profound transcriptional changes when overexpressed in T-cells, activating cellular programs involved in antigen presentation and prevention of apoptosis. As described herein, a platform was developed for testing combinatorial perturbations in T-cells, by co-expressing a gene of interest (e.g. LTBR) together with CRISPR sgRNAs targeting other genes, to map signaling networks in T-cells. Also demonstrated herein is that mRNA delivery of LTBR as an alternative to constitutive lentiviral expression, highlighting the translational potential of our screening approach.

In a certain embodiment, the expression cassette comprises a nucleic acid encoding LTBR, or a fragment thereof. LTBR (lymphotoxin-beta receptor), which encodes for tumor necrosis factor receptor superfamily member 3, is essential for the development and organization of secondary lymphoid tissues and chemokine release. A representative nucleic acid sequence of LTBR can be found at Accession ID NM_002342.3.

SEQ ID NO: 1
ATGCTCCTGCCTTGGGCCACCTCTGCCCCCGGCCTGGCCTGGGGGCCTCT
GGTGCTGGGCCTCTTCGGGCTCCTGGCAGCATCGCAGCCCCAGGCGGTGC
CTCCATATGCGTCGGAGAACCAGACCTGCAGGGACCAGGAAAAGGAATAC
TATGAGCCCCAGCACCGCATCTGCTGCTCCCGCTGCCCGCCAGGCACCTA
TGTCTCAGCTAAATGTAGCCGCATCCGGGACACAGTTTGTGCCACATGTG
CCGAGAATTCCTACAACGAGCACTGGAACTACCTGACCATCTGCCAGCTG
TGCCGCCCCTGTGACCCAGTGATGGGCCTCGAGGAGATTGCCCCCTGCAC
AAGCAAACGGAAGACCCAGTGCCGCTGCCAGCCGGGAATGTTCTGTGCTG
CCTGGGCCCTCGAGTGTACACACTGCGAGCTACTTTCTGACTGCCCGCCT
GGCACTGAAGCCGAGCTCAAAGATGAAGTTGGGAAGGGTAACAACCACTG
CGTCCCCTGCAAGGCCGGGCACTTCCAGAATACCTCCTCCCCCAGCGCCC
GCTGCCAGCCCCACACCAGGTGTGAGAACCAAGGTCTGGTGGAGGCAGCT
CCAGGCACTGCCCAGTCCGACACAACCTGCAAAAATCCATTAGAGCCACT
GCCCCCAGAGATGTCAGGAACCATGCTGATGCTGGCCGTTCTGCTGCCAC
TGGCCTTCTTTCTGCTCCTTGCCACCGTCTTCTCCTGCATCTGGAAGAGC
CACCCTTCTCTCTGCAGGAAACTGGGATCGCTGCTCAAGAGGCGTCCGCA
GGGAGAGGGACCCAATCCTGTAGCTGGAAGCTGGGAGCCTCCGAAGGCCC
ATCCATACTTCCCTGACTTGGTACAGCCACTGCTACCCATTTCTGGAGAT
GTTTCCCCAGTATCCACTGGGCTCCCCGCAGCCCCAGTTTTGGAGGCAGG
GGTGCCGCAACAGCAGAGTCCTCTGGACCTGACCAGGGAGCCGCAGTTGG
AACCCGGGGAGCAGAGCCAGGTGGCCCACGGTACCAATGGCATTCATGTC
ACCGGCGGGTCTATGACTATCACTGGCAACATCTACATCTACAATGGACC
AGTACTGGGGGGACCACCGGGTCCTGGAGACCTCCCAGCTACCCCCGAAC
CTCCATACCCCATTCCCGAAGAGGGGGACCCTGGCCCTCCCGGGCTCTCT
ACACCCCACCAGGAAGATGGCAAGGCTTGGCACCTAGCGGAGACAGAGCA
CTGTGGTGCCACACCCTCTAACAGGGGCCCAAGGAACCAATTTATCACCC
ATGAC

The full-length amino acid sequence of LTBR is SEQ ID NO: 2 (Uniprot P36941):

MLLPWATSAPGLAWGPLVLGLFGLLAASQPQAVPPYASENQTCRDQEKEY
YEPQHRICCSRCPPGTYVSAKCSRIRDTVCATCAENSYNEHWNYLTICQL
CRPCDPVMGLEEIAPCTSKRKTQCRCQPGMFCAAWALECTHCELLSDCPP
GTEAELKDEVGKGNNHCVPCKAGHFQNTSSPSARCQPHTRCENQGLVEAA
PGTAQSDTTCKNPLEPLPPEMSGTMLMLAVLLPLAFFLLLATVFSCIWKS
HPSLCRKLGSLLKRRPQGEGPNPVAGSWEPPKAHPYFPDLVQPLLPISGD
VSPVSTGLPAAPVLEAGVPQQQSPLDLTREPQLEPGEQSQVAHGTNGIHV
TGGSMTITGNIYIYNGPVLGGPPGPGDLPATPEPPYPIPEEGDPGPPGLS
TPHQEDGKAWHLAETEHCGATPSNRGPRNQFITHD

The LTBR protein can be divided into three regions, or domains: the extracellular domain (amino acids 31-227 of SEQ ID NO: 2); the transmembrane (or helical) domain (amino acids 228-248 of SEQ ID NO: 2); and the cytoplasmic (or intracellular) domain (amino acids 249-435 of SEQ ID NO: 2). The signal peptide of the immature protein is at amino acids 1-30 of SEQ ID NO: 2. When a domain is referred to herein, it is intended that variants, including N-terminal or C-terminal truncated variants, are included.

In certain embodiments, the expression cassette comprises a nucleic acid encoding a fragment of LTBR. In certain embodiments, the nucleic acid encodes a protein sequence having a deletion of amino acids 2-31, 32-41, 32-151, 32-180, 393-435, 377-435, 324-377, 297-435, or 262-435 as compared to the native protein (SEQ ID NO: 2). In certain embodiments, the nucleic acid encodes a protein sequence having a deletion in the N terminus. In certain embodiments, the nucleic acid encodes a protein sequence having a deletion in the C terminus. In one embodiment, the LTBR is has a deletion of residues 393-435. In certain embodiments, the LTBR has a deletion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, or at least 125 amino acids.

In certain embodiments, the expression cassette comprises a nucleic acid encoding a fragment that is a domain of LTBR. In certain embodiments, the nucleic acid encodes the extracellular domain of LTBR (amino acids 31-227 of SEQ ID NO: 2). In certain embodiments, the nucleic acid encodes the transmembrane domain of LTBR (amino acids 228-248 of SEQ ID NO. 2). In certain embodiments, the nucleic acid encodes the cytoplasmic (or intracellular) domain of LTBR (amino acids 249-435 of SEQ ID NO: 2). In other embodiments, the domain is a variant of one of the LTBR domains, including a variant that has a deletion. Desirable variants of the cytoplasmic domain include those with amino acids 249-378, 249-379, 249-380, 249-381, 249-382, 249-383, 249-384, 249-385, 249-386, 249-387, 249-388, 249-389, 249-390, 249-391, or 249-392 all of SEQ ID NO: 2. Further desirable variants include those with amino acids 249-378, 249-379, 249-380, 249-381, 249-382, 249-383, 249-384, 249-385, 249-386, 249-387, 249-388, 249-389, 249-390, 249-391, or 249-392 all of SEQ ID NO: 2 having 1, 2, 3, 4, 5, 6, 7, 8, 9, 10, 11, 12, 13, 14, or 15 amino acid substitutions as compared to SEQ ID NO: 2. In certain embodiments, the LTBR fragment comprises amino acids 249-396 of SEQ ID NO: 2. In certain embodiments, the LTBR fragment comprises amino acids 249-393 of SEQ ID NO: 2. In certain embodiments, the LTBR fragment comprises amino acids 249-387 of SEQ ID NO: 2. In certain embodiments, the LTBR fragment comprises amino acids 249-377 of SEQ ID NO: 2. In certain embodiments, the LTBR fragment comprises amino acids 262-435 of SEQ ID NO: 2. In certain embodiments, the LTBR fragment comprises amino acids 297-435 of SEQ ID NO: 2. In certain embodiments, the LTBR fragment comprises amino acids 324-435 of SEQ ID NO: 2. In certain embodiments, the LTBR fragment comprises amino acids 345-435 of SEQ ID NO: 2. In certain embodiments, the LTBR fragment comprises amino acids 358-435 of SEQ ID NO: 2.

In other embodiments, the expression cassette comprises a nucleic acid encoding two or more domains of LTBR. In one embodiment the nucleic acid encodes the cytoplasmic domain (or variant thereof) and the transmembrane domain of LTBR. In another embodiment, the nucleic acid encodes the cytoplasmic domain (or variant thereof), transmembrane domain, and extracellular domain of LTBR.

The fusion proteins described herein also include at least a fragment, including a domain, of second protein, different from the first. In certain embodiments, the second protein is a protein that is a component of a T cell receptor. In other embodiments, the second protein is a protein that interacts with T cell receptor. Such proteins include CD4, CD8A, CD8B, CD3E, CD3D, CD3G. and CD3Z.

In one embodiment a fusion protein includes an LTBR domain, or variant thereof, and CD4. In another embodiment a fusion protein includes an LTBR domain, or variant thereof, and CD8A and/or CD8B. In another embodiment a fusion protein includes an LTBR domain, or variant thereof, and CD3E. In another embodiment a fusion protein includes an LTBR domain, or variant thereof, and CD3D. In another embodiment a fusion protein includes an LTBR domain, or variant thereof, and CD3G. In another embodiment a fusion protein includes an LTBR domain, or variant thereof, and CD3Z. In certain embodiments, the LTBR domain is an intracellular domain. In certain embodiments, the LTBR intracellular domain comprises amino acids 249 to 435 of SEQ ID NO: 2, or variant thereof. In certain embodiments, the LTBR intracellular domain has a deletion in at least amino acids 393 to 435.

Various isoforms of the genes identified in Table 1 are known in the art. Some are described in Table 2 below. In another embodiment, an expression cassette is provided which includes the coding sequence for any of the alternative isoforms. Alternative coding sequences accounting to the degeneracy of the genetic code, including codon optimized coding sequences, for these genes can be identified by the person of skill in the art, and utilized as an alternative embodiment of the compositions and methods described herein.

TABLE 2

| Gene | | | Length | | Length | | |
|---|---|---|---|---|---|---|---|
| ID | Symbol | Transcript | (nt) | Protein | (aa) | Isoform | Gene name |
| 100 | ADA | NR_136160.2 | 1431 | | | | adenosine deaminase |
| 100 | ADA | NM_001322050.2 | 1380 | NP_001308979.1 | 228 | 2 | adenosine deaminase |
| 100 | ADA | NM_001322051.2 | 1424 | NP_001308980.1 | 339 | 3 | adenosine deaminase |
| 100 | ADA | NM_000022.4 | 1496 | NP_000013.2 | 363 | 1 | adenosine deaminase |
| 10538 | BATF | NM_006399.5 | 913 | NP_006390.1 | 125 | | basic leucine zipper ATF- |
| | | | | | | | like transcription factor |
| 1109 | AKR1C4 | NM_001818.5 | 1192 | NP_001809.4 | 323 | | aldo-keto reductase |
| | | | | | | | family 1 member C4 |
| 1388 | ATF6B | NM_001136153.2 | 2617 | NP_001129625.1 | 700 | b | activating transcription |
| | | | | | | | factor 6 beta |
| 1388 | ATF6B | NM_004381.5 | 2626 | NP_004372.3 | 703 | a | activating transcription |
| | | | | | | | factor 6 beta |
| 1622 | DBI | NM_001282635.3 | 804 | NP_001269564.1 | 104 | 1 | diazepam binding |
| | | | | | | | inhibitor, acyl-CoA |
| | | | | | | | binding protein |
| 1622 | DBI | NM_001282636.3 | 718 | NP_001269565.1 | 63 | 7 | diazepam binding |
| | | | | | | | inhibitor, acyl-CoA |
| | | | | | | | binding protein |
| 1622 | DBI | NM_001282633.3 | 680 | NP_001269562.1 | 104 | 1 | diazepam binding |
| | | | | | | | inhibitor, acyl-CoA |
| | | | | | | | binding protein |
| 1622 | DBI | NM_020548.9 | 675 | NP_065438.1 | 104 | 1 | diazepam binding |
| | | | | | | | inhibitor, acyl-CoA |
| | | | | | | | binding protein |
| 1622 | DBI | NM_001178043.4 | 594 | NP_001171514.1 | 97 | 6 | diazepam binding |
| | | | | | | | inhibitor, acyl-CoA |
| | | | | | | | binding protein |
| 1622 | DBI | NM_001178041.4 | 690 | NP_001171512.1 | 129 | 5 | diazepam binding |
| | | | | | | | inhibitor, acyl-CoA |
| | | | | | | | binding protein |
| 1622 | DBI | NM_001282634.3 | 650 | NP_001269563.1 | 104 | 1 | diazepam binding |
| | | | | | | | inhibitor, acyl-CoA |
| | | | | | | | binding protein |
| 1622 | DBI | NM_001178042.4 | 645 | NP_001171513.1 | 104 | 1 | diazepam binding |
| | | | | | | | inhibitor, acyl-CoA |
| | | | | | | | binding protein |
| 1622 | DBI | NM_001079862.4 | 564 | NP_001073331.1 | 87 | 3 | diazepam binding |
| | | | | | | | inhibitor, acyl-CoA |
| | | | | | | | binding protein |
| 1622 | DBI | NM_001352432.3 | 896 | NP_001339361.1 | 63 | 7 | diazepam binding |
| | | | | | | | inhibitor, acyl-CoA |
| | | | | | | | binding protein |
| 1622 | DBI | NM_001178017.3 | 756 | NP_001171488.1 | 148 | 4 | diazepam binding |
| | | | | | | | inhibitor, acyl-CoA |
| | | | | | | | binding protein |
| 1622 | DBI | NM_001079863.3 | 698 | NP_001073332.1 | 88 | 2 | diazepam binding |
| | | | | | | | inhibitor, acyl-CoA |
| | | | | | | | binding protein |
| 191 | AHCY | XM_011528657.2 | 1436 | XP_011526959.2 | 434 | X1 | adenosylhomocysteinase |
| 191 | AHCY | XM_017027709.2 | 1801 | XP_016883198.1 | 432 | X2 | adenosylhomocysteinase |
| 191 | AHCY | XM_011528658.3 | 1798 | XP_011526960.2 | 434 | X1 | adenosylhomocysteinase |
| 191 | AHCY | XM_011528656.3 | 1803 | XP_011526958.2 | 434 | X1 | adenosylhomocysteinase |
| 191 | AHCY | XM_011528659.1 | 2196 | XP_011526961.1 | 404 | X3 | adenosylhomocysteinase |
| 191 | AHCY | NM_001161766.2 | 2368 | NP_001155238.1 | 404 | 2 | adenosylhomocysteinase |
| 191 | AHCY | NM_001362750.2 | 1774 | NP_001349679.1 | 432 | 1 | adenosylhomocysteinase |
| 191 | AHCY | NM_000687.4 | 2150 | NP_000678.1 | 432 | 1 | adenosylhomocysteinase |
| 191 | AHCY | XM_017027710.2 | 2136 | XP_016883199.1 | 306 | X4 | adenosylhomocysteinase |
| 191 | AHCY | XM_005260317.2 | 2601 | XP_005260374.1 | 404 | X3 | adenosylhomocysteinase |
| 191 | AHCY | NM_001322085.2 | 3092 | NP_001309014.1 | 404 | 2 | adenosylhomocysteinase |
| 191 | AHCY | NM_001322084.2 | 3270 | NP_001309013.1 | 404 | 2 | adenosylhomocysteinase |
| 191 | AHCY | NM_001322086.2 | 3281 | NP_001309015.1 | 434 | 3 | adenosylhomocysteinase |
| 2819 | GPD1 | NM_005276.4 | 2887 | NP_005267.2 | 349 | 1 | glycerol-3-phosphate |
| | | | | | | | dehydrogenase 1 |
| 2819 | GPD1 | NM_001257199.2 | 2818 | NP_001244128.1 | 326 | 2 | glycerol-3-phosphate |
| | | | | | | | dehydrogenase 1 |
| 282616 | IFNL2 | NM_172138.2 | 951 | NP_742150.1 | 200 | | interferon lambda 2 |
| 338599 | DUSP29 | XM_011539747.2 | 4872 | XP_011538049.1 | 220 | X1 | dual specificity |
| | | | | | | | phosphatase 29 |
| 338599 | DUSP29 | NM_001003892.3 | 1135 | NP_001003892.1 | 220 | | dual specificity |
| | | | | | | | phosphatase 29 |
| 338599 | DUSP29 | NM_001384909.1 | 1267 | NP_001371838.1 | 220 | | dual specificity |
| | | | | | | | phosphatase 29 |
| 338599 | DUSP29 | XM_017016176.1 | 1284 | XP_016871665.1 | 112 | X2 | dual specificity |
| | | | | | | | phosphatase 29 |
| 3593 | IL12B | NM_002187.3 | 2364 | NP_002178.2 | 328 | | interleukin 12B |
| 4055 | LTBR | NM_001270987.2 | 2276 | NP_001257916.1 | 416 | 2 | lymphotoxin beta |
| | | | | | | | receptor |
| 4055 | LTBR | XM_005253688.2 | 2094 | XP_005253745.1 | 399 | X1 | lymphotoxin beta |
| | | | | | | | receptor |
| 4055 | LTBR | XM_006718983.3 | 1127 | XP_006719046.1 | 255 | X2 | lymphotoxin beta |
| | | | | | | | receptor |
| 4055 | LTBR | NM_002342.3 | 2134 | NP_002333.1 | 435 | 1 | lymphotoxin beta |
| | | | | | | precursor | receptor |
| 51184 | GPN3 | NM_001164372.2 | 1605 | NP_001157844.1 | 323 | 2 | GPN-loop GTPase 3 |
| 51184 | GPN3 | NM_016301.4 | 1457 | NP_057385.3 | 284 | 1 | GPN-loop GTPase 3 |
| 51184 | GPN3 | NM_001164373.2 | 1487 | NP_001157845.1 | 294 | 3 | GPN-loop GTPase 3 |
| 51184 | GPN3 | XM_017019394.1 | 1357 | XP_016874883.1 | 262 | X1 | GPN-loop GTPase 3 |
| 51258 | MRPL51 | NM_016497.4 | 898 | NP_057581.2 | 128 | | mitochondrial ribosomal |
| | | | | | | | protein L51 |
| 79026 | AHNAK | NM_024060.4 | 1036 | NP_076965.2 | 149 | 2 | AHNAK nucleoprotein |
| 79026 | AHNAK | NM_001620.3 | 18761 | NP_001611.1 | 5890 | 1 | AHNAK nucleoprotein |
| 79026 | AHNAK | XM_017018270.1 | 18556 | XP_016873759.1 | 5823 | X1 | AHNAK nucleoprotein |
| 79026 | AHNAK | NM_001346445.2 | 18697 | NP_001333374.1 | 5890 | 1 | AHNAK nucleoprotein |
| 79026 | AHNAK | NM_001346446.2 | 18713 | NP_001333375.1 | 5890 | 1 | AHNAK nucleoprotein |
| 810 | CALML3 | NM_005185.4 | 1811 | NP_005176.1 | 149 | | calmodulin like 3 |
| 9452 | ITM2A | NM_001171581.2 | 1484 | NP_001165052.1 | 219 | 2 | integral membrane |
| | | | | | | | protein 2A |
| 9452 | ITM2A | NM_004867.5 | 1616 | NP_004858.1 | 263 | 1 | integral membrane |
| | | | | | | | protein 2A |

Engineered T cell Receptors Containing LTBR Domains

The present disclosure provides nucleic acid sequences encoding engineered T cell receptors, e.g., T cell receptors (TCRs), that incorporate an LTBR domain. Also provided are chimeric antigen receptors (CAR) that incorporate an LTBR domain or variant thereof. Components of the TCR and CARs are further described herein. Also provided are the engineered TCRs and CARs, and modified T cells incorporating the same.

Also provided herein are nucleic acid sequences encoding engineered T cell receptors, e.g., T cell receptors (TCRs), that incorporate a domain or variant thereof from one of the genes of Table 3. Also provided are chimeric antigen receptors (CAR) that incorporate domain from one of the genes of Table 3. In certain embodiments, the domain is an intracellular domain from a gene selected from those of Table 3. Also provided are the engineered TCRs and CARs, and modified T cells incorporating the same. As described below, embodiments incorporating LTBR domains are set forth. However, for each embodiment described for LTBR, an embodiment is intended for each of the genes of Table 3.

TABLE 3

ABCC5
ACE
ACP2
ADAM2
AMIGO3
APCDD1
ART4
BDKRB2
C3AR1
CA12
CCR4
CD14
CD1B
CD2
CD244
CD300A
CD4
CD48
CD52
CD59
CD79B
CLDN4
CNR2
CRLF2
CRTAM
CSF1R
CXCR1
DCT
DPEP2
DYNAP
EDA
EMP3
ENPP1
EREG
ERMAP
EVA1C
EVI2A
F11R
F2RL1
F2RL2
FAP
FCGR2B
FNDC4
FOLR2
FZD10
GABRA5
GABRB1
GJB7
GPNMB
GPR17
GPR3
GPR37L1
GRID1
GRIK2
GRPR
GSG1
GYPC
HAVCR2
HEPACAM2
HLA-DOA
HLA-DPB1
HTR1D
IGF1R
IL18RAP
KCNJ12
KCNMB1
LAMP2
LHFPL5
LMAN2L
LMBRD1
LRRC25
LRRTM3
LRTM1
LTBR
LYPD4
LYPD6B
MAS1
MC3R
MMP16
MOG
MUC1
NCR3
OR10G3
OR10G6
OR10G9
OR10W1
OR10X1
OR13G1
OR1D5
OR1N2
OR1Q1
OR2G2
OR2H1
OR2M4
OR3A2
OR4D11
OR4K17
OR4X1
OR51G1
OR52W1
OR6B2
OR8H2
OXGR1
P2RX7
P2RY6
PDCD1
PIEZO1
PODXL2
PRND
PRPH2
PRSS8
PTCHD1
PTCHD4
RGR
RHCG
S1PR3
SCTR
SDC1
SEMA4G
SGCA
SLC10A6
SLC11A2
SLC14A1
SLC1A7
SLC26A1
SLC26AS
SLC37A4
SLC39A4
SLC41A2
SLC46A3
SLC4A4
SLC52A1
SLC6A3
SPPL2C
STIM1
SYNPR
TAAR5
TAS2R3
TCIRG1
TMEM132C
TMEM182
TMEM255A
TMEM37
TMIGD1
TMPRSS11E
TMPRSS6
TNFSF18
TRPV2
TRPV5
TSPAN1
TSPAN31
TSPAN9
VIPR1
VSTM1
VTCN1
ZDHHC5

Provided herein are nucleic acid molecules that comprise a coding sequence for any of the TCRs described herein modified to include a domain of LTBR or a gene of Table 3. Certain exemplary TCRs are provided in the sequence listing in SEQ ID Nos: 3-54. However, it is intended that nucleic acids encoding all the described TCRs, as well as the TCR proteins, are encompassed herewith.

The TCR is a disulfide-linked membrane-anchored heterodimer present on T cell lymphocytes, and the majority of T cells are αβ T cells having a TCR consisting of an alpha (a) chain and a beta (B) chain. Each chain comprises a variable (V) and a constant (C) domain, wherein the variable domain recognizes an antigen, or an MHC-presented peptide. TCRα and TCRβ chains with a known specificity or affinity for specific antigens, e.g., tumor antigens described herein, can be introduced to a T cell using the methods described herein. TCRα and TCRβ chains having a desired, e.g., increased, specificity or affinity for a particular antigen can be isolated using standard molecular cloning techniques known in the art. Other modifications that increase specificity, avidity, or function of the TCRs or the engineered T cells expressing the TCRs can be readily envisioned by the ordinarily skilled artisan, e.g., promoter selection for regulated expression, mutations in the antigen binding regions of the TCRα and TCRβ chains. Any isolated or modified TCRα and TCRβ chain can be operably linked to or can associate with one or more intracellular signaling domains described herein. Signaling can be mediated through interaction between the antigen-bound αβ heterodimer to CD3 chain molecules, e.g., CD3zeta (3).

A smaller subset of T cells expresses a TCR having a (γ) gamma chain and a delta (8) chain. Gamma-delta (γδ) T cells make up 3-10% of circulating lymphocytes in humans, and the V82+ subset can account for up to 95% of γδ T cells in blood. Vδ2+ cells recognize non-peptide epitopes and do not require antigen presentation by major histocompatibility complexes (“MHC”) or human leukocyte antigen (“HLA”). The majority of Vδ2+ T cells also express a Vγ9 chain and are stimulated by exposure to 5-carbon pyrophosphate compounds that are intermediates in mevalonate and non-mevalonate sterol/isoprenoid synthesis pathways. The response to isopentenyl pyrophosphate (5-carbon) is universal among healthy human beings. Another subset of γδ T cells, Volt, make up a much smaller percentage of the T cells circulating in the blood, but are commonly found in the epithelial mucosa and the skin, γδ T cells have several functions, including killing tumor cells and pathogen-infected cells. Stimulation through the γδ TCR improves the capacity for cellular cytotoxicity, cytokine secretion and other effector functions. The TCRs of γδ T cells have unique specificities and the cells themselves occur in high clonal frequencies, thus allowing rapid innate-like responses to tumors and pathogens. See, e.g., Park and Lee, Exp Mol Med. 2021 March; 53(3): 318-327., which is incorporated herein by reference.

In certain embodiments, a T cell comprises a nucleic acid sequence encoding a TCR, e.g., a TCR that targets a tumor antigen, that includes an LTBR domain. In certain embodiments, the TCR includes the LTBR intracellular domain, or variant thereof as described herein. In one embodiment, the variant has a deletion in at least amino acids 393 to 435 of SEQ ID NO: 2. In one embodiment, the variant of the intracellular domain includes amino acids 249-392 of SEQ ID NO: 2. However, additional residues may be deleted. Thus, desirable variants of the intracellular domain include those with a sequence of amino acids 249-378, 249-379, 249-380, 249-381, 249-382, 249-383, 249-384, 249-385, 249-386, 249-387, 249-388, 249-389, 249-390, 249-391, or 249-392 all of SEQ ID NO: 2. Further desirable variants include those with a sequence of amino acids 249-378, 249-379, 249-380, 249-381, 249-382, 249-383, 249-384, 249-385, 249-386, 249-387, 249-388, 249-389, 249-390, 249-391, or 249-392 all of SEQ ID NO: 2 having 1, 2, 3, 4, 5, 6, 7, 8, 9, 10, 11, 12, 13, 14, or 15 amino acid substitutions as compared to SEQ ID NO: 2.

In certain embodiments, a T cell comprises a nucleic acid sequence encoding a TCR, e.g., a modified TCR that targets a tumor antigen described herein, that includes a domain from the genes of Table 3. In certain embodiments, the TCR includes an intracellular domain from the genes of Table 3, or variant thereof as described herein

In one embodiment, the TCR comprises an LTBR intracellular domain fused to C-terminus of the TCR alpha chain. In another embodiment, the TCR comprises an LTBR intracellular domain fused to the C-terminus of the TCR beta chain. FIG. 25A, FIG. 29A. In one embodiment, the TCR comprises an LTBR intracellular domain fused to the C-terminus of the TCR delta chain. In another embodiment, the TCR comprises an LTBR intracellular domain fused to the C-terminus of the TCR gamma chain. In another embodiment, the TCR comprises an LTBR intracellular domain fused to the C-terminus of the CD3γ. In another embodiment, the TCR comprises an LTBR intracellular domain fused to the C-terminus of the CD3δ. In another embodiment, the TCR comprises an LTBR intracellular domain fused to the C-terminus of one or both CD3ε. In another embodiment, the TCR comprises an LTBR intracellular domain fused to the C-terminus of one or both CD3ζ. See FIGS. 25A and 25B.

In one embodiment, the TCR comprises an intracellular domain of a gene of Table 3 fused to C-terminus of the TCR alpha chain. In another embodiment, the TCR comprises an intracellular domain of a gene of Table 3 fused to the C-terminus of the TCR beta chain. FIG. 25A. In one embodiment, the TCR comprises an intracellular domain of a gene of Table 3 fused to the C-terminus of the TCR delta chain. In another embodiment, the TCR comprises an intracellular domain of a gene of Table 3 fused the C-terminus of the TCR gamma chain. In another embodiment, the TCR comprises an intracellular domain of a gene of Table 3 fused to the C-terminus of the CD3γ. In another embodiment, the TCR comprises an intracellular domain of a gene of Table 3 fused to the C-terminus of the CD3δ. In another embodiment, the TCR comprises an intracellular domain of a gene of Table 3 fused to the C-terminus of one or both CD3ε. In another embodiment, the TCR comprises an intracellular domain of a gene of Table 3 fused to the C-terminus of one or both CD34.

In another embodiment, the LTBR intracellular domain is fused to the C-terminal intracellular tail of CD4. In another embodiment, the LTBR intracellular domain is fused to the C-terminal intracellular tails of CD8α and/or CD8β. See FIG. 25C.

In another embodiment, an intracellular domain of a gene of Table 3 is fused to the C-terminal intracellular tail of CD4. In another embodiment, an intracellular domain of a gene of Table 3 is fused to the C-terminal intracellular tails of CD8α and CD8β.

In other embodiments, at least one domain of LTBR is delivered to a lymphocyte via direct modification of the endogenous genome. Various techniques for modification of the endogenous genome are known in the art, including CRISPR, zinc finger nucleases, TALENS, etc. See, e.g., Azangou-Khyavy et al. CRISPR/Cas: From Tumor Gene Editing to T Cell-Based Immunotherapy of Cancer, Front. Immunol., 29 Sep. 2020 | https://doi.org/10.3389/fimmu.2020.02062, which is incorporated herein by reference. In on embodiment, the LTBR intracellular domain, or fragment thereof, is inserted into the genome of a lymphocyte. In other embodiments, at least one domain of a gene of Table 3 is delivered to a lymphocyte via direct modification of the endogenous genome.

In certain embodiments, the TCR is a known TCR, such as those identified in FIG. 20, as modified as described herein to contain a domain of LTBR or a gene of Table 3 (or variant thereof). In certain embodiments, the TCR targets MART-1. Chodon T, et al, Adoptive transfer of MART-I T-cell receptor transgenic lymphocytes and dendritic cell vaccination in patients with metastatic melanoma. Clin Cancer Res. 2014 May 1; 20(9): 2457-65, doi: 10.1158/1078-0432.CCR-13-3017. Epub 2014 Mar. 14. PMID: 24634374; PMCID: PMC4070853. In other embodiments, the TCR targets MAGE A4. Hong et al, Phase I dose escalation and expansion trial to assess the safety and efficacy of ADP-A2M4 SPEAR T cells in advanced solid tumors. ASCO Meeting Library, 2020 ASCO Virtual Scientific Program, J Clin Oncol 38:2020 (suppl; abstr 102). In other embodiments, the TCR targets WT1. Chapuis A G, et al. T cell receptor gene therapy targeting WT1 prevents acute myeloid leukemia relapse post-transplant. Nat Med. 2019 July; 25(7):1064-1072, doi: 10.1038/s41591-019-0472-9. Epub 2019 Jun. 24. PMID: 31235963; PMCID: PMC6982533. In other embodiments, the TCR targets MR1. Crowther, M. D., Dolton. G., Legut, M, et al. Genome-wide CRISPR-Cas9 screening reveals ubiquitous T cell cancer targeting via the monomorphic MHC class I-related protein MRI. Nat Immunol 21, 178-185 (2020), https://2/10.10384 590-019-0578-8. In other embodiments, the TCR targets E6. In other embodiments, the TCR targets E7. In other embodiments, the TCR targets KK-LC-1. In other embodiments, the TCR targets NY-ESO-1. In other embodiments, the TCR targets MAGE A3. In other embodiments, the TCR targets GD-2. In other embodiments, the TCR targets P53. In other embodiments, the TCR targets LAGE-A1. In other embodiments, the TCR targets GP100.

In any of the embodiments described herein, a modified TCR can be substituted for a CAR described herein to generate a T cell. An engineered TCR described herein can be substituted for a CAR in any of the embodiments described herein.

### Chimeric Antigen Receptors (CARs) Containing LTBR Domains

The term “chimeric antigen receptor” or alternatively a “CAR” refers to a recombinant polypeptide construct comprising at least an extracellular antigen binding domain, a stalk/hinge, a transmembrane domain, and a cytoplasmic signaling domain (also referred to as an intracellular signaling domain) comprising a functional signaling domain derived from a stimulatory molecule as defined below. See Jayaraman et al, CAR-T design: Elements and their synergistic function, eBioMedicine, 58:102931 (August 2020), which is incorporated herein by reference. In some embodiments, the stimulatory molecule is TCR zeta, FcR gamma, FcR beta, CD3 gamma. CD3 delta. CD3 epsilon, CD5, CD22, CD79a, CD79b. CD66d. 4-1BB, or CD3-zeta. In a particular embodiment, the stimulatory molecule is the zeta chain associated with the T cell receptor complex As used herein for ease of reference, when referring to a stimulatory molecule, the term CD3ζ (may be used. However, it is intended that a similar embodiment is provided in which the CD3ζ is swapped for another suitable stimulatory molecule. In one embodiment, the stimulatory molecule is 4-1BB. In one embodiment, the stimulatory molecule is CD28. In another embodiment, the stimulatory molecule is LTBR and the stimulatory signaling domain includes the LTBR intracellular domain, or variant thereof. In another embodiment, the stimulatory molecule is a gene of Table 3, and the stimulatory signaling domain includes an intracellular domain of a gene of Table 3.

In one embodiment, the cytoplasmic signaling domain further comprises one or more functional signaling domains derived from at least one costimulatory molecule as defined below (also referred to as a “costimulatory signaling domain”). In one embodiment, the costimulatory molecule is chosen from a costimulatory molecule described herein, e.g., OX40, CD27, CD28, CD30, CD40, PD-1, CD2. CD7, CD258, NKG2C, B7-H3, a ligand that binds to CD83, ICAM-1, LFA-1 (CD11a/CD18), ICOS and 4-1BB (CD137), or any combination thereof. In certain embodiments, the costimulatory molecule is LTBR, and the costimulatory signaling domain includes the LTBR intracellular domain, or variant thereof. In another embodiment, the costimulatory molecule is a gene of Table 3, and the costimulatory signaling domain includes an intracellular domain of a gene of Table 3.

As described herein, the CAR itself comprises one or more LTBR domains. The LTBR domain(s) may, in some embodiments, replace a domain from an existing CAR construct, such as those described in FIG. 19. In other embodiments, the LTBR domain(s) is/are included in addition to the domains from an existing CAR construct, such as those described in FIG. 19.

As described herein, the CAR itself comprises one or more domains from a gene of Table 3. The domain(s) from the gene of Table 3 may, in some embodiments, replace a domain from an existing CAR construct, such as those described in FIG. 19. In other embodiments, the domain(s) from the gene of Table 3 is/are included in addition to the domains from an existing CAR construct, such as those described in FIG. 19.

In certain embodiments, the stalk and the transmembrane are from the same molecule, e.g., LTBR, CD8, or CD28. In other embodiments, the stalk and the transmembrane are from different molecules, e.g., CD8 stalk and LTBR™, CD28 stalk and LTBR™, etc.

In one embodiment the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the scFv domain during cellular processing and localization of the CAR to the cellular membrane.

In one embodiment, the CAR comprises a chimeric fusion protein comprising an extracellular antigen recognition domain, a transmembrane domain, and an intracellular signaling domain comprising a functional signaling domain derived from LTBR, or variant thereof. Without wishing to be bound by theory, T cells engineered with CARs lacking a CD3zeta chain do not induce specific cytokine secretion in the presence of CD19+ leukemia cells. However, these cells induce NFκB-induced genes (as demonstrated by CD74 expression (see FIG. 24E), and do induce IFNγ and IL2 secretion when stimulated with CD3 and CD28 antibodies (FIGS. 24C and 24D).

In certain embodiments, in addition to the LTBR signaling domain, the transmembrane domain is also derived from LTBR. In certain embodiments, in addition to the LTBR signaling domain and (optionally) the transmembrane domain, the stalk is also derived from LTBR.

In one embodiment, the CAR comprises a chimeric fusion protein comprising an extracellular antigen recognition domain, a transmembrane domain, and an intracellular signaling domain comprising a functional signaling domain derived LTBR, or variant thereof.

In one embodiment, the CAR comprises a chimeric fusion protein comprising an extracellular antigen recognition domain, a transmembrane domain, an intracellular signaling domain comprising a functional cosignaling domain derived from LTBR, or variant thereof, and a functional signaling domain derived from a stimulatory molecule, e.g., CD3 zeta chain. The placement of the LTBR cosignaling domain may be varied depending on the desired function of the CAR. In certain embodiments, the LTBR cosignaling domain, or variant thereof is placed between the transmembrane domain and the signaling domain (e.g., CD3C). In other embodiments, the CD3C is placed between the transmembrane domain and the LTBR cosignaling domain, or variant thereof.

In certain embodiments, in addition to the signaling domain, the transmembrane domain is also derived from a gene of Table 3. In certain embodiments, in addition to the signaling domain and (optionally) the transmembrane domain, the stalk is also derived from a gene of Table 3.

In one embodiment, the CAR comprises a chimeric fusion protein comprising an extracellular antigen recognition domain, a transmembrane domain, and an intracellular signaling domain comprising a functional signaling domain derived from a gene of Table 3, or variant thereof.

In one embodiment, the CAR comprises a chimeric fusion protein comprising an extracellular antigen recognition domain, a transmembrane domain, an intracellular signaling domain comprising a functional cosignaling domain derived from LTBR, or variant thereof, at least one other functional cosignaling domain, and a functional signaling domain derived from a stimulatory molecule, e.g., CD3zeta chain. The placement of the LTBR cosignaling domain may be varied depending on the desired function of the CAR. In certain embodiments, the LTBR cosignaling domain, or variant thereof is placed between the transmembrane domain and the other cosignaling domain(s) (e.g., CD28 or 4-1BB). In certain embodiments, the LTBR cosignaling domain, or variant thereof is placed between the other cosignaling domain(s) and the signaling domain (e.g., CD3ζ). In other embodiments, the LTBR cosignaling domain, or variant thereof is placed downstream of the signaling domain (e.g., CD3ζ). See, e.g., FIG. 21A and FIG. 21B.

In certain embodiments, in addition to the LTBR signaling domain, the transmembrane domain is also derived from LTBR. In certain embodiments, in addition to the LTBR signaling domain and (optionally) the transmembrane domain, the stalk is also derived from LTBR.

In one embodiment, the CAR comprises a chimeric fusion protein comprising an extracellular antigen recognition domain, a transmembrane domain, an intracellular signaling domain comprising a functional cosignaling domain derived from a gene of Table 3, or variant thereof, at least one other functional cosignaling domain, and a functional signaling domain derived from a stimulatory molecule, e.g., CD3zeta chain. The placement of the cosignaling domain may be varied depending on the desired function of the CAR. In certain embodiments, the cosignaling domain from a gene of Table 3, or variant thereof is placed between the transmembrane domain and the other cosignaling domain(s) (e g., CD28 or 4-1BB). In certain embodiments, the cosignaling domain from a gene of Table 3, or variant thereof is placed between the other cosignaling domain(s) and the signaling domain (e.g., CD3ζ). In other embodiments, the cosignaling domain from a gene of Table 3, or variant thereof is placed downstream of the signaling domain (e.g., CD3ζ).

The present disclosure provides nucleic acid sequences, e.g., a DNA or an RNA construct, that encode any of the CARs described herein. This also refers to nucleic acid sequences encoding a known CAR such as one of those shown in FIG. 19, but modified to include an LTBR domain or variant thereof, as described herein.

The present disclosure provides nucleic acid sequences, e.g., a DNA or an RNA construct, that encode any of the CARs described herein. This also refers to nucleic acid sequences encoding a known CAR such as one of those shown in FIG. 19, but modified to include a domain of a gene of Table 3 or variant thereof, as described herein.

In certain embodiments, the CAR targets CD19. In certain embodiments, the CAR is a known CAR, such as one of those shown in FIG. 19, but modified to include an LTBR domain (or gene of Table 3) or variant thereof, as described herein. In one embodiment, the CAR is a modified axicabtagene ciloleucel. In another embodiment, the CAR is a modified Brexucabtagene autoleucel. In another embodiment, the CAR is a modified Tisagenlecleucel. In another embodiment, the CAR is a modified Lisocabtagene maraleucel. In certain embodiments, the CAR targets B-cell maturation antigen (BCMA). In another embodiment, the CAR is a modified Idecabtagene vicleucel. In one embodiment, the CAR is a modified ciltacabtagene autoleucel. In certain embodiments, the CAR targets mesothelin. In certain embodiments, the CAR targets ROR1. In certain embodiments, the CAR targets B7-H3. In certain embodiments, the CAR targets CD33. In certain embodiments, the CAR targets EGFR806. In certain embodiments, the CAR targets IL13Rα2. In certain embodiments, the CAR targets GD2. In certain embodiments, the CAR targets HER2. In certain embodiments, the CAR targets Glypican 3. In certain embodiments, the CAR targets CD7. In certain embodiments, the CAR targets NY-ESO-1. In certain embodiments, the CAR targets CD30. In certain embodiments, the CAR targets MAGE-A1. In certain embodiments, the CAR targets LMP2. In certain embodiments, the CAR targets PD1. In certain embodiments, the CAR targets mutant KRAS G12V. In certain embodiments, the CAR targets CD20. In certain embodiments, the CAR targets CD22. In certain embodiments, the CAR targets CD171. In certain embodiments, the CAR targets CD123. In certain embodiments, the CAR targets CD38. In certain embodiments, the CAR targets CD10. In certain embodiments, the CAR targets BAFFR. In certain embodiments, the CAR targets PSMA. In certain embodiments, the CAR targets mucin (TnMUC1). Posey AD Jr, et al, Engineered CAR T Cells Targeting the Cancer-Associated Tn-Glycoform of the Membrane Mucin MUC1 Control Adenocarcinoma. Immunity. 2016 Jun. 21; 44(6):1444-54, doi: 10.1016/j.immuni.2016.05.014. PMID: 27332733: PMCID: PMC5358667. In certain embodiments, the CAR targets CD70. See, Srinivasan et al, 1972 Investigation of ALLO-316: A Fratricide-Resistant Allogeneic CAR T Targeting CD70 As a Potential Therapy for the Treatment of AML. 62^{nd} ASH Annual Meeting and Exposition, Dec. 5-8, 2020. In certain embodiments, the CAR targets TRIB1C. Maciocia P M, et al. Targeting the T cell receptor β-chain constant region for immunotherapy of T cell malignancies. Nat Med. 2017 December: 23(12):1416-1423, doi: 10.1038/nm.4444. Epub 2017 Nov. 13. PMID: 29131157.

Exemplary sequences for the CARs and TCRs used in the Examples are provided in the sequence listing in SEQ ID Nos: 3-54. Other exemplary antibody sequences, useful for the antigen recognition domain, are provided in the sequence listing in SEQ ID Nos: 63-83.

| SEQ ID | | |
|---|---|---|
| NO | Name | Type |
| 63 | synthetic anti-mesothelin monoclonal antibody HN1 VH | DNA |
| 64 | synthetic anti-mesothelin monoclonal antibody HN1 VH | AA |
| 65 | synthetic anti-mesothelin monoclonal antibody HN1 VL | DNA |
| 66 | synthetic anti-mesothelin monoclonal antibody HN1 VL | AA |
| 67 | synthetic anti-mesothelin monoclonal antibody HN2 VH | DNA |
| 68 | synthetic anti-mesothelin monoclonal antibody HN2 VH | AA |
| 69 | synthetic anti-mesothelin monoclonal antibody HN2 VL | DNA |
| 70 | synthetic anti-mesothelin monoclonal antibody HN2 VL | AA |
| 71 | AntiCD19 VH (24) | DNA |
| 72 | AntiCD19 VL (26) | DNA |
| 73 | AntiCD19 VL (40) | DNA |
| 74 | AntiCD38 hScFv (5) | DNA |
| 75 | AntiCD38 hScFv (8) | AA |
| 76 | AntiCD38 hScFv (10) | AA |
| 77 | AntiCD20 VL (156) | DNA |
| 78 | AntiCD20 VH (145) | DNA |
| 79 | AntiCD22 VL (690) | AA |
| 80 | AntiCD22 VH (680) | AA |
| 81 | AntiCD33 VL (71) | AA |
| 82 | AntiCD33 VH (62) | AA |
| 83 | AntiCD33 scFv (44) | AA |

Other chimeric antigen receptors as modified herein, include those useful for treatment for autoimmune disease, such as are chimeric autoantigen receptors (CAAR). Such CAARs include DSG3-CAART and MuSK-CAART. Others may be known in the art or may be designed by the person of skill.

The present disclosure provides nucleic acid sequences, e.g., a DNA or an RNA construct, that encode any of the TCRs described herein. This also refers to nucleic acid sequences encoding a known TCR such as one of those shown in FIG. 20, but modified to include an LTBR domain (or gene of Table 3) or fragment thereof, as described herein. Various other engineered T cell receptors are known in the art or may be designed by the person of skill. Such TCRs include those currently being tested clinically, such as those identified in FIG. 20.

The expression cassettes referred to herein include a nucleic acid molecule which encodes one or more biologically useful nucleic acid sequences (e.g., a gene cDNA encoding a fusion protein, CAR, TCR, mRNA, etc.) and regulatory sequences operably linked thereto which direct or modulate transcription, translation, and/or expression of the nucleic acid sequence(s) and its gene product(s). Operably linked sequences include both regulatory sequences that are contiguous with the nucleic acid sequence and regulatory sequences that act in trans or at a distance to control the sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region comprising a polyadenylation site, among other elements. Thus, in addition to the coding sequences for the fusion protein, CAR, or TCR the expression cassette may also include expression control sequences.

The expression control sequences include a promoter. In some embodiments, its it is desirable to utilize a promoter having high transcriptional activity. Certain strong constitutive promoters are known in the art and include, without limitation, the CMV promoter, the EF-la promoter, CBG promoter. CB7 promoter, etc. Alternatively, other promoters, such as regulatable (inducible) promoters [see, e.g., WO 2011/126808 and WO 2013/049493, incorporated by reference herein], or a promoter responsive to physiologic cues may be utilized. In certain embodiments, the inducible promoter is activated in response to T cell stimulation. In certain embodiments, the promoter is an NFAT, API. NFκB, or IRF4 promoter.

The expression cassette may also include, in certain embodiments, one or more IRES or 2A sequence(s) to allow for expression of multiple coding sequences from the same expression cassette. As exemplified herein, in one embodiment, a TCR directed to NY-ESO-1 is provided in which an LTBR intracellular domain is expressed contiguously with the TCRα (or TCRβ) chain. See, FIG. 5A, in a lentiviral vector which includes 2A sequences. Construction of such cassettes and vectors are known in the art, and are described herein in the Examples. See, e.g., Sack et al. Profound Tissue Specificity in Proliferation Control Underlies Cancer Drivers and Aneuploidy Patterns. Cell. 2018 Apr. 5; 173(2):499-514.e2 and Yang et al. A public genome-scale lentiviral expression library of human ORFs, Nat Methods. 2011 August; 8(8): 659-661, which are incorporated herein by reference.

Provided herein, in certain aspects, are compositions which include modified lymphocytes which comprise the nucleic acids and/or expression cassettes described herein. In one embodiment, the host lymphocyte is a T cell. In another embodiment, the host lymphocyte is a natural killer (NK) cell. In certain embodiments, the composition comprises a population of cells which includes a mixed population of lymphocytes (e.g., alpha beta T cells and NK T cells). In other embodiments, the composition comprises cells which includes a population which is enriched for a particular lymphocyte population.

As used herein, the phrase “T cell” refers to a lymphocyte that expresses a T cell receptor molecule. T cells include human alpha beta (αβ) T cells and human gamma delta (γδ) T cells. T cells include, but are not limited to, naive T cells, stimulated T cells, primary T cells (e.g., uncultured), cultured T cells, immortalized T cells, helper T cells, cytotoxic T cells, memory T cells, regulatory T cells, natural killer T cells, combinations thereof, or subpopulations thereof. T cells can be CD4+, CD8+, or CD4+ and CD8+. T cells can also be CD4−, CD8−, or CD4− and CD8−. T cells can be helper cells, for example helper cells of type T_{H}1, T_{H}2, T_{H}3. T_{H}9, T_{H}17, or T_{FH}. T cells can be cytotoxic T cells. T cells can also be regulatory T cells. Regulatory T cells (Tregs) can be FOXP3+ or FOXP3−. T cells can be alpha/beta T cells or gamma/delta T cells. In some cases, the T cell is a CD4+CD25^{hi}CD127^{lo} regulatory' T cell. In some cases, the T cell is a regulatory T cell selected from the group consisting of type 1 regulatory (Tr1). T_{H}3, CD8+CD28−, Treg 17, and Qa−1 restricted T cells, or a combination or sub-population thereof. In some cases, the T cell is a FOXP3⁺ T cell. In some cases, the T cell is a CD4⁺CD251^{lo}CD127^{hi} effector T cell. In some cases, the T cell is a CD4⁺CD25^{lo} CD127^{hi}CD45RA^{hi}CD45RO-naive T cell. In some cases, the T cell is a Vγ9V82 T cell. In some embodiments, the T cell expresses a viral antigen. In other embodiments, the T cell expresses a cancer antigen. A T cell can be a recombinant T cell that has been genetically manipulated.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

### Methods

### Methods of Making Modified Host Cells

Also provided herein are methods of making the modified cells and compositions containing modified cells as described herein. Methods of modifying cells, e.g., lymphocytes, to introduce an exogenous sequence, such as an expression cassette or expression vector comprising a coding sequence for a fusion protein, a CAR, or TCR, or more than one of these sequences, are known in the art. For example, see, e.g., WO 2016/109410 A2, which is incorporated herein by reference. In certain embodiments, more than one exogenous sequence is introduced.

By the term “modified” as used herein, is meant a changed state or structure of a molecule or cell of the invention. Molecules may be modified in many ways, including chemically, structurally, and functionally. Cells may be modified through the introduction of nucleic acids. Modifying can refer to altering expression of a gene in a lymphocyte, for example, by introducing an exogeneous nucleic acid that encodes the gene.

The lymphocytes provided herein can be genetically modified, e.g., by transfection, transduction, or electroporation, to express a nucleic acid sequence encoding a fusion protein. TCR, or CAR, as described herein. Depending on the clinical context, e.g., patient's condition or condition to be treated, prolonged or permanent expression of the gene and/or, e.g., for robust and long-lasting CAR activity, e.g., anti-tumor activity, may be desirable. In such embodiments, the lymphocytes are genetically modified, e.g., transduced, e.g., virally transduced, using vectors comprising nucleic acid sequences encoding a gene disclosed herein to confer a desired effector function. In other embodiments, transient expression of the gene is desirable. In such embodiments, the use of, e.g., mRNA or a regulatable promoter to express the effector-enhancing gene, may be used.

Methods of introducing and expressing genes into a cell are known in the art. In the context of an expression vector, the vector can be readily introduced into a host cell, e.g., mammalian, bacterial, yeast, or insect cell by any known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012. MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Viral vectors can be derived from lentivirus, poxviruses, herpes simplex virus I, adenoviruses, and adeno-associated viruses, and the like.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of targeted delivery of nucleic acids are available, such as delivery of polynucleotides with targeted nanoparticles or other suitable sub-micron sized delivery system. In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant nucleic acid sequence in the host cell, a variety of assays may be performed. Such assays include, for example. Southern and Northern blotting, RT-PCR and PCR, biochemical assays, such as detecting the presence or absence of a particular peptide, e.g., by immunological means (ELISAs and Western blots)

In certain embodiments, an expression vector is provided which includes the coding sequence for a fusion protein comprising an LTBR domain and a domain from a protein that is not LTBR. In other embodiments, the expression vector includes the coding sequence for one or more components of a CAR or TCR. In other embodiments, the expression vector includes the coding sequence for one or more components of a CAR or TCR that includes one or more LTBR domains. In other embodiments, the expression vector includes the coding sequence for one or more components of a CAR or TCR that includes one or more domains of a gene of Table I Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. In one embodiment, the expression vector is a lentivirus. If more than one expression vector is utilized, each expression vector may be individually selected from amongst those known in the art.

Provided herein is a method of making a population of immune effector cells (e.g., T cells, NK cells) that are modified to express a fusion protein, TCR, or CAR as described herein. Methods for making such immune cells include introducing an exogenous nucleic acid encoding a LTBR-domain fusion protein into the cell. Also provided herein is a method of making a population of immune effector cells (e.g. T cells. NK cells) that are modified to express a CAR or TCR that includes one or more LTBR domains. Methods for making such immune cells include introducing an exogenous nucleic acid encoding the CAR or TCR into the cell. In certain embodiments, immune effector cells comprising the fusion proteins described herein are long lived and/or resistant to apoptosis.

In the examples described below, a method of making modified T cells is described for convenience. However, alternative embodiments are envisioned using other kinds of immune cells, e.g., NK T cells or NK cells. Suitable methods are known in the art.

Briefly, an exemplary method includes providing a population of immune effector cells (e.g., T cells), and optionally, removing T regulatory cells, e.g., CD25+ T cells, from the population. In one embodiment, the population of immune effector cells are autologous to the subject who the cells will be administered to for treatment. In one embodiment, the population of immune effector cells are allogeneic to the subject who the cells will be administered to for treatment. In one embodiment, the T regulatory cells, e.g., CD25+ T cells, are removed from the population using an anti-CD25 antibody, or fragment thereof, or a CD25-binding ligand, e.g., IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein. In one embodiment, the T regulatory cells, e.g., CD25+ T cells, are removed from the population using an anti-CD25 antibody molecule, or fragment thereof. In another embodiment, CD25+ cells are not removed.

Another exemplary method includes providing a population of immune effector cells (e.g., T cells), and enriching the population for CD8+ cells and subsequently, enriching the population for CD4+ cells. In one embodiment, population is enriched for CD8+ and CD4+ T cells using an anti-CDS and anti-CD4 antibody, or fragment thereof, or a CD8-binding ligand or CD4-binding ligand. In one embodiment, the anti-CD4 or anti-CD8 antibody, or fragment thereof, or CD4 or anti-CD8-binding ligand is conjugated to a substrate, e.g., a bead, or is otherwise coated on a substrate, e.g., a bead. In one embodiment, the anti-CD4 antibody or anti-CD8, or fragment thereof, is conjugated to a substrate as described herein.

In one embodiment, the method further comprises transducing a cell from the population with one or more vectors comprising a nucleic acid encoding a fusion protein. CAR, or TCR as described herein. In one embodiment, the vector is selected from the group consisting of a DNA, a RNA, a plasmid, a lentivirus vector, adenoviral vector, or a retrovirus vector. In one embodiment, the cell from the population of T cells is transduced with a vector once, e.g., within one day after population of immune effector cells are obtained from a blood sample from a subject, e.g., obtained by apheresis. In one embodiment, the method further comprises generating a population of RNA-engineered cells transiently expressing exogenous RNA from the population of T cells. The method comprises introducing an in vitro transcribed RNA or synthetic RNA into a cell from the population, where the RNA comprises a nucleic acid encoding an LTBR domain-containing fusion protein, a TCR, or CAR.

In another embodiment, the method further comprises transducing a cell from the population with one or more vectors comprising a nucleic acid encoding a CAR or TCR that includes one or more LTBR domains as described herein. In one embodiment, the vector is selected from the group consisting of a DNA, a RNA, a plasmid, a lentivirus vector, adenoviral vector, or a retrovirus vector. In one embodiment, the cell from the population of T cells is transduced with a vector once, e.g., within one day after population of immune effector cells are obtained from a blood sample from a subject, e.g., obtained by apheresis. In one embodiment, the method further comprises generating a population of RNA-engineered cells transiently expressing exogenous RNA from the population of T cells. The method comprises introducing an in vitro transcribed RNA or synthetic RNA into a cell from the population, where the RNA comprises a nucleic acid encoding a CAR or TCR that includes one or more LTBR domains as described herein.

In one embodiment, modified cells described herein are expanded. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded in culture for 3 or 4 days, and the resulting cells are more potent than the same cells expanded in culture for 9 days under the same culture conditions. Potency can be defined, e g, by various T cell functions, e.g., proliferation, target cell killing, cytokine production, activation, migration, or combinations thereof.

### Methods of Treatment

Also provided herein, in certain aspects, are methods of treating cancer in a subject. In certain embodiments, the method includes administering to the subject a cell that expresses a fusion protein as described herein such that the cancer is treated in the subject. In certain embodiments, the cell further expresses a CAR. In other embodiments, the cell further expresses a TCR. As used herein in describing the methods of treatment, LTBR is utilized as an exemplary effector-enhancing gene, for convenience. However, in alternative embodiments, the other genes of Table 3 are also utilized in modified CAR or TCR containing cells in the methods. In one embodiment, the method includes obtaining cells from a patient, modifying the cells as described herein, and administering the cells to the patient.

In certain embodiments, the method includes administering to the subject a cell that expresses a CAR or TCR that includes one or more LTBR domains as described herein such that the cancer is treated in the subject. In one embodiment, the method includes obtaining cells from a patient, modifying the cells as described herein, and administering the cells to the patient.

In certain embodiments, the method includes administering to the subject a cell that expresses a CAR or TCR that includes one or more domains from a gene of Table 3 as described herein such that the cancer is treated in the subject. In one embodiment, the method includes obtaining cells from a patient, modifying the cells as described herein, and administering the cells to the patient.

The term “cancer” as used herein refers to any disease, condition, trait, genotype or phenotype characterized by unregulated cell growth or replication. In certain embodiments, administration of the compositions disclosed herein, e.g., according to the methods disclosed herein, treats a cancer. In certain embodiments, the cancer is selected from the group consisting of adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin. Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, hepatocellular carcinoma (HCC), non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor, secondary cancers caused by cancer treatment, and any combination thereof. An example of a cancer that is treatable by the modified cell (e.g., LTBR CART or LTBR TCR-T cell, or LTBR-containing CART or TCR-T cell) is a hematological cancer. In one aspect, a hematologic cancer including but is not limited to leukemia (such as acute myelogenous leukemia, chronic myelogenous leukemia, acute lymphoid leukemia, chronic lymphoid leukemia and myelodysplastic syndrome) and malignant lymphoproliferative conditions, including lymphoma (such as multiple myeloma, non-Hodgkin's lymphoma, Burkitt's lymphoma, and small cell- and large cell-follicular lymphoma). In other embodiments, a hematologic cancer can include minimal residual disease, MRD, e.g., of a leukemia, e.g., of AML or MDS. Other cancers include breast cancer, lung cancer, prostate cancer, colorectal cancer, esophageal cancer, stomach cancer, bladder cancer, pancreatic cancer, kidney cancer, cervical cancer, liver cancer, ovarian cancer, and testicular cancer. In other embodiments, the cancer is a solid tumor cancer. In certain embodiments, the cancer is one of those listed in FIG. 19 or FIG. 20.

In certain embodiments, the CAR is selected from Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kyrmriah®).

Autoimmune diseases are conditions arising from abnormal immune attack to the body, and they substantially increase the morbidity, mortality and healthcare costs worldwide. As T cells play a key role in the process of autoimmune diseases, engineered T-cell therapy has emerged and is also regarded as a potential approach to overcome current roadblocks in the treatment of autoimmune diseases. Either self-reactive or autoantibodies play a key role in the process of autoimmune diseases. Thus, engineering T cells to express a chimeric autoantibody receptor (CAAR) is a strategy for treatment for autoimmune disease. In one embodiment, the CAR comprises a CAAR. See, e.g., Zhang et al, Chimeric antigen receptor T-cell therapy beyond cancer: current practice and future prospects, Immunotherapy, 2020 September; 12(13):1021-1034, doi: 10.2217/imt-2020-0009. Epub 2020 Jul. 30, which is incorporated herein by reference. Autoimmune diseases include Pemphigus vulgaris (PV) (e.g., DSG3-CAAR-T) and lupus (e.g., MuSK-CAAR-T)). Other autoimmune diseases include type 1 diabetes, autoimmune thyroid disease, rheumatoid arthritis (RA), inflammatory bowel disease, colitis, systemic lupus erythematosus, and multiple sclerosis (MS). Sec, e.g., Chen et al, Immunotherapy Deriving from CAR-T Cell Treatment in Autoimmune Diseases, Journal of Immunology Research Volume 2019 Dec. 31, 2019, which is incorporated herein by reference. Other conditions treatable with the compositions described herein include graft-versus-host disease (GVHD) and transplant rejection.

In another embodiment, the subject bas a virally-driven cancer. In certain embodiments, the virally-driven cancer is selected from the following:

| Virus | Conditions |
|---|---|
| Epstein-Barr Virus (EBV) | Burkitt's Lymphoma |
| Hepatitis B Virus (HBV) | Liver Cancer |
| Hepatitis C Virus (HCV) | Liver Cancer |
| Human Herpesvirus 8 (HH8) | Kaposi's Sarcoma |
| Human Papillomavirus (HPV) | Cervical Cancer, Head and |
| | Neck Cancers, Anal, Oral, |
| | Pharyngeal, and Penile Cancers |
| Human T-cell Lymphotropic | Adult T-cell Leukemia |
| Virus 1 (HTLV) | |
| Merkel Cell Polyomavirus | Skin Cancer (Merkel Cell Carcinoma) |

In one aspect, the methods comprise administering to the subject in need thereof an effective amount of an effector-enhancing gene-expressing cell (e.g., LTBR CART or LTBR TCR-T cell) or LTBR-containing CAR/TCR expressing cell (LTBR-containing CART or TCR-T cell) described herein in combination with an effective amount of another therapy. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

An effector-enhancing gene-expressing cell (e.g., LTBR CART or LTBR TCR-T cell) or LTBR-containing CAR/TCR expressing cell (LTBR-containing CART or TCR-T cell) described herein and the at least one additional therapeutic agent can be administered simultaneously, in the same or in separate compositions, or sequentially. For sequential administration, the effector-enhancing gene-expressing cell (e.g., LTBR CART or LTBR TCR-T cell) cell described herein can be administered first, and the additional agent can be administered second, or the order of administration can be reversed.

The effector-enhancing gene-expressing cell (e.g., LTBR CART or LTBR TCR-T cell) or LTBR-containing CAR/TCR expressing cell (LTBR-containing CART or TCR-T cell) and/or other therapeutic agents, procedures or modalities can be administered during periods of active disorder, or during a period of remission or less active disease. The effector-enhancing gene-expressing cell (e.g., LTBR CART or LTBR TCR-T cell) or LTBR-containing CAR/TCR expressing cell (LTBR-containing CART or TCR-T cell) can be administered before the other treatment, concurrently with the treatment, post-treatment, or during remission of the disorder.

When administered in combination, the effector-enhancing gene-expressing cell (e.g., LTBR CART or LTBR TCR-T cell) or LTBR-containing CAR/TCR expressing cell (LTBR-containing CART or TCR-T cell) and the additional agent (e.g., second or third agent), or all, can be administered in an amount or dose that is higher, lower or the same than the amount or dosage of each agent used individually, e.g., as a monotherapy. In certain embodiments, the administered amount or dosage of the effector-enhancing gene-expressing cell (e.g., LTBR CART or LTBR TCR-T cell) or LTBR-containing CAR/TCR expressing cell (LTBR-containing CART or TCR-T cell), the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the effector-enhancing gene-expressing cell (e.g., LTBR CART or LTBR TCR-T cell) or LTBR-containing CAR/TCR expressing cell (LTBR-containing CART or TCR-T cell), the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, a effector-enhancing gene-expressing cell (e.g., LTBR CART or LTBR TCR-T cell) or LTBR-containing CAR/TCR expressing cell (LTBR-containing CART or TCR-T cell) described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxan, fludarabine, cyclosporin, FK506, rapamycin, mycophenolic acid, steroids, FR901228, cytokines, irradiation, or a peptide vaccine, such as that described in Izumoto et al. 2008 J Neurosurg 108:963-971.

In certain instances, an effector-enhancing gene-expressing cell (e.g., LTBR CART or LTBR TCR-T cell) or LTBR-containing CAR/TCR expressing cell (LTBR-containing CART or TCR-T cell) as described herein are combined with other therapeutic agents, such as other anti-cancer agents, anti-allergic agents, anti-nausea agents (or anti-emetics), pain relievers, cytoprotective agents, and combinations thereof.

In one embodiment, an effector-enhancing gene-expressing cell (e.g., LTBR CART or LTBR TCR-T cell) or LTBR-containing CAR/TCR expressing cell (LTBR-containing CART or TCR-T cell) as described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine), an mTOR inhibitor, a TNFR glucocorticoid induced TNFR related protein (GITR) agonist, a proteasome inhibitor (e.g., aclacinomyein A, gliotoxin or bortezomib), an immunomodulator such as thalidomide or a thalidomide derivative (e.g., lenalidomide), or a checkpoint inhibitor (e.g., a PD-1 or PD-L1 inhibitor, e.g., Pembrolizumab (Keytruda), Nivolumab (Opdivo), Cemiplimab (Libtayo). Atezolizumab (Tecentriq), Avelumab (Bavencio), Durvalumab (Imfinzi)).

General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-UR), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin (R). Rubex®), etoposide (Vepesid (R), fludarabine phosphate (Fludara®), 5-fluorouracil (Adrucil®), Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar (R), L-asparaginase (ELSPAR®), leucovorin calcium, melphalan (Alkcran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Treatment with a combination of a chemotherapeutic agent and an effector-enhancing gene-expressing cell (e.g., LTBR CART or LTBR TCR-T cell) or LTBR-containing CAR/TCR expressing cell (LTBR-containing CART or TCR-T cell) described herein can be used to treat a hematologic cancer described herein, e.g., AML. In embodiments, the combination of a chemotherapeutic agent and an effector-enhancing gene-expressing cell (e.g., LTBR CART or LTBR TCR-T cell) or LTBR-containing CAR/TCR expressing cell (LTBR-containing CART or TCR-T cell) is useful for targeting, e.g., killing, cancer stem cells, e.g., leukemic stem cells, e.g., in subjects with AML. In embodiments, the combination of a chemotherapeutic agent and an effector-enhancing gene-expressing cell (e.g., LTBR CART or LTBR TCR-T cell) or LTBR-containing CAR/TCR expressing cell (LTBR-containing CART or TCR-T cell) is useful for treating minimal residual disease (MRD) MRD refers to the small number of cancer cells that remain in a subject during treatment, e.g., chemotherapy, or after treatment. MRD is often a major cause for relapse. The present invention provides a method for treating cancer, e.g., MRD, comprising administering a chemotherapeutic agent in combination with an effector-enhancing gene-expressing cell (e.g., LTBR CART or LTBR TCR-T cell) or LTBR-containing CAR/TCR expressing cell (LTBR-containing CART or TCR-T cell), e.g., as described herein.

In an embodiment, the chemotherapeutic agent is administered prior to administration of the effector-enhancing gene-expressing cell (e.g., LTBR CART or LTBR TCR-T cell) or LTBR-containing CAR/TCR expressing cell (LTBR-containing CART or TCR-T cell). In chemotherapeutic regimens where more than one administration of the chemotherapeutic agent is desired, the chemotherapeutic regimen is initiated or completed prior to administration of the effector-enhancing gene-expressing cell (e.g., LTBR CART or LTBR TCR-T cell) or LTBR-containing CAR/TCR expressing cell (LTBR-containing CART or TCR-T cell). In embodiments, the chemotherapeutic agent is administered at least 1 day, 2 days, 3 days, 4 days, 5 days, 6 days, 7 days, 8 days, 9 days, 10 days, 11 days, 12 days, 13 days, 14 days, 15 days, 20 days, 25 days, or 30 days prior to administration of the effector-enhancing gene-expressing cell (e.g., LTBR CART or LTBR TCR-T cell) or LTBR-containing CAR/TCR expressing cell (LTBR-containing CART or TCR-T cell). In embodiments, the chemotherapeutic regimen is initiated or completed at least 1 day, 2 days, 3 days, 4 days, 5 days. 6 days, 7 days, 8 days. 9 days, 10 days, 11 days, 12 days. 13 days, 14 days, 15 days. 20 days. 25 days, or 30 days prior to administration of the effector-enhancing gene-expressing cell (e.g., LTBR CART or LTBR TCR-T cell) or LTBR-containing CAR/TCR expressing cell (LTBR-containing CART or TCR-T cell).

When “an immunologically effective amount,” “an anti-tumor effective amount,” “a tumor-inhibiting effective amount,” or “effective amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 104 to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J, of Med. 319:1676, 1988). As used herein, the term “effective amount” of an agent is that amount sufficient to effect beneficial or desired results, for example, clinical results, and, as such, an “effective amount” depends upon the context in which it is being applied. For example, in the context of administering an agent that treats a tumor, an effective amount of an agent is, for example, an amount sufficient to reduce or decrease a size of a tumor or to inhibit a tumor growth, as compared to the response obtained without administration of the agent. The term “effective amount” can be used interchangeably with “effective dose,” “therapeutically effective amount.” or “therapeutically effective dose.”

Also provided herein is a method of vaccinating a subject with a combination vaccine including at least two nucleic acid sequences encoding at least one effector-enhancing gene and at least one viral protein. In one embodiment, the effector-enhancing gene is LTBR. In another embodiment, the viral protein is a coronavirus spike protein. Some embodiments provide vaccines comprising an RNA polynucleotide having an open reading frame encoding an effector-enhancing gene, an RNA polynucleotide having an open reading frame encoding an effector-enhancing gene a viral protein, and a pharmaceutically acceptable carrier or excipient, formulated within a cationic lipid nanoparticle (LNP) The vaccines described herein (e.g., LNP-encapsulated mRNA vaccines) produce prophylactically- and/or therapeutically-efficacious levels, concentrations and/or titers of antigen-specific antibodies in the blood or serum of a vaccinated subject. Sec, e.g., US 2018/0311336A1, which is incorporated herein by reference in its entirety.

As used herein, the term “treatment,” and variations thereof such as “treat” or “treating.” refers to clinical intervention in an attempt to alter the natural course of the individual being treated and can be performed either for prophylaxis or during the course of clinical pathology. Desirable effects of treatment include, but are not limited to, preventing or reducing the occurrence or recurrence of disease, alleviation of symptoms, diminishment of any direct or indirect pathological consequences of the disease, decreasing the rate of disease progression, amelioration or palliation of the disease state, and remission or improved prognosis. In certain embodiments, compositions described herein are used to delay development of a disease or to slow the progression of a disease.

Likewise, as used herein, the term “treatment of cancer” or “treating cancer” can be described by a number of different parameters including, but not limited to, reduction in the size of a tumor in an animal having cancer, reduction in the growth or proliferation of a tumor in an animal having cancer, preventing metastasis or reducing the extent of metastasis, and/or extending the survival of an animal having cancer compared to control. In certain embodiments, treatment results in a reduced risk of distant recurrence or metastasis.

With regard to the description of the inventions provided herein, it is intended that each of the compositions described, is useful, in another embodiment, in the methods of the invention. In addition, it is also intended that each of the compositions herein described as useful in the methods, is, in another embodiment, itself an embodiment of the invention.

The following examples are provided for the purpose of illustration only and the invention should in no way be construed as being limited to this example but rather should be construed to encompass any and all variations that become evident as a result of the teachings provided herein.

### EXAMPLES

### Example 1: Materials and Methods

### Isolation and Culture of Primary Human T Cells

Standard buffy coats containing peripheral blood from de-identified healthy donors were collected by and purchased from the New York Blood Center under an IRB-exempt protocol. All donors provided informed consent. Peripheral blood mononuclear cells (PBMC) were isolated from buffy coats using Lymphoprep (Stemcell) gradient centrifugation. For most assays, CD8⁺ and CD4⁺ were isolated sequentially from the same donor. First, CD8⁺ T cells were isolated by magnetic positive selection using the Easy Sep Human CD8 Positive Selection Kit II (Stemcell). Then, CD4⁺ T cells were isolated from the resulting flowthrough by negative magnetic selection using the Easy Sep Human CD4⁺ T cell Isolation Kit (Stemcell), γδ T cells were isolated by magnetic negative selection using the EasySep Human Gamma/Delta T cell Isolation Kit (Stemcell). Immediately after isolation. T cells were resuspended in T cell medium, which consisted of Immunocult-XF T cell Expansion Medium (Stemcell) supplemented with 10 ng ml⁻¹ recombinant human IL-2 (Stemcell).

Activation of T cells was performed with Immunocult Human CD3/CD28 T cell Activator (Stemcell) using 25 μl per 10⁶ cells per ml. Typically. T cells were transduced with concentrated lentivirus 24 h after isolation. For some experiments, T cells were electroporated with in-vitro-transcribed mRNA 24 h after isolation or with Cas9 protein 48 h after isolation. At 72 h after isolation, lentivirally transduced T cells were selected with 2 μg ml⁻¹ puromycin.

Every 2-3 days, T cells were either split or had the medium replaced to maintain a cell density of 1×10⁶−2×10⁶ cells per ml. Lentivirally transduced T cells were maintained in medium containing 2 μg ml⁻¹ puromycin for the duration of culture. T cells were used for phenotypic or functional assays between 14 and 21 days after isolation, or cryopreserved in Bambanker Cell Freezing Medium (Bulldog Bio), γδ T cells were further purified before functional assays using anti-Vγ9) PE antibody (Biolegend) and anti-PE microbeads (Miltenyi Biotec) according to the manufacturer's recommendations, in the presence of dasatinib, a protein kinase inhibitor, to prevent activation-induced cell death resulting from TCR cross-linking⁴². PBMCs from patients with diffuse large B cell lymphoma were obtained from the Perlmutter Cancer Center under a protocol approved by the Perlmutter Cancer Center Institutional Review Board (S14−02164).

### Vector Design and Molecular Cloning

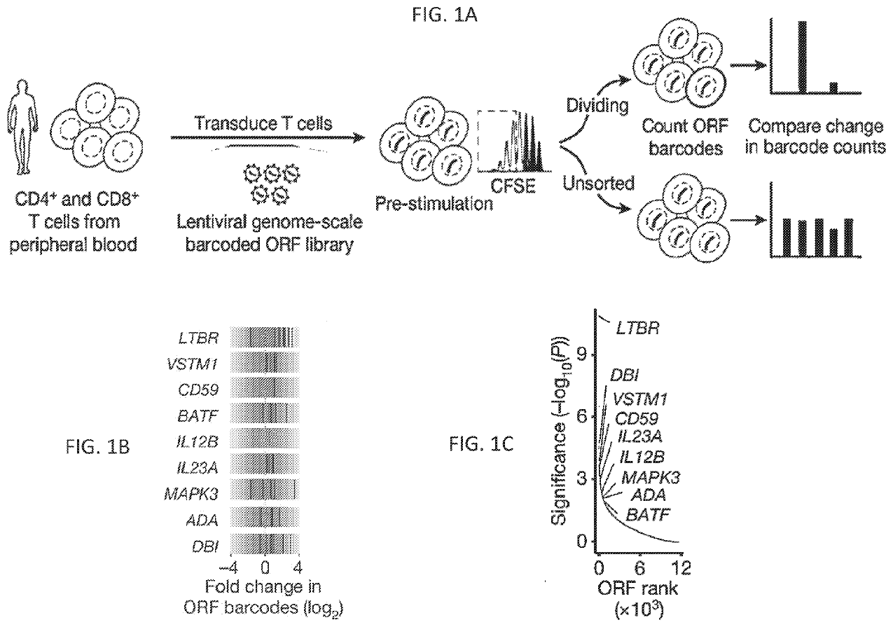

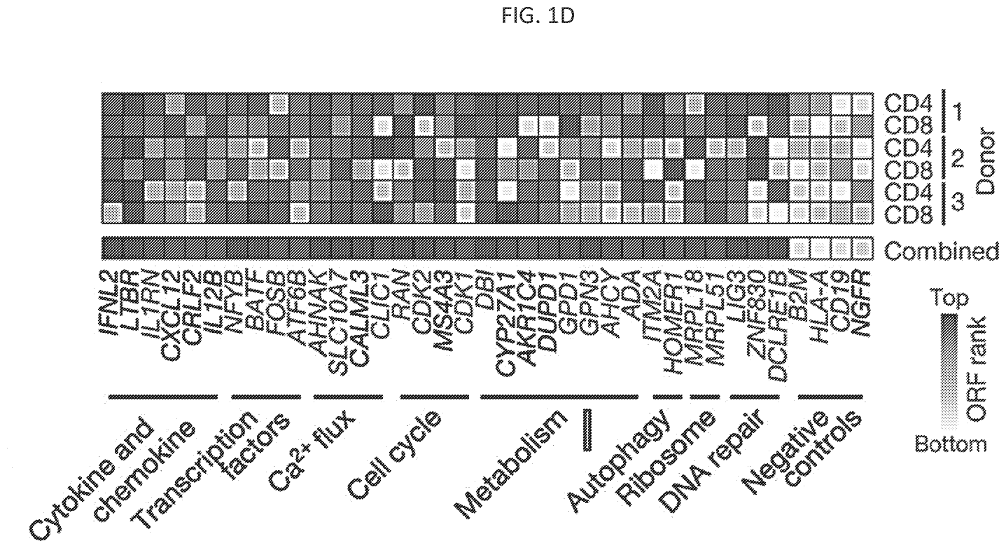

All vectors used were cloned using Gibson Assembly (NEB). For the experiments shown in FIG. 1A-FIG. 1D, we used the lentiviral backbone from the pHAGE plasmid¹⁴. For all other experiments, the backbone from lentiCRISPRv2 (Addgene 52961) was used. ORFs were PCR-amplified for cloning from the genome-scale library used in the screen.

After adding Gibson overhangs by PCR, ORFs and P2A-puro were inserted into XbaI- and EcoRI-cut lentiCRISPRv2. The sgRNA cassette was removed from JentiCRISPRv2 using PacI and NheI digest. For LTBR overexpression and knockout experiments, the sgRNA cassette was not removed. CARs were synthesized as gBlocks (IDT). For CAR-ORF cloning, CAR-P2A-puro-T2A (partial) were first inserted into XbaI- and EcoRI-cut lentiCRISPRv2. For subsequent ORF insertion, the plasmid was cut with HpaI located within the partial T2A and EcoRI The following vectors were deposited to Addgene: pOT_01 (lenti-EFS-LTBR-2A-puro, Addgene 181970), pOT_02 (lenti-EFS-tNGFR-2A-puro, Addgene 181971), pOT_03 (lenti-EFS-FMC6.3-28z-2A-puro-2A-LTBR, Addgene 181972), pOT_04 (lenti-EFS-FMC6.3-BBz-2A-puro-2A-LTBR, Addgene 181973), pOT_05 (lenti-EFS-FMC6.3-28z-2A-puro-2A-tNGFR, Addgene 181974) and pOT_06 (lenti-EFS-FMC6.3-BBz-2A-puro-2A-tNGFR, Addgene 181975).

### Nuclease and CRISPR Guide RNA Design

All sgRNAs were designed using the GUIDES webtool⁴³. We selected guides that target initial protein-coding exons (with the preference for targeting protein family domains enabled in GUIDES) as well as minimizing off-target and maximizing on-target scores. For Cas9 nuclease nucleofection, we used purified sNLS-SpCas9-sNLS nuclease (Aldevron).

Preparation of ORF library plasmids for paired-end sequencing We re-amplified a previously described genome-scale ORF library¹⁴ using Endura electrocompetent cells (Lucigen). The identity of ORFs and matched barcodes was confirmed by paired-end sequencing. In brief, the plasmid was first linearized with I-SceI meganuclease, which cuts downstream of the barcode. Then, the linearized plasmid was tagmented using TnY transposase⁴⁴. Then, the fragmented plasmid was amplified in a PCR reaction, using a forward primer binding to a handle introduced by TnY and a reverse primer binding to a sequence downstream of the barcode. All transposons and PCR primer oligonucleotides were synthesized by IDT. The resulting amplicon was sequenced on a NextSeq 500. The forward read (containing the ORF) was mapped to GRCh38.101 CDS transcriptome annotations using STAR v.2.7.3a (map quality ≥10)⁴⁵. Using the paired-end read, we also captured the 24 nucleotide barcode downstream of the constant plasmid sequence. We tabulated ORF-barcode combinations and further curated this table by eliminating any spurious pairs that might be due to sequencing or PCR error. Specifically, a permutation test was performed to identify the maximum number of ORF-barcode combinations expected by random chance, after which we only kept ORF-barcode combinations with a count that exceeded this maximum number We excluded all non-coding elements from the reference and then collapsed barcodes that were within a Levenshtein distance less than 2.

### Cell Culture

HEK293FT cells were obtained from Thermo Fisher Scientific and cultured in Dulbecco's modified Eagle's medium (DMEM) supplemented with 10% Serum Plus-II (Thermo Fisher Scientific). Nalm6, Jurkat and BxPC3 cells were obtained from ATCC and cultured in RPMI-1640 supplemented with 10% Scrum Plus-II. Capan-2 cells were obtained from ATCC and cultured in McCoy's medium supplemented with 10% Serum Plus-II. For γδco-incubation experiments, cell lines were pre-treated with 50 μM zoledronic acid (Sigma) for 24 h. Cell lines were routinely tested for mycoplasma using MycoAlert PLUS (Lonza) and found to be negative. Cell lines were not authenticated in this study.

### Lentivirus Production

We produced lentivirus by co-transfecting third-generation lentiviral transfer plasmids together with packaging plasmid psPAX2 (Addgene 12260) and envelope plasmid pMD2.G (Addgene 12259) into HEK293FT cells, using polyethyleneimine linear MW 25000 (Polysciences). After 72 h, we collected the supernatants, filtered them through a 0.45-μm Steriflip-HV filter (Millipore) and concentrated the virus using Lentivirus Precipitation Solution (Alstem). Concentrated lentivirus was resuspended in T cell medium containing IL-2 and stored at-80 Cc.

### Pooled ORF Library Screening

For pooled ORF library screening, CD4⁺ and CD8⁺ T cells were isolated from a minimum of 500×10⁶ PBMCs from 3 healthy donors. The amount of lentivirus used for transduction was titrated to result in 20-30% transduction efficiency, to minimize the probability of multiple ORFs being introduced into a single cell. The cells were maintained in T cell medium containing 2 μg ml-1 puromycin and counted every 2-3 days to maintain a cell density of 1×10⁶-2×10⁶ cells per ml. On day 14 after isolation, T cells were collected, counted, labelled with 5 μM CFSE (Biolegend) and stimulated with CD3/CD28 Activator (Stemcell) at 1.56 μl per 1×10⁶ cells. An aliquot of cells representing 1,000>coverage of the library was frozen down at this step to be used as a pre-stimulation control. After 4 days of stimulation, cells were collected and an aliquot of cells representing 1,000× coverage of the library was frozen down to be used as a pre-sort control. The remaining cells were stained with LIVE/DEAD Violet cell viability dye (Thermo Fisher Scientific), and CFSE^{low} cells (corresponding to the bottom 15% of the distribution) were sorted using a Sony SH800S cell sorter. Genomic DNA was isolated, and two rounds of PCR to amplify ORF barcodes and add Illumina adaptors were performed⁴⁶.

### Pooled ORF Screen Analysis

For most of the analyses, equal numbers of reads from all three donors were combined per bin before trimming and alignment. The barcodes were mapped to the reference library after adaptor trimming with Cutadapt v. 1.13 (-m 24-e 0.1—discard-untrimmed) using Bowtie v. 1.1.2 (-v 1-m 1—best—strata)+7.48. All subsequent analyses were performed in RStudio v.1.1.419 with R 4.0.0.2. To calculate individual barcode enrichment, barcode counts were normalized to the total number of reads per sample (with pseudocount added) and log₂-transformed. To calculate ORF enrichment, raw barcode counts were first collapsed by genes before normalization and log₂ transformation.

We performed enrichment analyses at both the barcode and gene level. Statistical analysis on barcode enrichment was performed using MAGeCK⁴⁹, comparing CFSE^{low} samples to corresponding inputs (pre-stimulation), using CD4⁺ and CD8⁺ as replicates. Statistical analysis on ORF enrichment was performed using DESeq2⁵⁰. We obtained raw gene counts by collapsing barcodes into corresponding genes. CFSE^{low} samples were compared to corresponding inputs (both pre-stimulation and pre-sort), using CD4⁺ and CD8⁺ as replicates. GO enrichment (biological process) on genes passing DESeq2 criteria (log₂-transformed fold change >0.5, P_{adj}<0.05) was performed using the topGO package⁵¹. For the genes enriched in the CFSE^{low} screen (DESeq2 analysis), we overlapped these genes with differentially expressed genes after CD3/CD28 stimulation using data from the Database of Immune Cell eQTLs. Expression, Epigenomics (DICE: https://dice-database.org/)⁴¹. For differentially expressed genes, we used the following DICE datasets: ‘T cell, CD4, naive’ versus ‘T cell, CD4, naive [activated]’, ‘T cell. CD8, naive’ versus ‘T cell, CD8, naïve [activated]’. Significant differential expression was as given in the DICE dataset (P_{adj}<0.05).

### Proliferation Assays

Transduced T cells were collected at day 14 after isolation, counted and plated at 2.5×10⁴ cells per well in a round bottom 96-well plate, in 2 sets of triplicate wells per transduction. One set of triplicate wells was cultured in Immunocult-XF T cell Expansion Medium supplemented with 10 ng ml⁻¹ IL-2 and another set of triplicate wells was further supplemented with 1.56 μl CD3/CD28 Activator per 1 ml of medium. The cells were cultured for 4 days, and then were collected and stained with LIVE/DEAD Violet cell viability dye. Before flow cytometric acquisition, the cells were resuspended in D-PBS with 10% v/v Precision Counting Beads (Biolegend). For quantification, the number of viable cell events was normalized to the number of bead events per sample. Then, for each ORF the normalized number of viable cells in wells supplemented with CD3/CD28 Activator was divided by the mean number of viable cells in control wells to quantify T cell proliferation. To enable comparisons between donors and CD4⁺/CD8⁺ T cells, the proliferation of T cells transduced with a given ORF was finally normalized to the proliferation of a matched tNGFR control.

In addition to the counting beads assay, we also measured proliferation using a dye dilution assay. For this assay, transduced T cells were collected at day 14 after isolation, washed with D-PBS and then labelled with 5 μM CellTrace Yellow (CTY) in D-PBS for 20 min at room temperature. The excess dye was removed by washing with a fivefold excess of RPMI-1640 supplemented with 10% Serum Plus-II. The labelled cells were then plated at 2.5×10⁴ cells per well on a round bottom 96-well plate. One set of triplicate wells was cultured in supplemented Immunocult-XF T cell Expansion Medium (that is, without IL-2) and another set of triplicate wells was supplemented with 10 ng ml⁻¹ IL-2 and 1.56 μl CD3/CD28 Activator per 1 ml of medium. The cells were cultured for 4 days, and then were collected and stained with LIVE/DEAD Violet cell viability dye. For quantification of the proliferation index, events were first gated on viable T cells in FlowJo (Treestar) and exported for further analysis in R/RStudio using the flowFit and flow Core packages⁵². Unstimulated cells were used to determine the parent population size and position to account for differences in staining intensity between different samples. These fitted parent population parameters were then used to fit the CTY profiles of matched stimulated samples, modelled as Gaussian distributions assuming log₂-distanced peaks as a result of cell division and dye dilution. Fitted CTY profiles were inspected visually for concordance with the original CTY profiles and used to calculate the proliferation index. The proliferation index is defined as the sum of cells in all generations divided by the computed number of parent cells present at the beginning of the assay.

### Flow Cytometry for Cell-Surface and Intracellular Markers

For CD25 (IL2RA) and CD154 (CD40L) quantification, T cells were restimulated with CD3/CD28 Activator (6.25 μl per 10⁶ cells) for 6 h (CD154 staining in CD8⁺) or for 24 h before staining (CD25 staining in both CD4⁺ and CD8⁺, and CD154 staining in CD4⁺). For Ki-67 and 7-amino-actinomycin D (7-AAD) staining, T cells were rested overnight in Immunocult-XF T cell Expansion Medium without IL-2 and then activated with CD3/CD28 Activator (25 μl per 10⁶ cells) for 24 h. In other cases, T cells were stained without stimulation. For detection of secreted proteins. T cells were stimulated for 24 h with CD3/CD28 Activator (25 μl per 10⁶ cells) (LTA. LIGHT), and protein transport inhibitors brefeldin A (5 μg ml⁻¹) and monensin (2 μM) were included for the last 6 h of stimulation (IL12B, LTA, LIGHT).

First, the cells were collected, washed with D-PBS and stained with LIVE/DEAD Violet cell viability dye for 5 min at room temperature in the dark, followed by surface antibody staining for 20 min on ice. After surface antibody staining (where applicable) the cells were washed with PBS and acquired on a Sony SH800S cell sorter or taken for intracellular staining. For intracellular staining, the cells were resuspended in an appropriate fixation buffer. The following fixation buffers were used for specific protein detection: Fixation Buffer (Biolegend) for IL12B and MS4A3 staining: True-Nuclear Transcription Factor Fix (Biolegend) for BATF, TCF1 and FLAG staining; and FoxP3/Transcription Factor Fixation Reagent, (eBioscience) for Ki-67. After resuspension in the fixation buffer, cells were incubated at room temperature in the dark for 1 h. Following the incubation, the cells were washed twice in the appropriate permeabilization buffer. The following permeabilization buffers were used: Intracellular Staining Permeabilization Wash Buffer (Biolegend) for IL12B and MS4A3 staining: True-Nuclear Perm Buffer (Biolegend) for BATF, TCF1 and FLAG staining; and FoxP3/Transcription Factor Permeabilization Buffer (eBioscience) for Ki-67. After permeabilization, the cells were stained with the specific antibody or isotype control for 30 min in the dark at room temperature. Finally, the cells were washed twice in the appropriate permeabilization buffer and acquired on a Sony SH800S flow cytometer. For cell-cycle analysis, the cells were further stained with 0.5 μg ml⁻¹ 7-AAD for 5 min immediately before acquisition. Gating was performed using appropriate isotype, fluorescence minus one and biological controls. Typically, 5.000-10,000 live events were recorded per sample.

### Flow Cytometry Detection of Phosphorylated Proteins

T cells were rested for 24 h in in Immunocult-XF T cell Expansion Medium without IL-2 before detection of phosphorylated proteins. The rested cells were stimulated with CD3/CD28 Activator (25 μl per 10⁶ cells) for the times indicated in the corresponding figure. Immediately after the stimulation period, the cells were fixed with a 1:1 volume ratio of the pre-warmed Fixation Buffer (Biolegend) for 15 min at 37° C., and washed twice with the cell staining buffer (D-PBS+2% FBS). As per the manufacturer's protocol, the cells were resuspended in the residual volume and permeabilized in 1 ml of pre-chilled True-Phos Perm Buffer (Biolegend) while vortexing. The cells were incubated in the True-Phos Perm Buffer for 60 min at −20° C. After permeabilization the cells were washed twice with the cell staining buffer and stained with anti-CD4, anti-CD8, anti-RELA and anti-phospho-RELA antibodies (or isotype controls) for 30 min at room temperature. After staining, the cells were washed twice in the cell staining buffer and acquired on a Sony SH800S cell sorter. Gating was performed on CD4⁺ or CD8⁺ cells, and the levels of RELA and phospho-RELA were determined using appropriate isotype and biological controls.

### Western Blot Detection of Proteins and Phosphorylated Proteins

T cells expressing tNGFR or LTBR, resting or stimulated for 15 min with CD3/CD28 Activator (25 μl per 10⁶ cells), were collected, washed with 1×D-PBS and lysed with TNE buffer (10 mM Tris-HCl, pH 7.4, 150 mM NaCl, 1 mM EDTA, 1% Nonidet P-40) in the presence of a protease inhibitor cocktail (Bimake B14001) and a phosphatase inhibitor cocktail (Cell Signaling Technologies 5872S) for 1 h on ice. Cell lysates were spun for 10 min at 10,000 g, and the protein concentration was determined with the BCA assay (Thermo Fisher Scientific). Equal amounts of cell lysates (25 mg) were denatured in Tris-Glycine SDS Sample buffer (Thermo Fisher Scientific) and loaded on a Novex 4-12 or 4-20% Tris-Glycine gel (Thermo Fisher Scientific). The PageRuler pre-stained protein ladder (Thermo Fisher Scientific) was used to determine the protein size. The gel was run in Ix Tris-Glycine-SDS buffer (IBI Scientific) for about 120 min at 120 V. Proteins were transferred on a nitrocellulose membrane (BioRad) in the presence of prechilled 1× Tris-Glycine transfer buffer (Thermo Fisher Scientific) supplemented with 20% methanol for 100 min at 100 V.

Immunoblots were blocked with 5% skimmed milk dissolved in 1×PBS with 1% Tween-20 (PBST) and incubated overnight at 4° C.′ separately with the following primary antibodies: rabbit anti-GAPDH (0.1 mg ml⁻¹, Cell Signaling, 2118S), mouse anti-IKKα (1:1,000 dilution, Cell Signaling. 3G12), rabbit anti-IKKβ (1:1,000 dilution, Cell Signaling, D30C6), rabbit anti-NF-κB p65 (1:1,000 dilution, Cell Signaling, D14E12), rabbit anti-phospho-NF-κB p65 Ser536 (1:1,000 dilution. Cell Signaling, 93H1), mouse anti-IκBα (1:1,000 dilution, Cell Signaling, L35A5), rabbit anti-phospho-IκBα Ser32 (1:1,000 dilution, Cell Signaling, 14D4), rabbit anti-NF-κB p100/p52 (1:1,000 dilution, Cell Signaling, 4882) and rabbit anti-RELB (1:1,000 dilution. Cell Signaling, C1E4). After the primary antibody, the blots were incubated with IRDye 680RD donkey anti-rabbit (0.2 mg ml⁻¹, LI-COR 926-68073) or with IRDye 800CW donkey anti-mouse (0.2 mg ml⁻¹, LI-COR 926-32212). The blots were imaged using Odyssey CLx (LI-COR) and quantified using ImageJ v. 1.52.

### Quantification of Cytokine Secretion

For measurement of secreted IFNγ and IL-2, T cells were first collected and rested for 24 h in medium without IL-2. Then, they were counted, plated at 2.5×10⁴ cells per well in a round bottom 96-well plate and incubated in medium without IL-2, with or without CD3/CD28 Activator (25 μl per 10⁶ cells) for 24 h. Then, cell supernatants were collected, diluted and used for cytokine quantification with an enzyme-linked immunosorbent assay (Human IL-2 or IFNγ DuoSet, R&D Systems), using an Infinite F200 Pro (Tecan) plate reader. Multiplexed quantification of secreted cytokines and chemokines in resting or stimulated T cells was performed using the Human Cytokine/Chemokine 48-Plex Discovery Assay Array (Eve Technologies).

### T Cell Killing Assays

CD19⁺ Nalm6 cells were first transduced with a lentiviral vector encoding EGFPd2PEST-NLS and a puromycin resistance gene⁵³. The transduced cells were kept in puromycin selection throughout the culture, to maintain stable EGFP expression, and puromycin was only removed from the medium before the killing assay. T cells were transduced with a vector encoding a CAR specific for CD19, using either a CD28 stalk, CD28 transmembrane and CD28 signaling domain or CD8 stalk and CD8 transmembrane domain with 4-1BB signaling domain, and CD3ζ signaling domain⁵⁴. Fourteen days after transduction, transduced T cells were combined with 5×10⁴ Nalm6 GFP⁺ cells in triplicate at indicated effector: target ratios in a flat 96-well plate pre-coated with 0.01% poly-1-ornithine (EMD Millipore) in Immunocult medium without IL-2. The wells were then imaged using an Incucyte SX1, using 20× magnification and acquiring 4 images per well every 2 h for up to 120 h. For each well, the integrated GFP intensity was normalized to the 2 h time point, to allow the cells to fully settle after plating.

In Vitro mRNA Preparation

The template for in vitro transcription was generated by PCR from a plasmid encoding LTBR or tNGFR with the resulting amplicon including a T7 promoter upstream of the ORF. The purified template was then used for in vitro transcription with capping and poly-A tailing using the HiScribe T7 ARCA mRNA Kit with Capping (NEB).

### Primary T Cell Nucleofection

Activated T cells were nucleofected with in-vitro-transcribed mRNA at 24 h after activation or with Cas9 protein at 48 h after activation. The cells were collected, washed twice in PBS and resuspended in P3 Primary Cell Nucleofector Solution (Lonza) at 5×10⁵ cells per 20 μl. Immediately after resuspension, 1 μg mRNA or 10 μg Cas9 (Aldevron) were added (not exceeding 10% v/v of the reaction) and the cells were nucleofected using the E0-115 program on a 4D-Nucleofector (Lonza). After nucleofection the cells were resuspended in pre-warmed Immunocult medium with IL-2 and recovered at 37° C., with 5% CO₂ for 20 min. After recovery, the cells were plated at 1×10⁶ cells per ml and used in downstream assays.

### OverCITE-Seq Sample Preparation and Sequencing

For single-cell sequencing. CD8⁺ T cells were individually transduced with ORFs and kept, separately, under puromycin selection for 14 days. Then, transduced cells were combined and split into two conditions: one was cultured for 24 h only in the presence of IL-2; the other was further supplemented with 6.25 μl CD3/CD28 Activator per 10⁶ cells. After stimulation, the cells were collected, counted and resuspended in staining buffer (2% BSA+0.01% Tween-20 in PBS) at 2×10⁷ cells per ml. Then, 10% (v/v) Human TruStain FcX Fc Receptor Blocking Solution (Biolegend) was added, and the cells were incubated at 4° C., for 10 min. After Fc receptor blocking, the cell concentration was adjusted to 5×10⁶ cells per ml and the stimulated and unstimulated cells were split into 4 conditions each. Each condition received a different oligonucleotide-conjugated (barcoded) cell hashing antibody to allow for pooling of different conditions in the same 10× Genomics Chromium lane²³. After 20 min co-incubation on ice, the cells were washed 3 times with staining buffer and counted using Trypan blue exclusion. Cell viability was typically around 95%.

Then, cells stained with different hashing antibodies were pooled together at equal numbers and stained with the following oligonucleotide-conjugated (barcoded) antibodies for quantification of cell surface antigens: CD11c (0.1 μg), CD14 (0.2 μg), CD16 (0, 1 μg), CD19 (0.1 μg), CD56 (0.2 μg), CD3 (0.2 μg), CD45 (0.01 μg), CD45RA (0.2 μg), CD45RO (0.2 μg), CD4 (0.1 μg), CD8(0.1 μg), CD25 (0.25 μg), CD69 (0.25 μg) and NGFR (0.25 μg) (TotalSeq-C, Biolegend). The cells were stained for 30 min on ice, washed 3 times with staining buffer, resuspended in PBS and filtered through a 40-μm cell strainer. The cells were then counted and the concentration was adjusted to 1×10⁶ ml⁻¹. For loading into the 10× Genomics Chromium, 3× 10⁴ cells were combined with Chromium Next GEM Single Cell 5′ v2 Master Mix (10× Genomics) supplemented with a custom reverse primer binding to the puromycin resistance cassette for boosting ORF transcript capture at the reverse transcription stage. The custom reverse primer was added at a 1:3 ratio to the poly-d′T primer included in the Master Mix.

For cDNA amplification, additive primers for amplification of sample hashing and surface antigen barcodes were included²³, as well as a nested reverse primer binding to the puromycin resistance cassette downstream of the ORF. Following cDNA amplification, SPRI beads were used for size selection of resulting PCR products: small-size (fewer than 300 bp) sample hashing and surface antigen barcodes were physically separated from larger cDNA and ORF amplicons for downstream processing. Sample hashing and surface antigen barcodes were also processed²². Amplified cDNA was then separated into three conditions, for construction of the gene expression library, of TCR library and ORF library. The ORF library was processed similarly to the af TCR library, using nested reverse primers binding downstream of the ORF. The quality of produced libraries was verified on BioAnalyzer using the High Sensitivity DNA kit (Agilent). The libraries were sequenced on a NextSeq 500. For the gene expression library, more than 25,000 reads per cell were generated. For other libraries, more than 5,000 reads per cell were generated.

### OverCITE-Seq Data Analysis

Gene expression unique molecular identifier (UMI) count matrices and TCR clonotypes were derived using 10× Genomics Cell Ranger 3.1.0. Hashtag oligo (HTO) and antibody UMI count matrices were generated using kallisto v.0.46.0⁵⁵ and bustools v.0.39.3⁵⁶. ORF reads were first aligned to plasmid references using Bowtie2 v.2.2.8⁵⁷ and indexed to the associated ORF, after which kallisto and bustools were used to generate UMI count matrices. All modalities were normalized using a centred log ratio (CLR) transformation. Cell doublets and negatives were identified using the HTODemux⁵⁸ function and then excluded from downstream analysis. The UMI cut-off quantile for HTODemux was optimized to maximize singlet recovery using grid search with values between 0 and 1. ORF singlets were identified using MULTIseqDemux⁵⁹. We then excluded cells with low-quality gene expression metrics and removed cells with fewer than 200 unique RNA features or greater than 5% of reads mapping to the mitochondrial transcriptome.

Count matrices were then loaded into and analyzed with Seurat v.4.0.1⁶⁰. Cell cycle correction and scaling of gene expression data was performed using the CellCycleScoring function with default genes, followed by scaling the data using the ScaleData function Principal component (PC) optimization of the scaled and corrected data was then performed using JackStraw⁶¹, in which we selected all PCs up to the first non-significant PC to use in clustering. Clustering of cells was performed using a shared nearest neighbor (SNN)-based clustering algorithm and visualized using UMAP dimensional reduction⁶² to project cluster PCs into 2D space. Cluster marker analysis was performed using the FindAllMarkers function with the hypothesis set defined as positive and negative markers present in at least 25% of cluster cells and with a log 2-transformed fold change threshold of 0.25 as compared to non-cluster cells. Differential expression analysis of ORFs was performed using DESeq2⁵⁰ to identify genes up and downregulated in ORF-expressing cells as compared to NGFR (control) cells, with differential expression defined as those with q<0.1 calculated using the Storey method⁶³.

### Bulk RNA-Seq and Analysis

CD4⁺ and CD8⁺ LTBR-or tNGFR-transduced T cells were stimulated for 24 h with CD3/CD28 Activator (25 μl per 10⁶ cells) or left unstimulated (n=3 biological replicates). Total RNA was extracted using the Direct-zol RNA purification kit (Zymo). The 3′-enriched RNA-seq library was prepared as described before⁶⁴. In brief, RNA was reverse-transcribed using SMARTScribe Reverse Transcriptase (Takara Bio) and a poly (dT) oligo containing a partial Nextera handle. The resulting cDNA was then PCR-amplified for 3 cycles using One Taq polymerase (NEB) and tagmented for 5 min at 55° C., using homemade transposase To Y⁴⁴. Immediately afterwards, the tagmented DNA was purified on a MinElute column (Qiagen) and PCR-amplified using OneTaq polymerase and barcoded primers for 12 cycles. The PCR product was purified using a dual (0.5×-0.8×) SPRI clean-up (Agencourt) and the size distribution was determined using Tapestation (Agilent). Samples were sequenced on a NextSeq 500 (Illumina) using a v2.5 75-cycle kit (paired end). Paired-end reads were aligned to the transcriptome (human Ensembl v.96 reference⁶⁵) using kallisto v.0.46.055 and loaded into RStudio 1.1.419 with R 4.0.0.2 using the tximport packaged⁶⁶. Differential gene expression analysis was performed using DESeq2⁵⁰. GO enrichment (biological process) on genes passing DESeq2 criteria (log:-transformed fold change >1. Padj <0.05) was performed using the topGO package⁵¹.

### ATAC-Seq Library Preparation

CD8+LTBR and tNGFR T cells were stimulated for 24 h with CD3/CD28 Activator (25 μl per 10⁶ cells) or left unstimulated (n=2 biological replicates). We performed bulk ATAC-seq as previously described⁴⁴. In brief, cell membranes were lysed in the RSB buffer (10 mM Tris-HCL pH 7.4, 3 mM MgCl₂, 10 mM NaCl) with 0.1% IGEPAL freshly added. After pipetting up and down, nuclei were isolated by centrifugation at 500 g for 5 min at 4° C. After discarding the supernatant, the nuclei were resuspended in the Tagmentation DNA (TD) Buffer⁴⁴ with homemade transposase TnY protein⁴⁴ and incubated at 37° C., for 30 min. After purification on a MinElute column (Qiagen), the tagmented DNA was PCR-amplified using a homemade Pfu X7 DNA polymerase⁴⁴ and barcoded primers for 12 cycles. The PCR product was purified via a 1.5/SPRI clean-up (Agencourt) and checked for a characteristic nucleosome banding pattern using TapeStation (Agilent). Samples were sequenced on a NextSeq 500 (Illumina) using the v2.5 75-cycle kit (single end).

### ATAC-Seq Analysis

Single-end reads were aligned to the Gencode hg38 primary assembly⁶⁷ using Bowtie2 v.2.4.4⁵⁷. We then used SAMtools v. 1.9⁶⁸ to filter out alignments with low-mapping quality (MAPQ<30) and subsequently to sort and index the filtered BAM files⁶⁸. Read duplicates were removed using Picard v.4.1.8.1⁶⁹. Peaks were called using MACS3 v.3.0.0⁷⁰ with default parameters (-g 2.7e9-q 0.05).

To construct the union feature space (‘union peaks’) used for much of the downstream analyses, we began by performing intersections on pairs of biological replicate narrowPeak files using BEDTools v.2.29.0 (using bedtools intersect), keeping only those peaks found in both replicates⁷¹. After marking the shared peaks between replicates, we used bedtools merge to consolidate the biological replicates at each shared peak (at least 1 bp overlap). In this new peak BED file, each shared peak includes all sequence found under the peak in either of the biological replicates. Next, we took the union of each of these peak files (LTBR resting. LTBR stimulated, tNGFR resting, tNGFR stimulation); we combined any peaks with at least 1 bp overlap. Using the union peaks, we generated a peak read count matrix (union peaks×ATAC samples), in which each entry in the matrix corresponds to the number of reads overlapping that peak in the specified sample—we term this the per-peak ATAC matrix. The overlapping reads are taken directly from the BAM files (converted to BED) that provide an alignment for each sample. Thus, the matrix includes a column for each biological replicate. Although samples had minimal differences in aligned reads, we normalized each entry in the matrix by the number of reads that overlapped the TSS regions in each sample. In this manner, any difference in read or alignment depth between samples would be normalized appropriately. In addition to the per-peak ATAC matrix, we also constructed a per-gene ATAC matrix as follows: we assigned a gene's total ATAC reads as the sum of normalized reads from the per-peak ATAC matrix for all peaks within 3 kb of a gene's start or end coordinates.

We imported these two ATAC matrices (per-peak and per-gene) into R v.4.1.1 for gene and peak enrichment analysis using DESeq2 v. 1.32.0. For comparison between ATAC-seq and RNA-seq, we used a statistical threshold of adjusted P value <0.05 and either log 2-transformed fold change >0 (for increases in ATAC or RNA) or logs-transformed fold change <0 (for decreases in ATAC or RNA). For transcription factor-motif analysis we used Chrom-VAR v. 1.14.072 as follows: For each of the test versus control conditions, we constructed Summarized Experiment objects using column and sample subsets of the per-peak matrix and the union feature space. We used the matchMotifs function to annotate transcription factormotifs. We computed enrichment deviations between test and control conditions using the computeDeviations function. To produce read pile-up tracks at specific genomic loci, we pooled de-duplicated reads from biological replicates (BAM) using samtools merge. We converted these pooled-replicate BAM files to big Wig files by using the bamCoverage function from deeptools v.3.4.2 and setting the scaleFactor to the relative number of TSSs found in the pooled biological replicates compared to all other sample aggregates73. Using the big Wig files, read pileups were plotted with pyGenomeTracks v.3.6⁷⁴.

Finally, we performed k-means clustering on ATAC peaks near genes with increased chromatin accessibility. First, using DEseq2 on the ATAC per-gene matrix, we identified genes with log 2-transformed fold change >1 and adjusted P value <0.05 (that is, genes with increased chromatin accessibility) in either of two comparisons: (1) LTBR stimulated versus tNGFR stimulated: (2) LTBR resting versus tNGFR resting. After identifying these genes, we isolated all accessibility peaks in the per-peak ATAC matrix within 3 kb of the gene body; this subset of peaks from the per-peak ATAC matrix was used as input for the clustering. Then, using deeptools (computeMatrix and plotHeatmap functions) on this subset of ATAC peaks, we performed k-means clustering with k=4 clusters and 6 kb read windows.

### Statistical Analysis

Data between two groups were compared using a two-tailed unpaired Student's t-test or the Mann-Whitney test as appropriate for the type of data (depending on the normality of the distribution). Unless otherwise indicated, a P value less than or equal to 0.05 was considered statistically significant for all analyses, and not corrected for multiple comparisons. In cases in which multiple comparison corrections were necessary, we adjusted the P value using the Benjamini-Hochberg method. All group results are represented as mean±s.e.m., if not stated otherwise. Statistical analyses were performed in Prism (GraphPad) and RStudio (Rstudio PBC). Flow cytometry data were analyzed using FlowJo v. 10.7.1 (Treestar).

### Example 2: Genome-Scale Screen for Synthetic Drivers of T-Cell Proliferation

We performed a genome-scale gain-of-function screen in primary human CD4⁺ and CD8⁺ T cells, using a lentiviral library of barcoded human ORFs. We show that T cells with the strongest proliferation phenotypes are enriched for both known and unknown regulators of the immune response, many of which are not typically expressed by peripheral T cells. We validate top-ranked ORFs in cells from screen-independent donors and further demonstrate that these ORFs not only drive T cell proliferation but also increase the expression of activation markers and the secretion of key proinflammatory cytokines. To gain more comprehensive insight into the mechanism of action of these genes, we develop a single-cell sequencing approach coupled with direct ORF capture. We identify LTBR—one of the top-ranked ORFs not expressed by lymphocytes—as a key driver of profound transcriptional and epigenetic remodeling through increased NF-κB signaling, which results in a marked increase in the secretion of proinflammatory cytokines and resistance to apoptosis. Finally, we show that top-ranked ORFs potentiate antigen-specific T cell functions, in the context of CD19-directed CAR T cells and broadly tumor-reactive γδ T cells from healthy donors and patients with blood cancer.

### Genome-Scale ORF Screen in T Cells

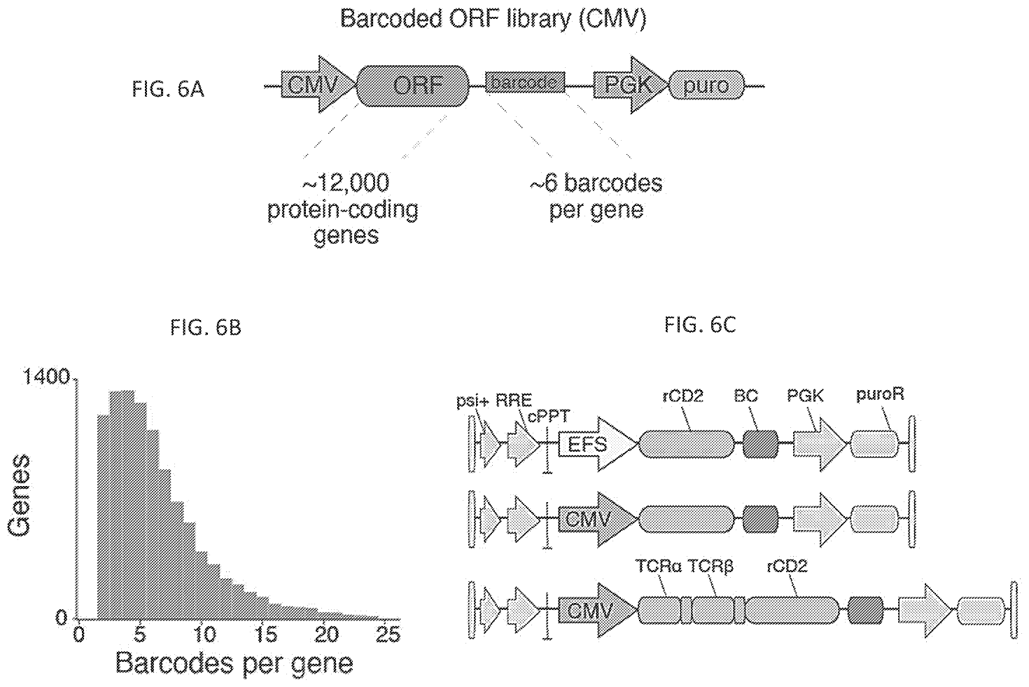

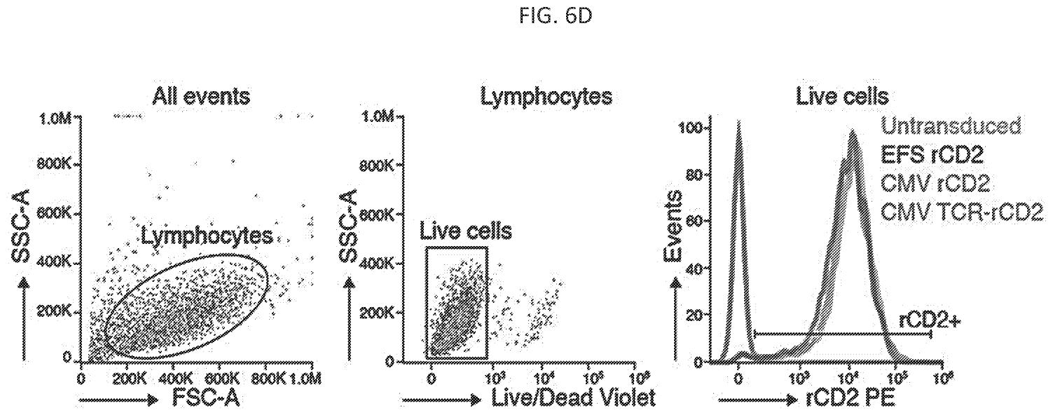

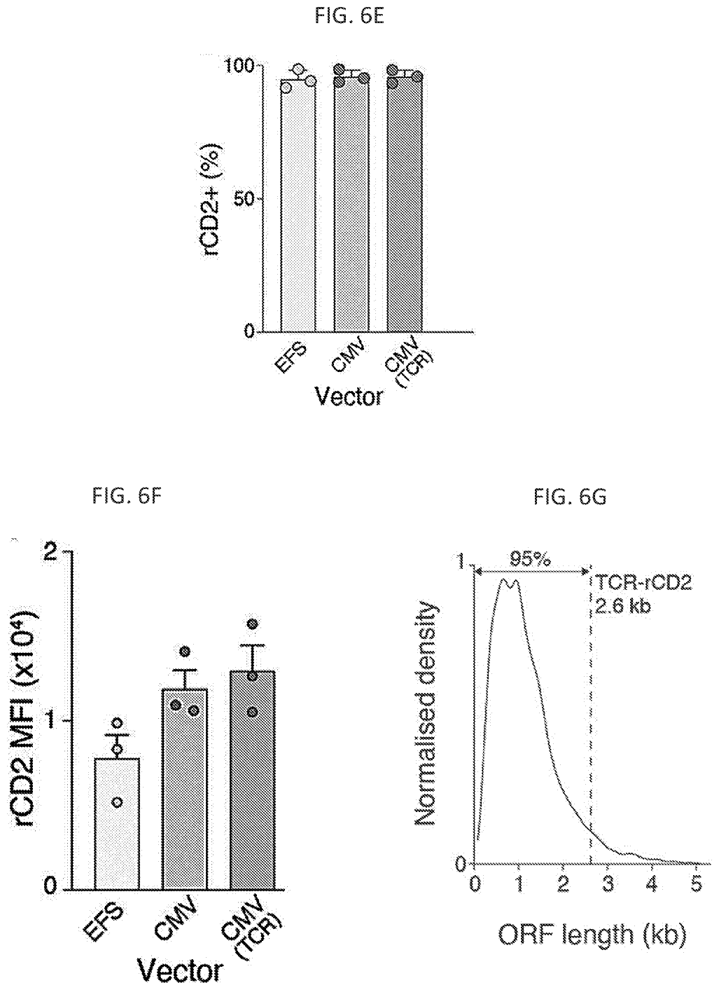

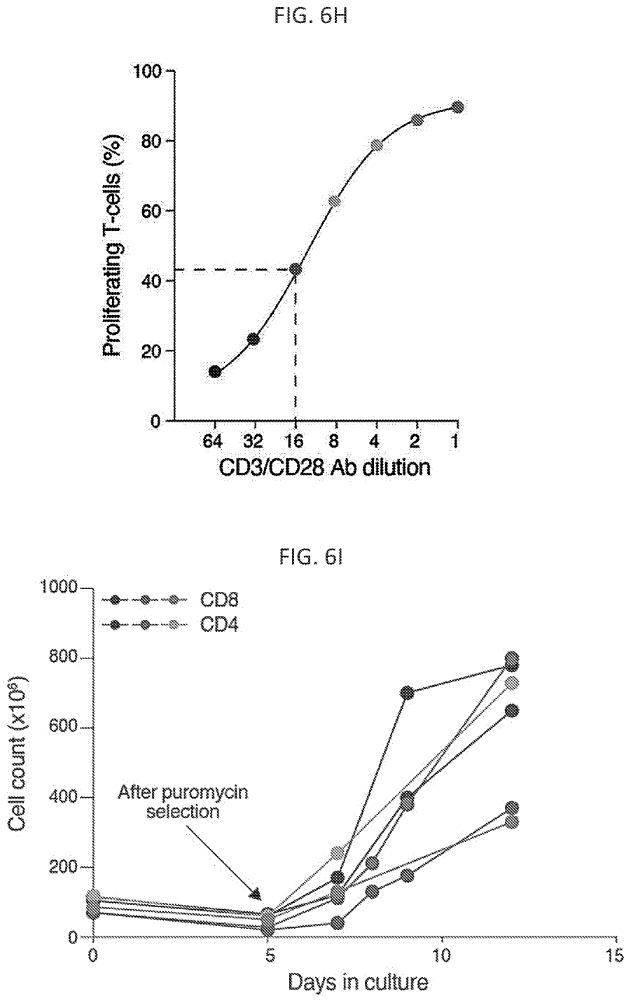

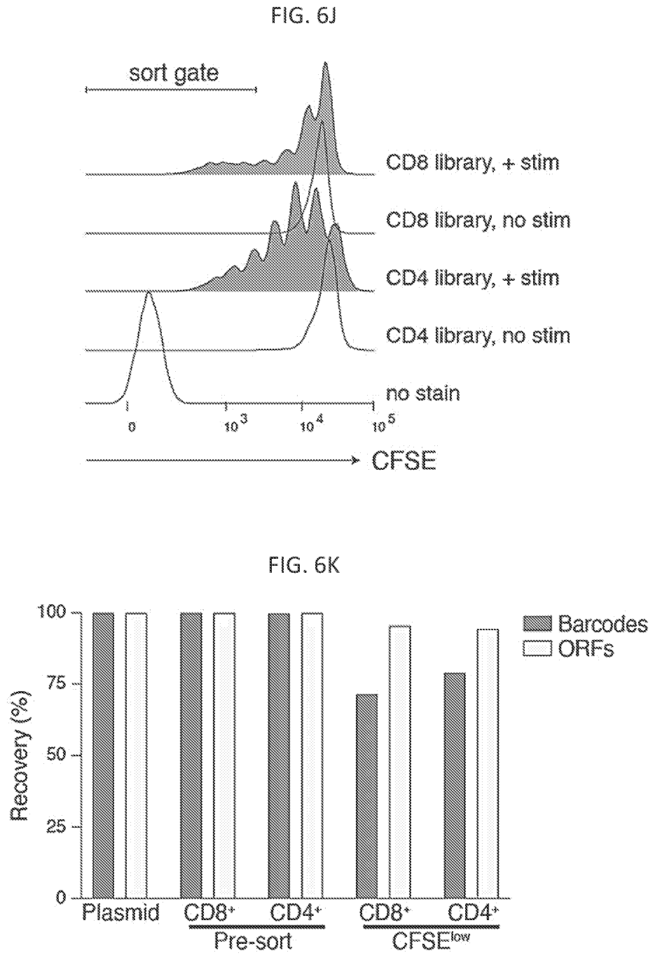

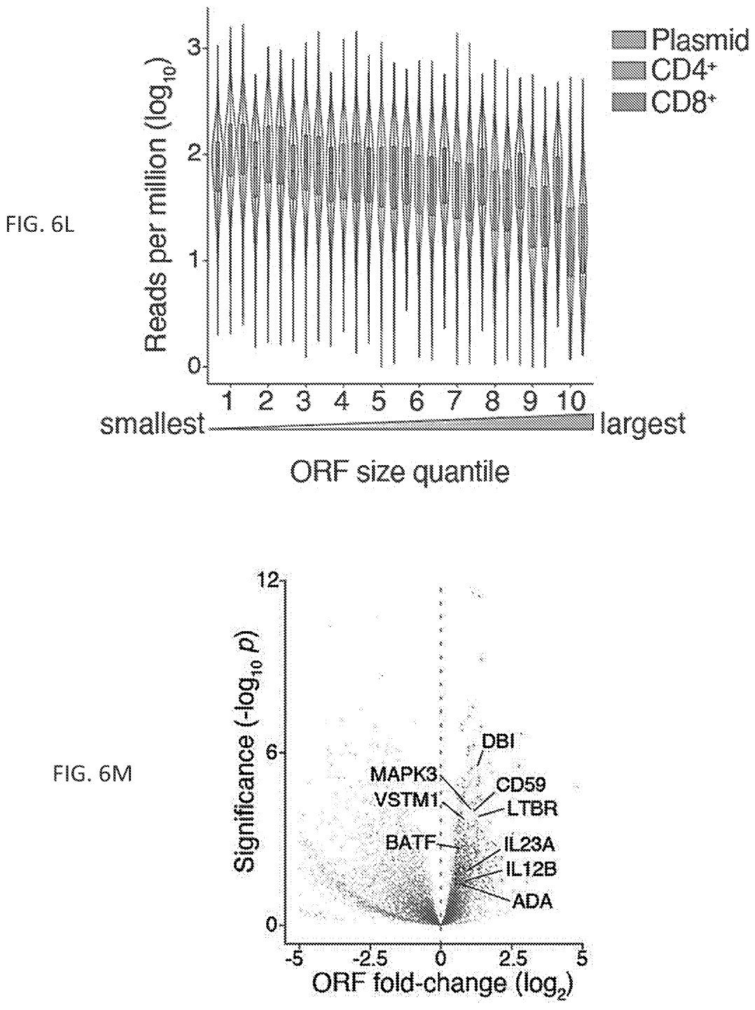

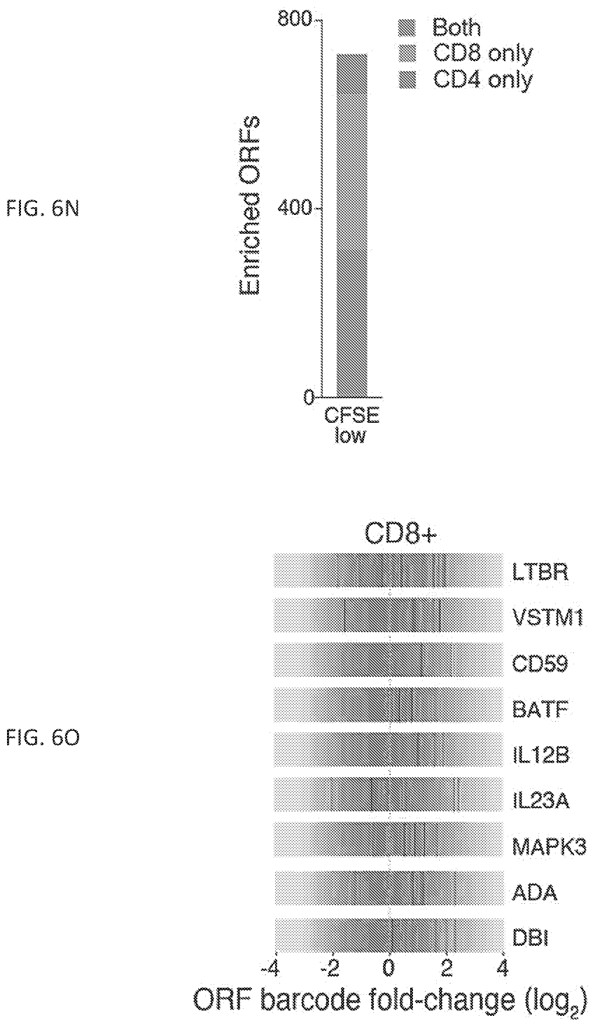

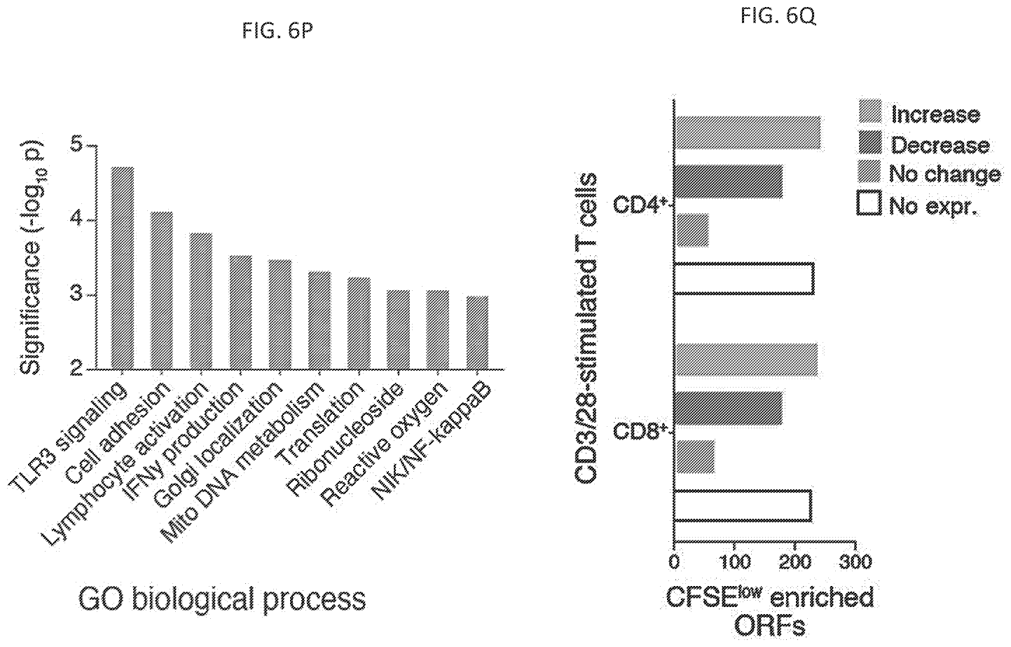

To avoid relying on constitutive expression of large bacterial proteins or chromatin accessibility in the vicinity of target genes¹³, we decided to use a lentiviral library of human ORFs; this library contains nearly 12,000 full-length genes, with around 6 barcodes per gene¹⁴ (FIG. 1A, FIG. 6A-FIG. 6G). Previously, genome-scale loss-of-function screens in human T cells have focused on either CD4⁺ or CD8⁺ T cells. However, both CD4⁻ and CD8⁺ T cells are required for durable tumor control in adoptive therapies^{15,16}, as further exemplified by FDA approvals of anti-CD19 CAR T cells with a defined 1:1 CD4⁺ and CD8⁺ ratio¹. Thus, we decided to use the ORF library to discover genes that boost the proliferation of both CD4⁺ and CD8+ T cells in response to T cell receptor (TCR) stimulation (FIG. 1A, FIG. 6H-FIG. 6J).

We transduced the lentiviral ORF library into CD4⁺ and CD8⁺ T cells from three healthy donors, and after a brief period in culture (14 days) we restimulated the cells to identify drivers of proliferation in response to TCR stimulation. We were able to capture the majority of individual ORF barcodes, and nearly all ORFs, including the largest ones (FIG. 6K, FIG. 6L). Comparing the relative frequencies of genes in the most highly proliferative cells to unsorted cells, we found an enrichment of genes that are known to participate in immune processes among the top-ranked ORFs (FIG. 6M, FIG. 6N). We identified MAPK3 (encoding ERK1), a critical mediator of T cell functions¹⁷, the co-stimulatory molecule CD59¹⁸, the transcription factor BATF, and cytokines that are known to promote T cell proliferation, such as IL12B and IL23A19. In fact, two recent studies showed that overexpression of IL 12B and BATF boosts proliferation, cytotoxicity and cytokine secretion in CAR T cells^{19,20}.

Each ORF in the library is linked to an average of six DNA barcodes (FIG. 6B). To increase confidence in our top-ranked ORFs from the pooled screen, we assessed the enrichment of individual barcodes corresponding to a given ORF in proliferating CD4⁺ and CD8⁺ cells (FIG. 6B, FIG. 6C, FIG. 6O). For the majority of ORFs, multiple individual barcodes for each gene were enriched in the highly proliferating population, thus suggesting that the observed enrichment does not stem from spurious clonal outgrowth or PCR bias. Surprisingly, the most significantly enriched gene was lymphotoxin-β receptor (LTBR), a gene that is broadly expressed in stromal and myeloid cells but completely absent in lymphocytes.

Overall, the enriched ORFs spanned a range of diverse biological processes. Among the top-enriched Gene Ontology (GO) biological processes were lymphocyte proliferation, interferon-γ (IFNγ) production and NF-κB signaling (FIG. 6P). We observed that enriched ORFs showed only a slight preference for genes endogenously upregulated by T cells during stimulation with CD3 and CD28 (CD3/CD28), and in fact were represented in all classes of differential expression (FIG. 6Q). This result highlights the capacity of the pooled ORF screen to discover genes that enable T cell proliferation but that are not expressed normally during CD3/CD28-mediated activation and proliferation. For subsequent validation, we decided to test a broad range of ORFs that function in diverse pathways of relevance to T cell fitness, and that showed different modes of endogenous regulation (FIG. 1E).

### Top ORFs Enhance T Cell Functions

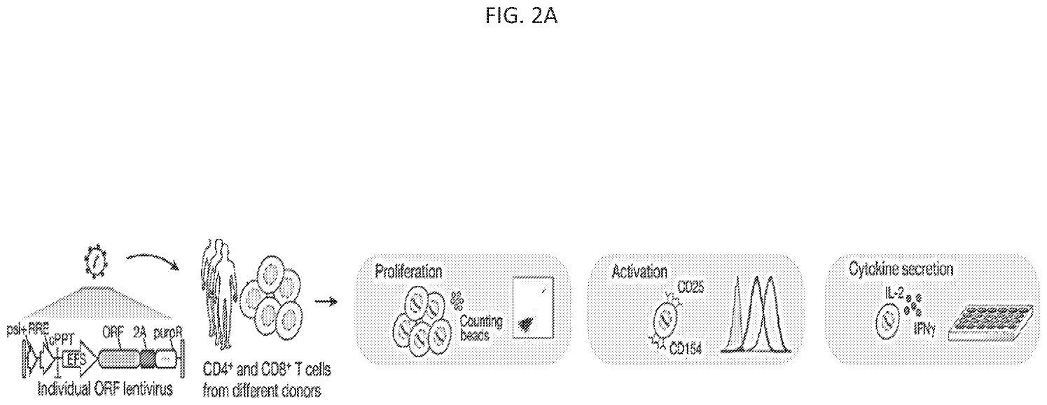

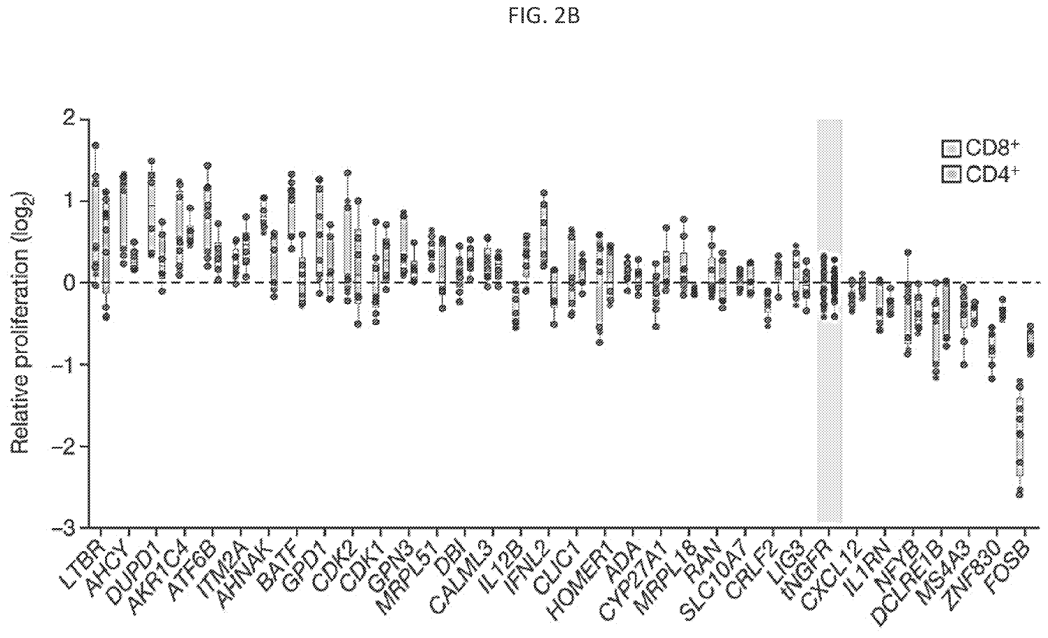

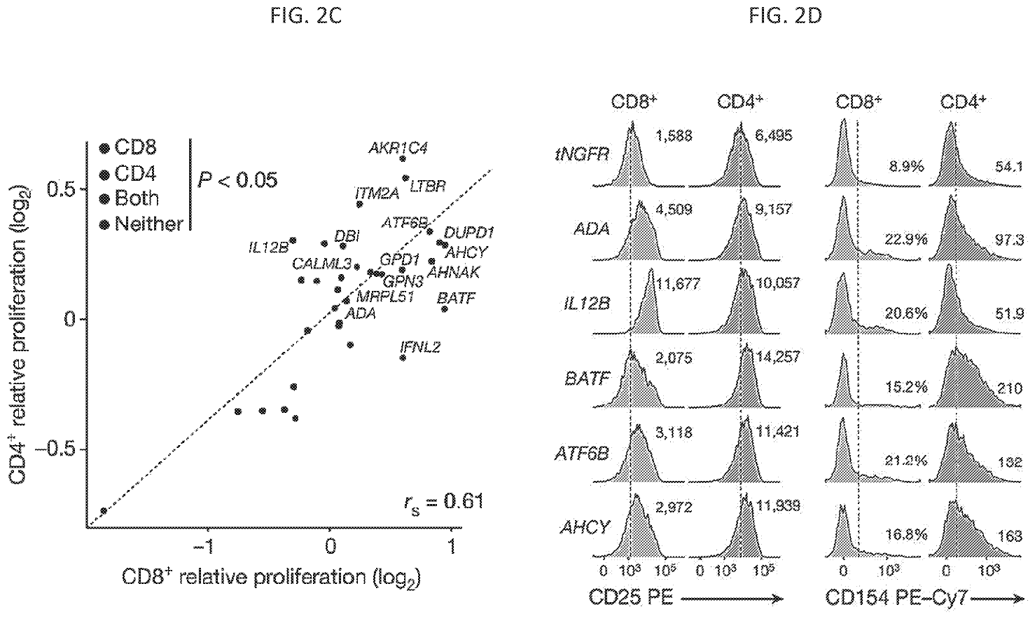

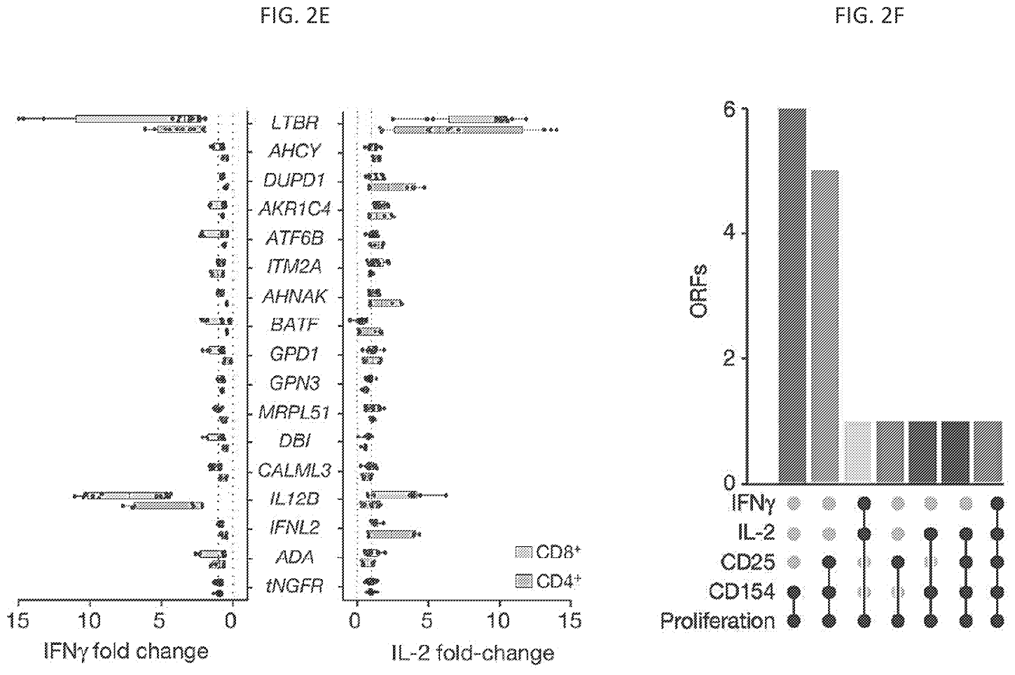

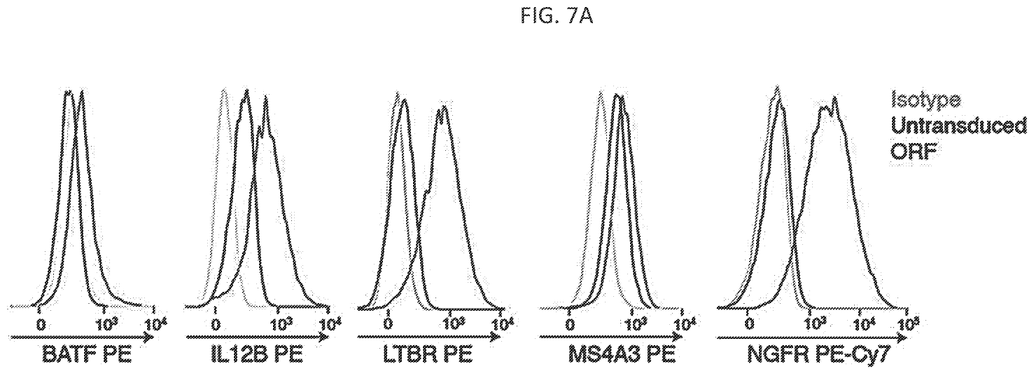

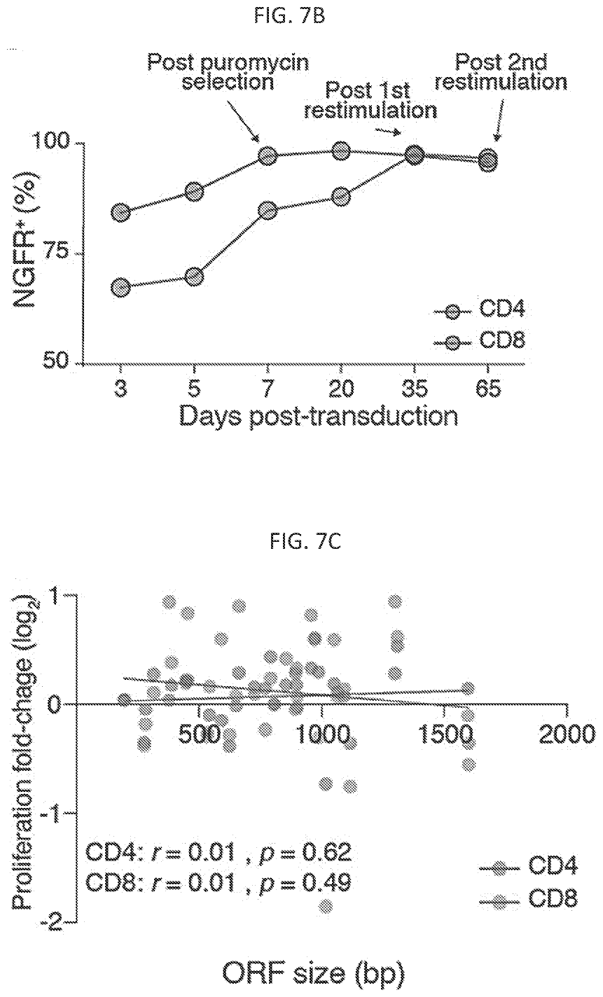

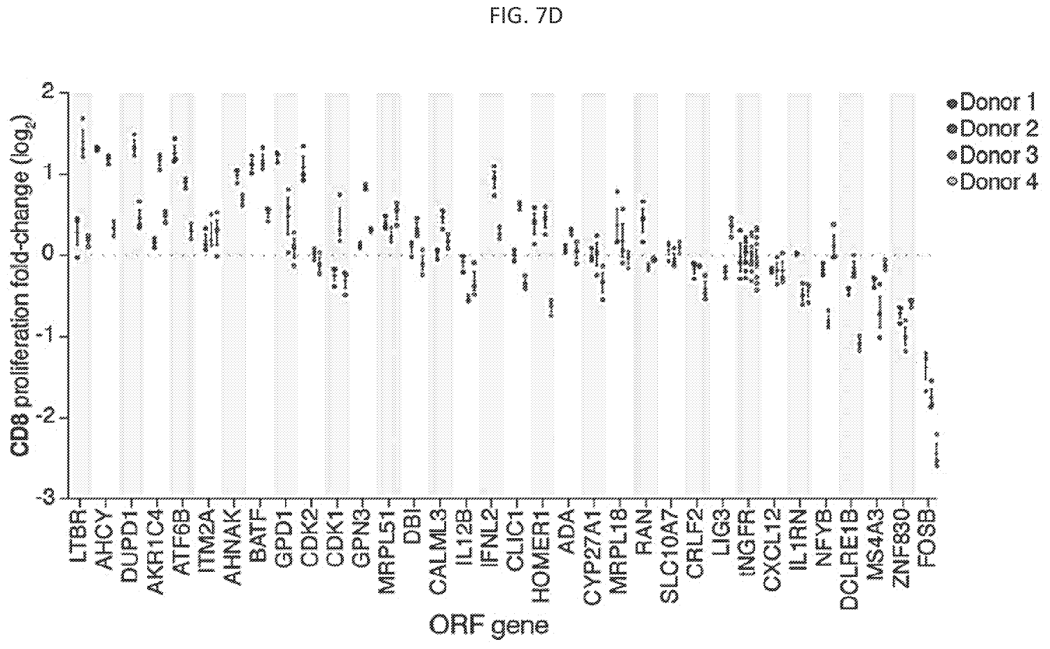

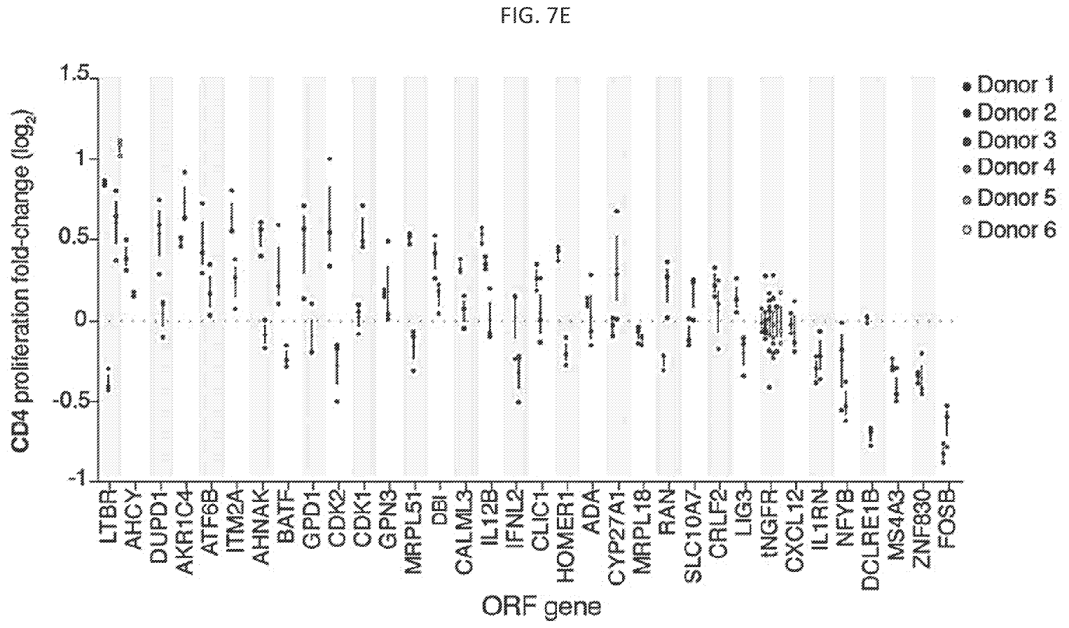

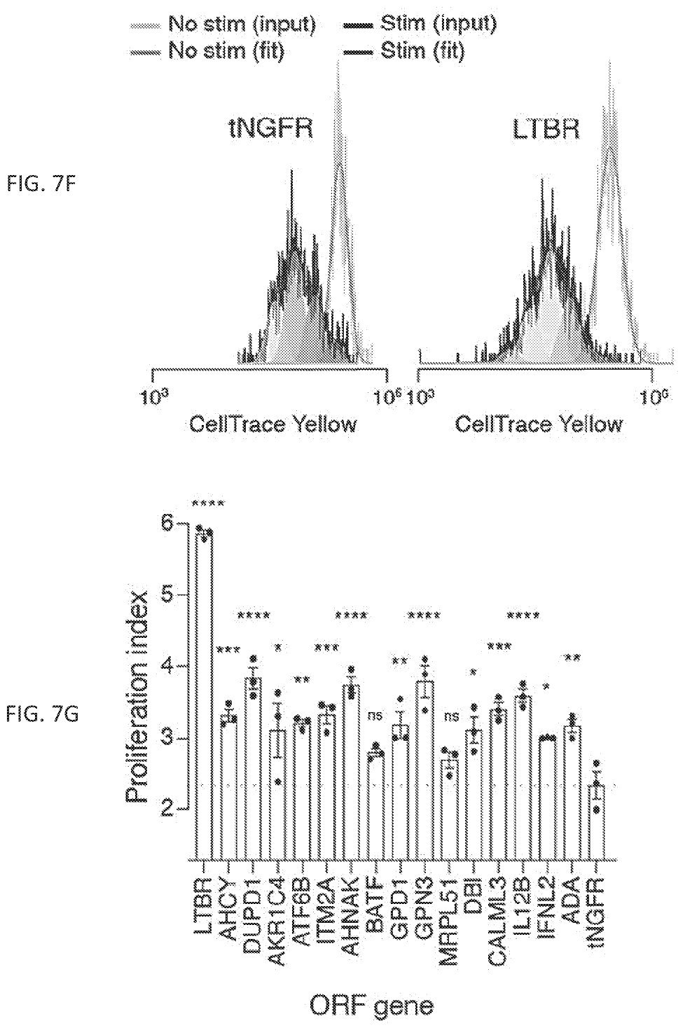

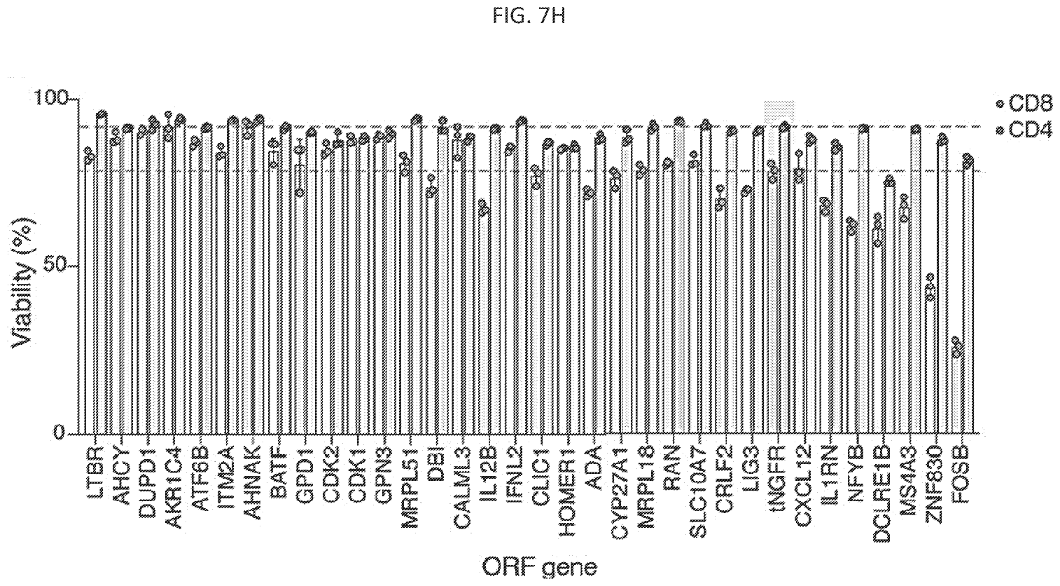

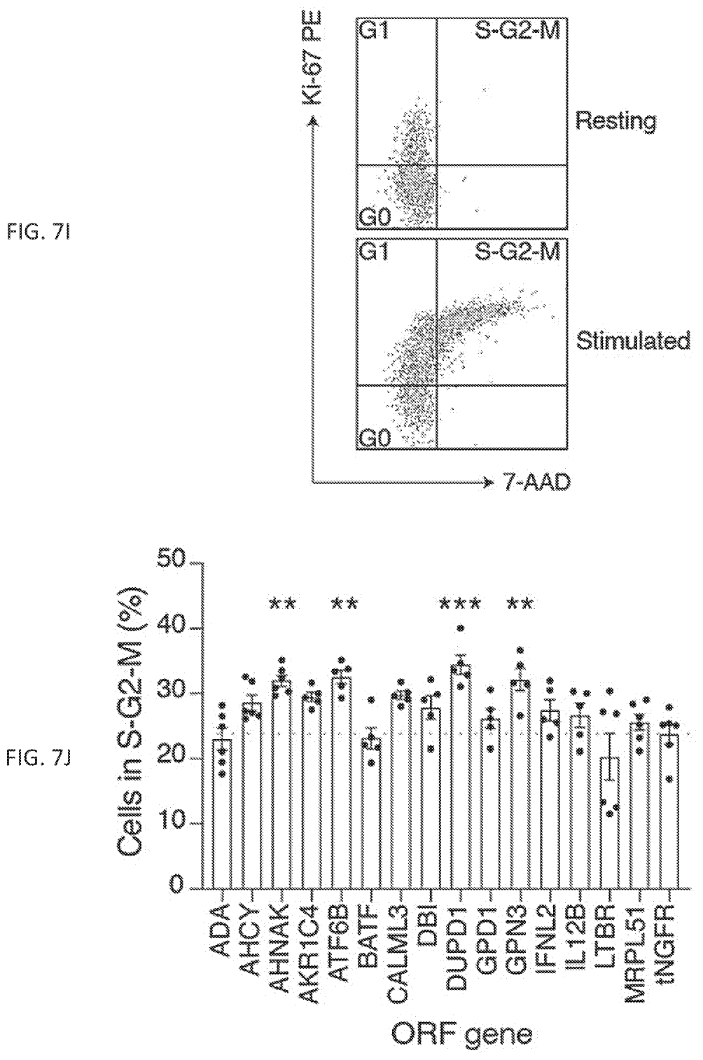

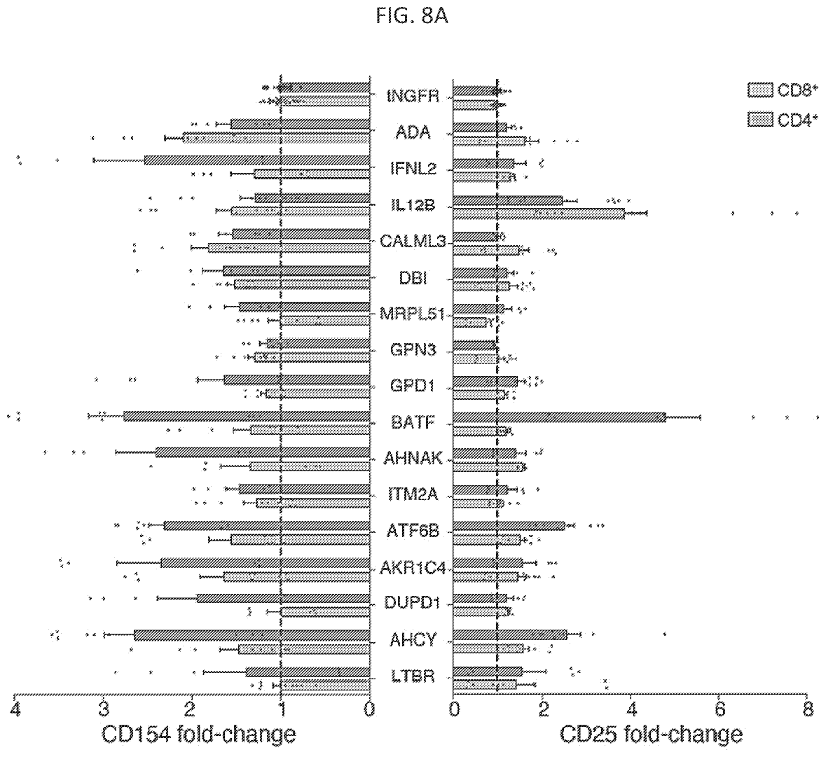

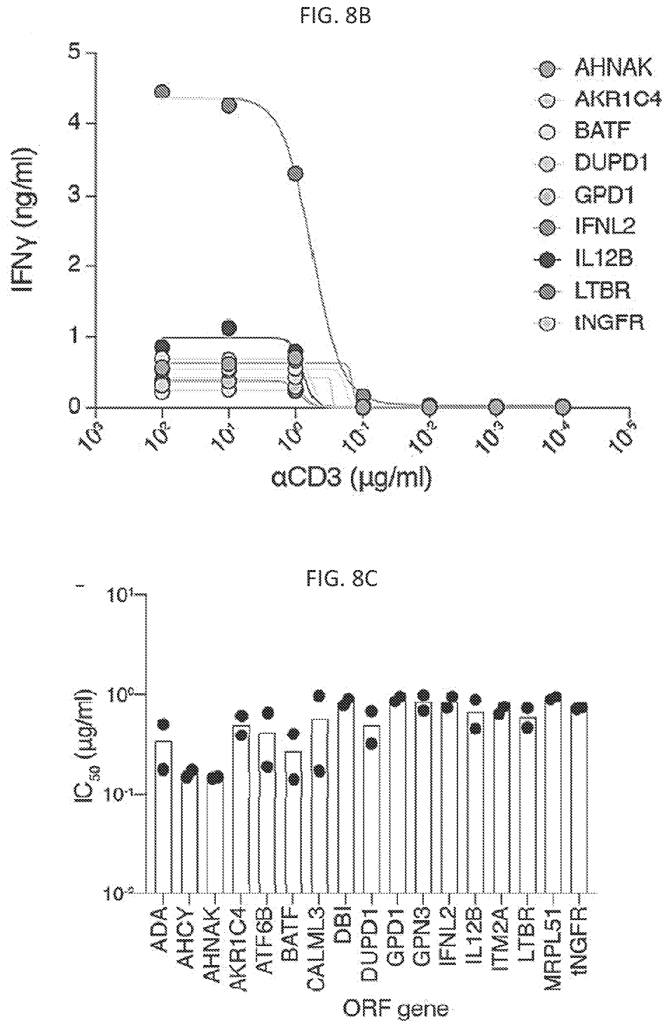

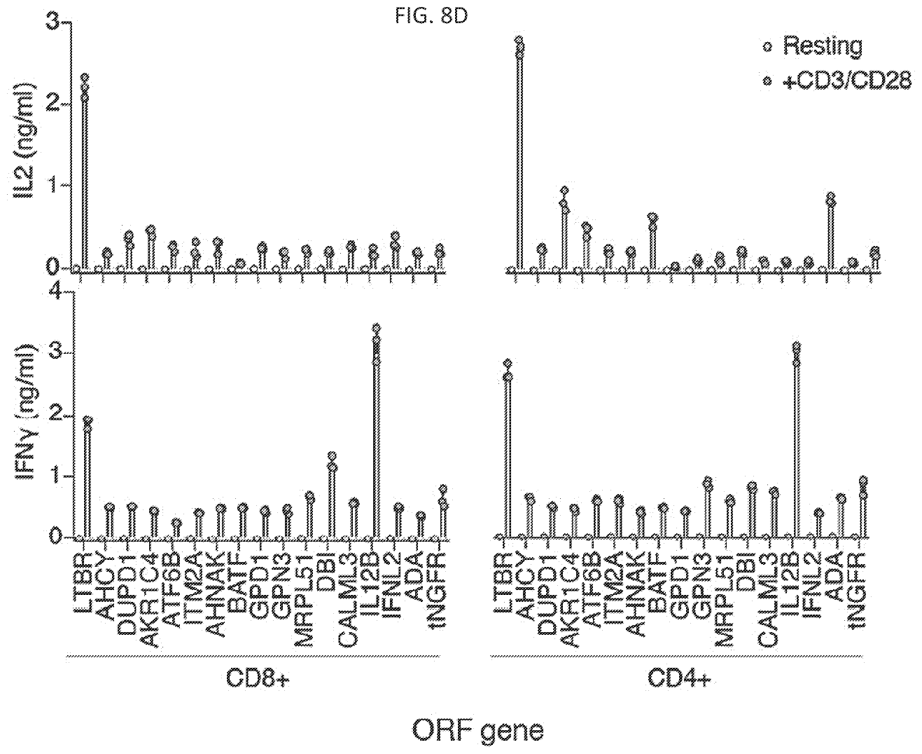

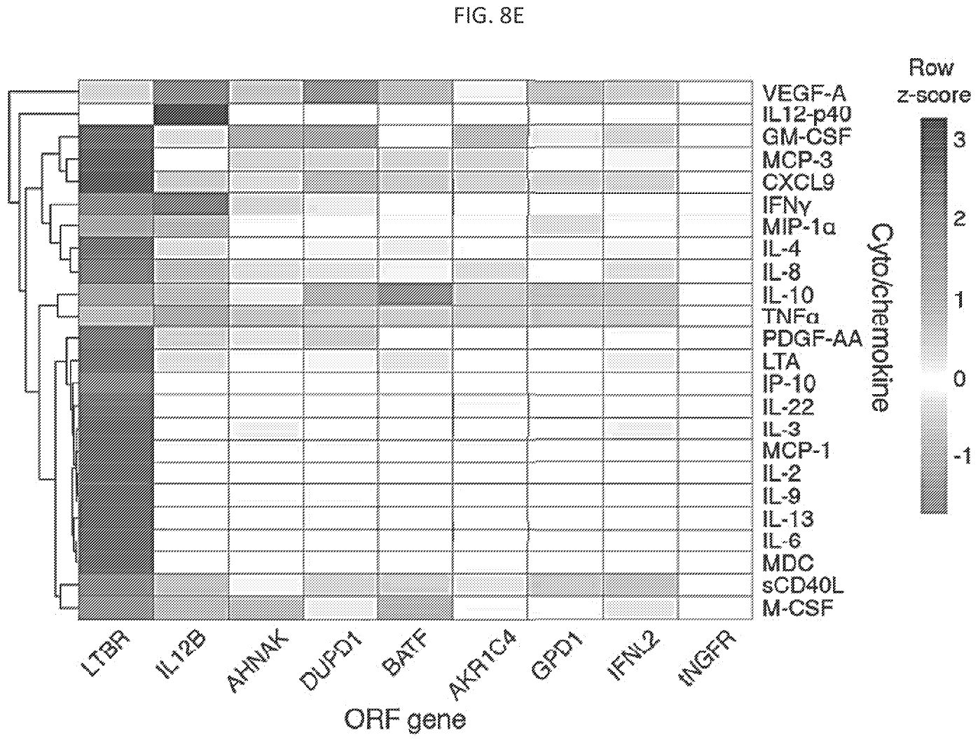

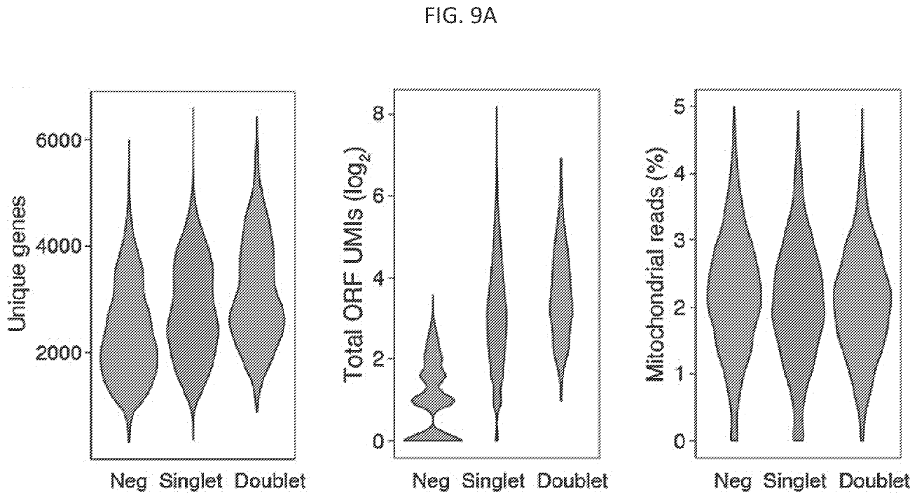

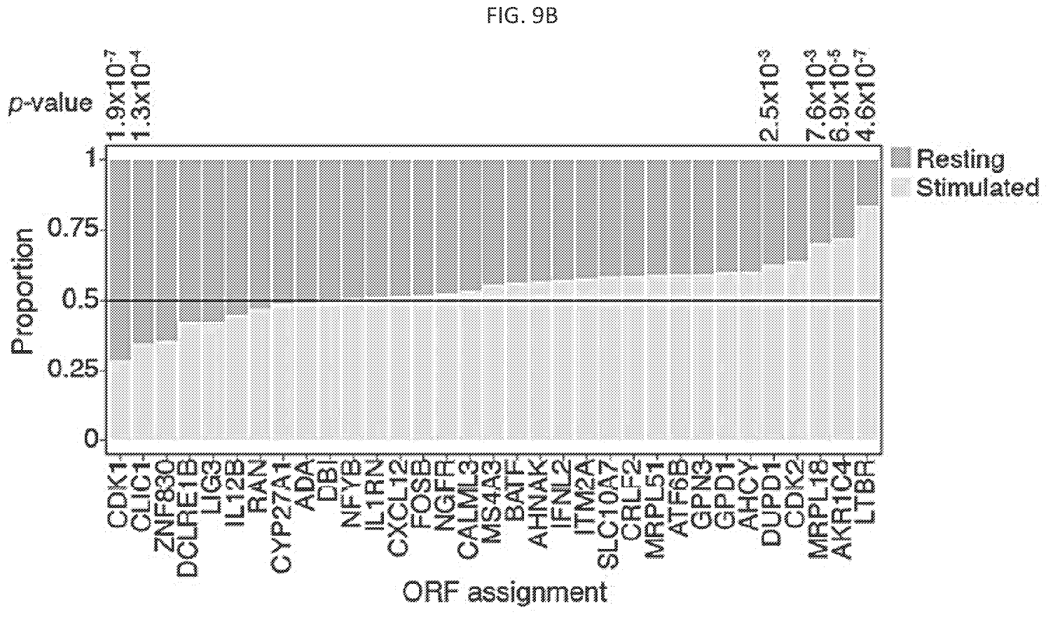

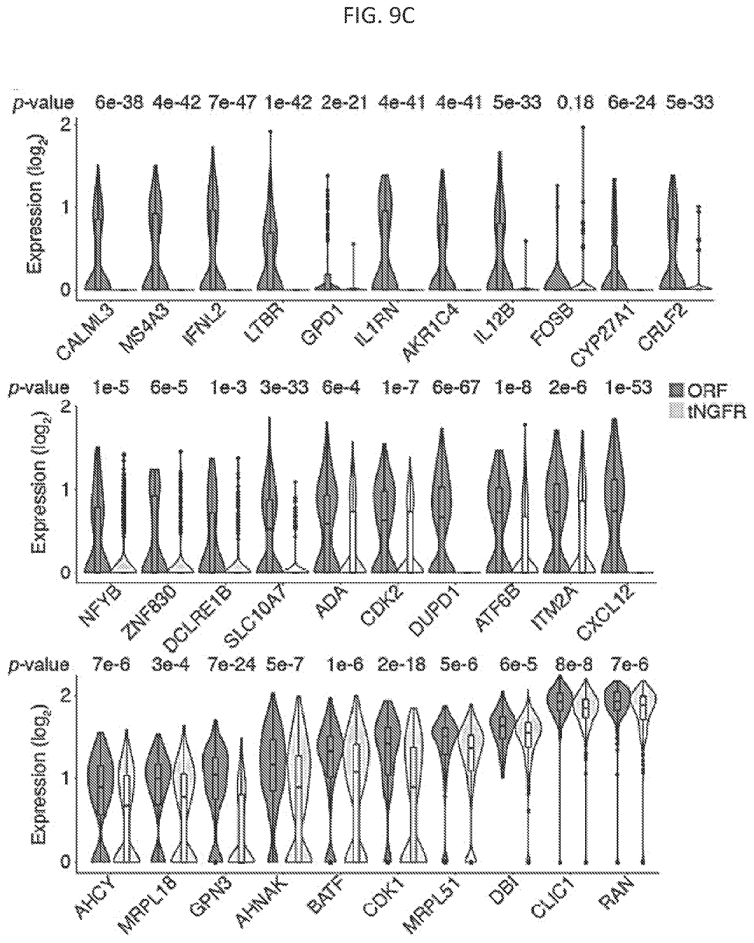

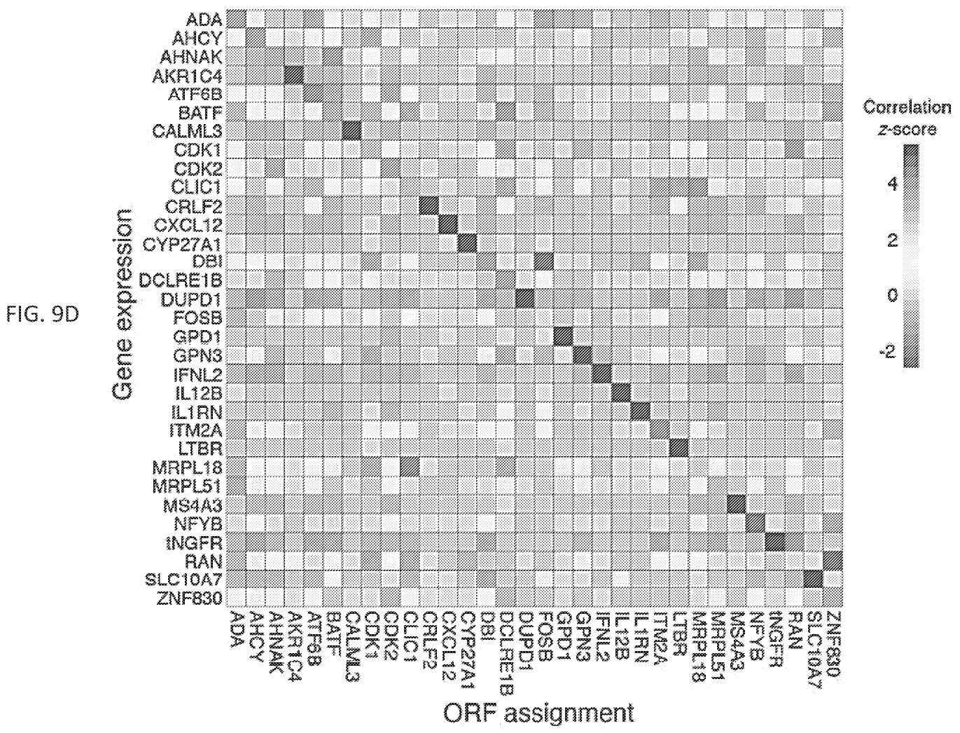

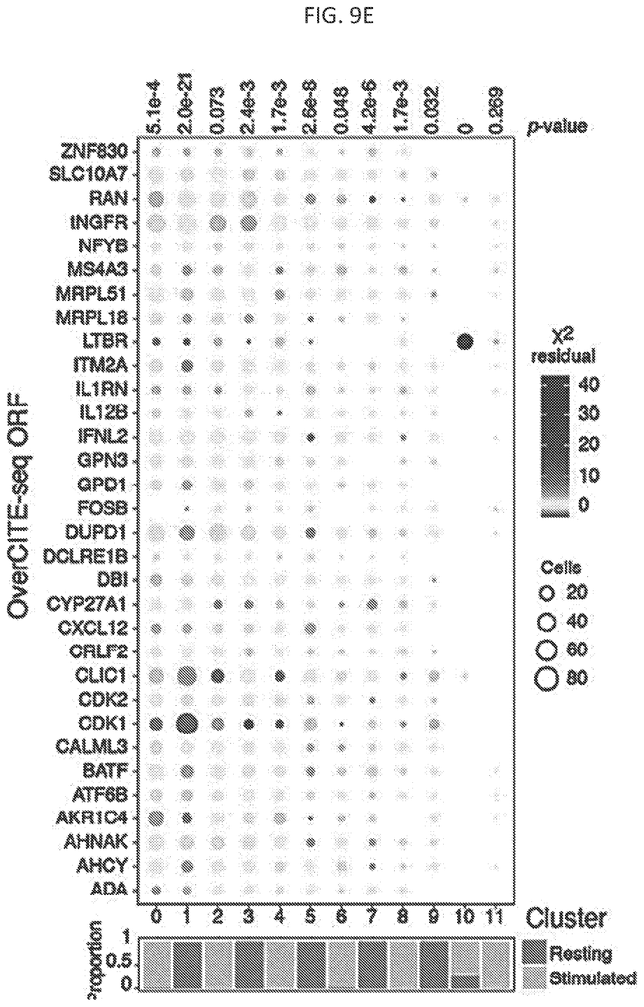

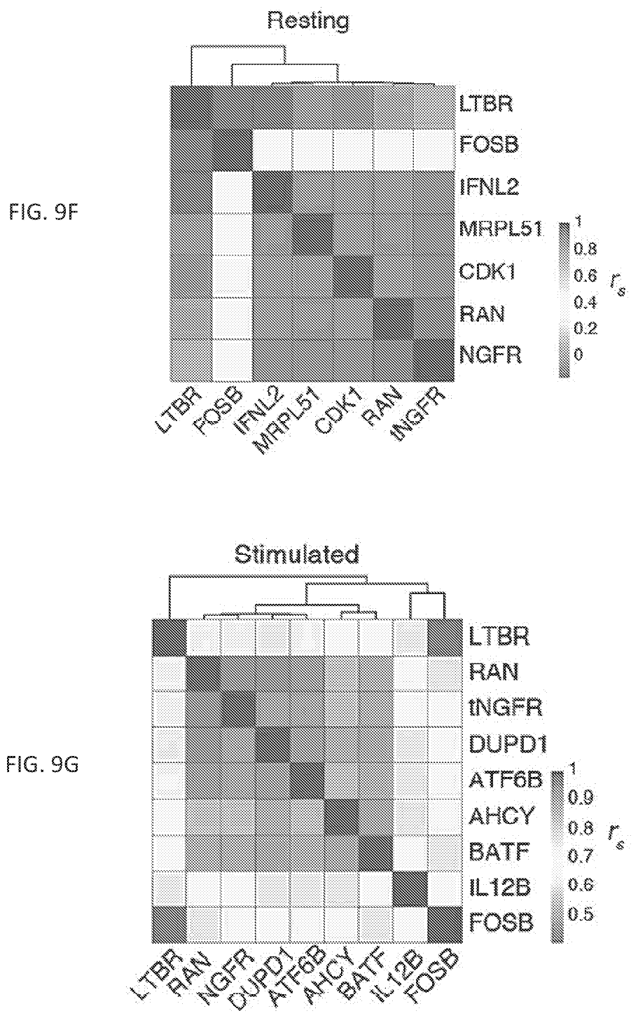

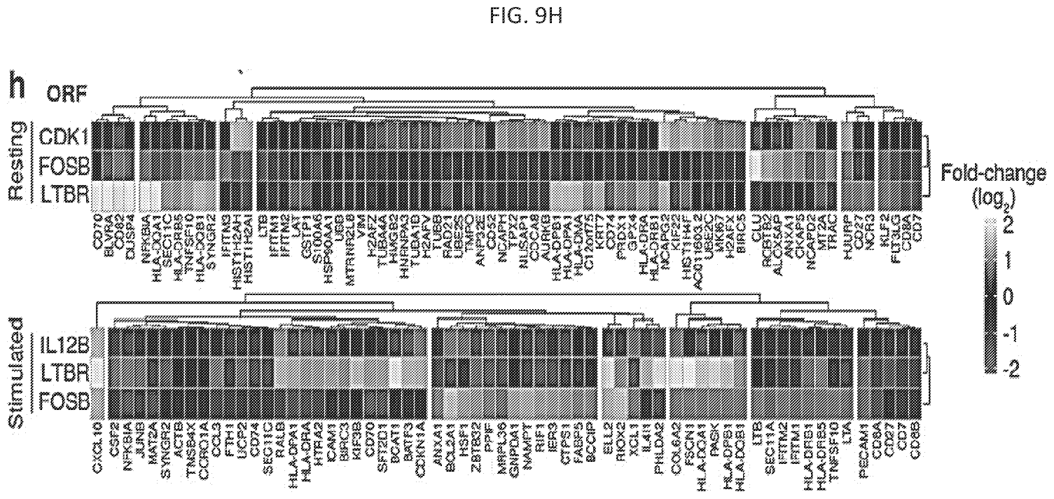

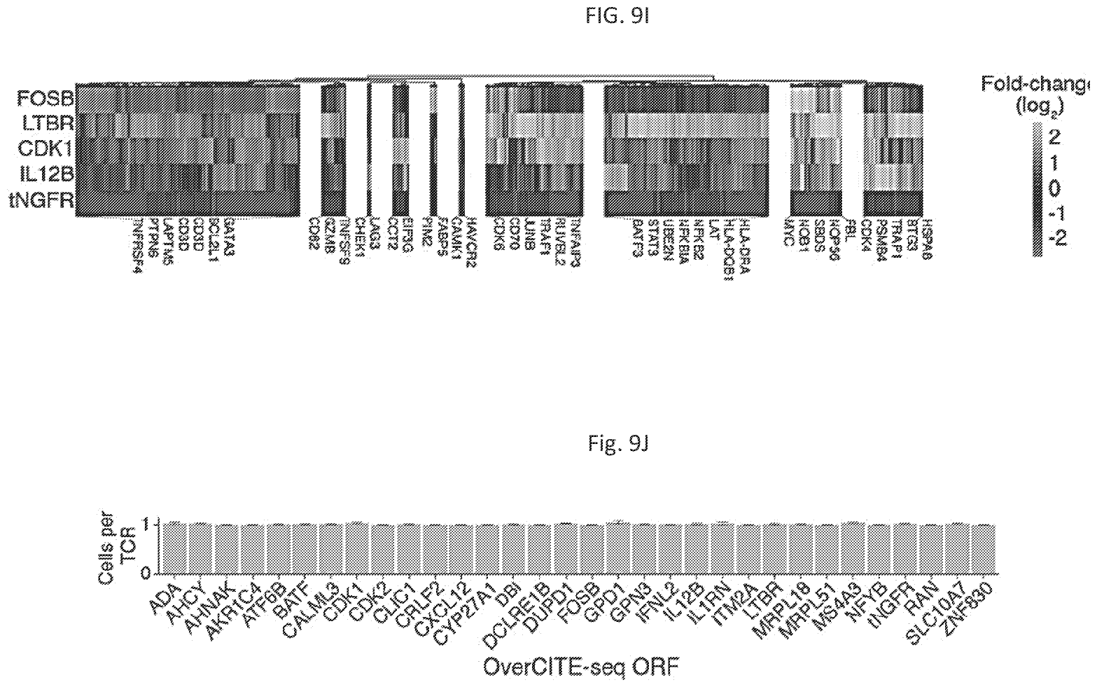

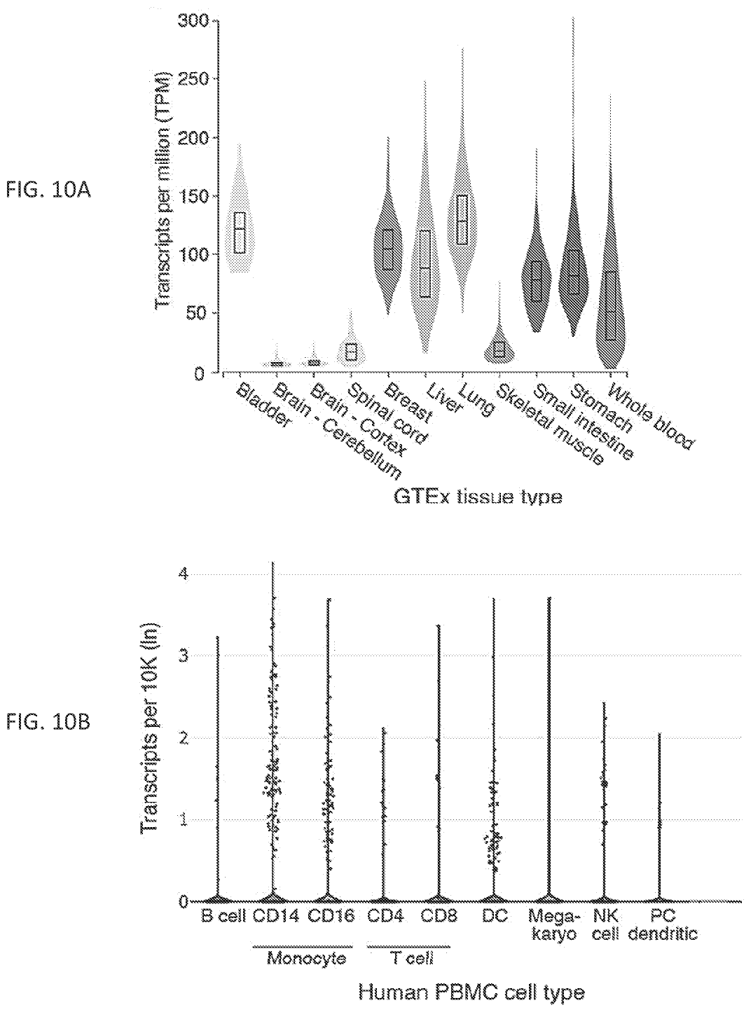

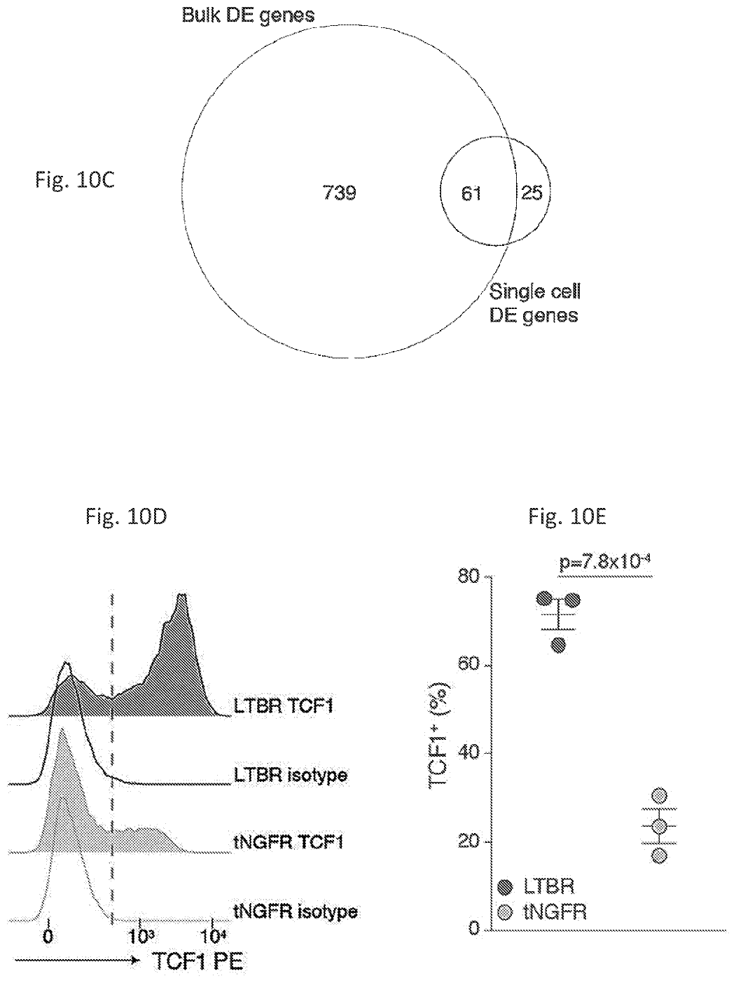

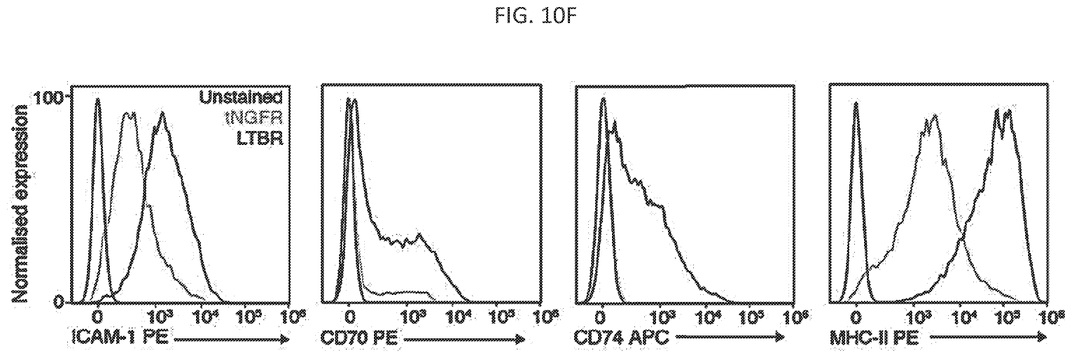

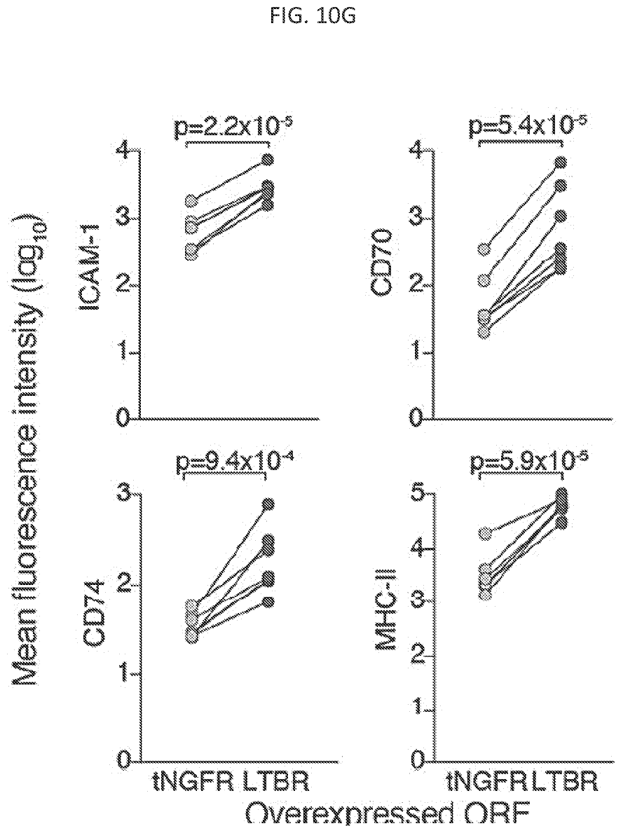

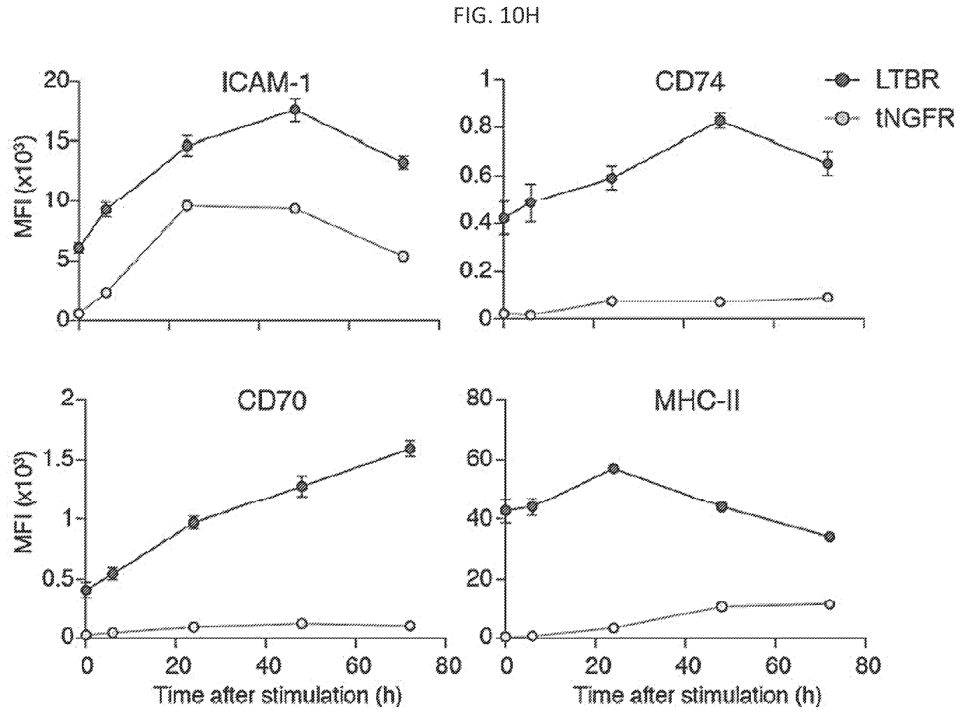

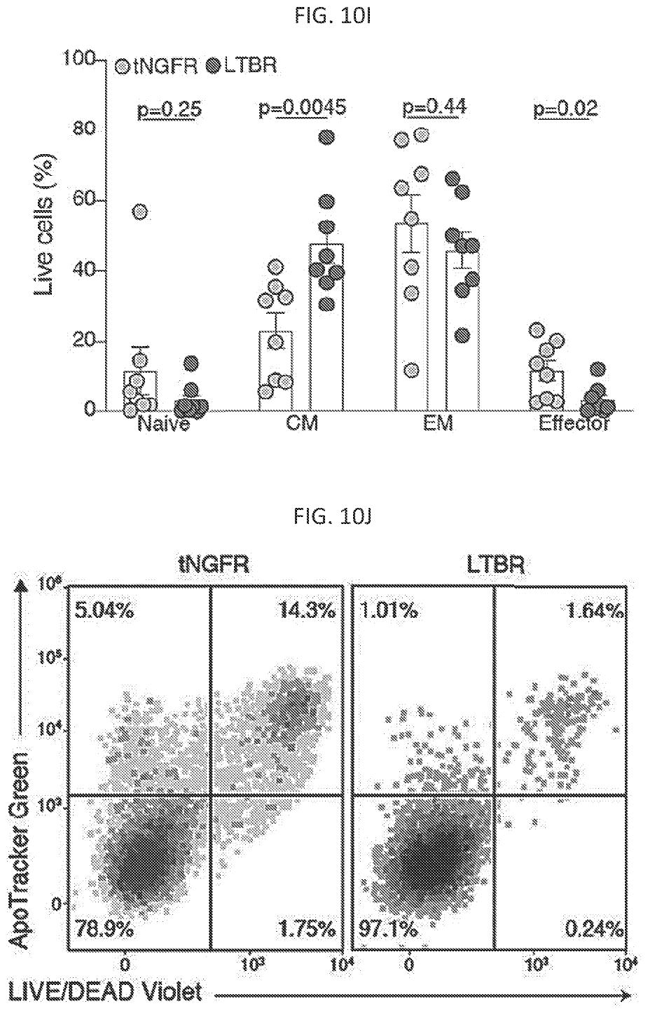

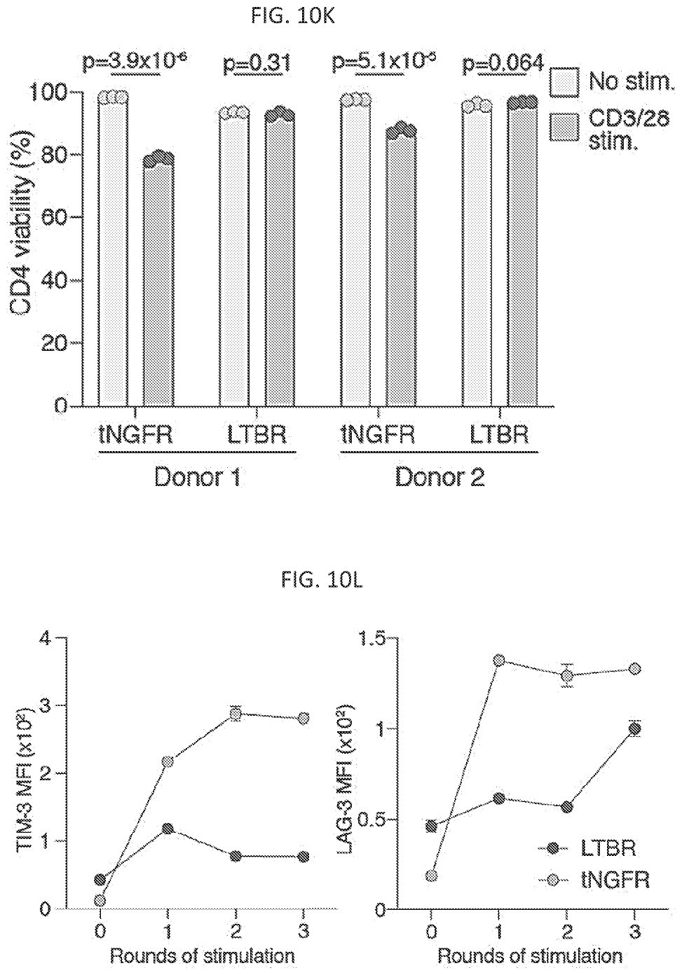

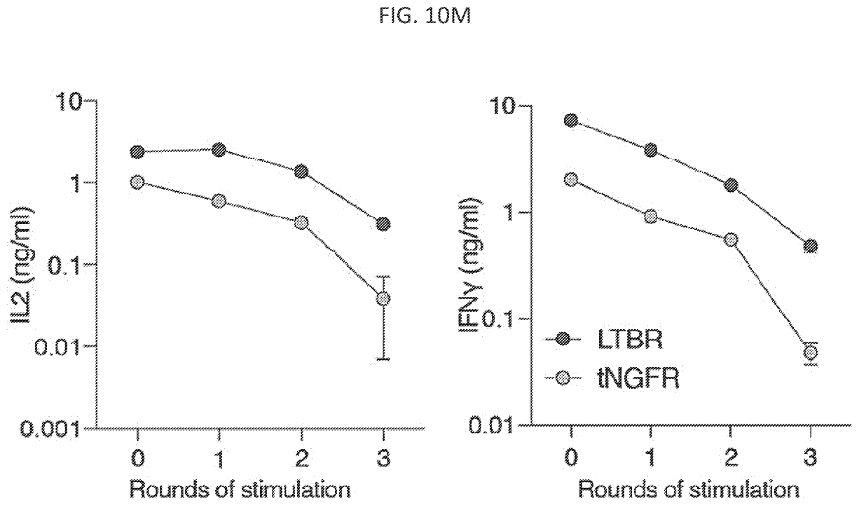

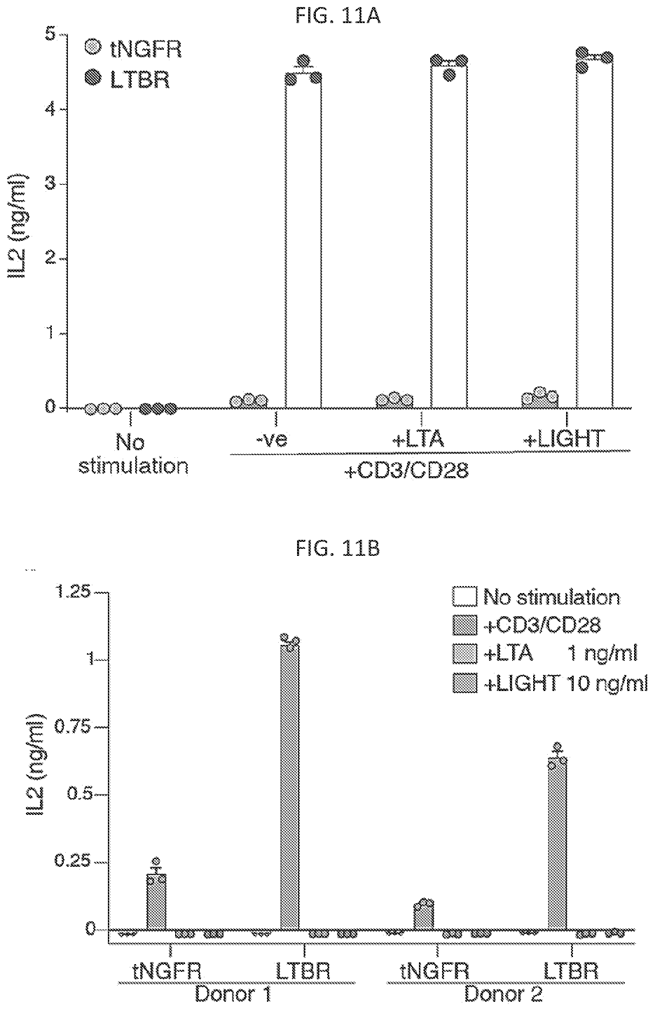

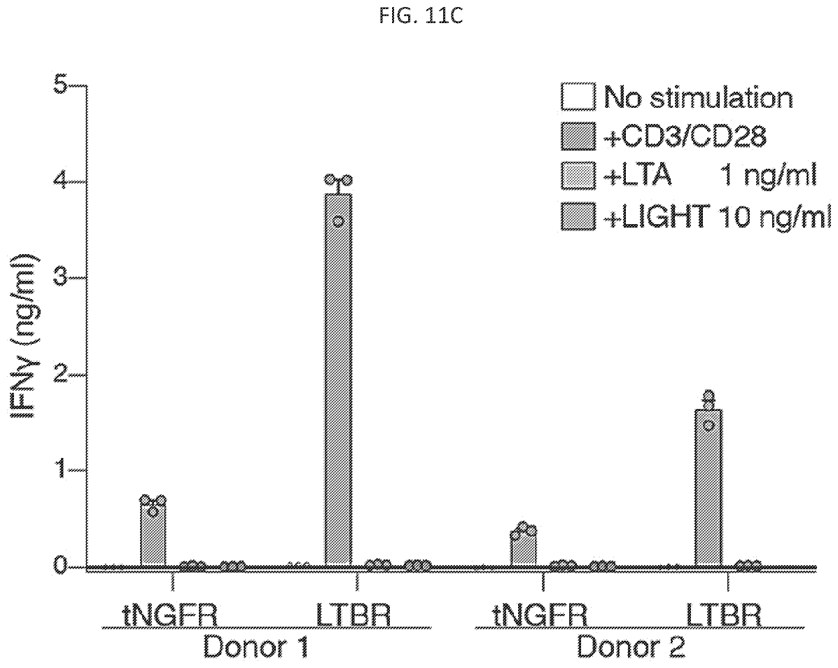

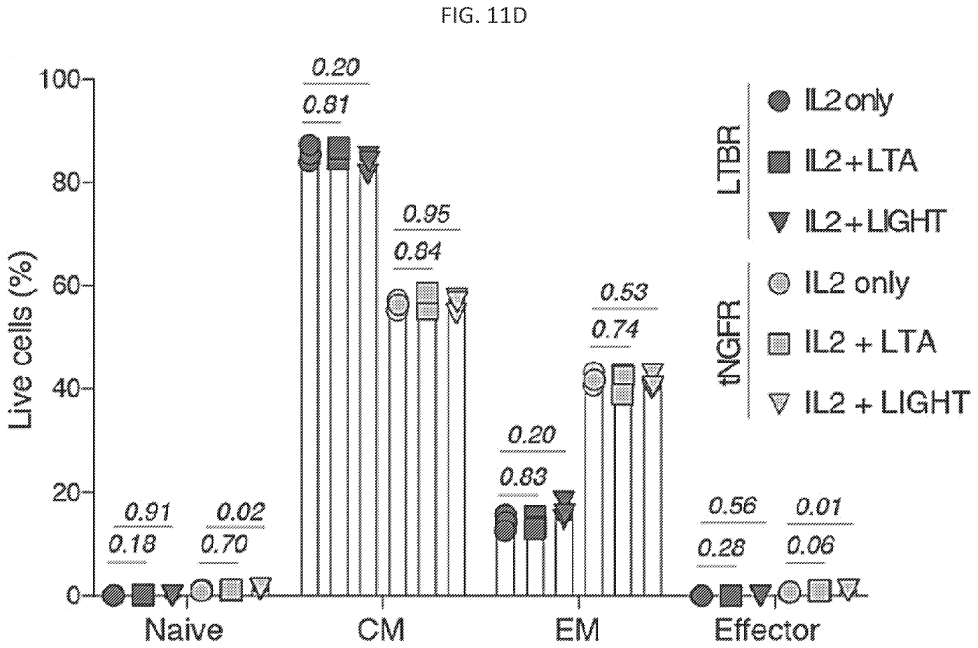

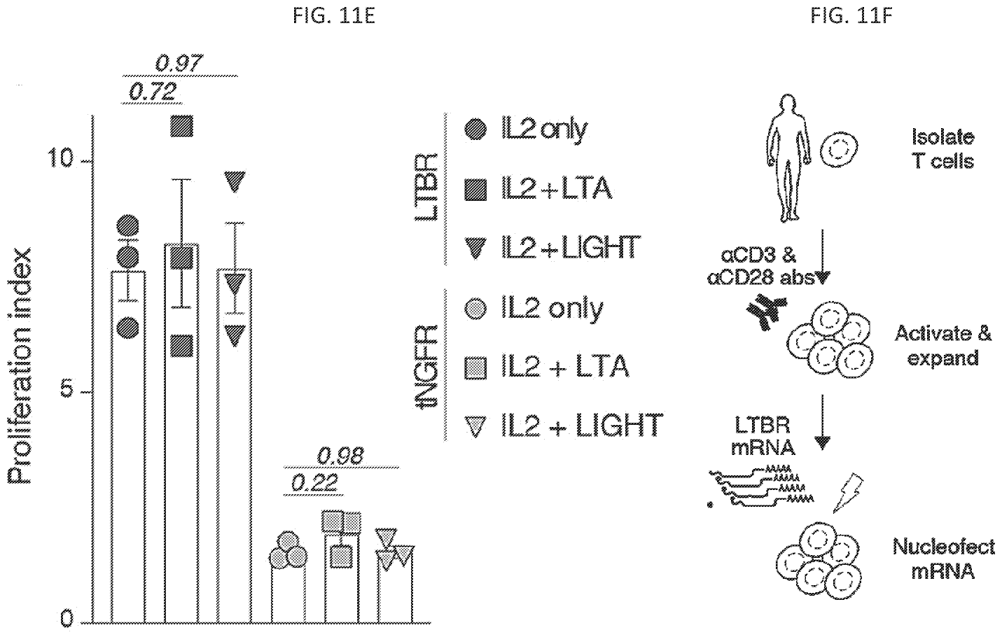

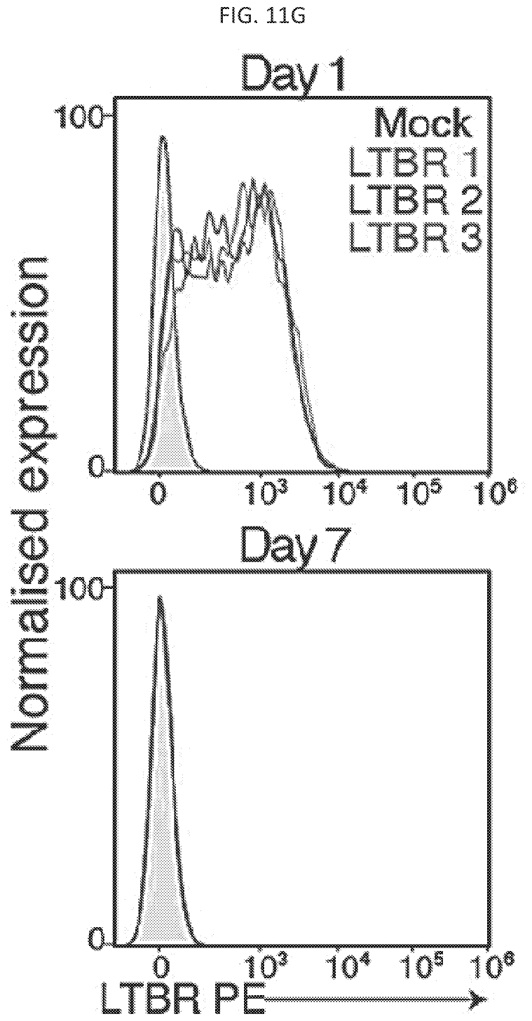

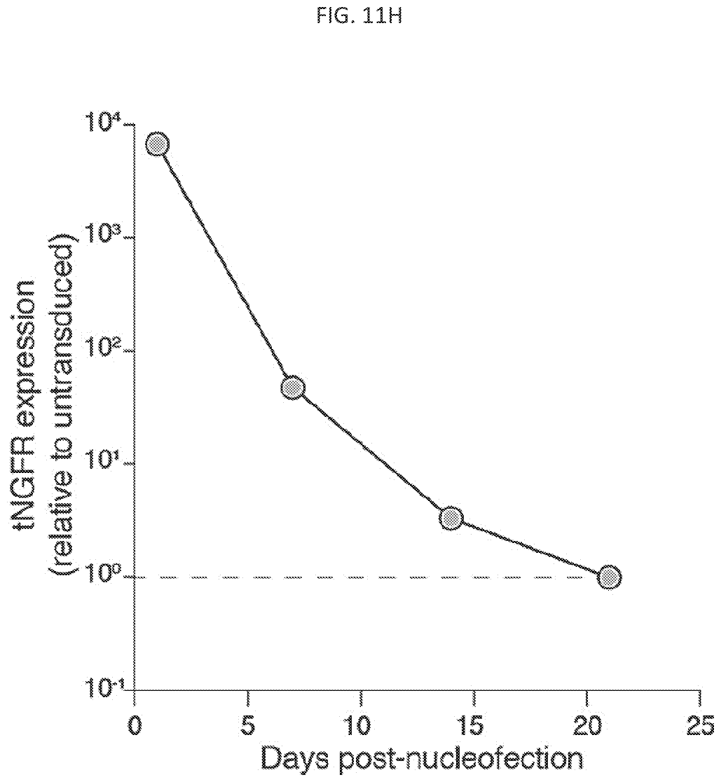

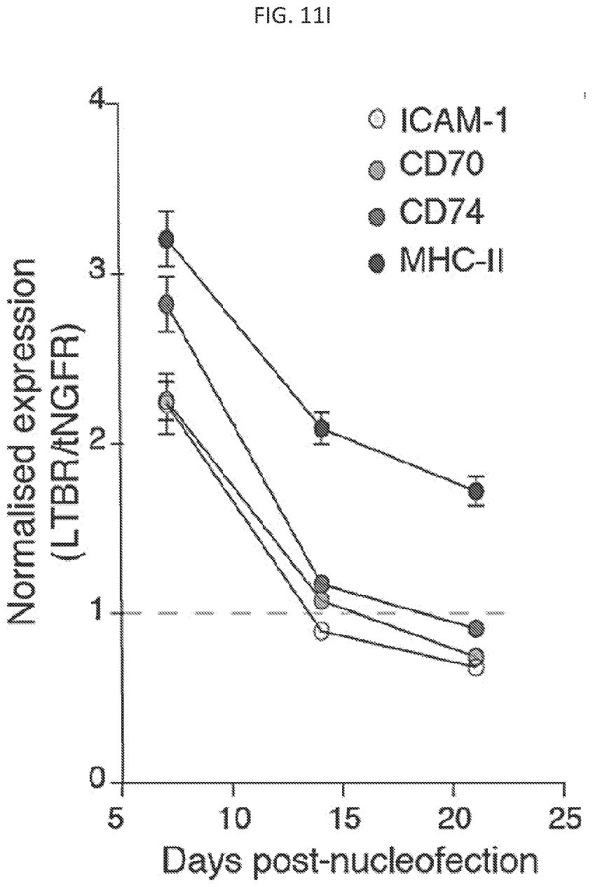

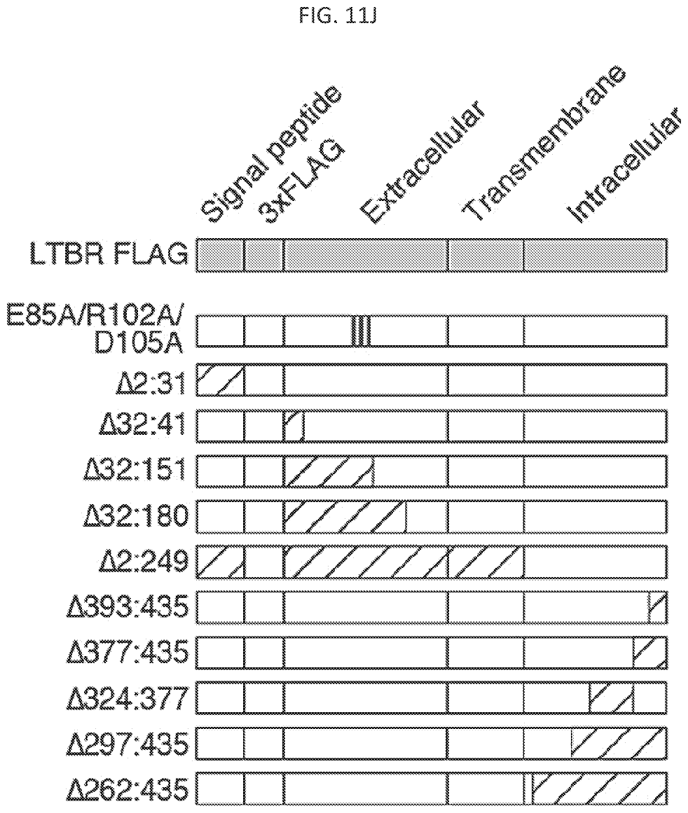

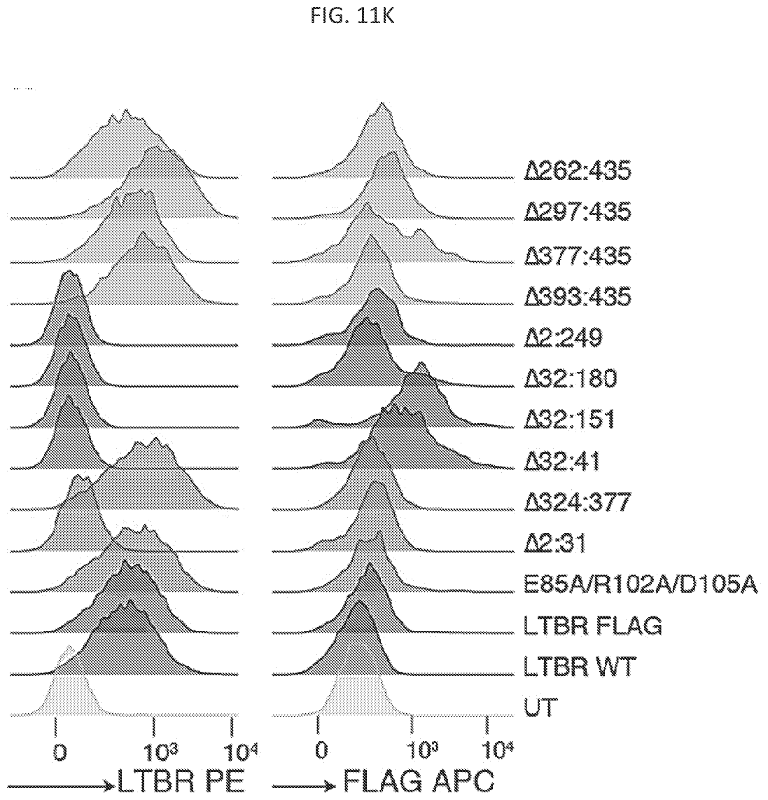

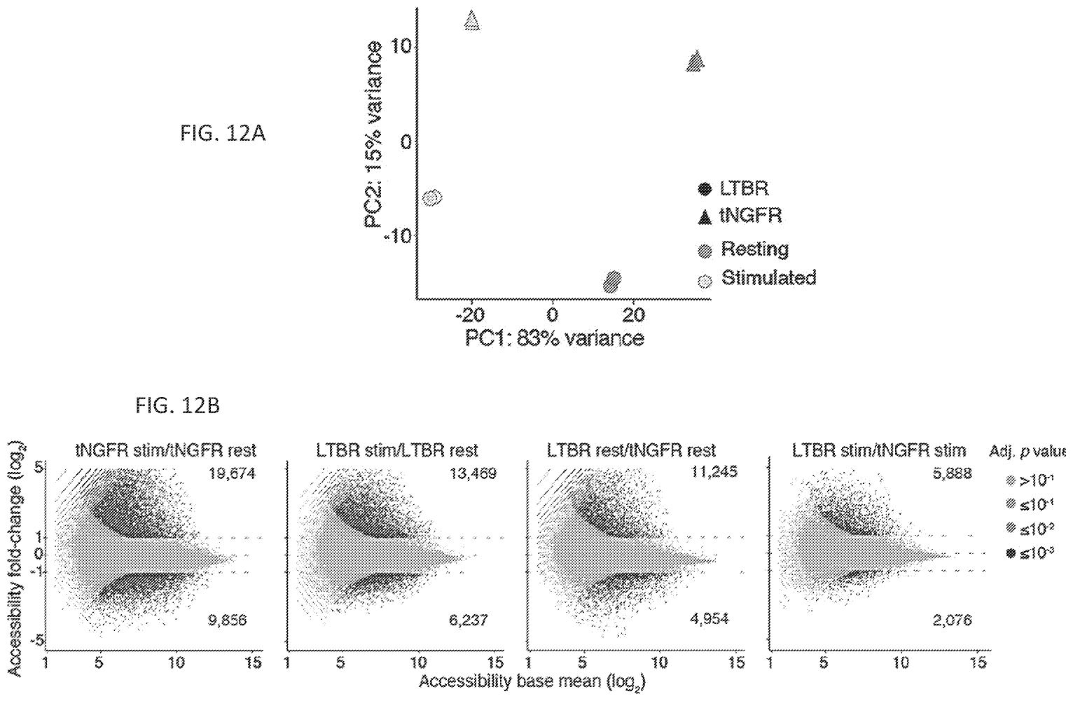

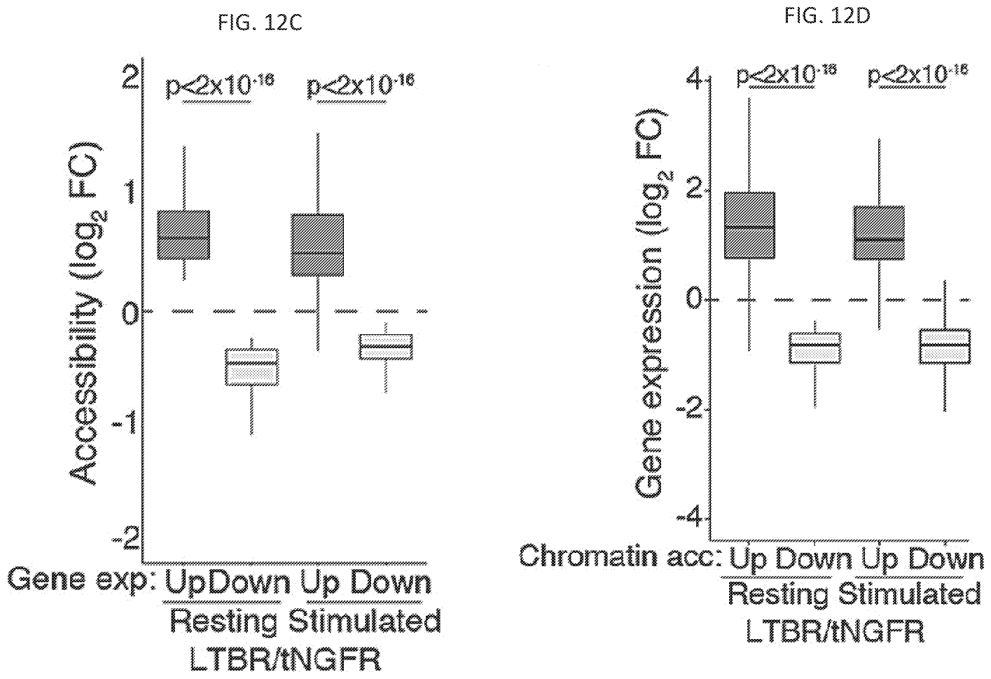

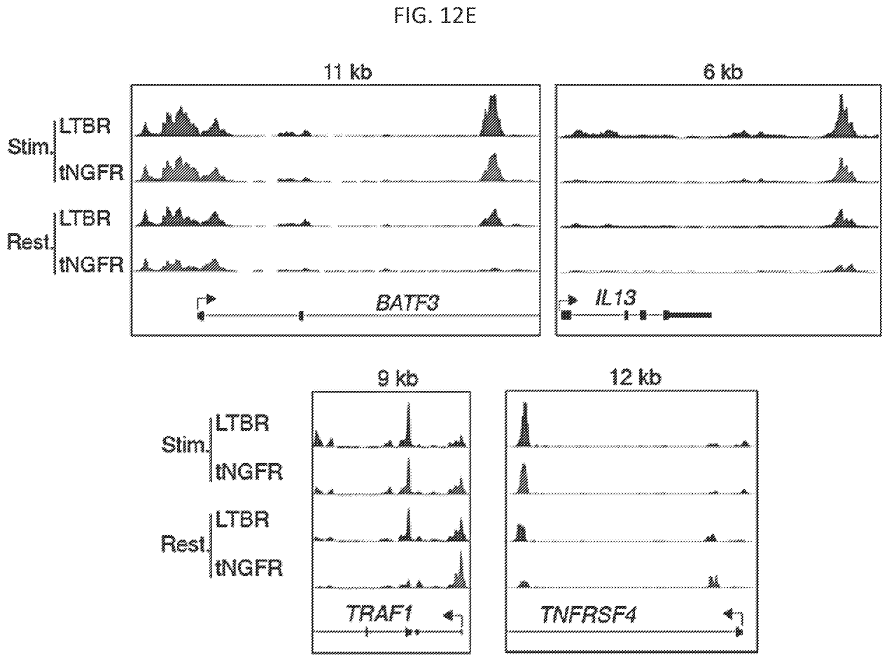

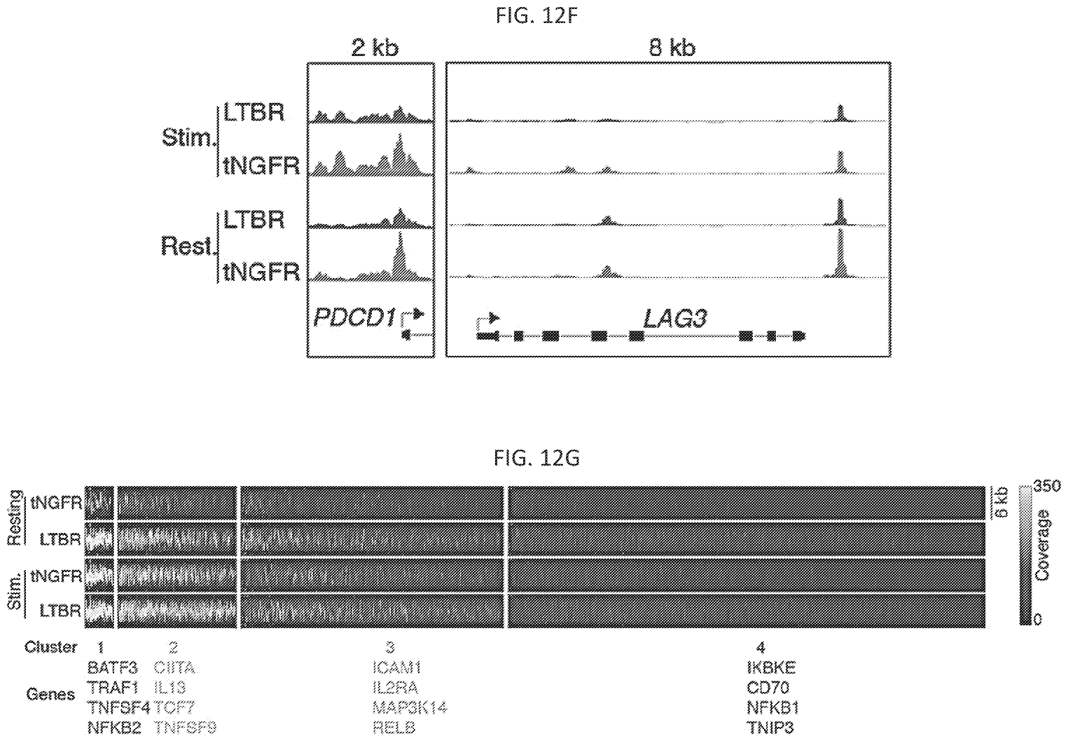

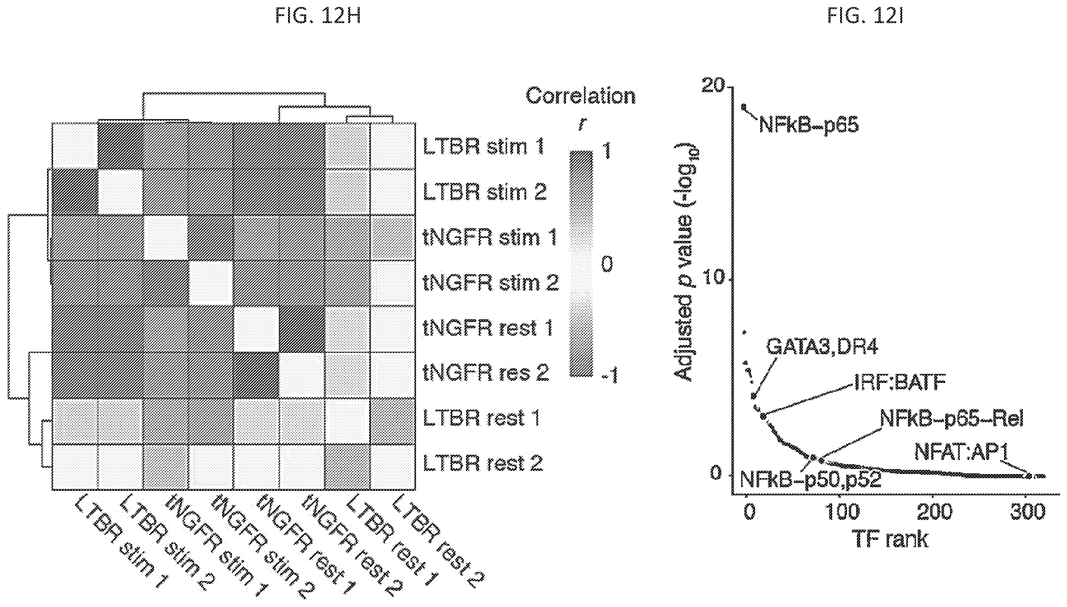

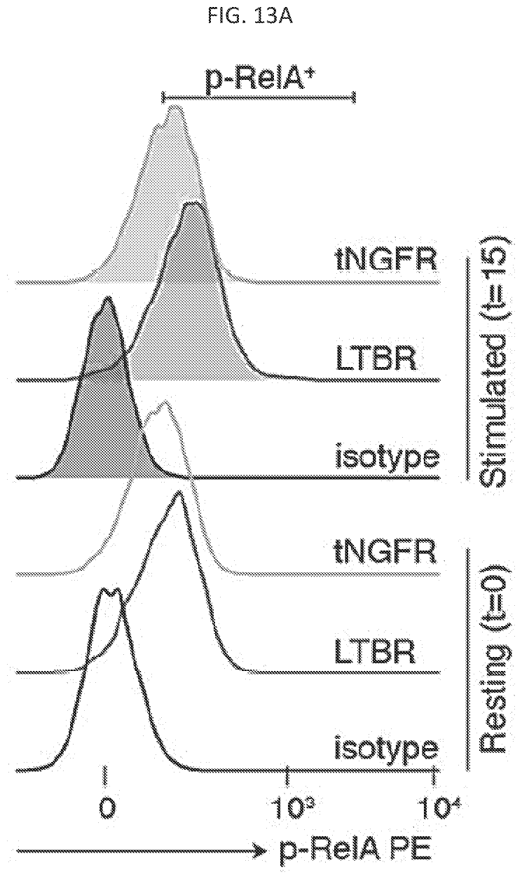

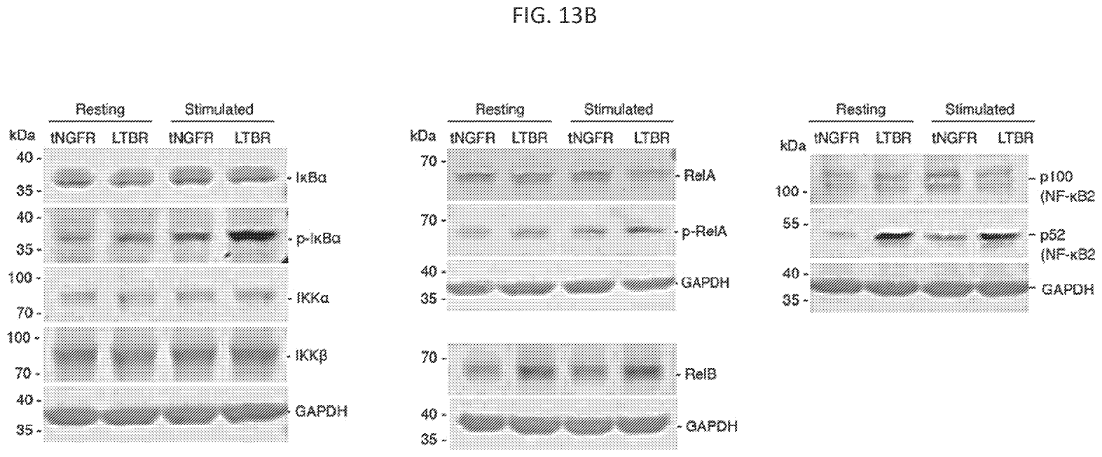

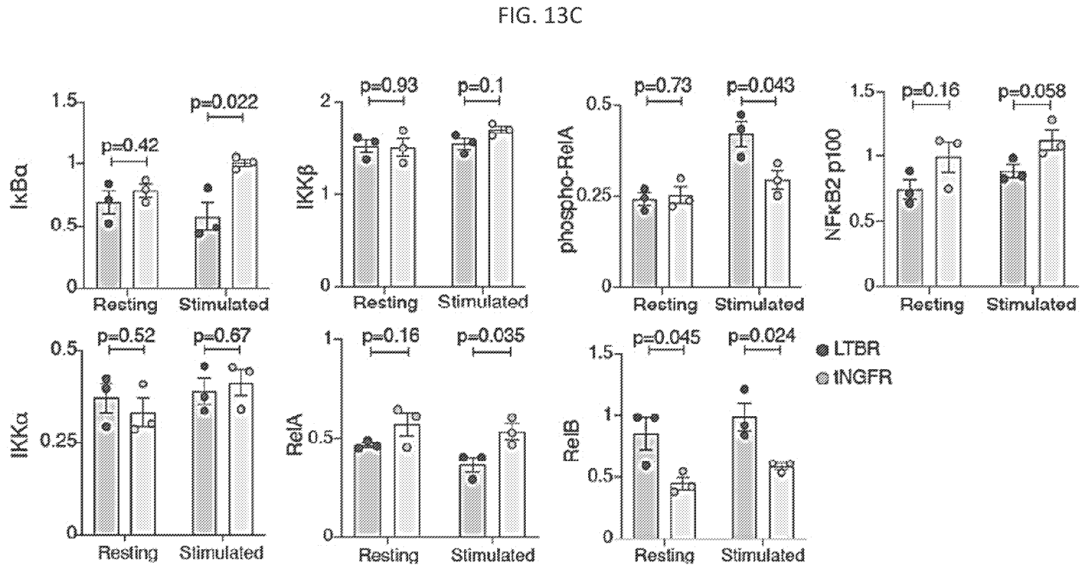

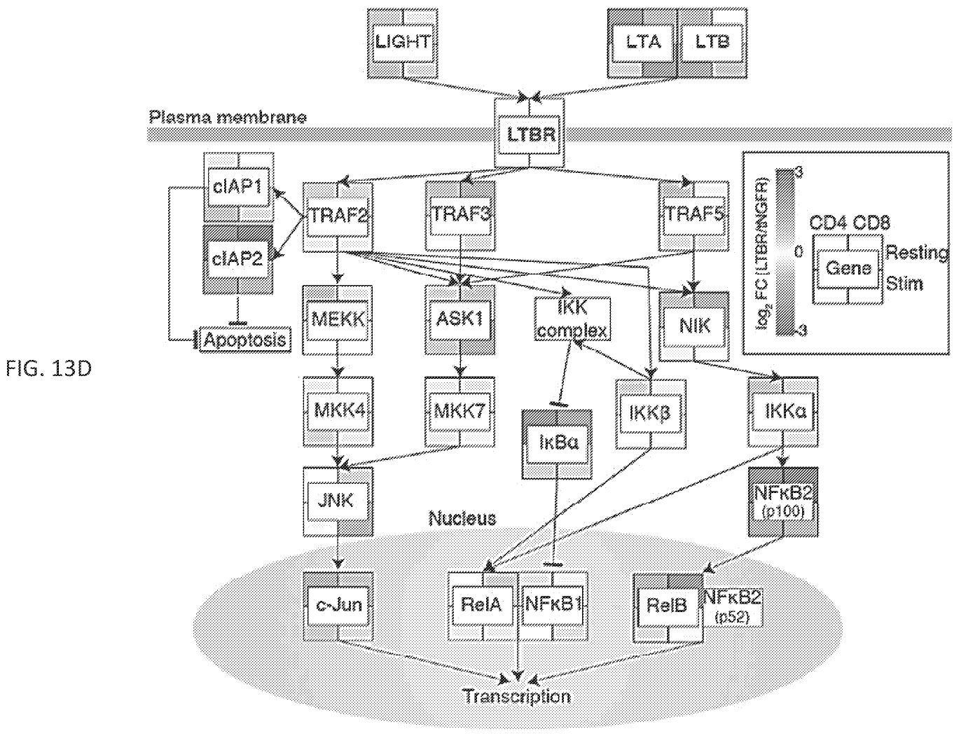

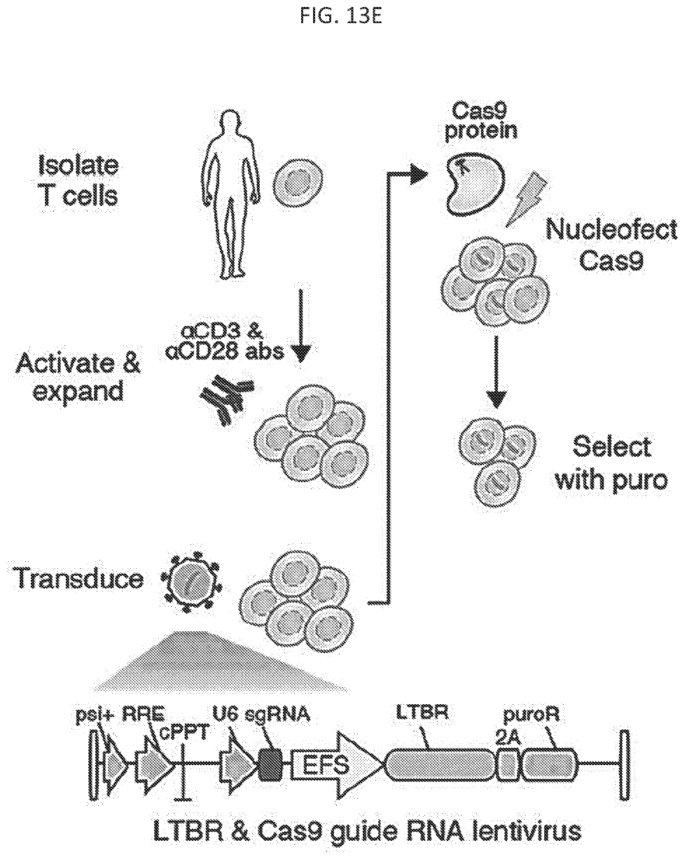

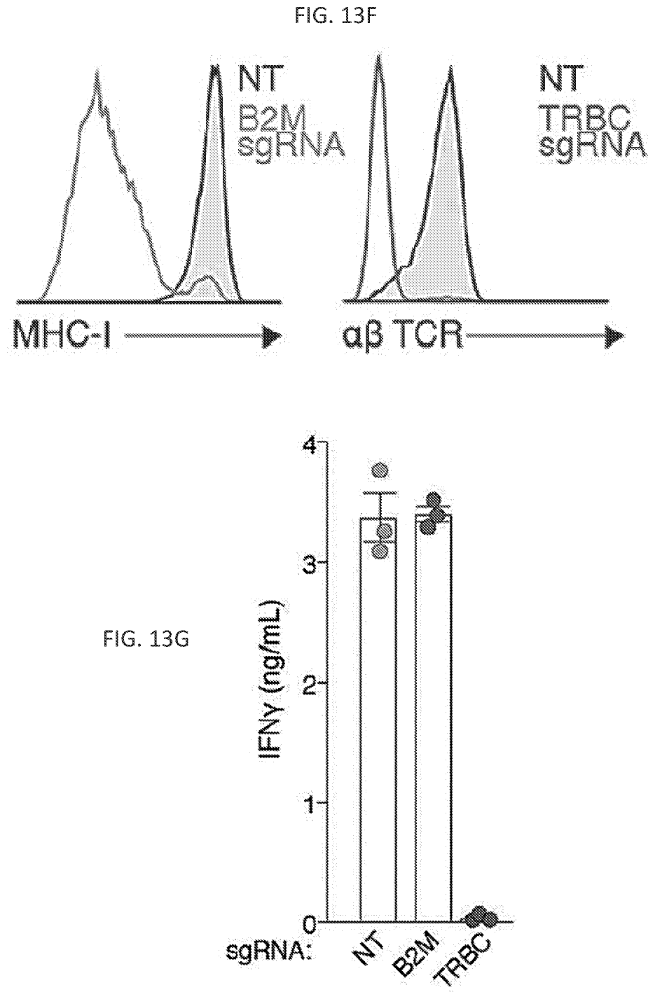

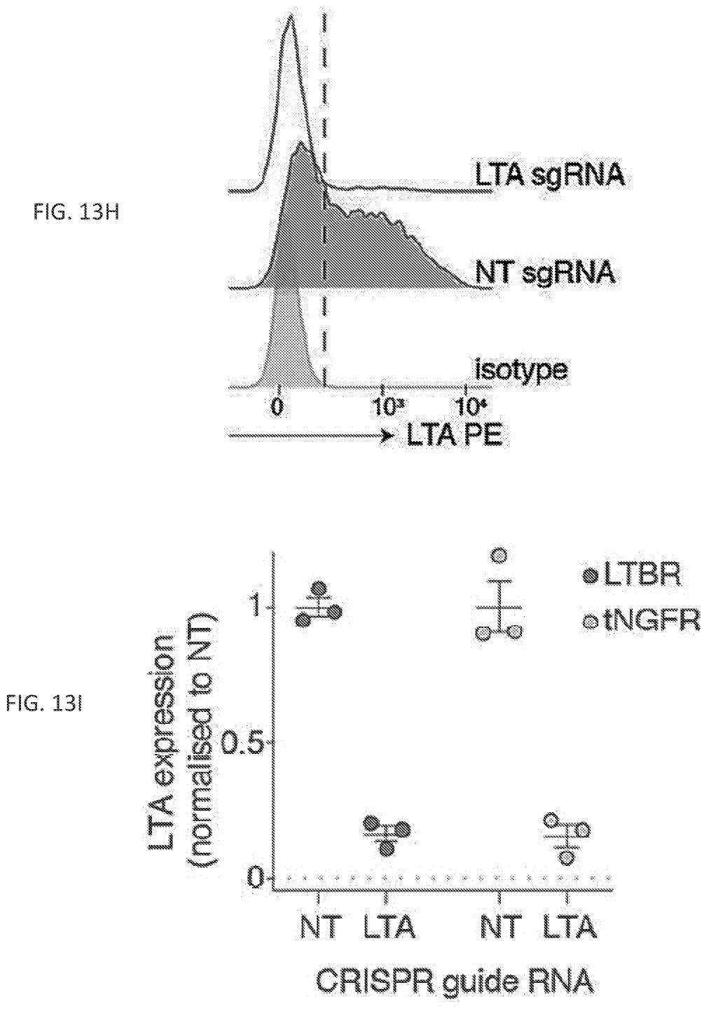

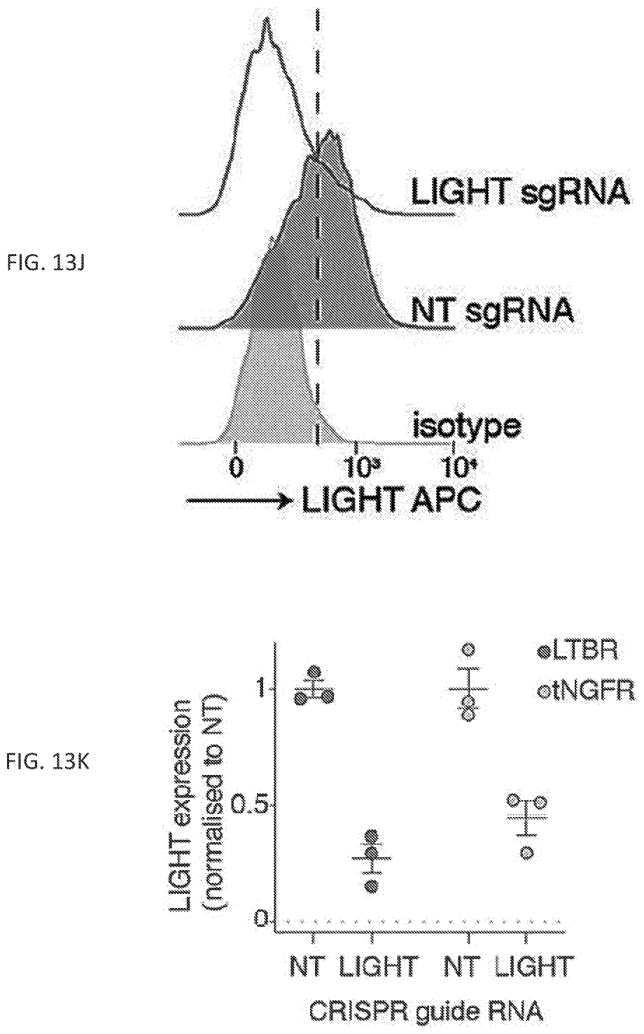

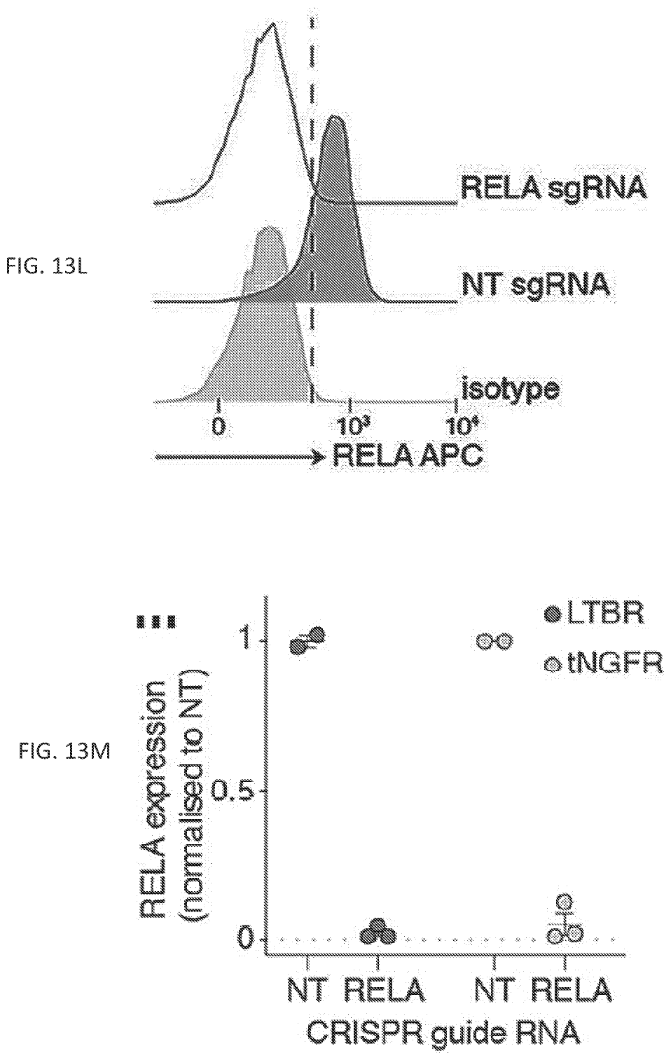

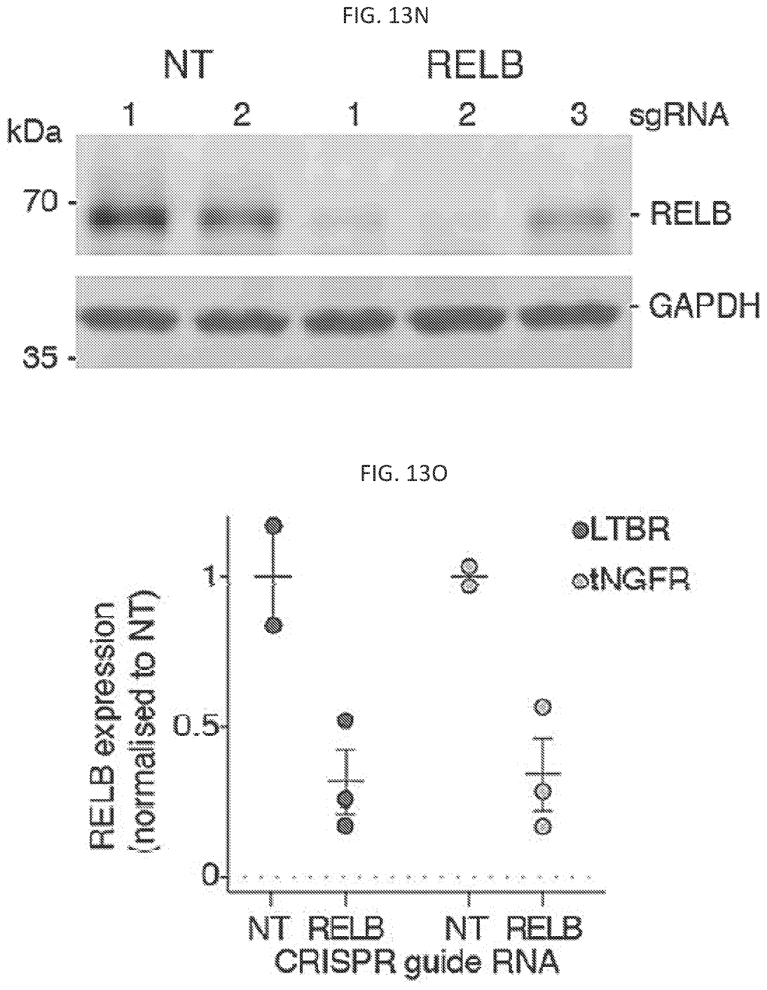

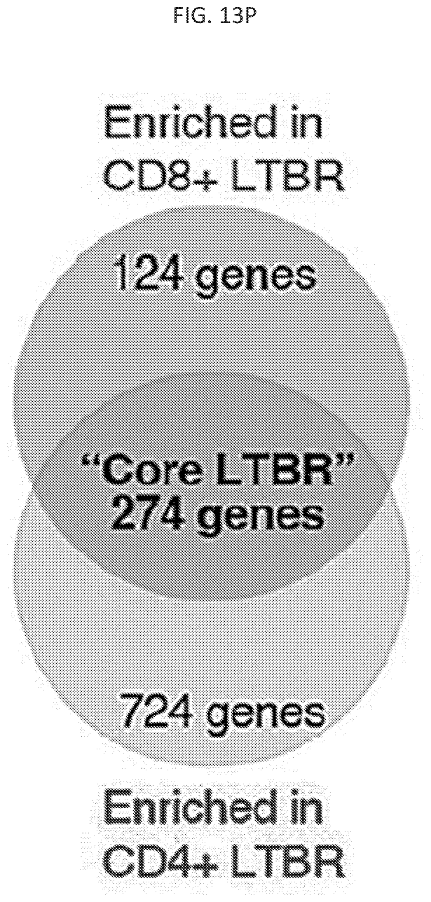

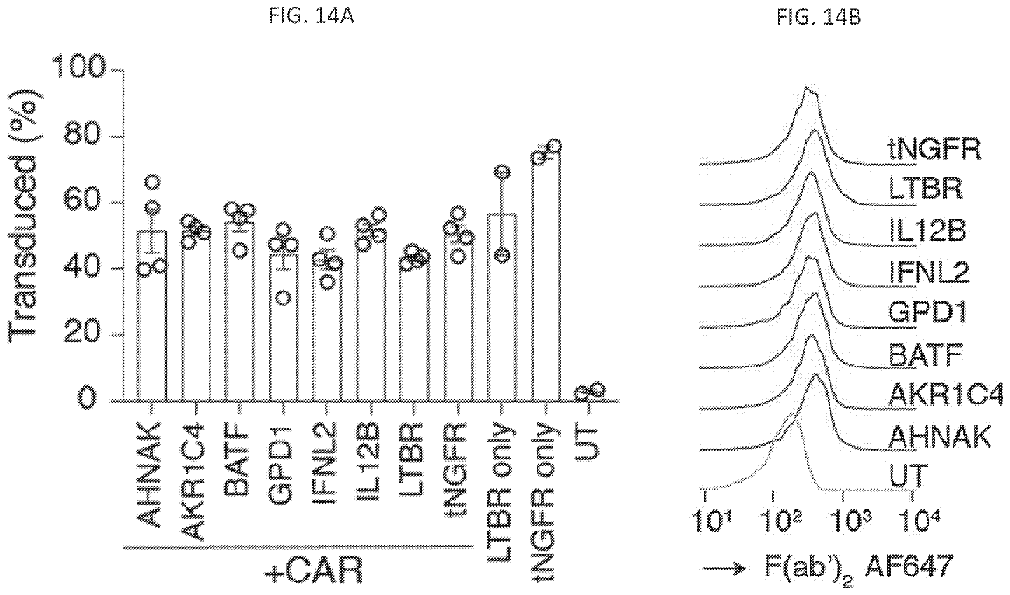

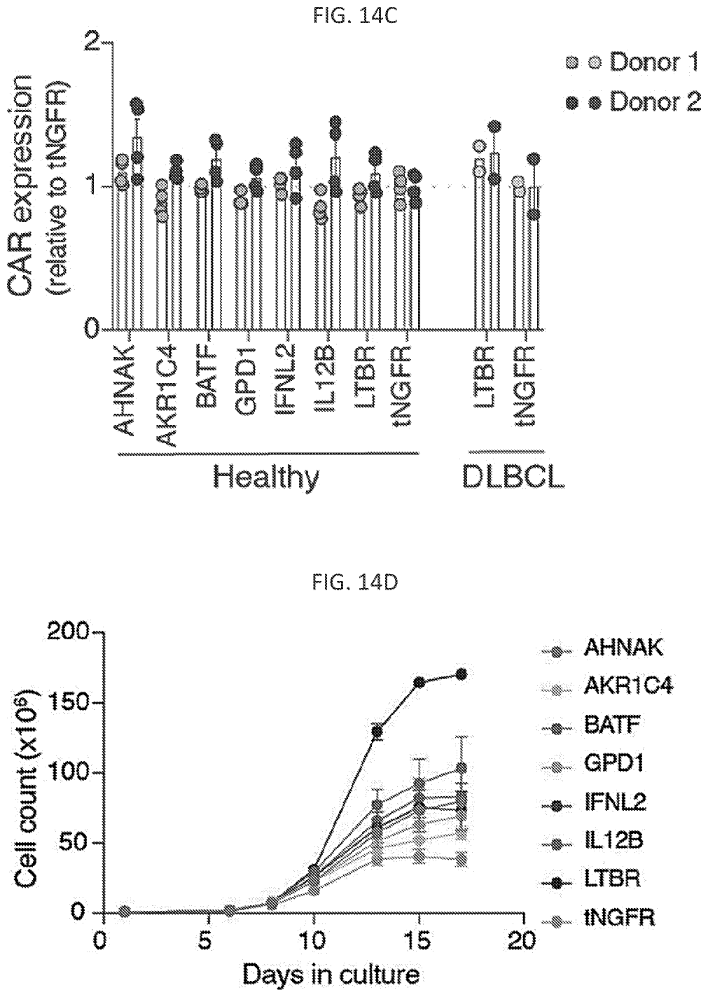

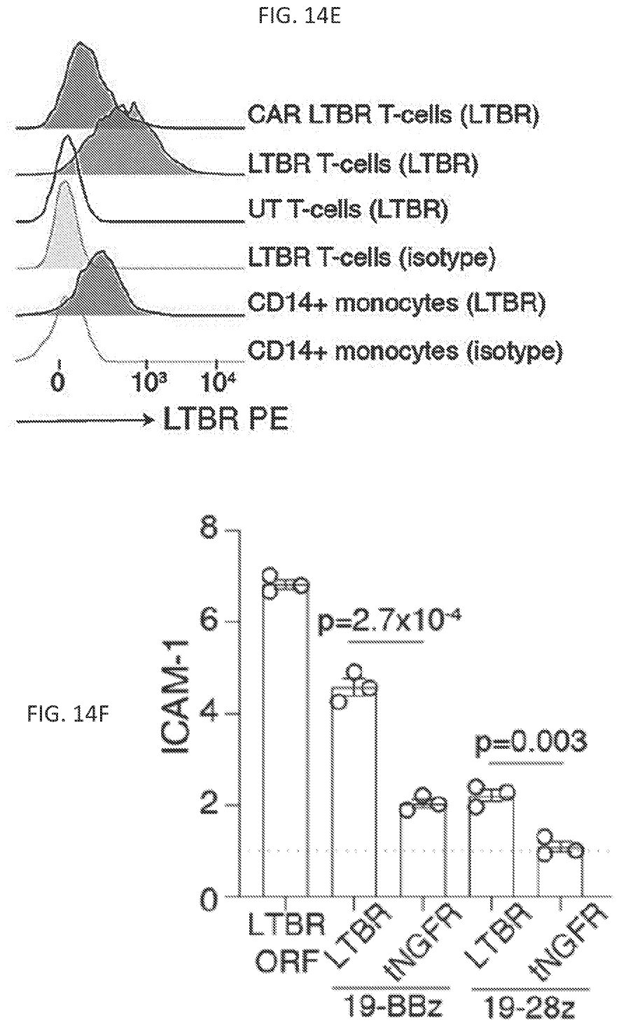

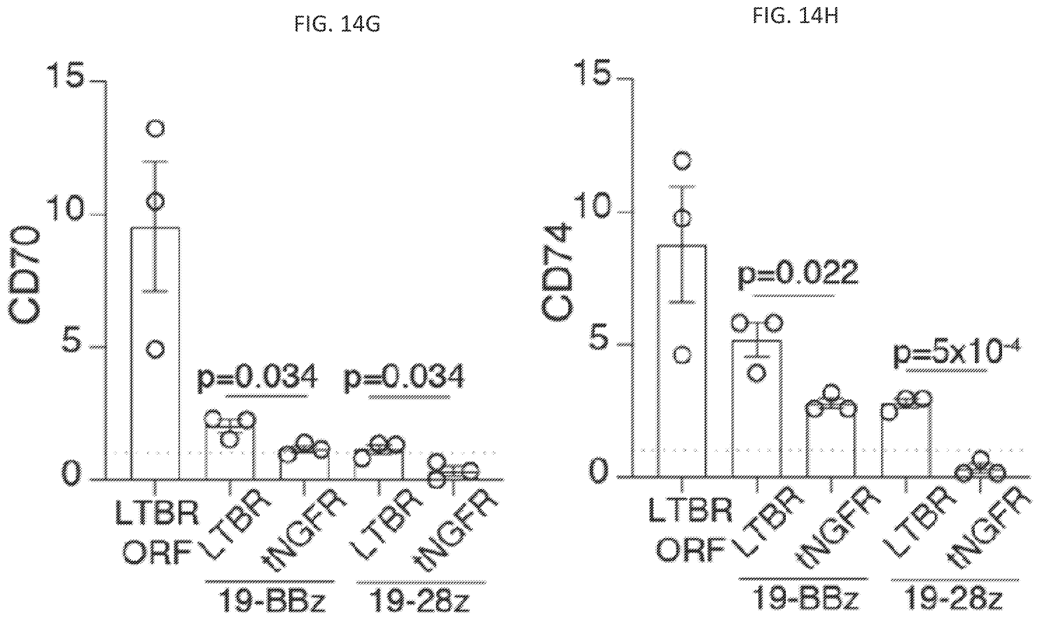

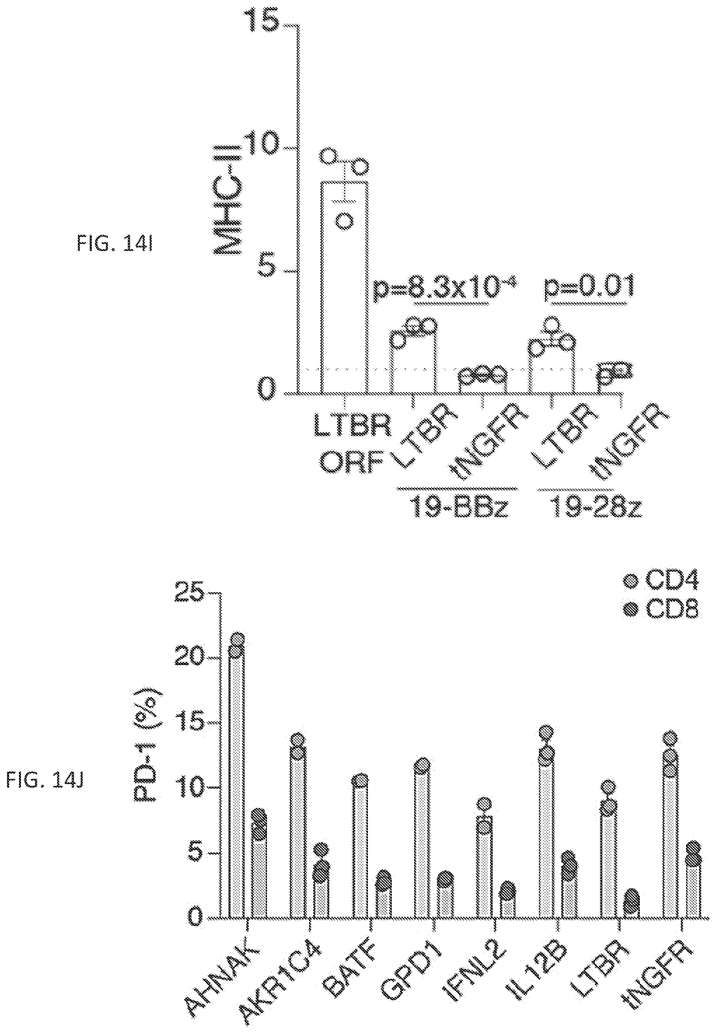

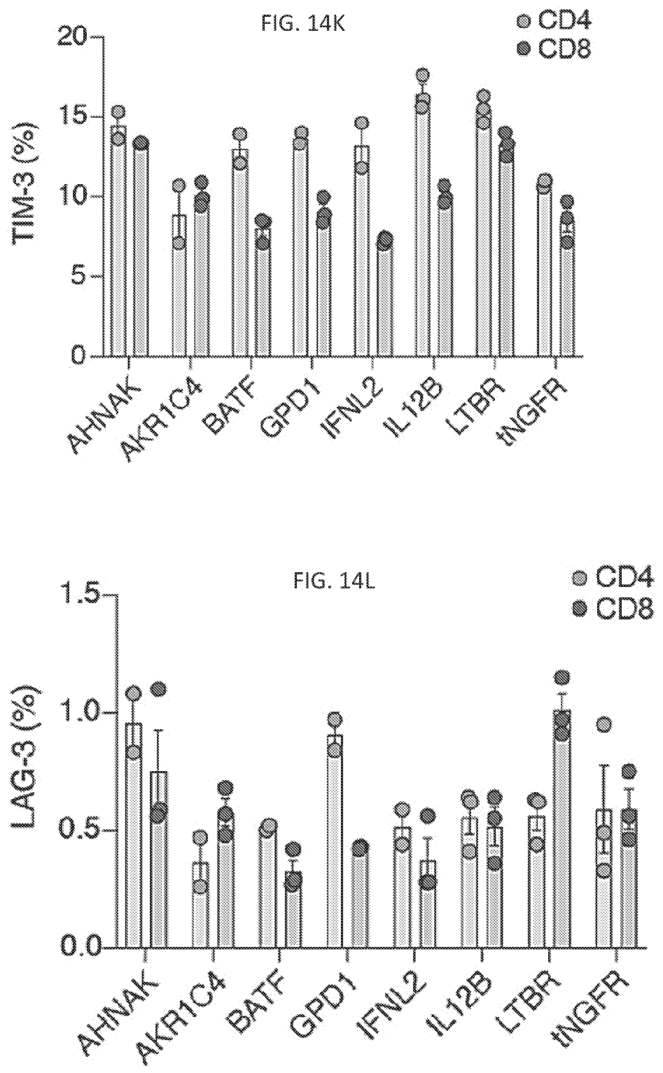

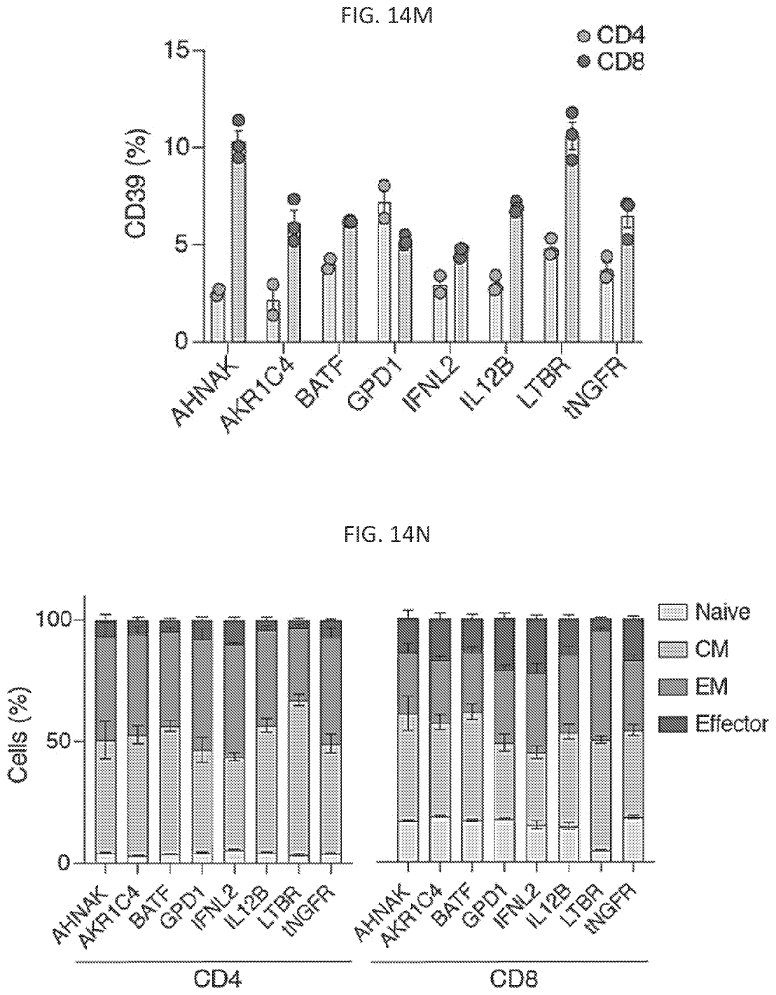

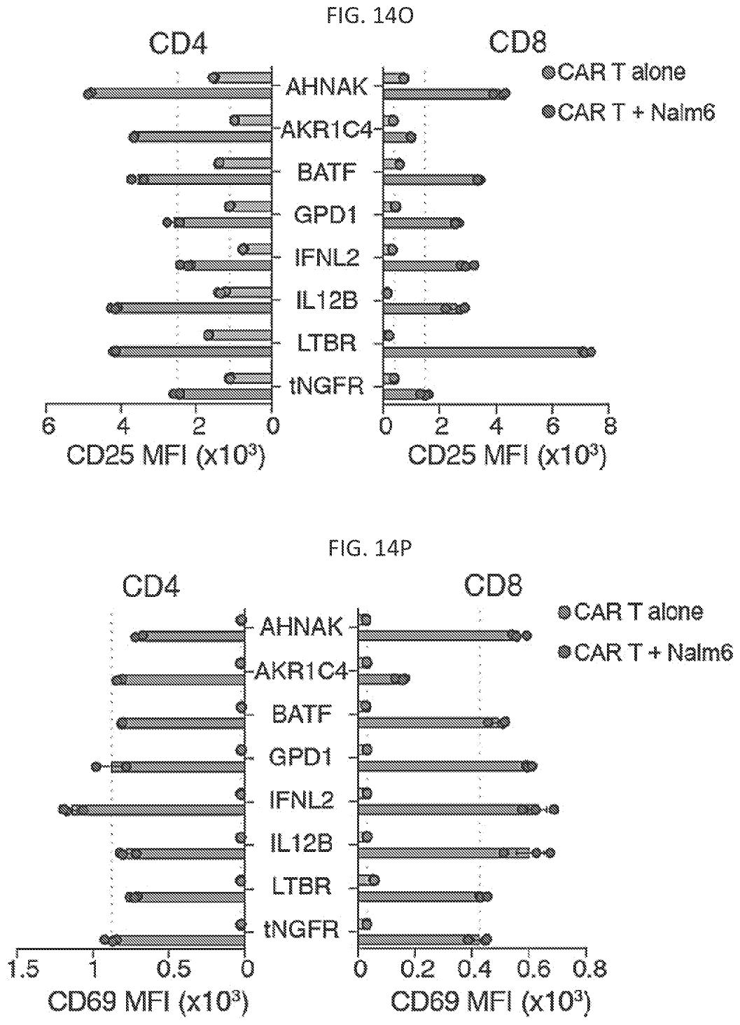

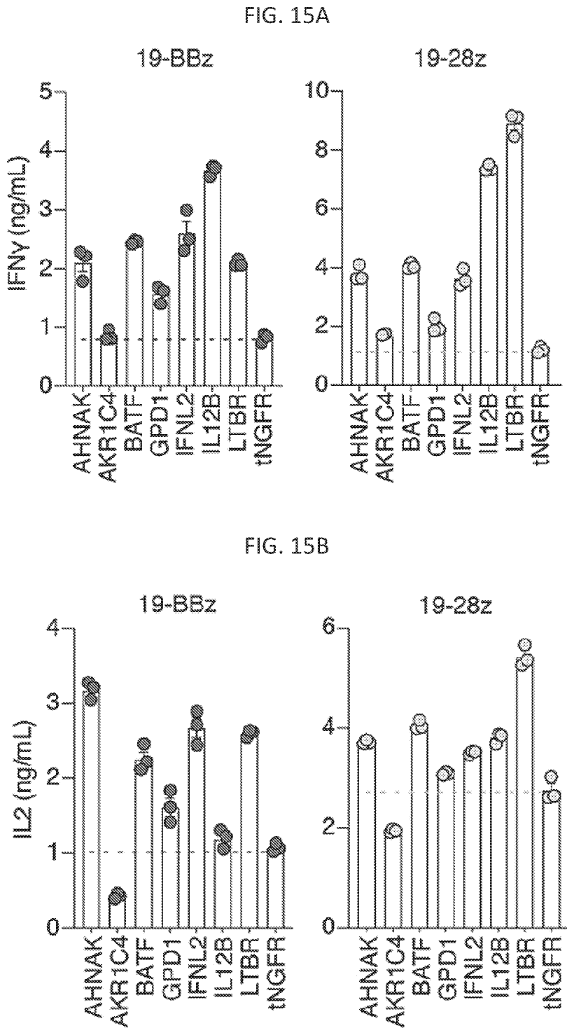

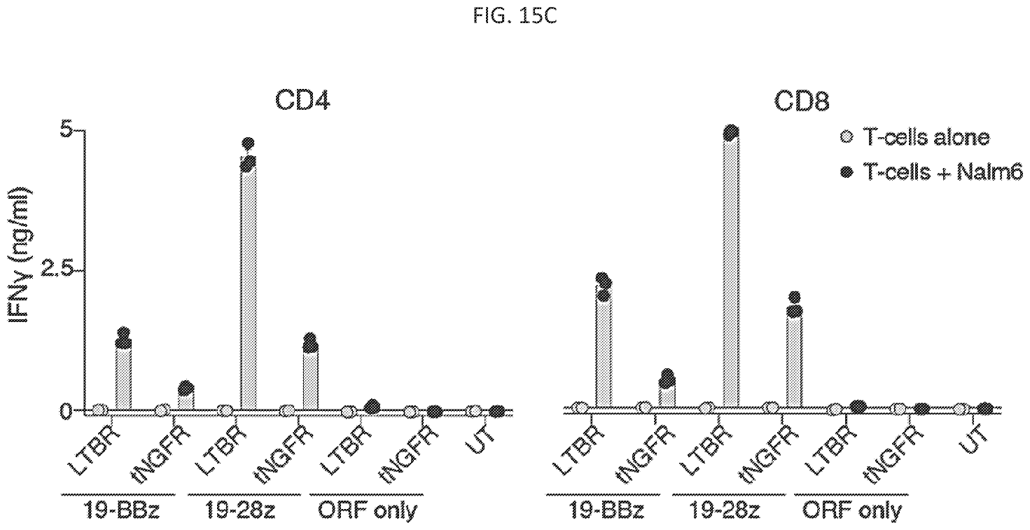

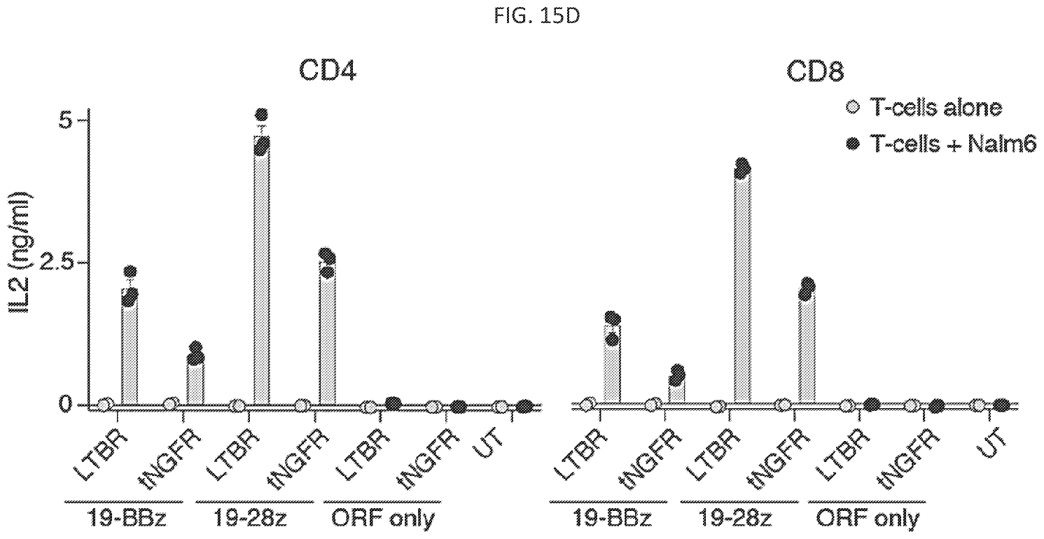

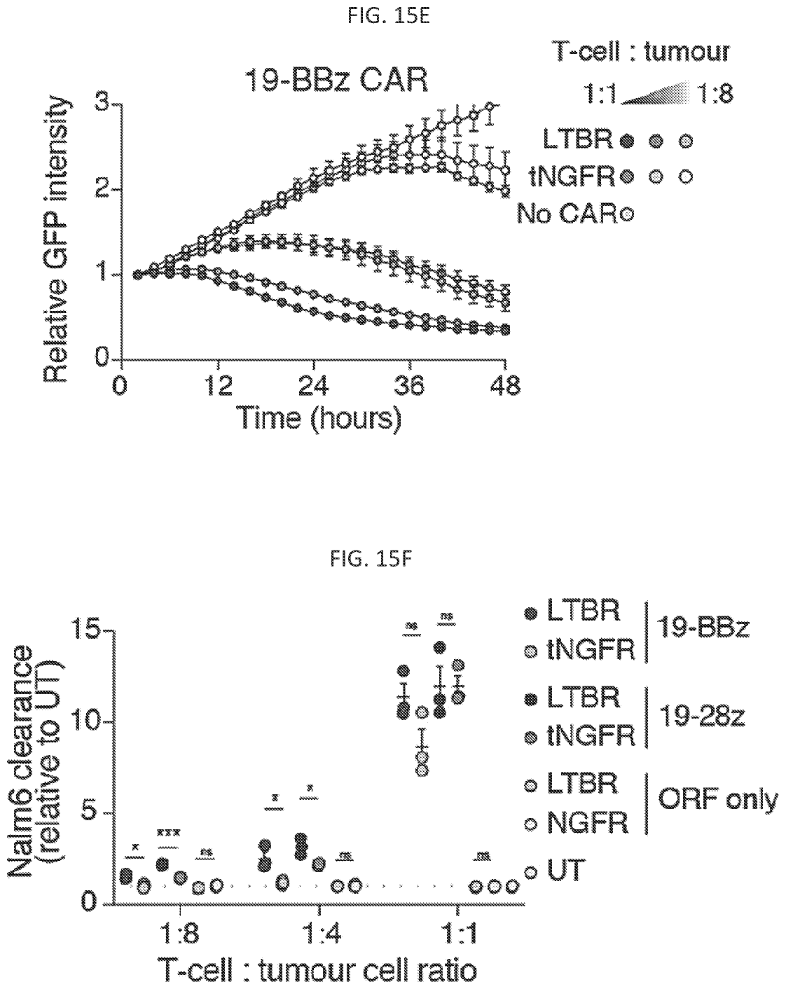

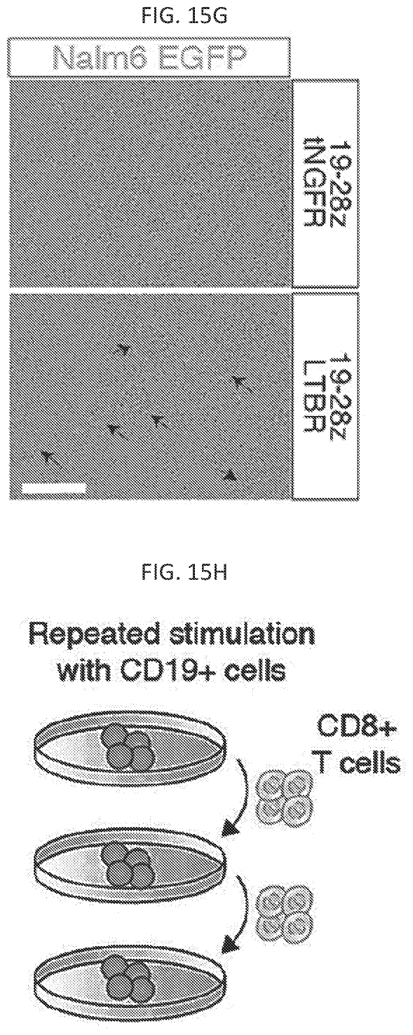

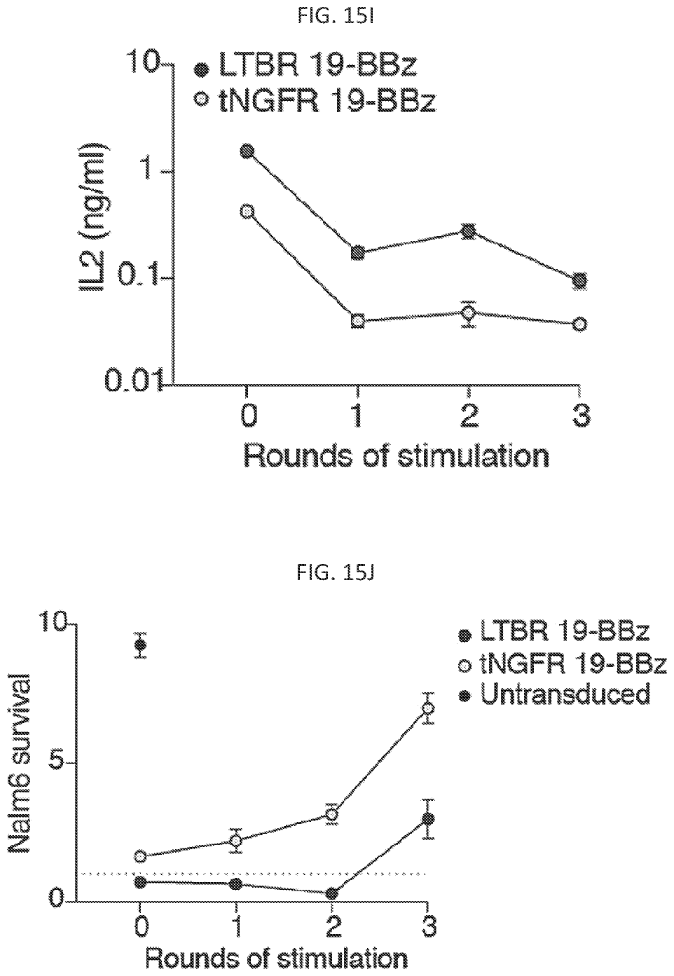

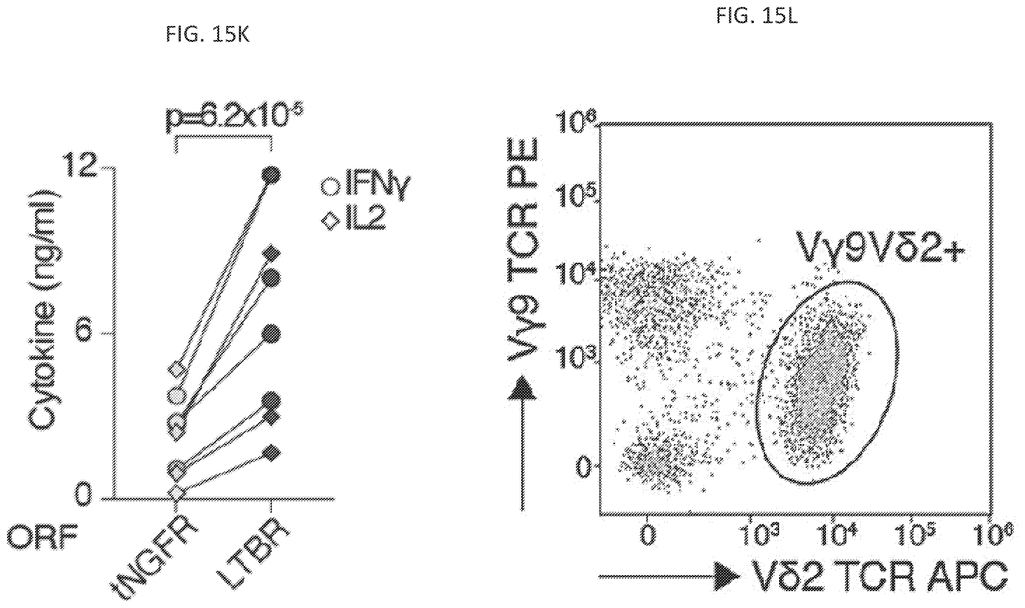

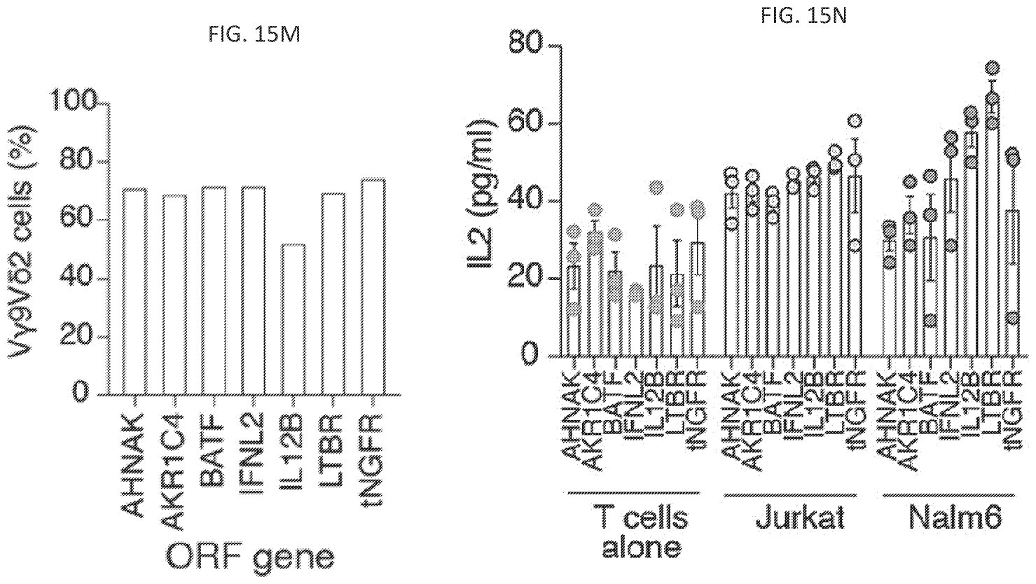

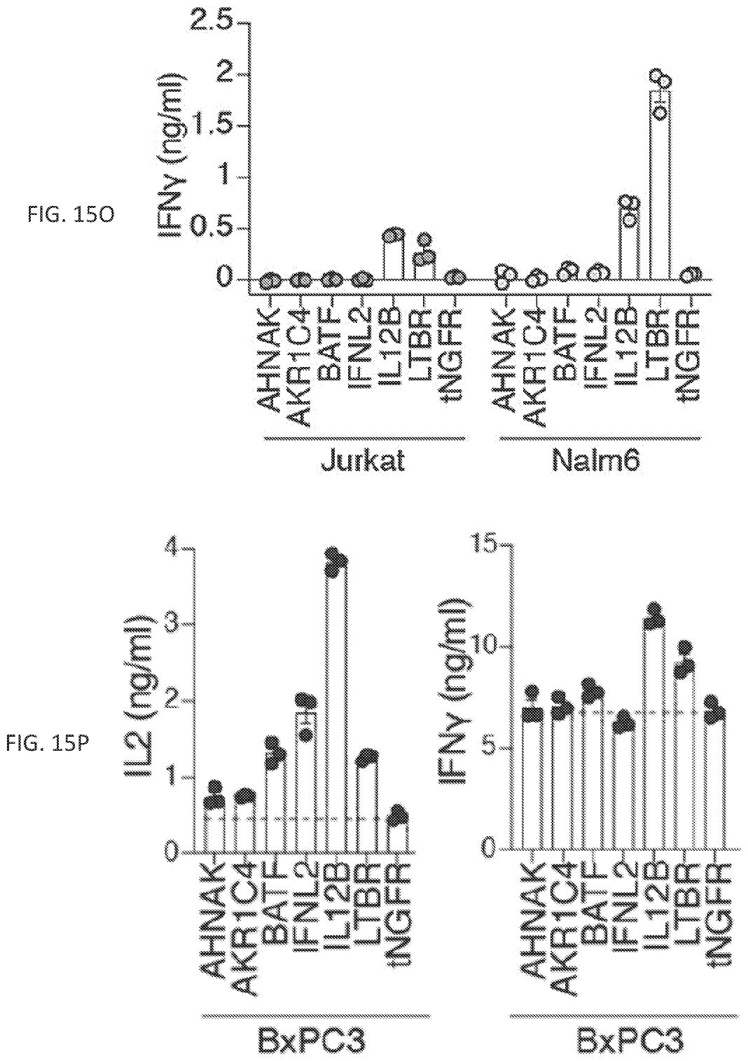

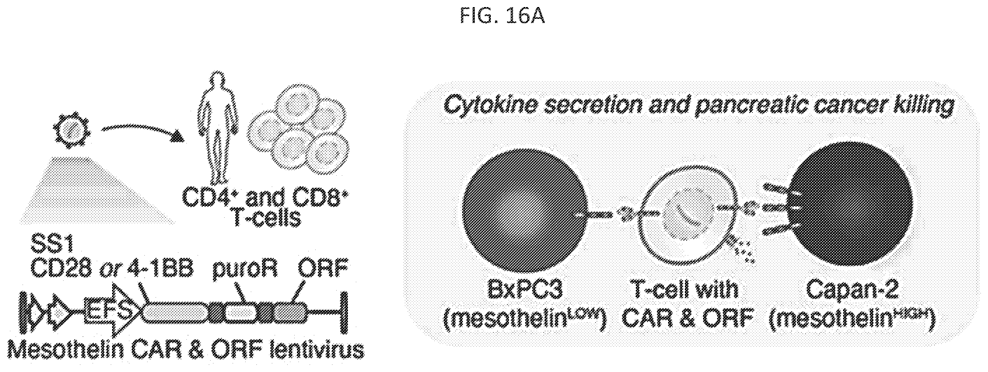

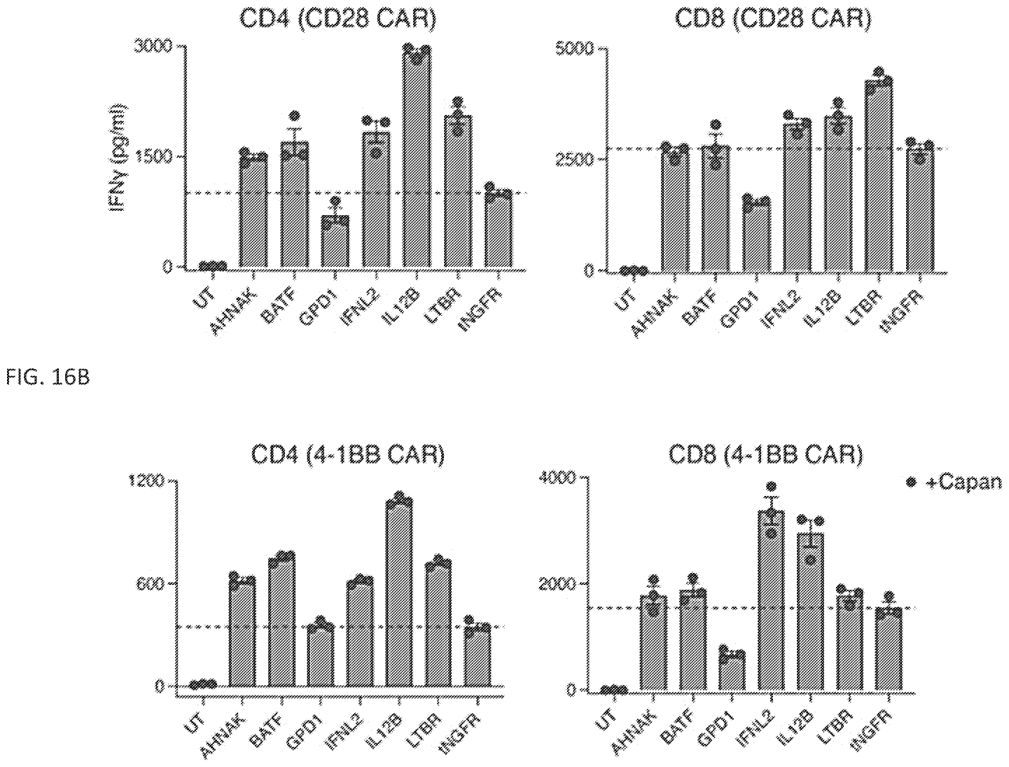

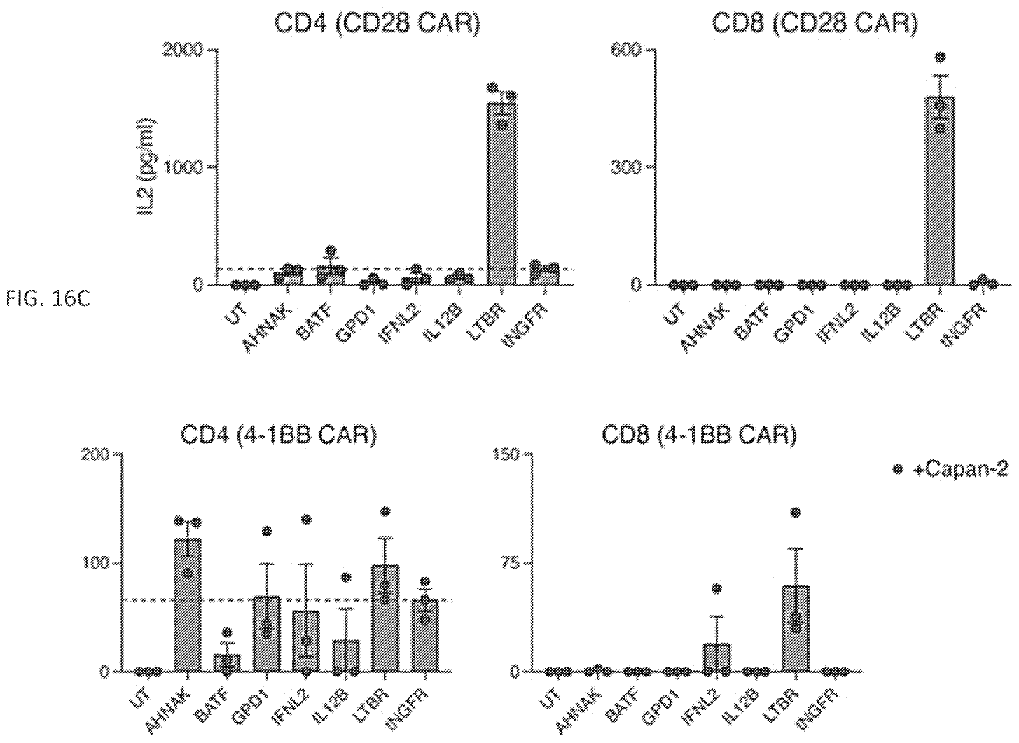

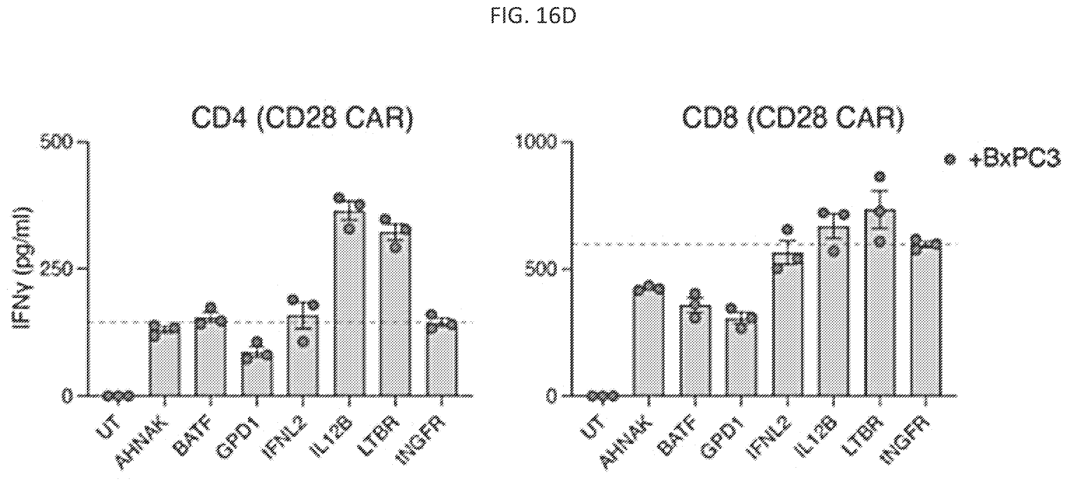

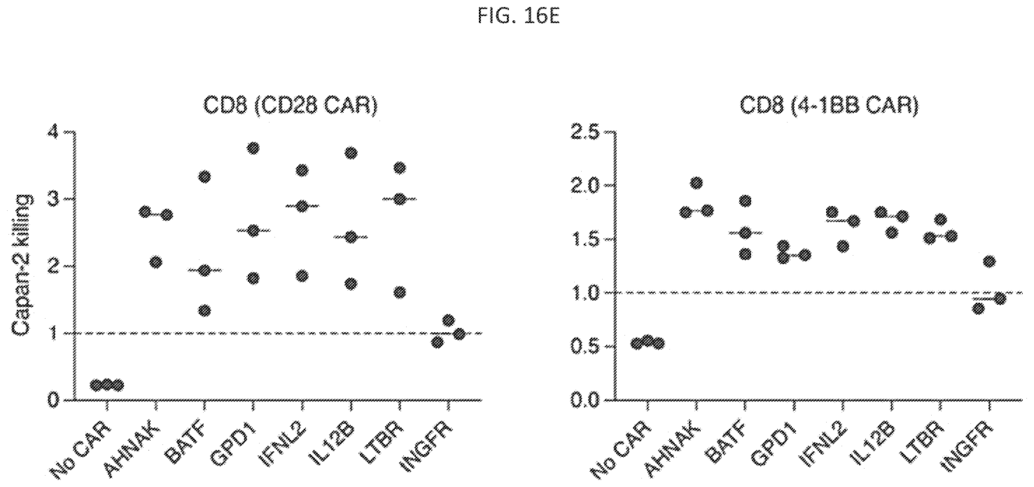

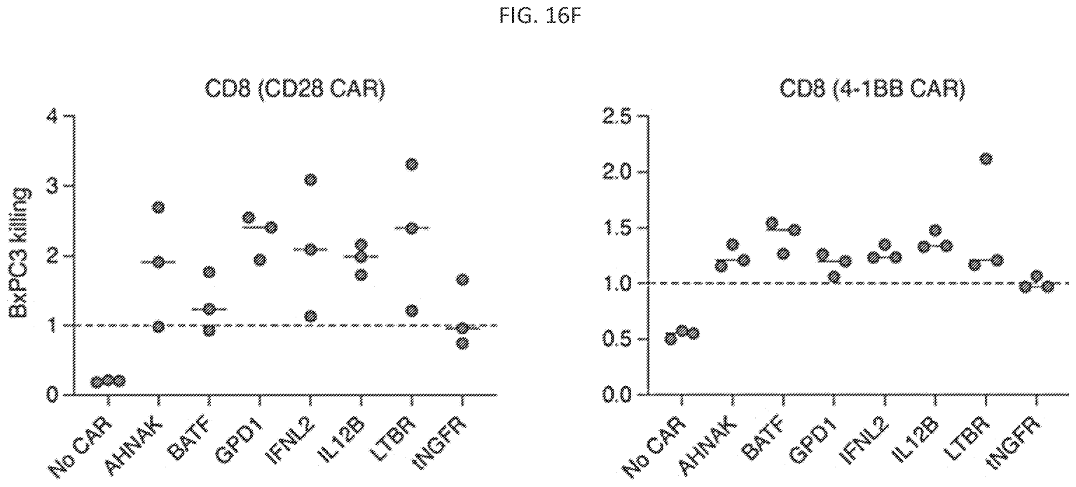

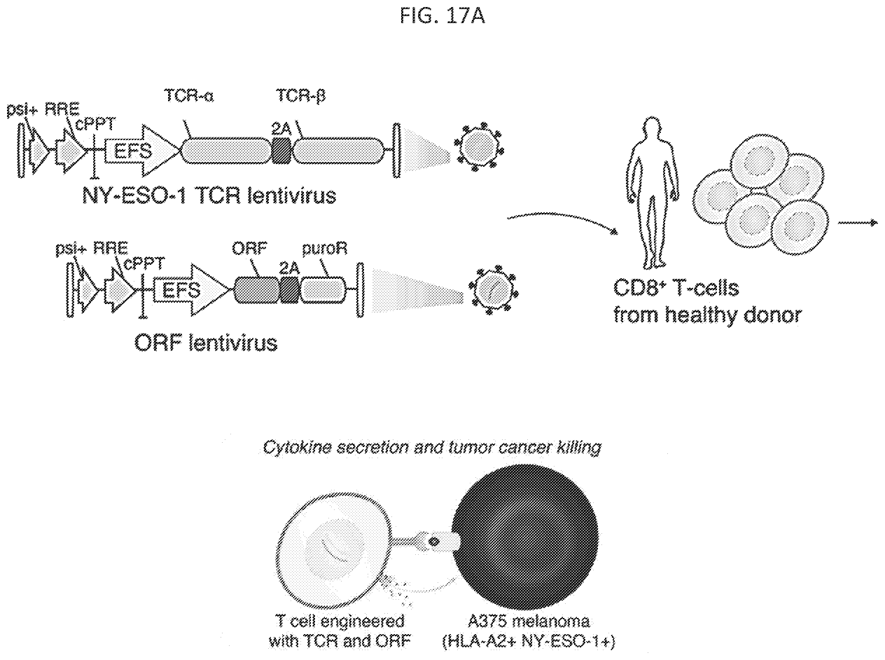

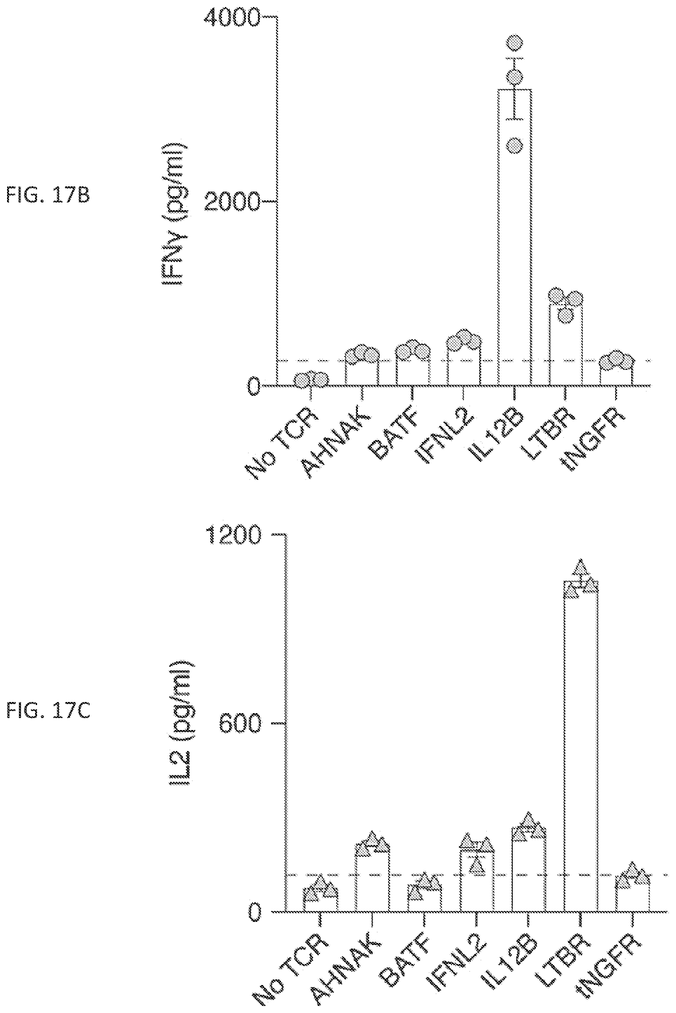

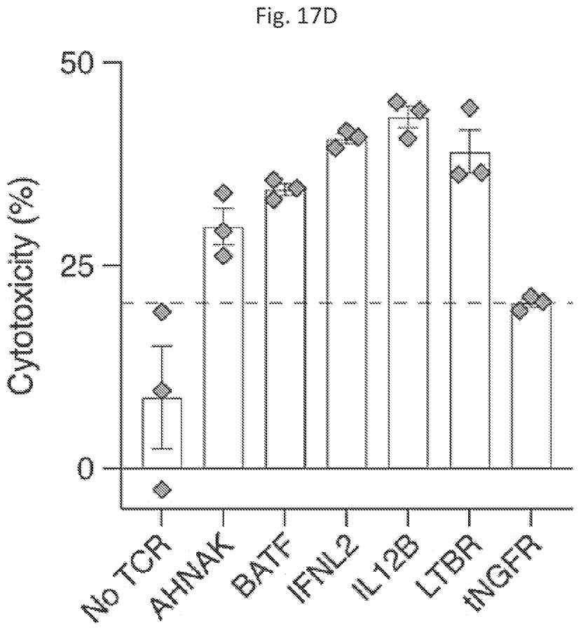

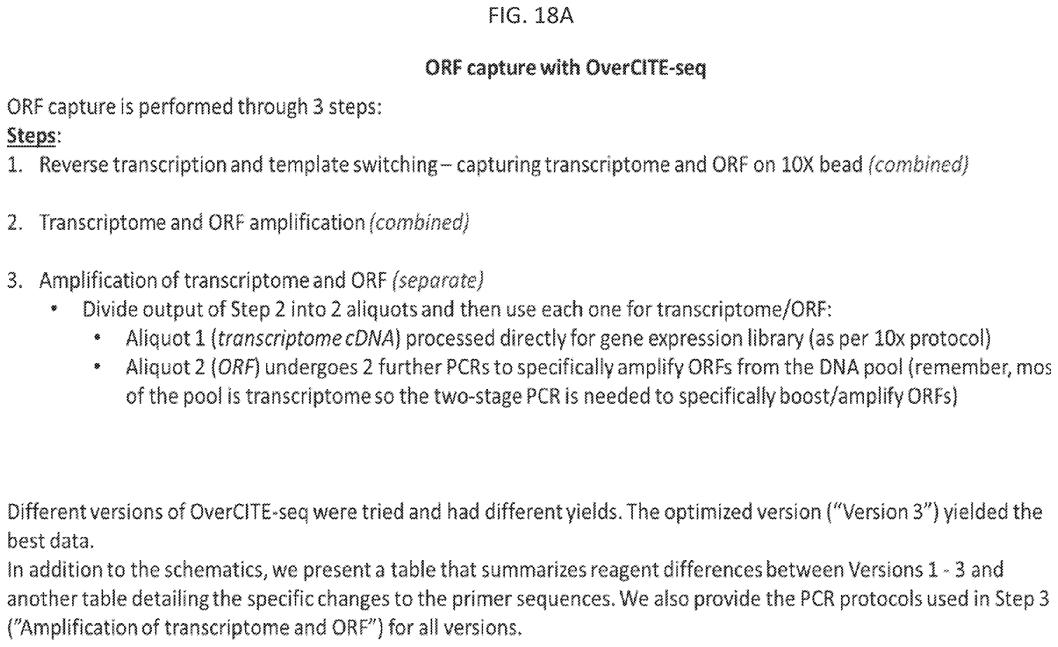

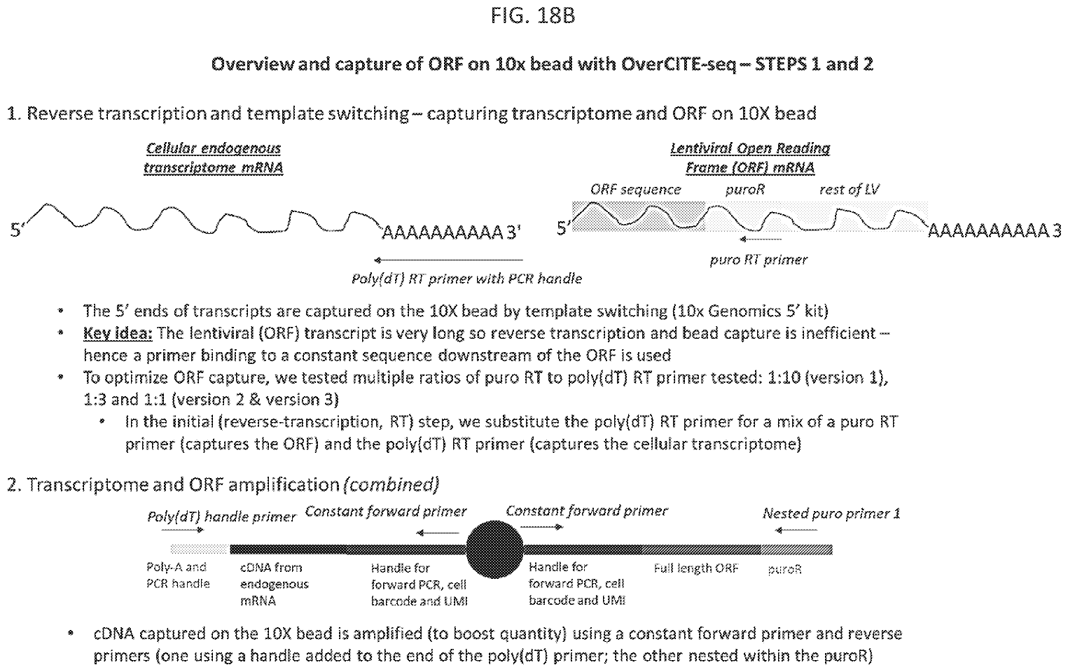

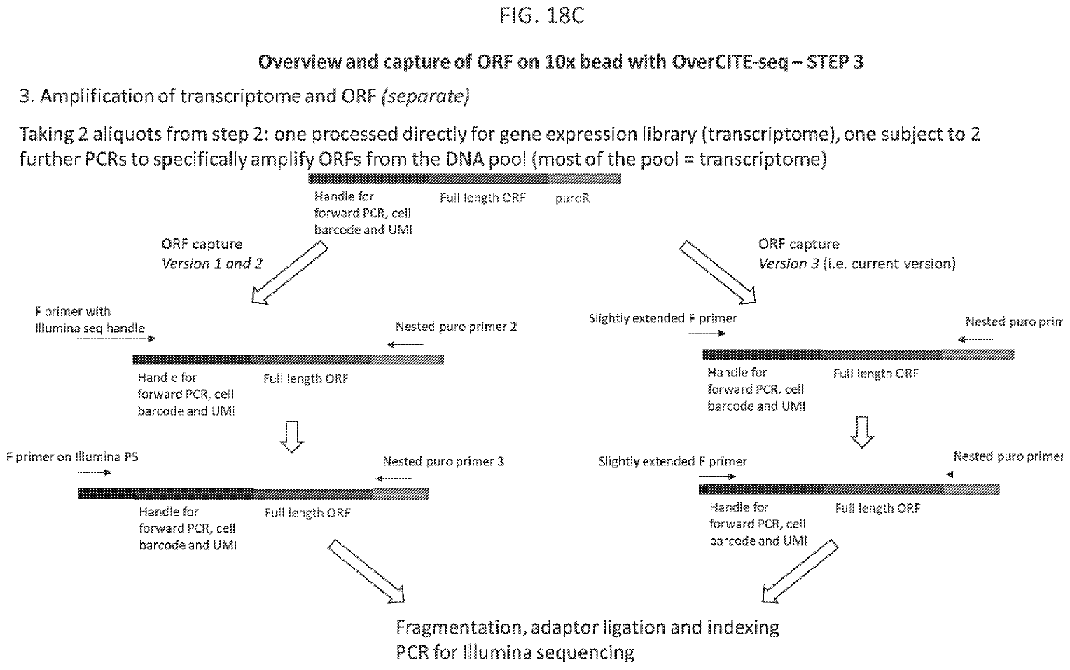

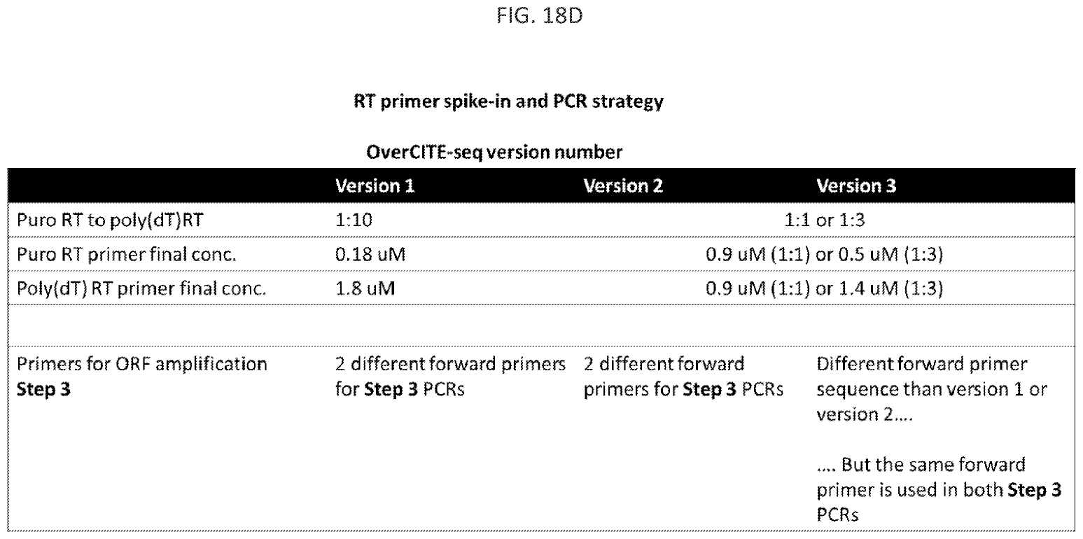

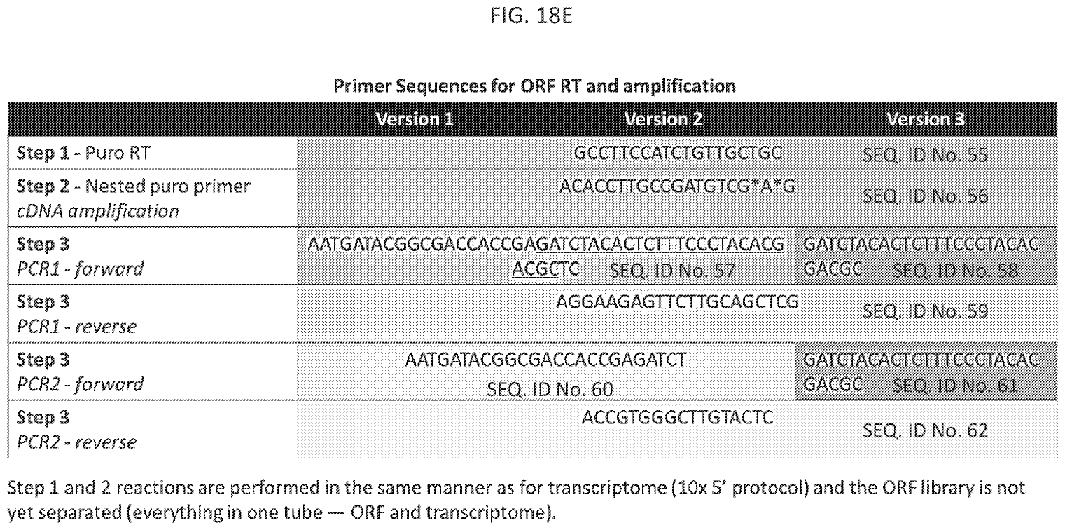

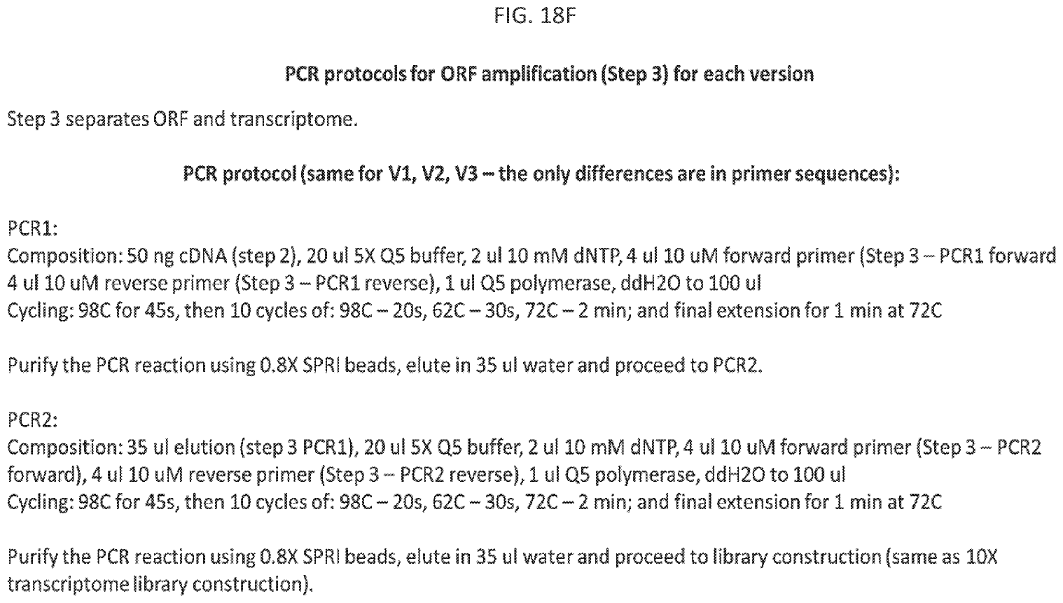

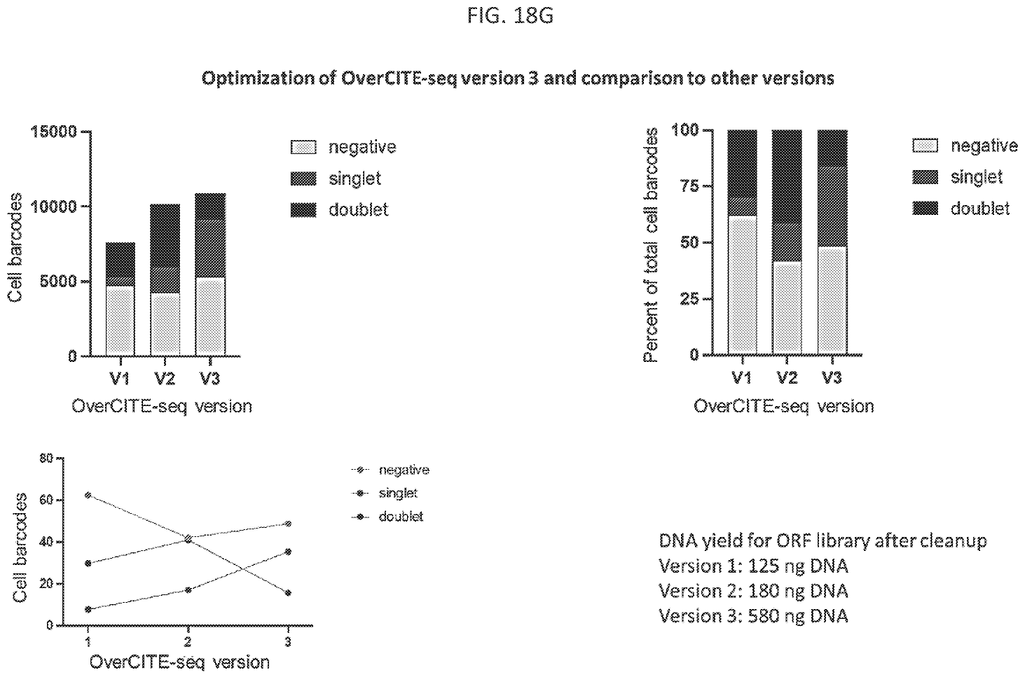

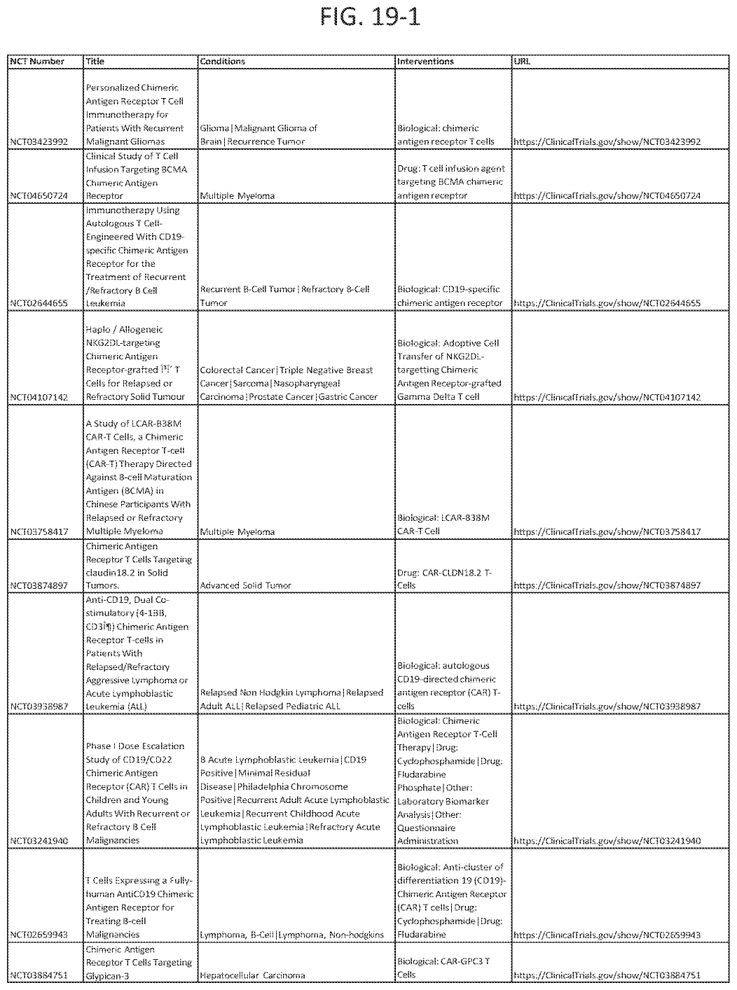

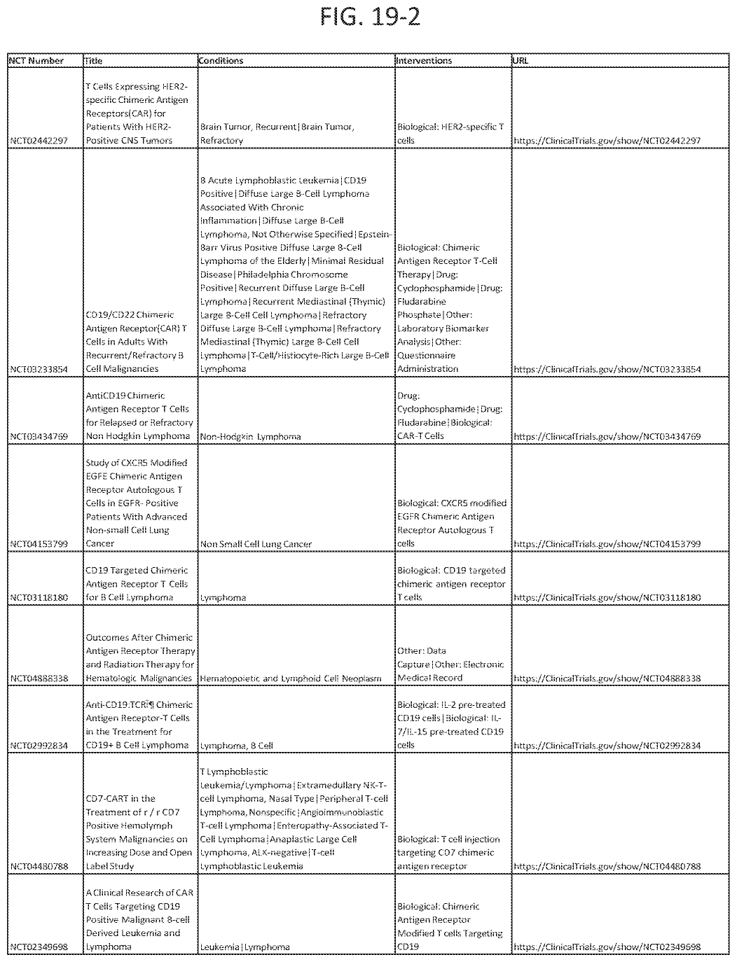

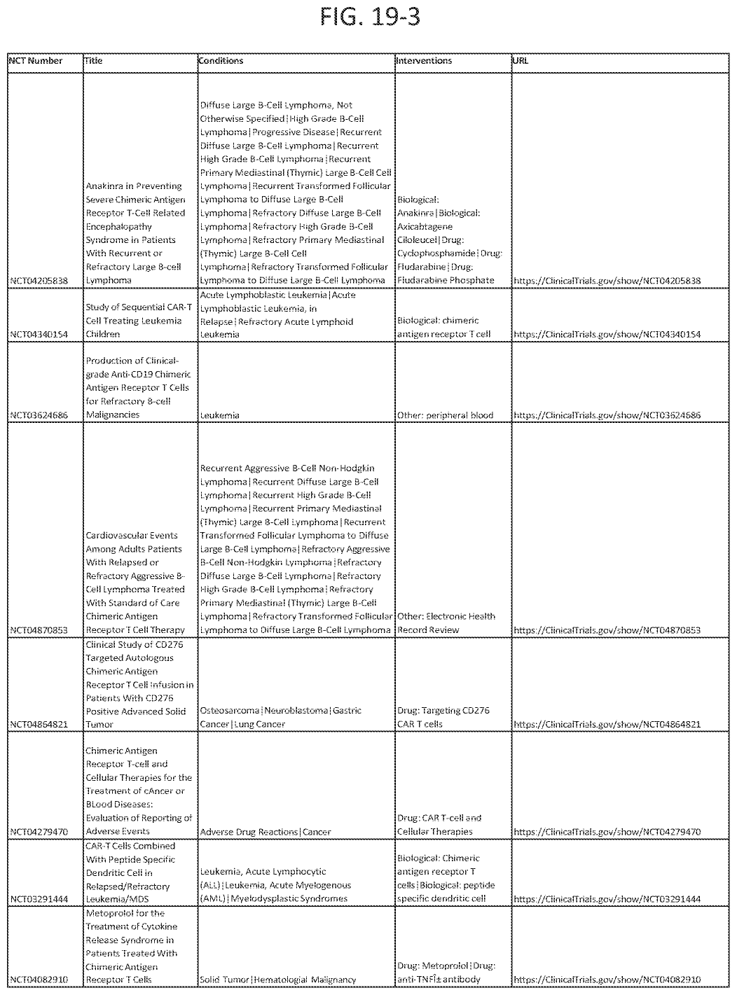

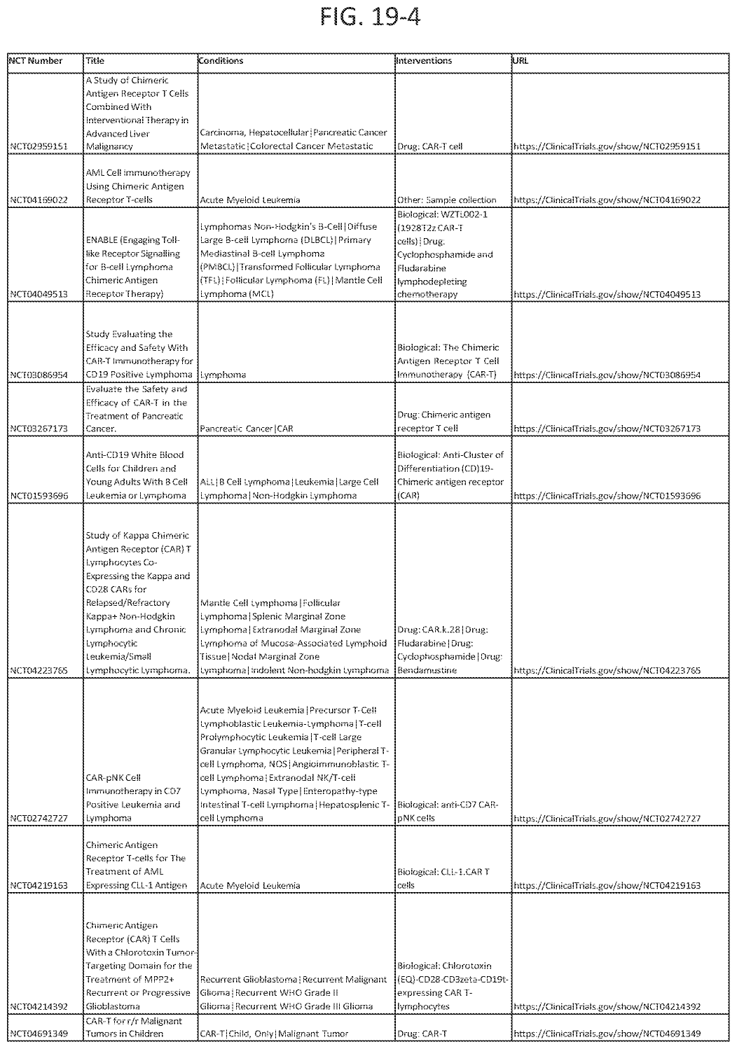

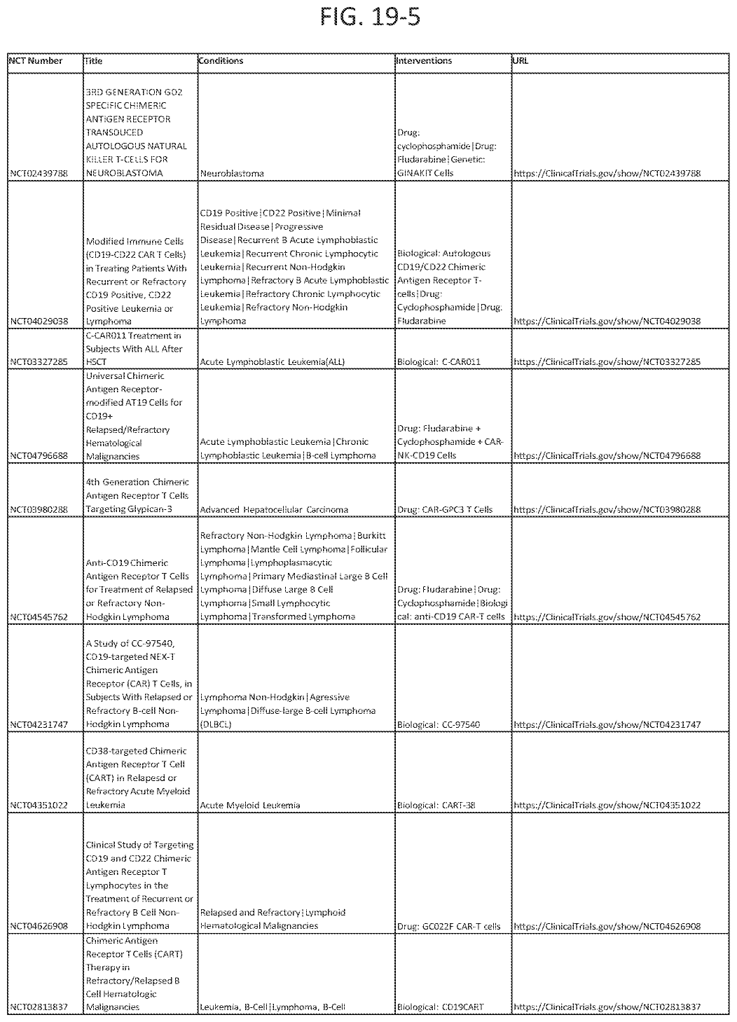

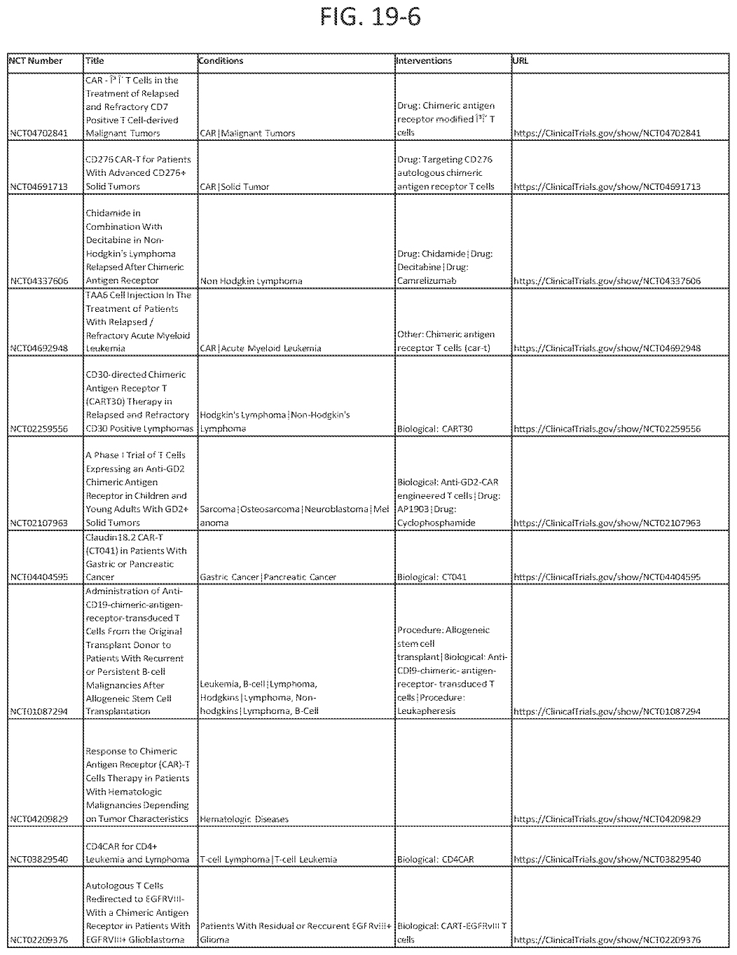

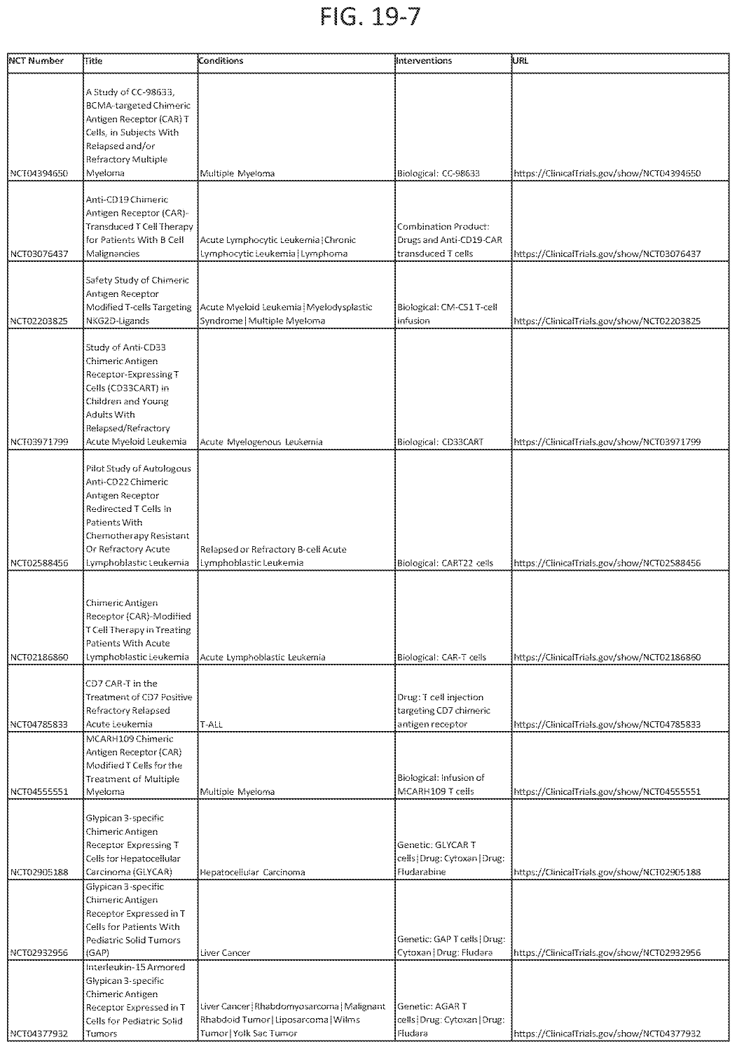

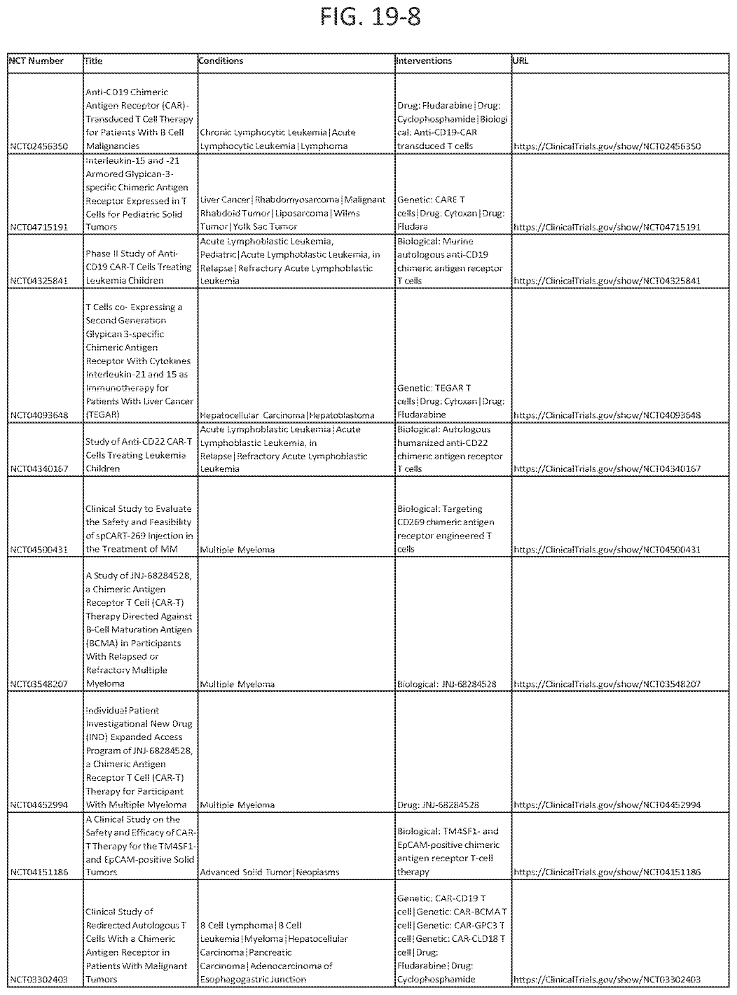

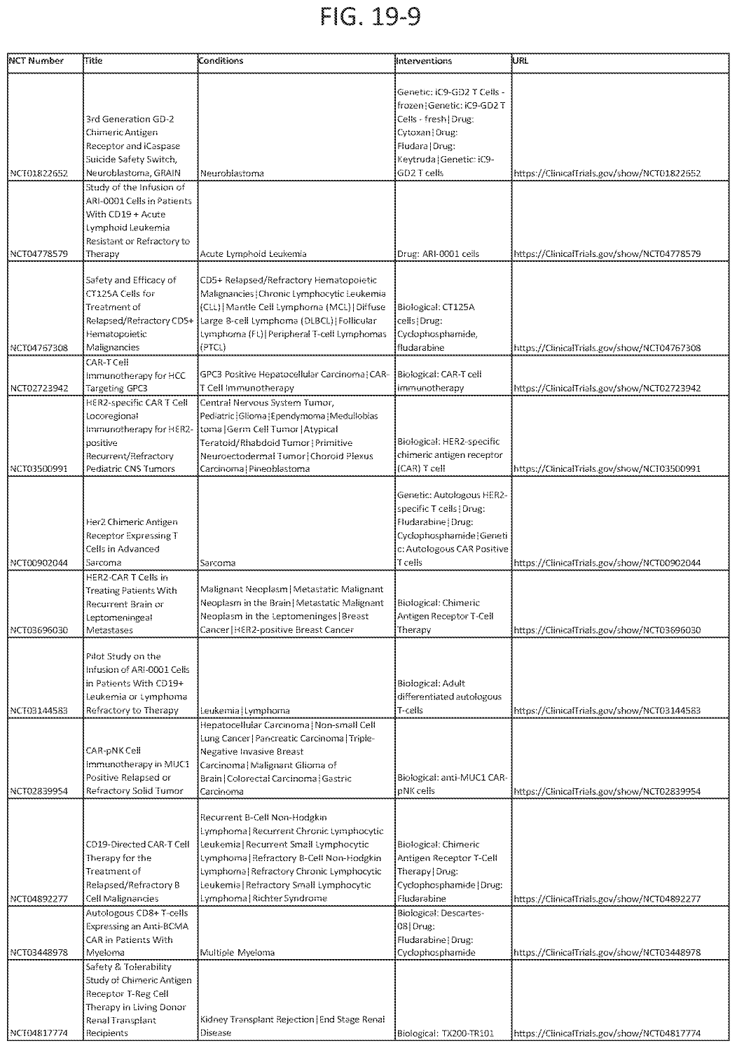

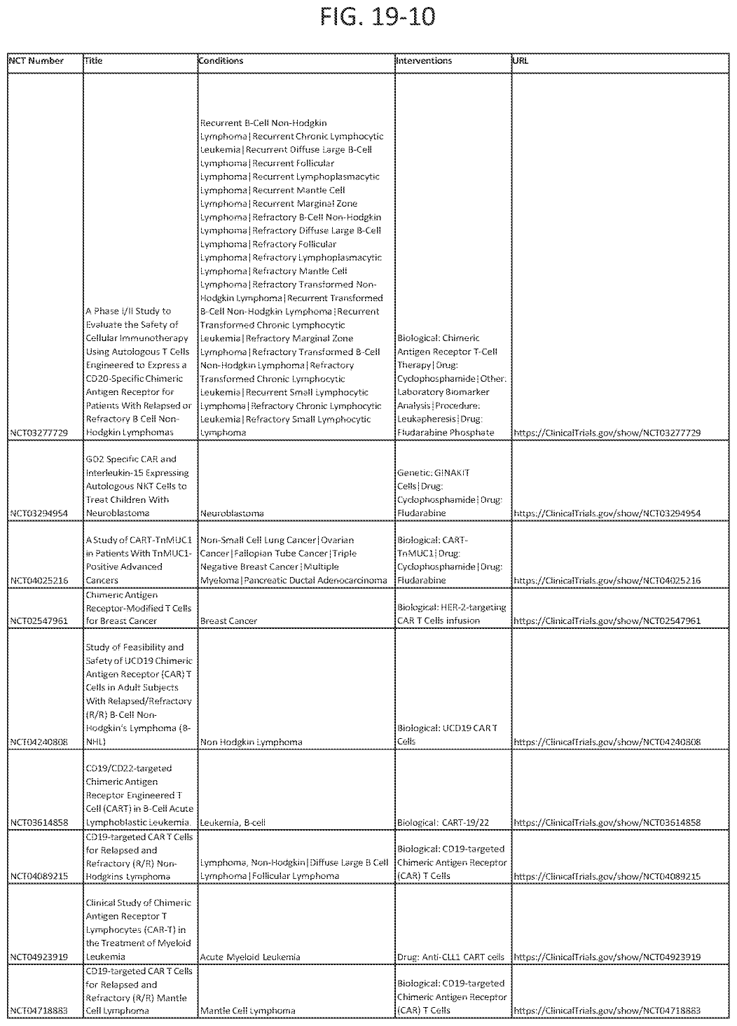

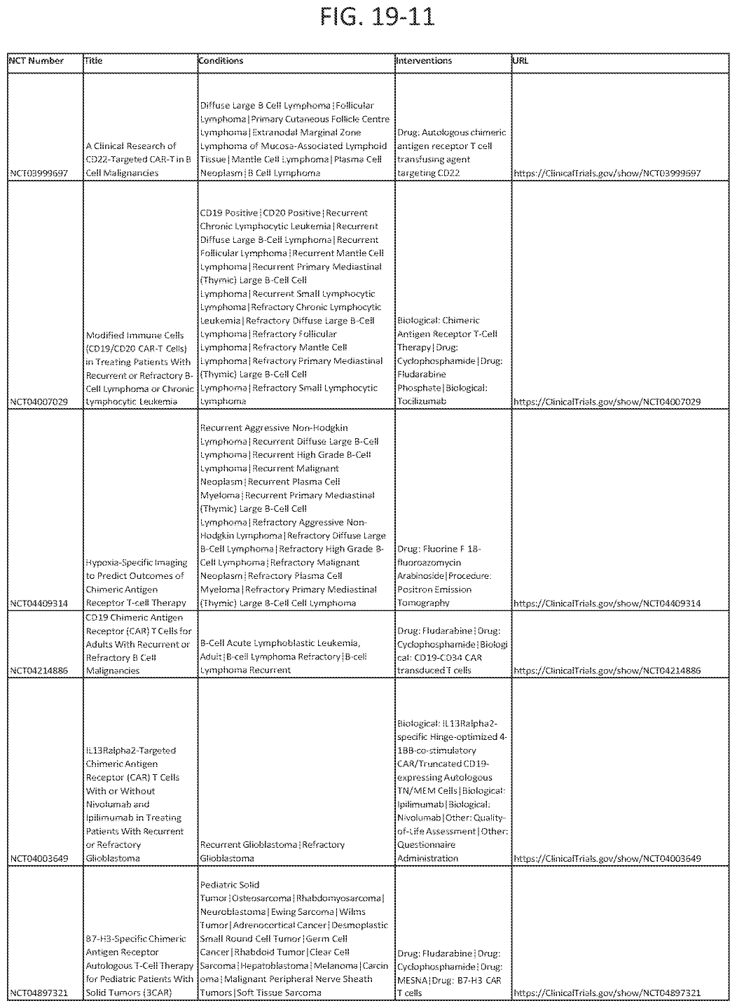

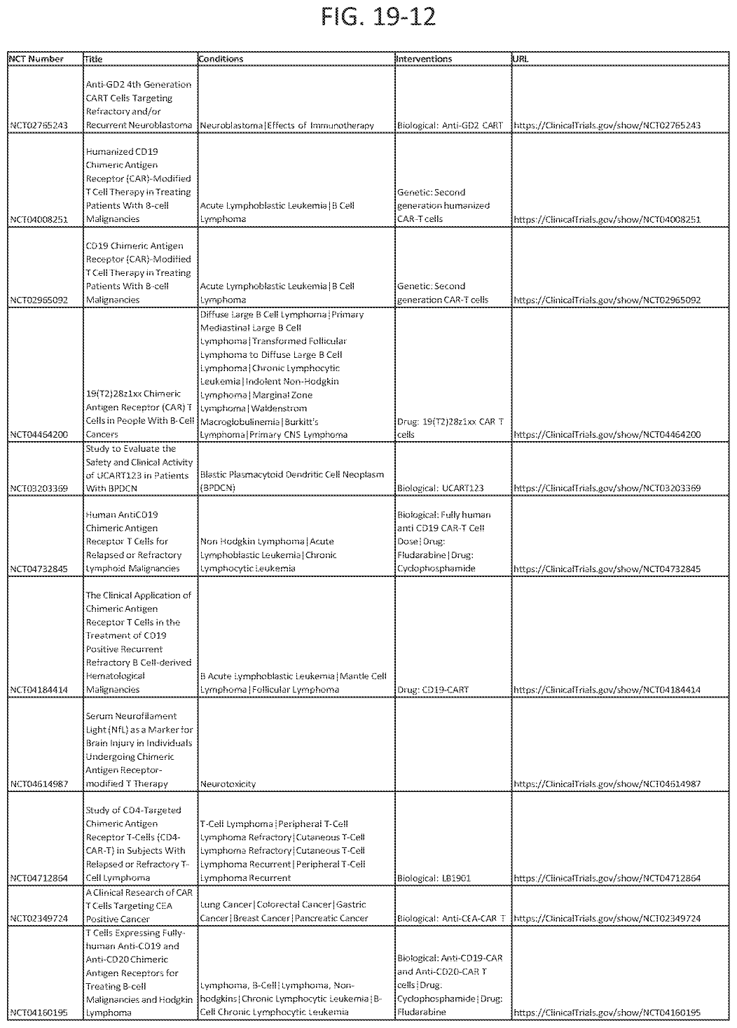

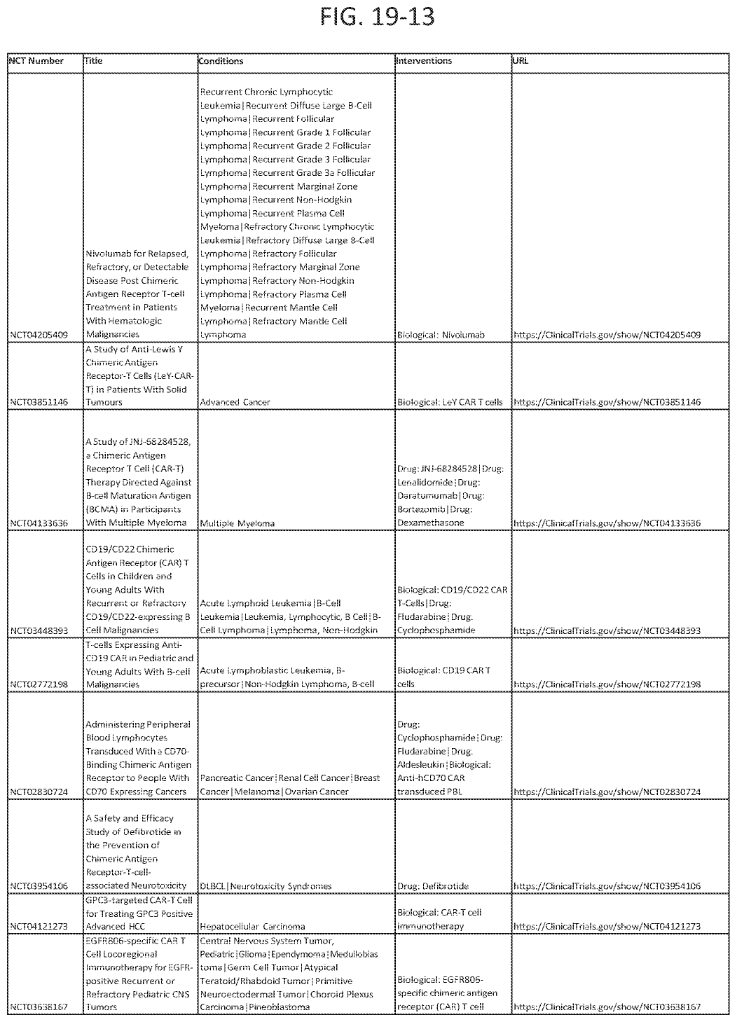

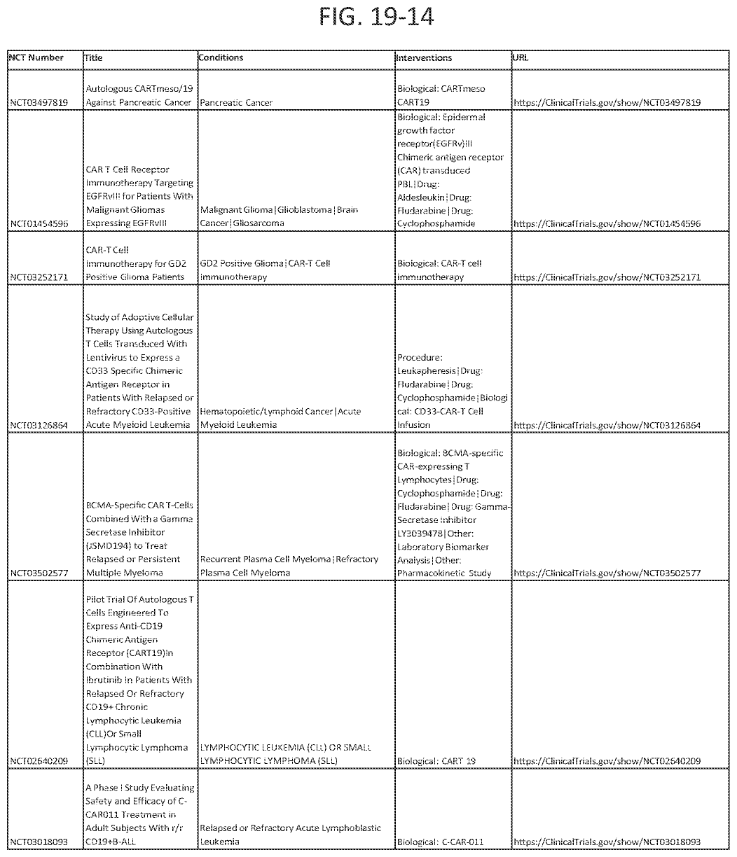

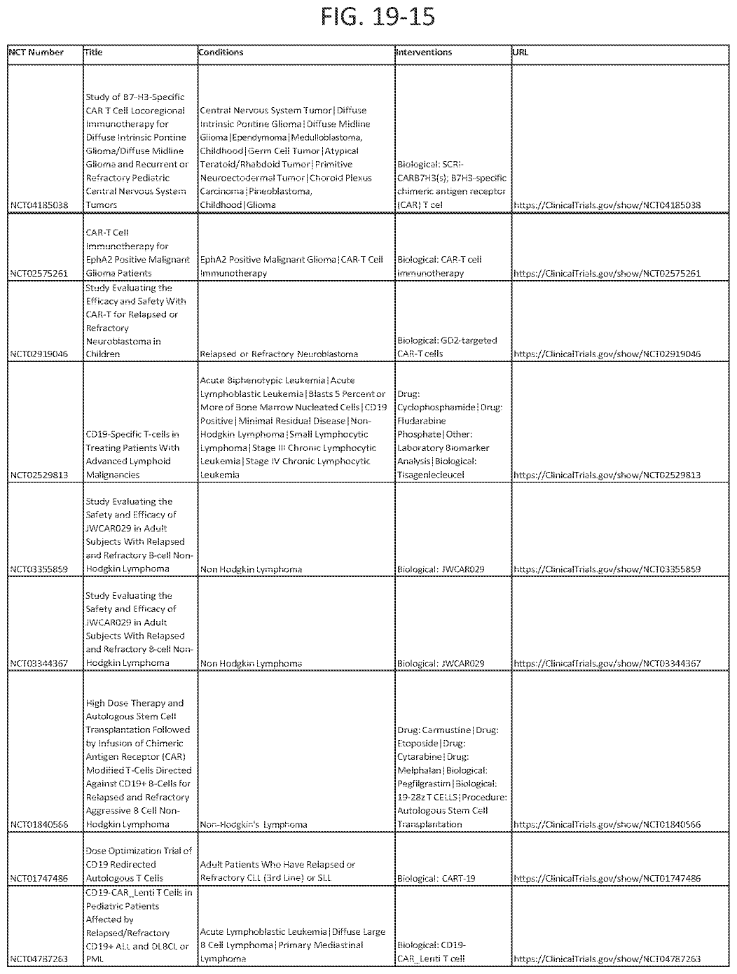

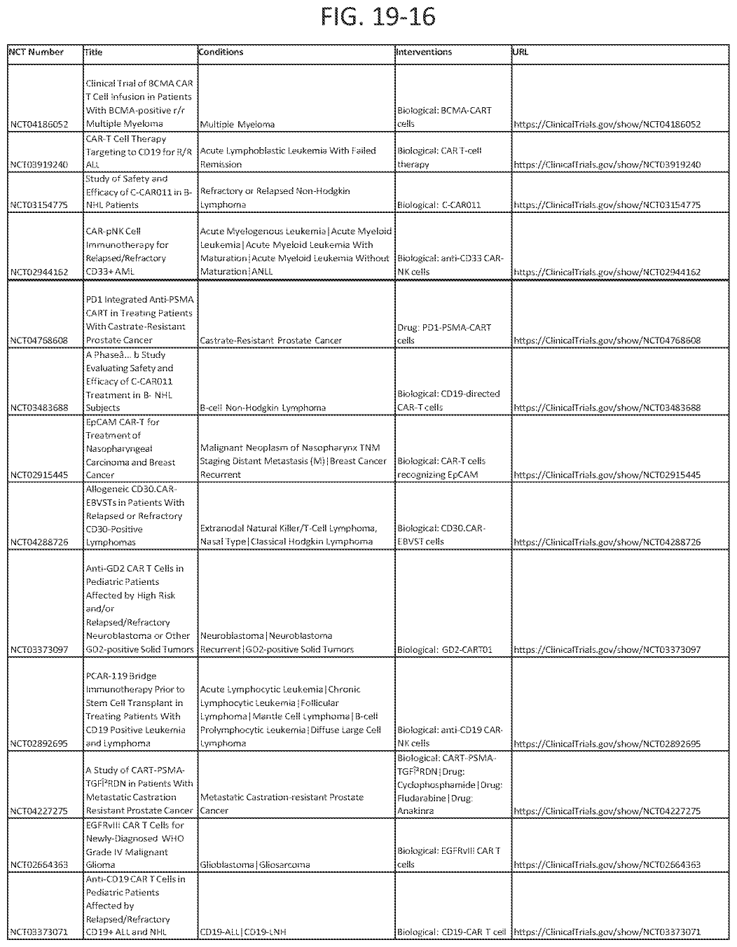

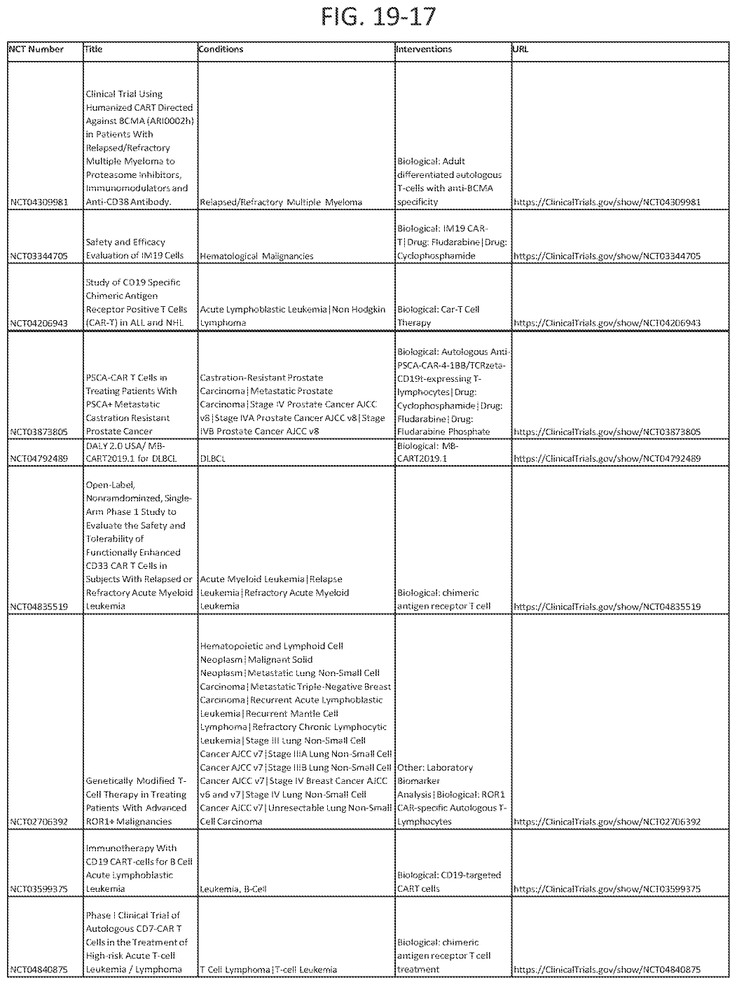

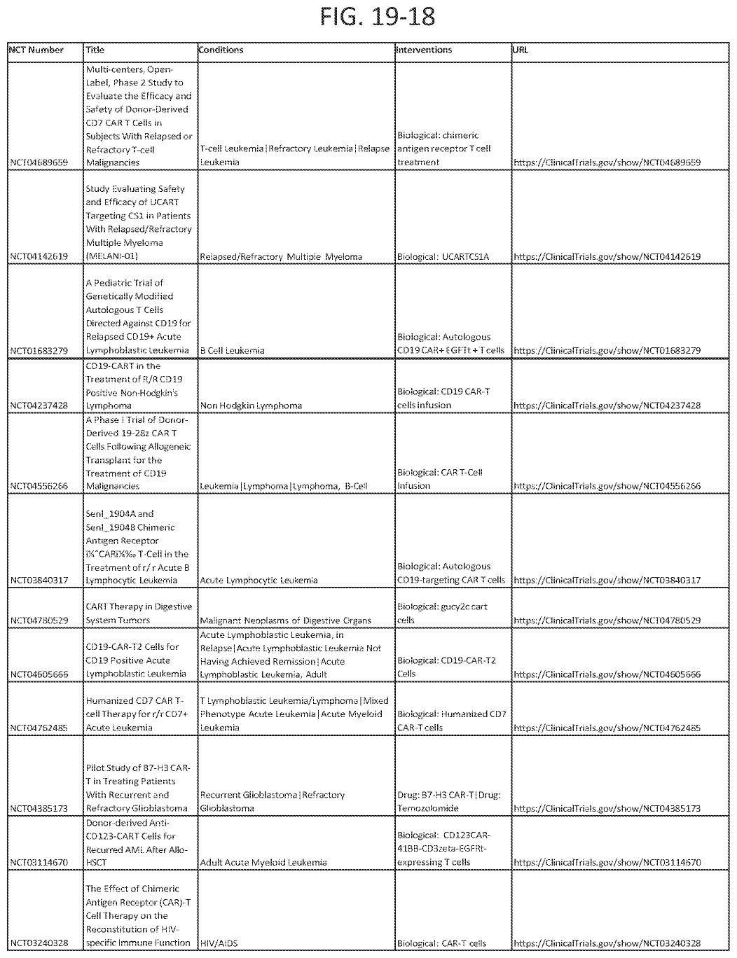

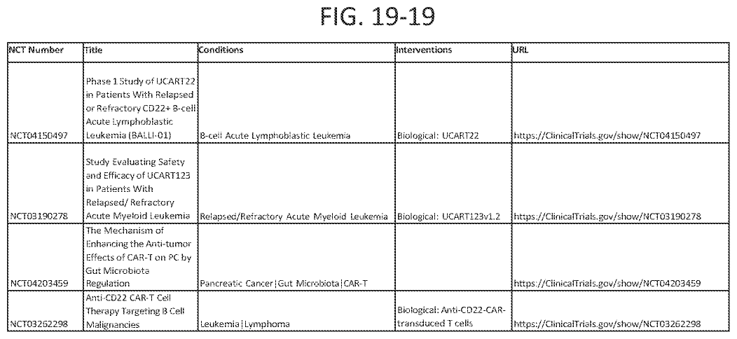

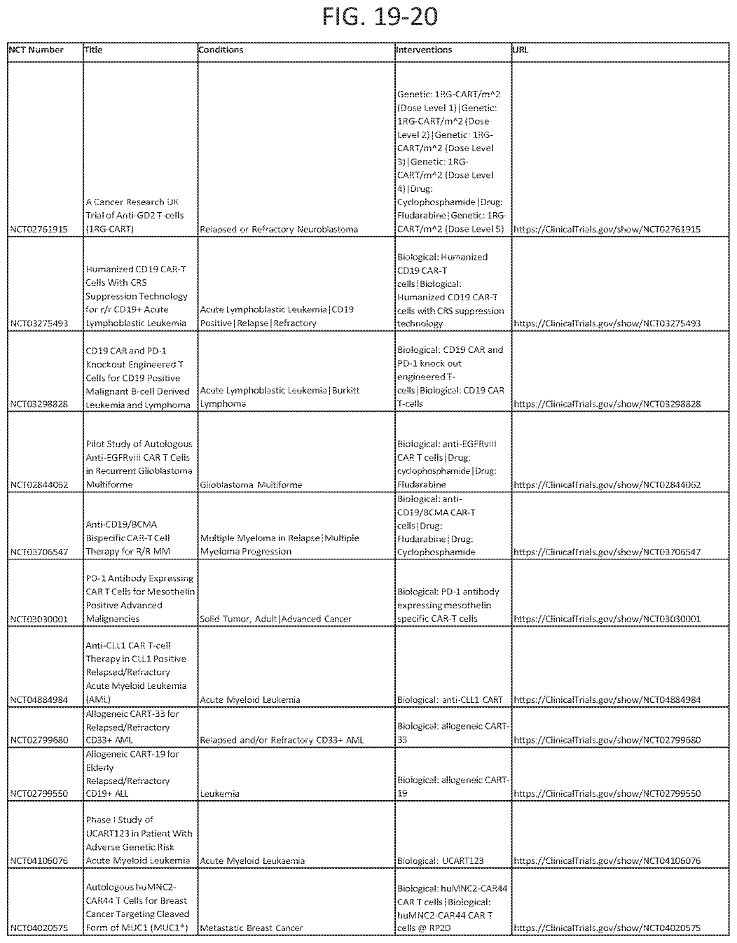

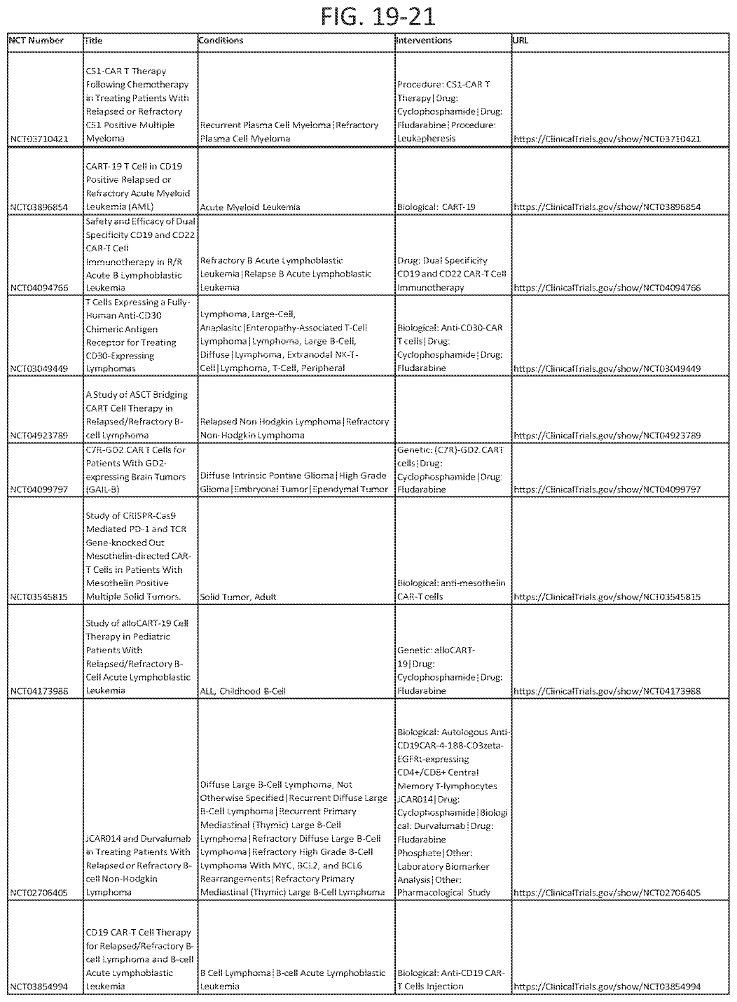

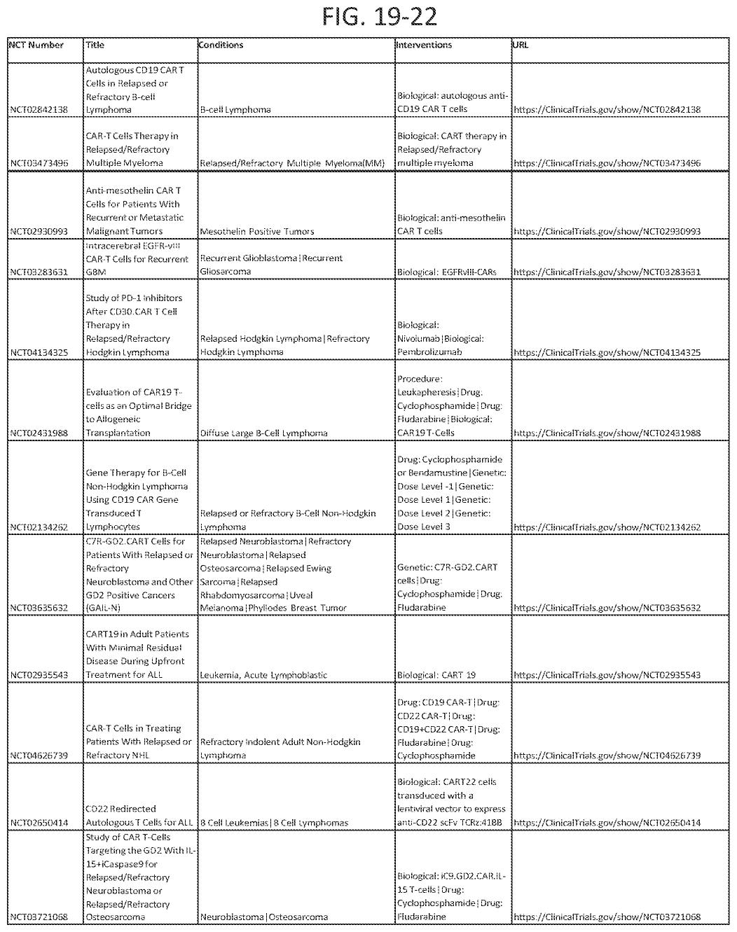

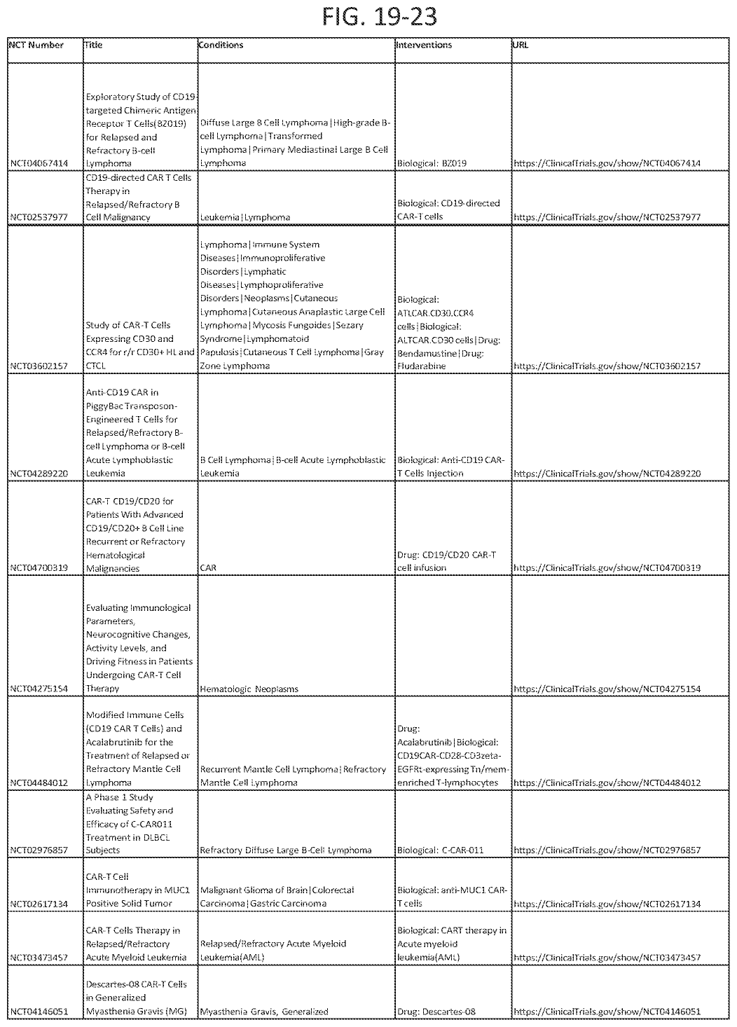

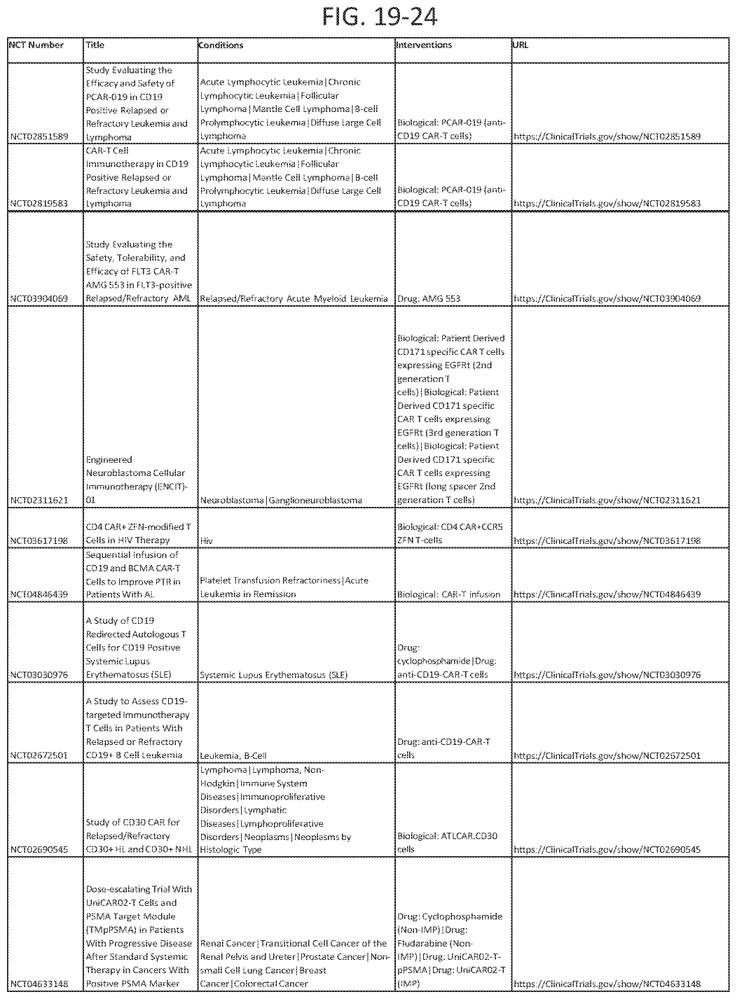

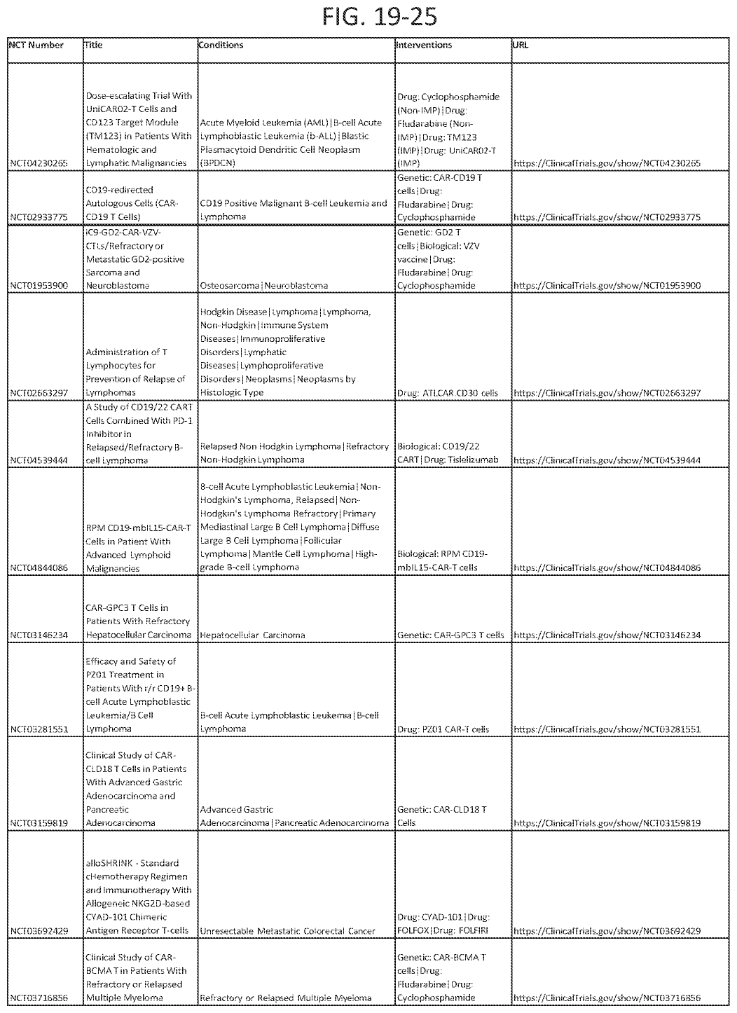

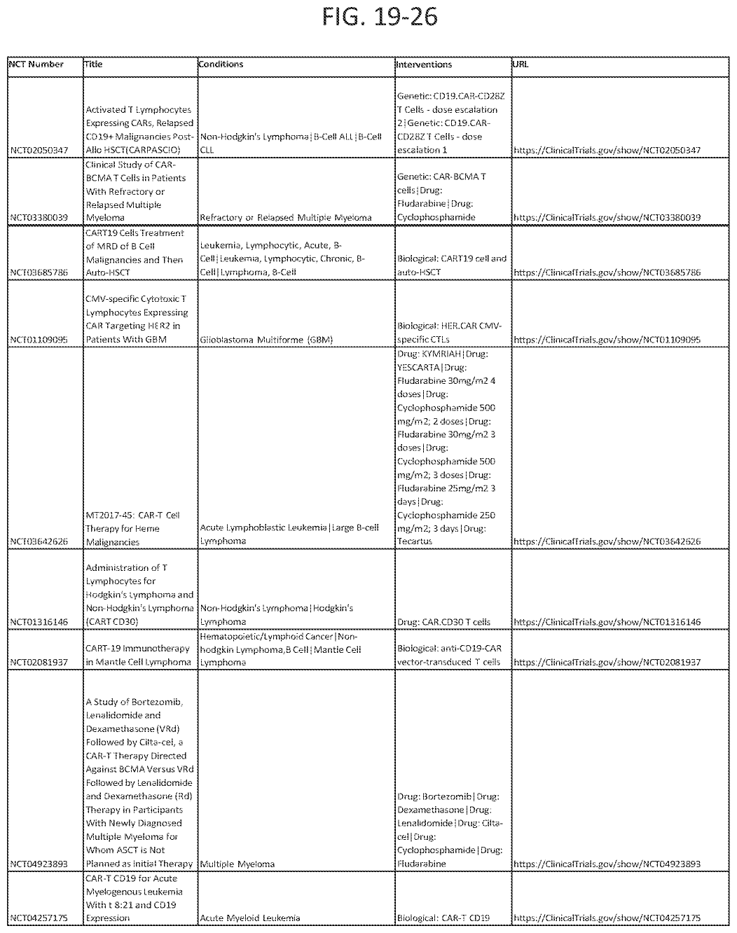

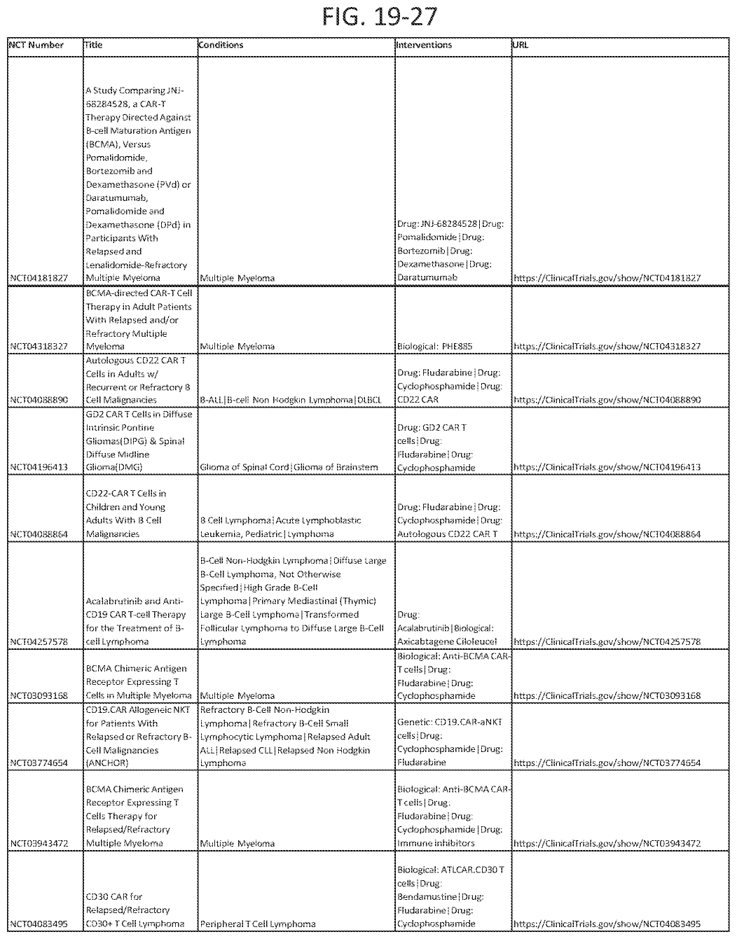

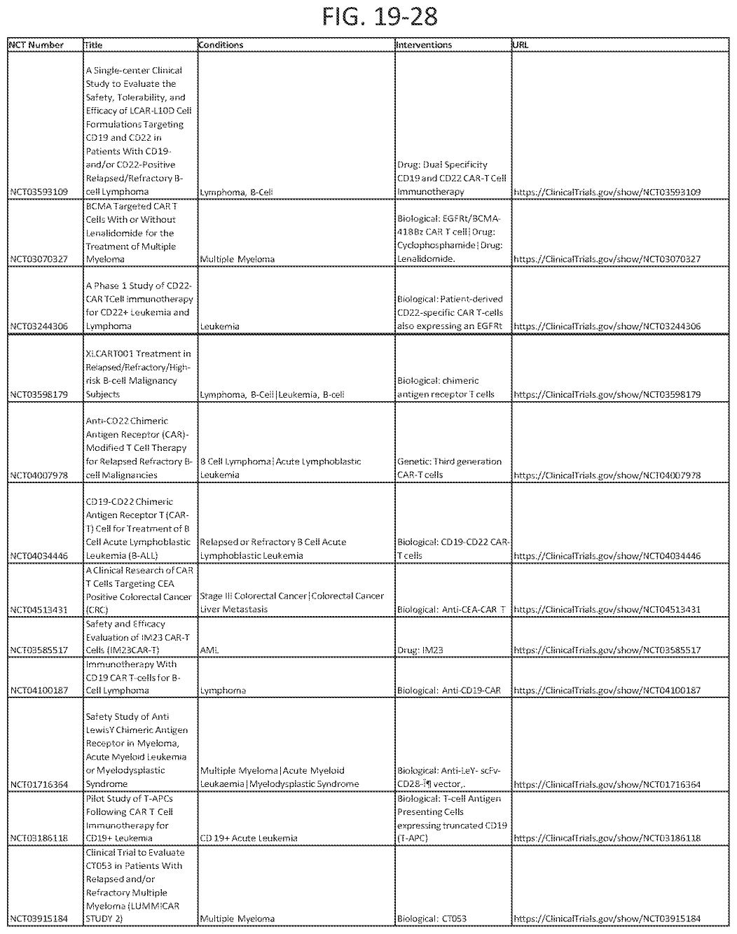

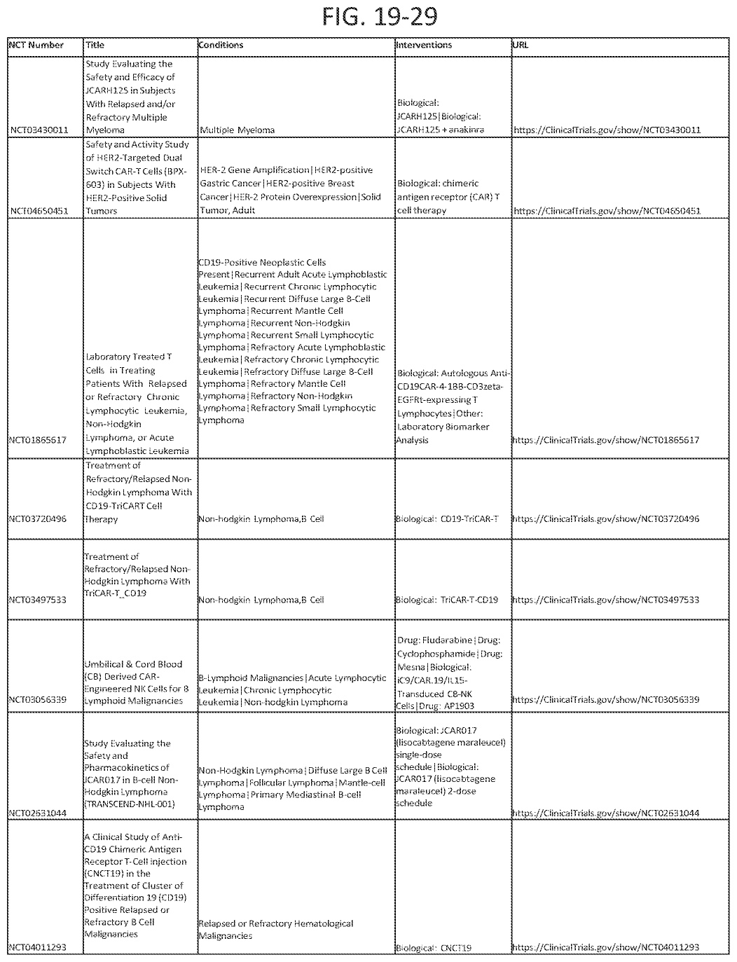

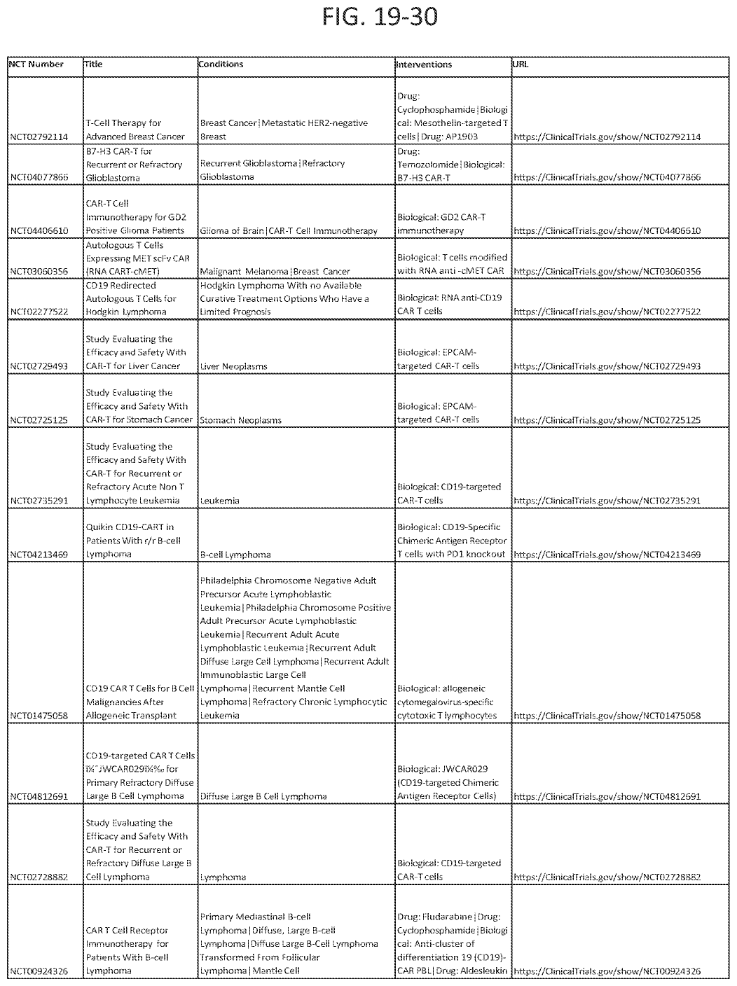

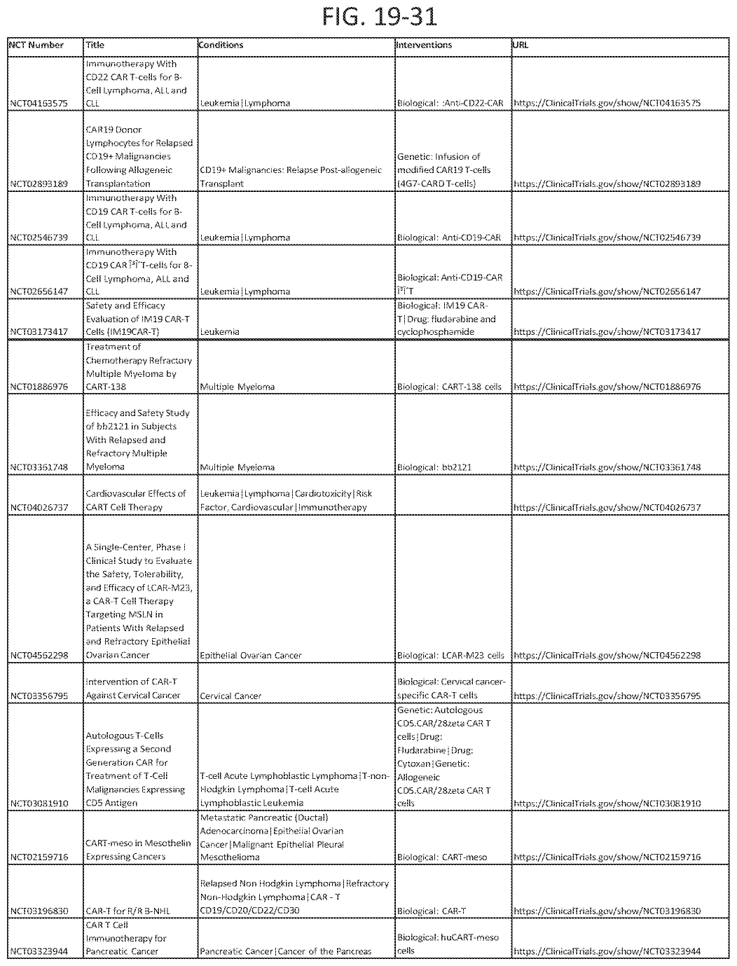

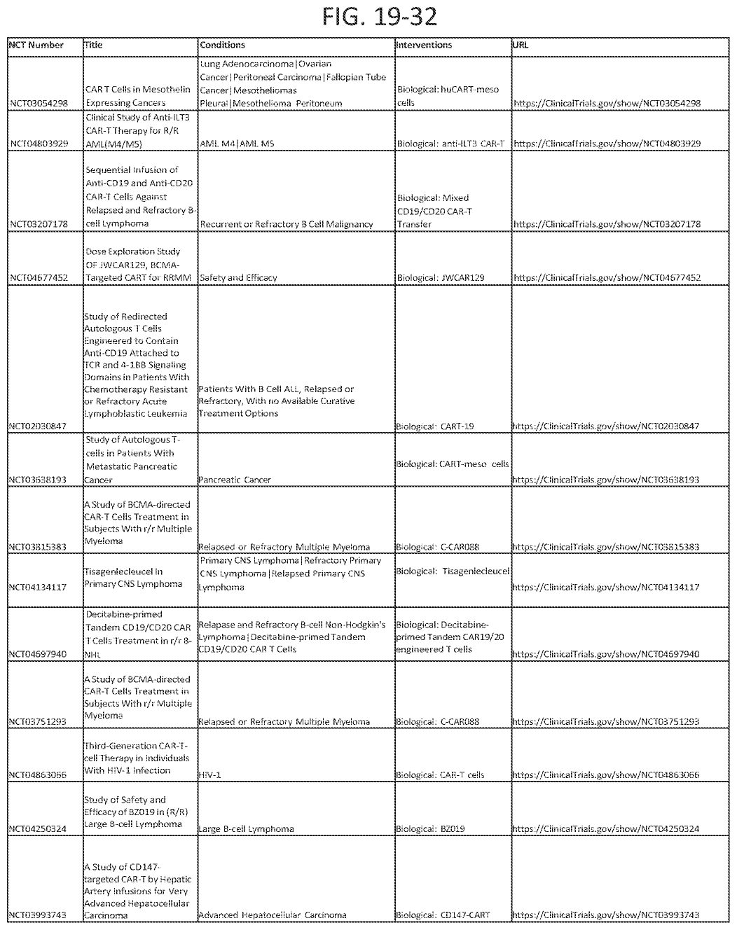

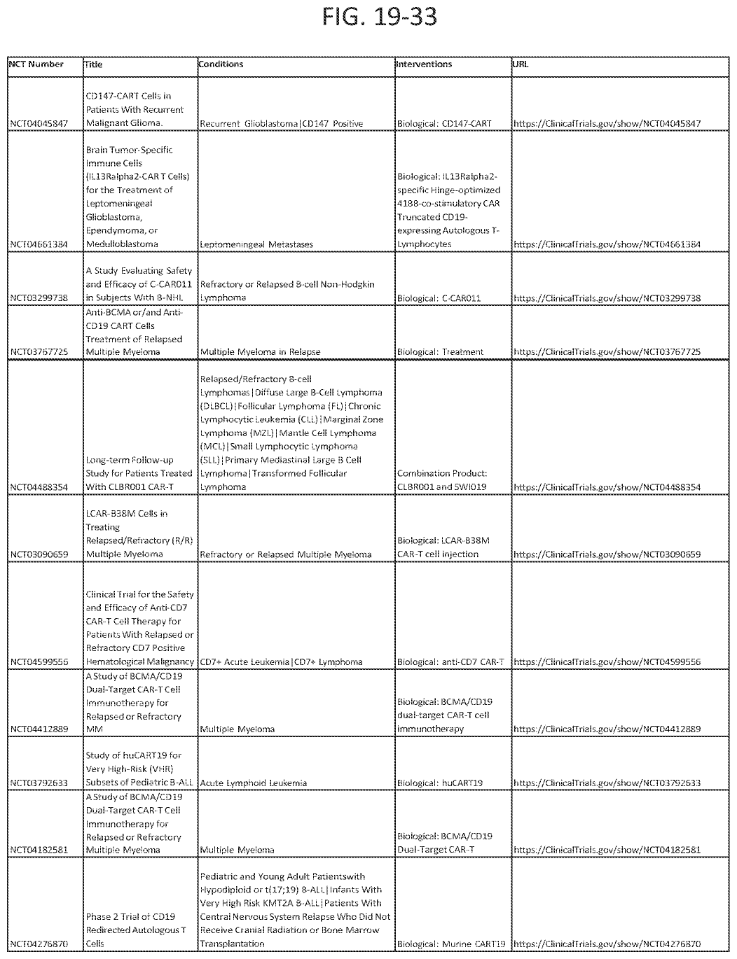

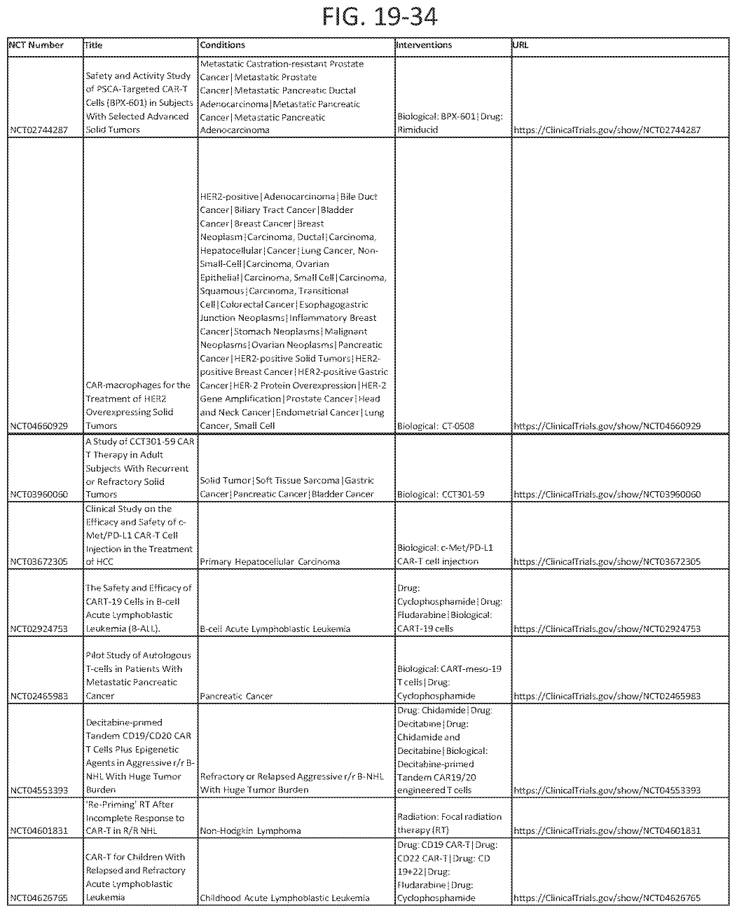

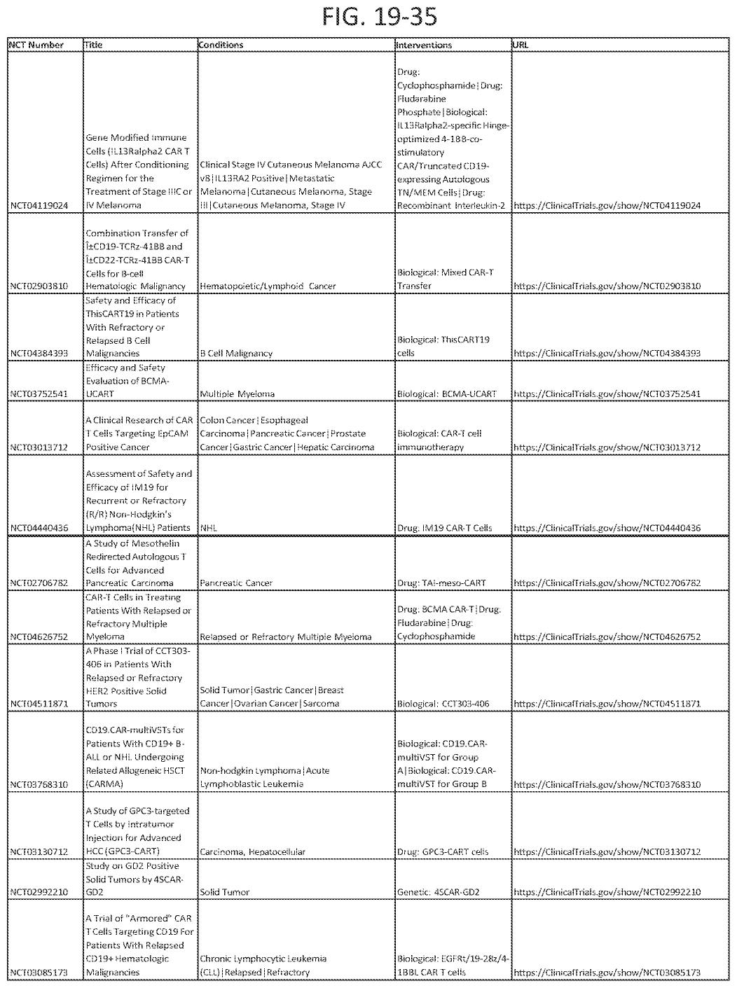

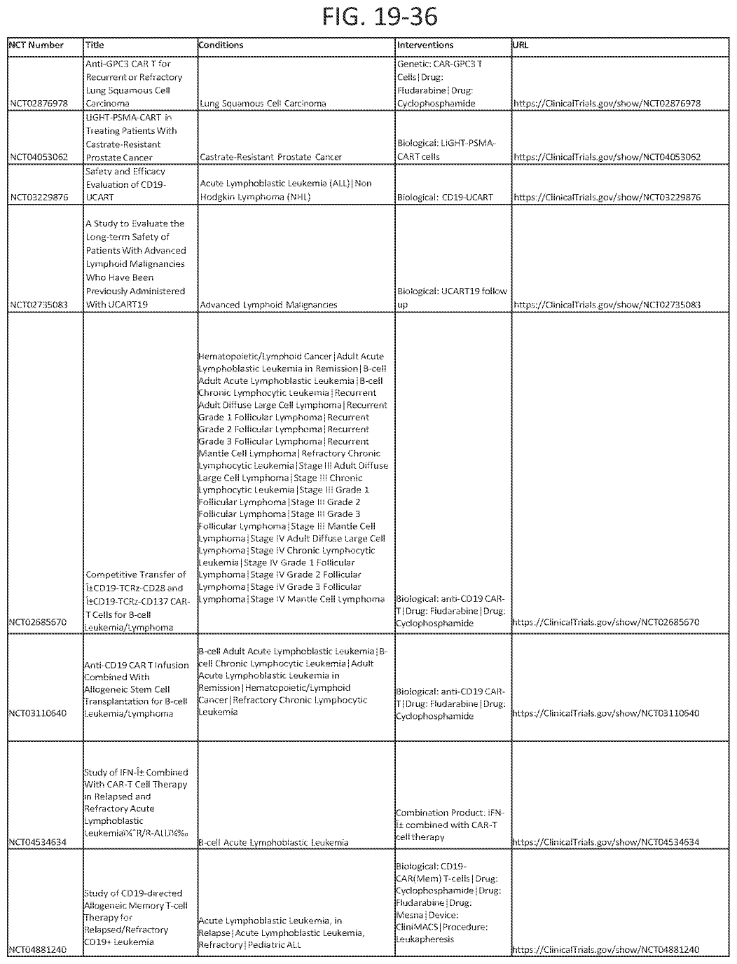

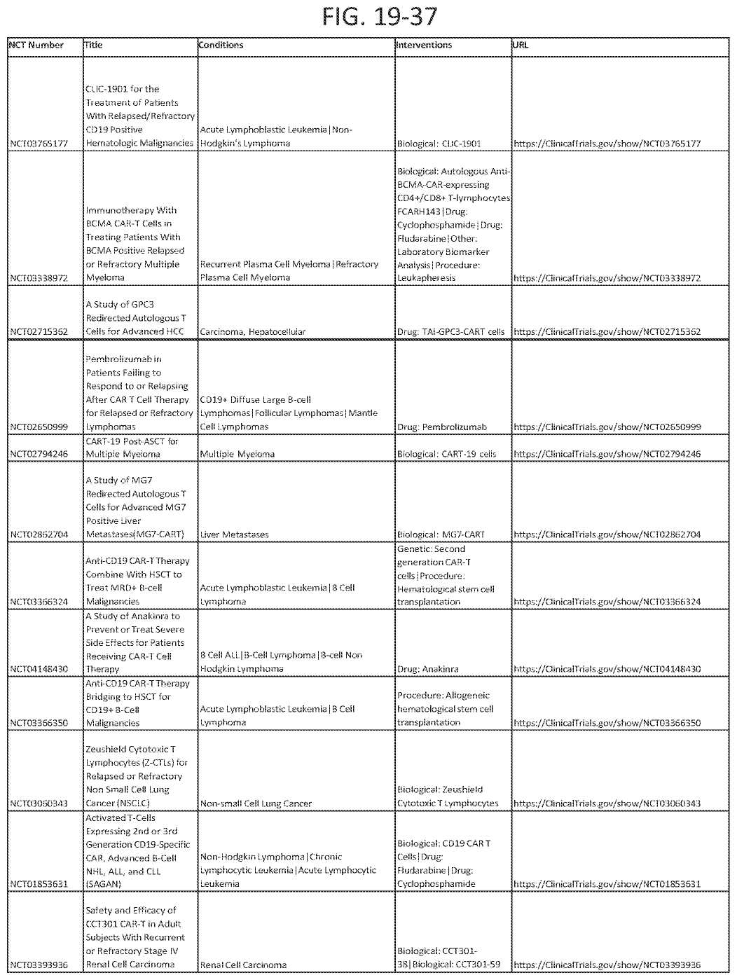

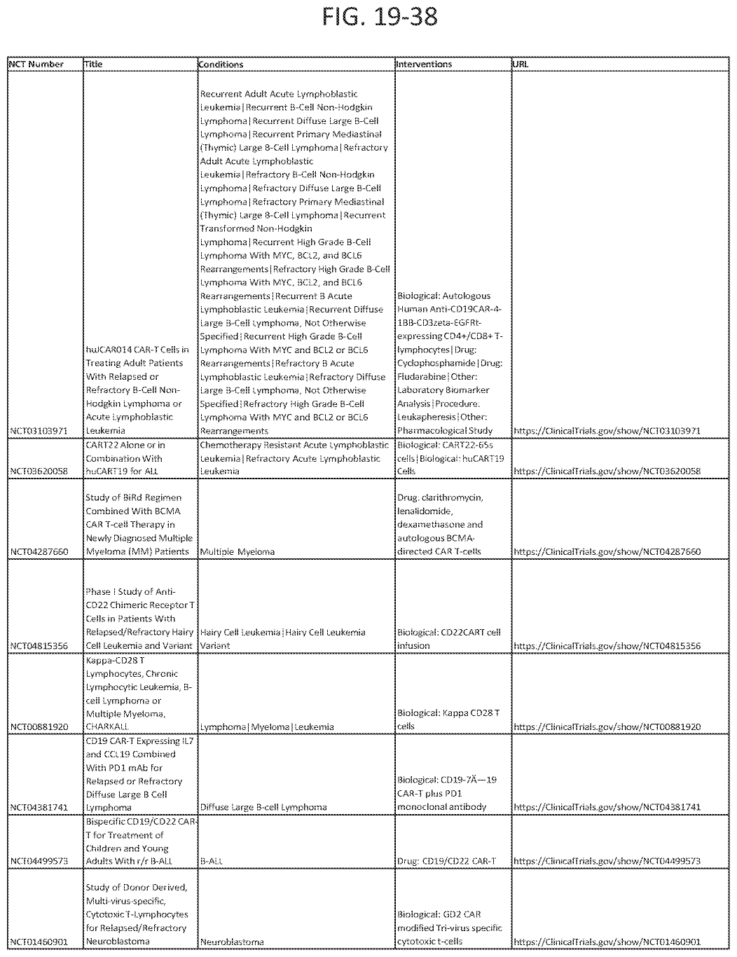

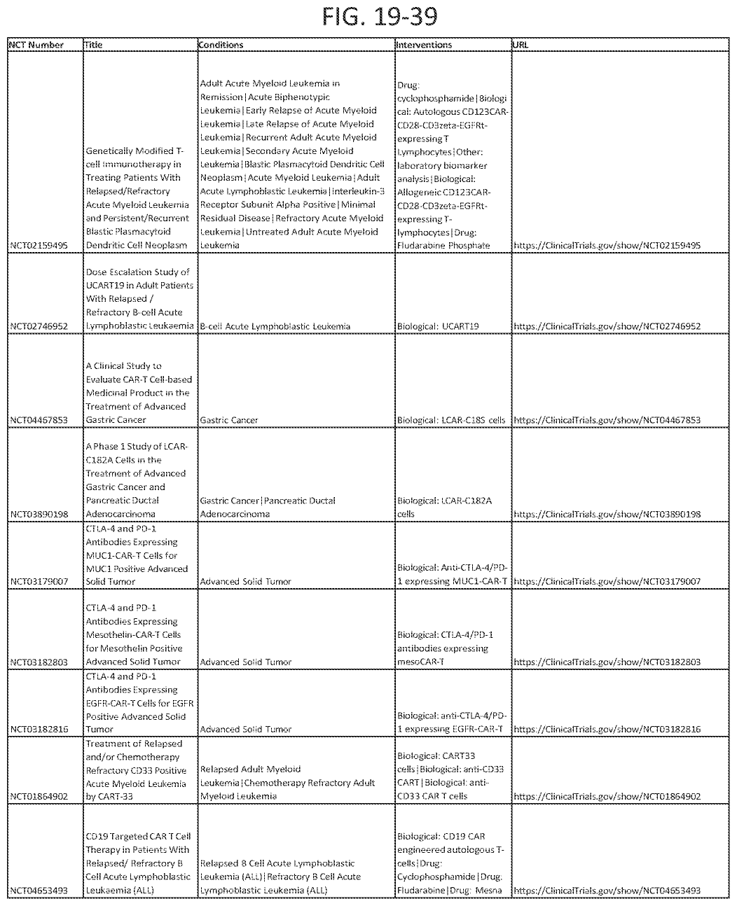

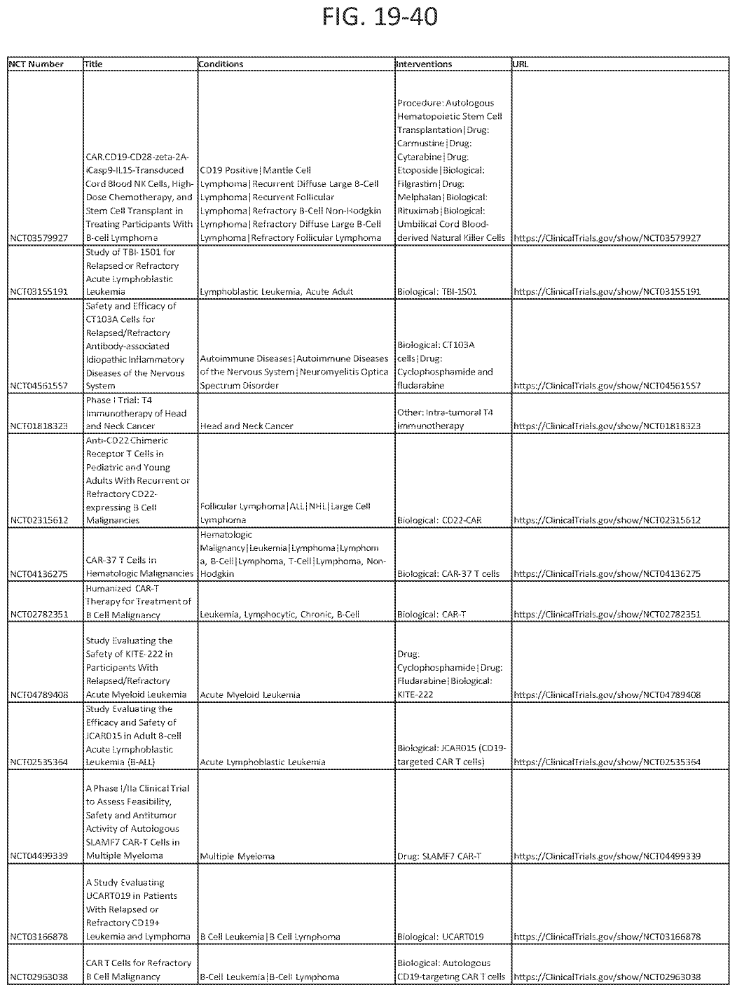

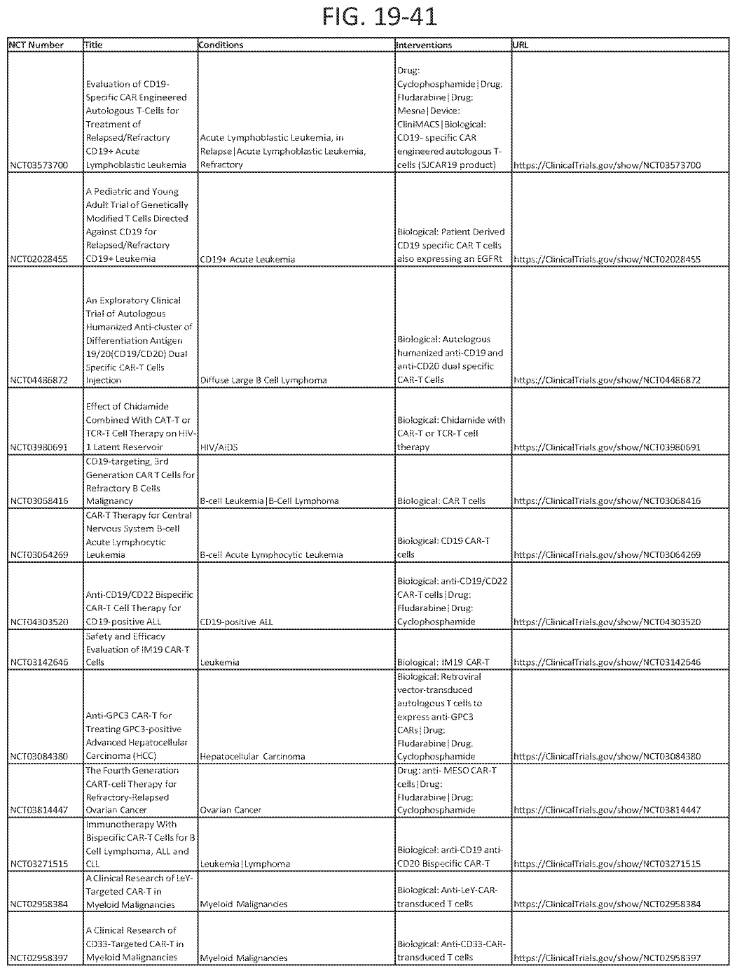

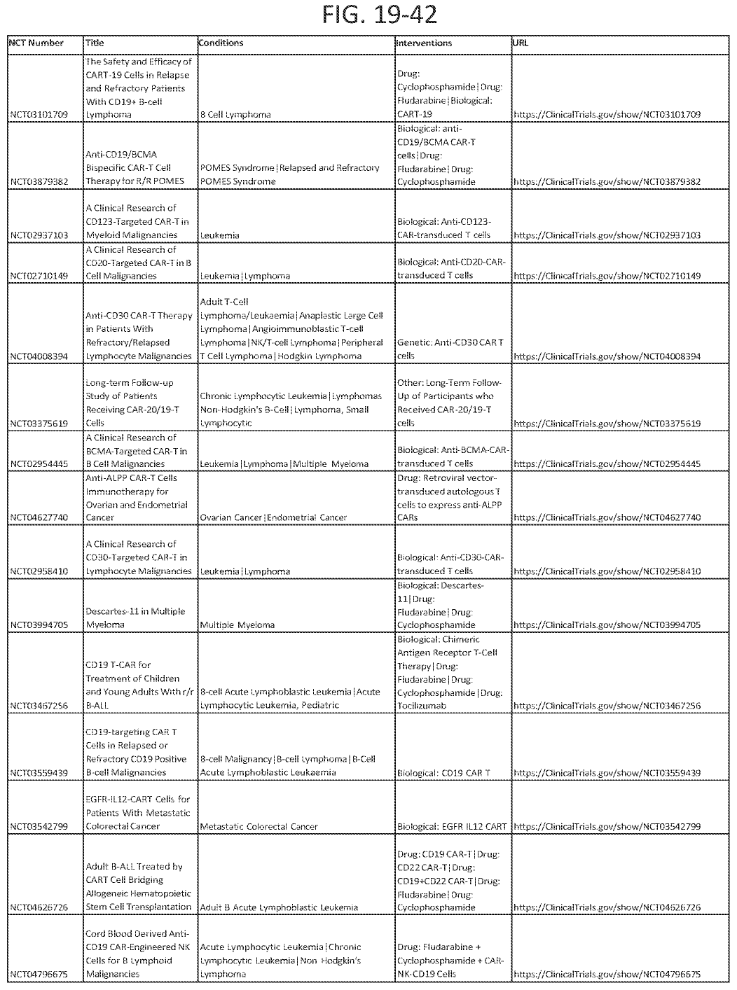

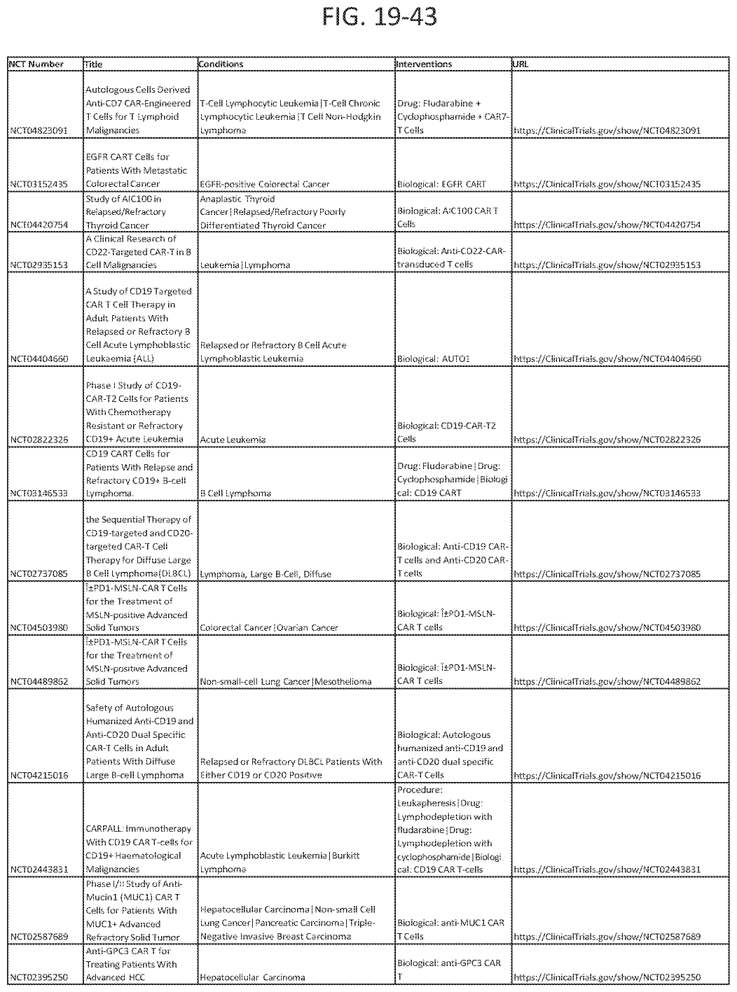

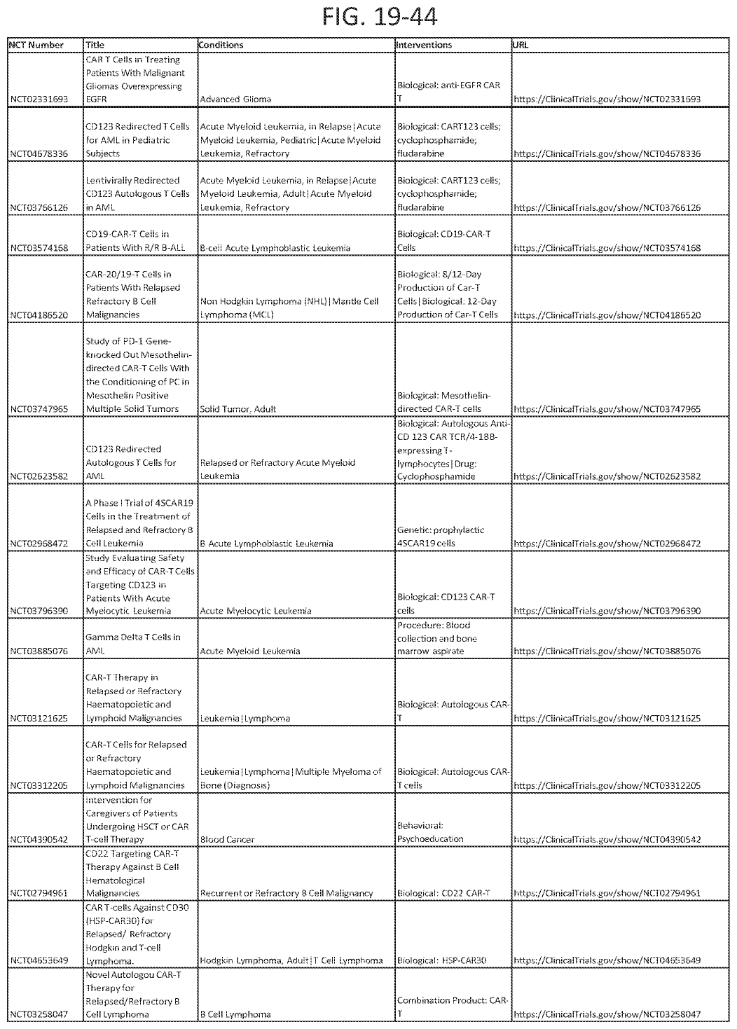

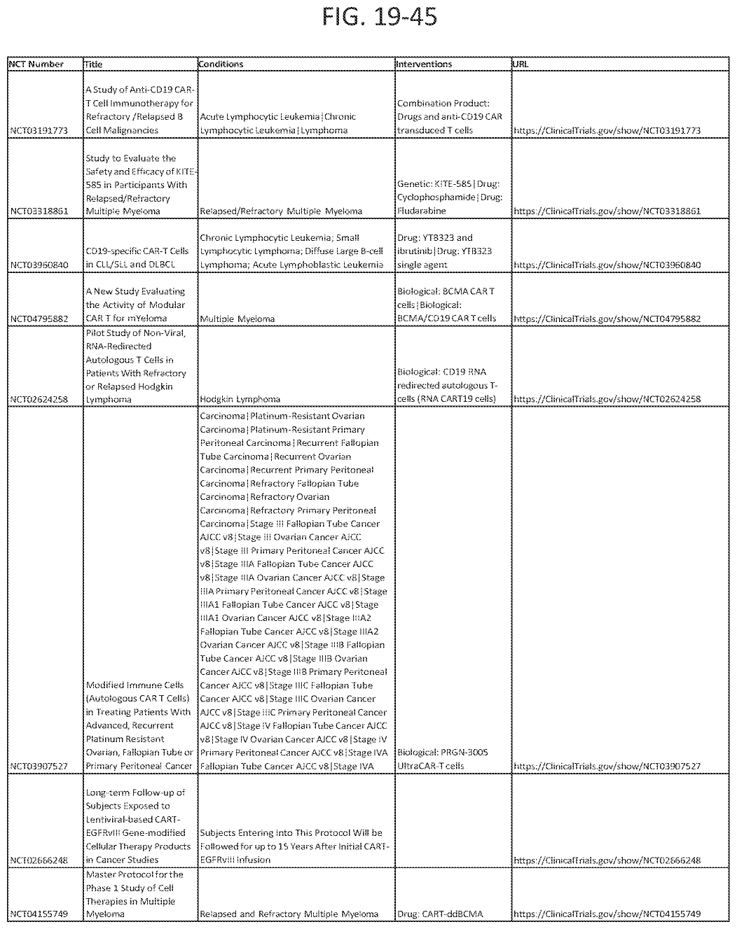

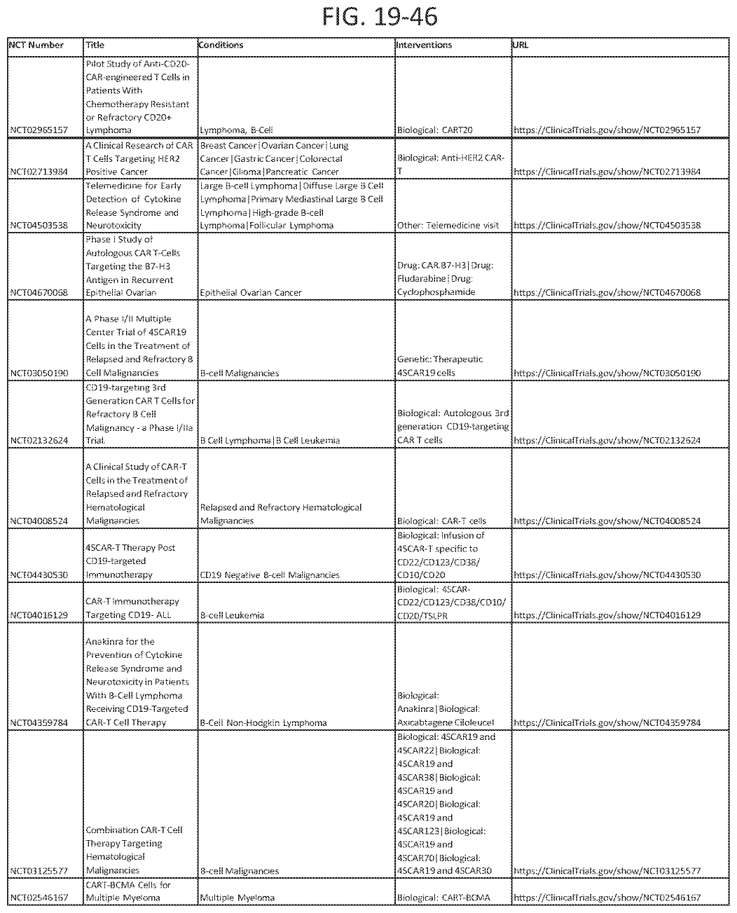

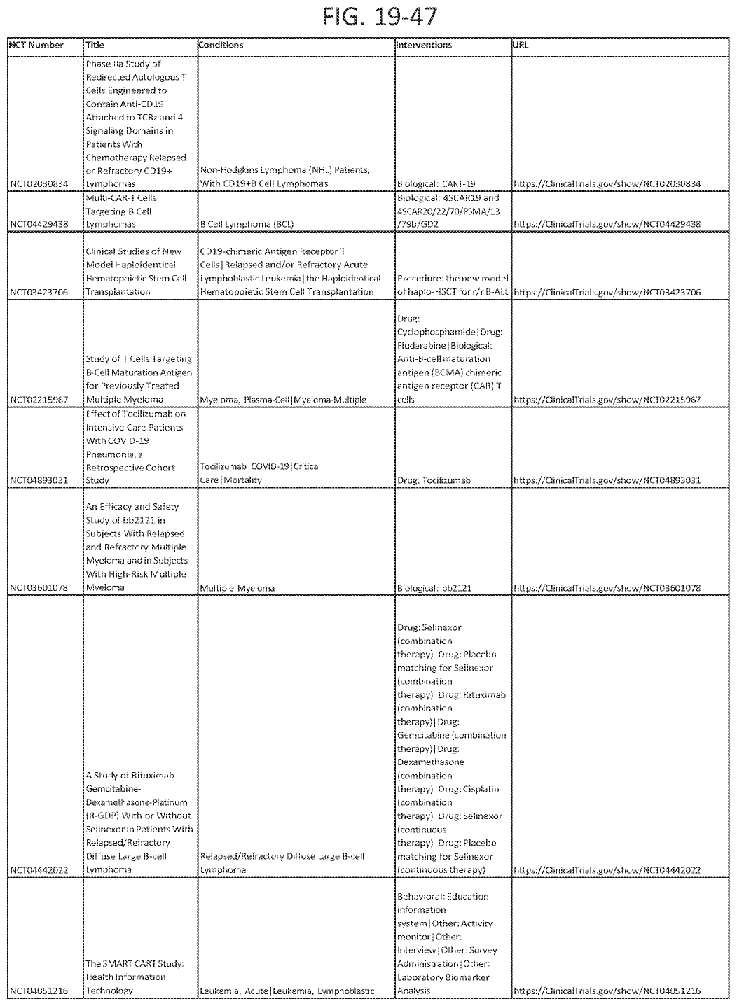

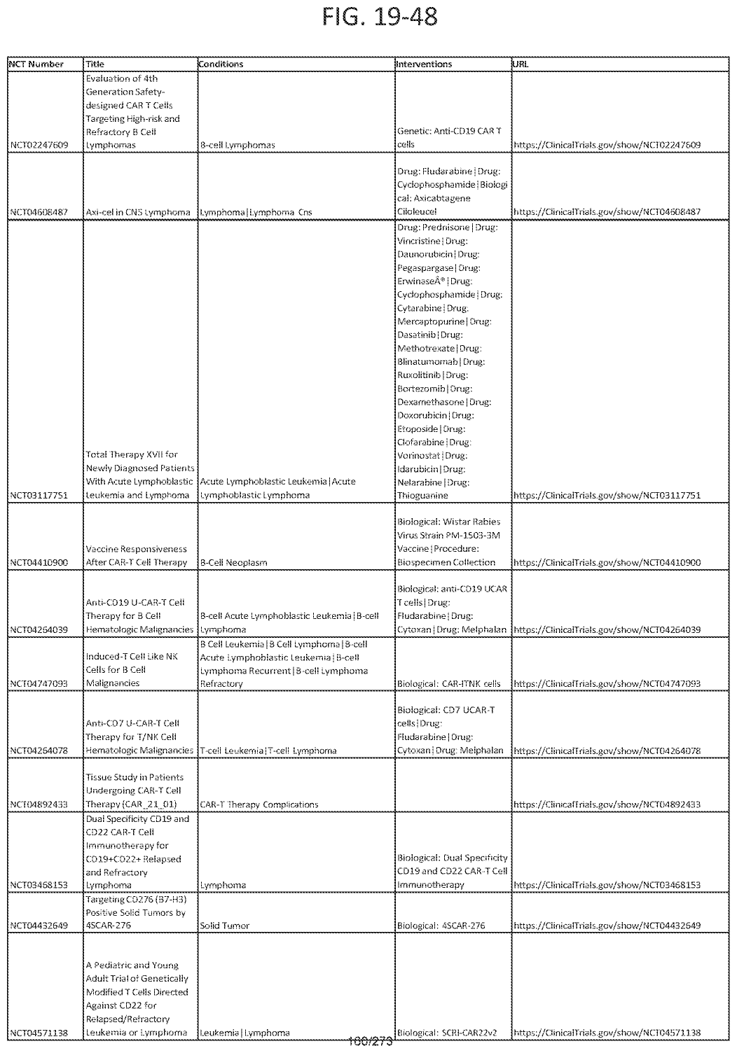

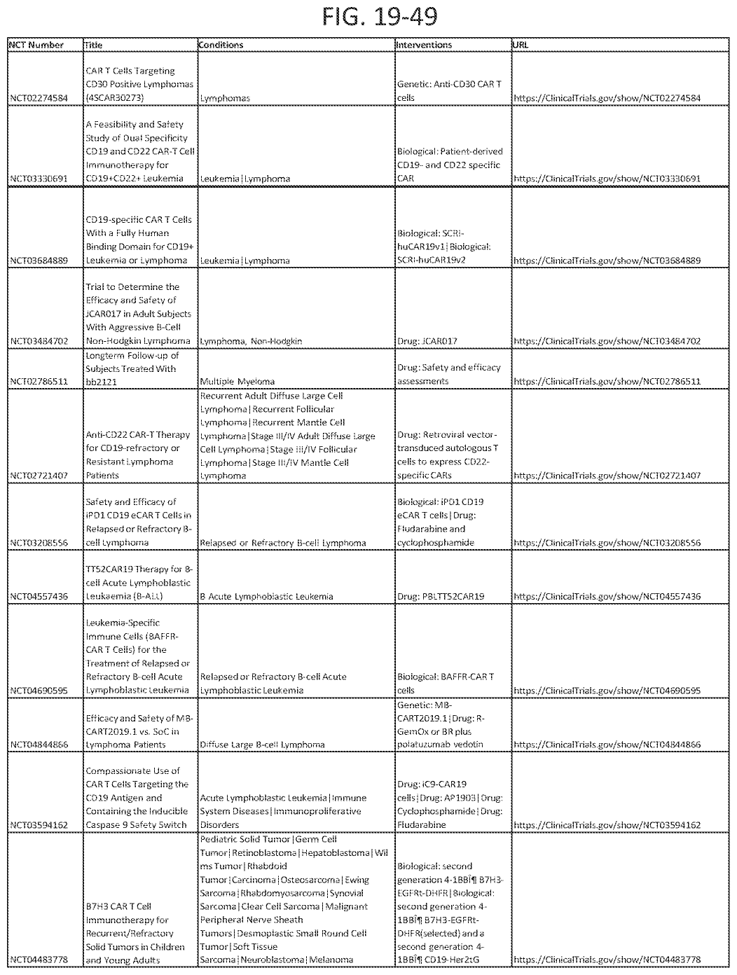

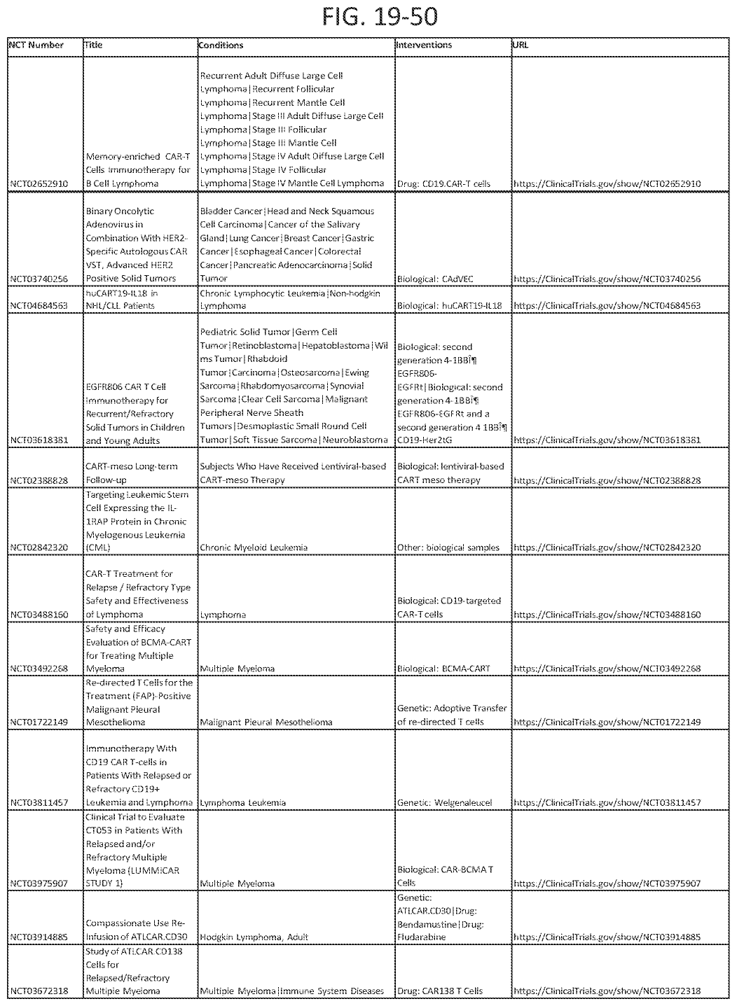

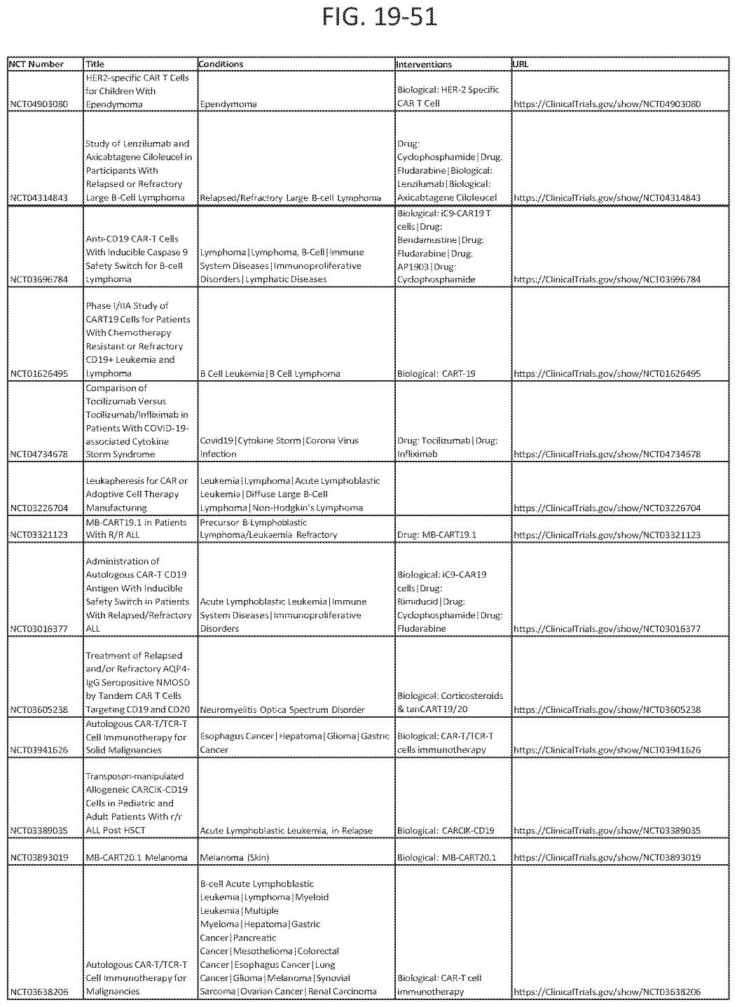

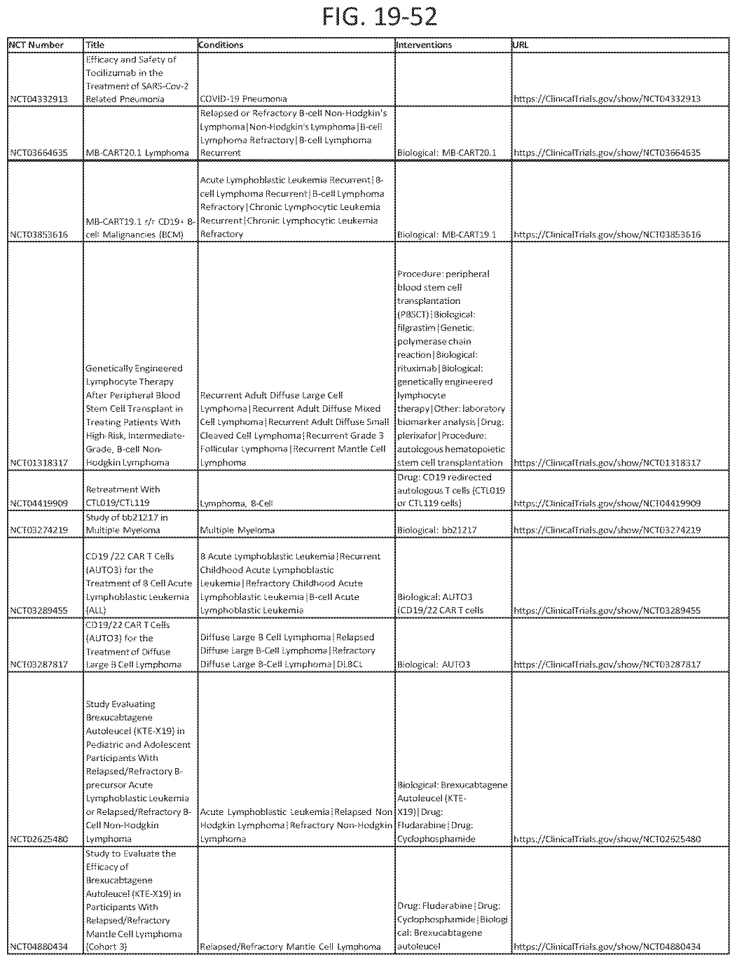

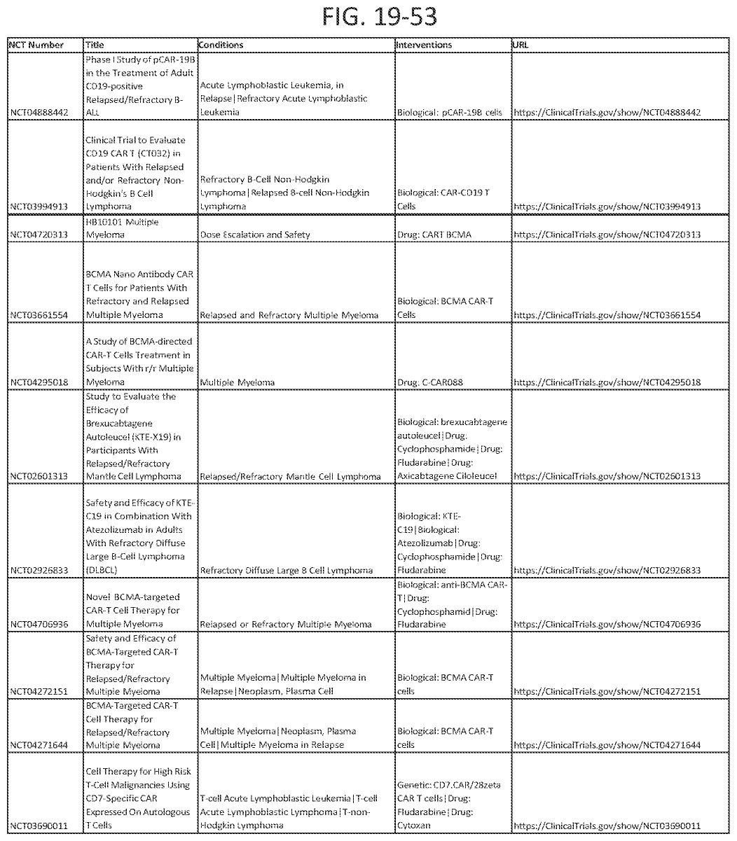

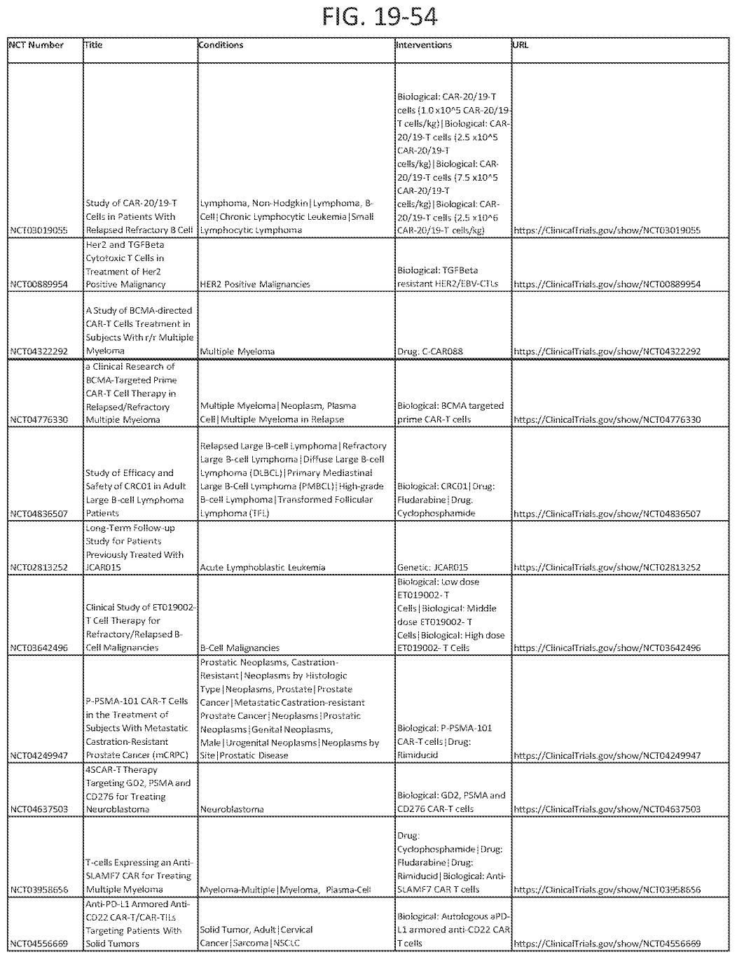

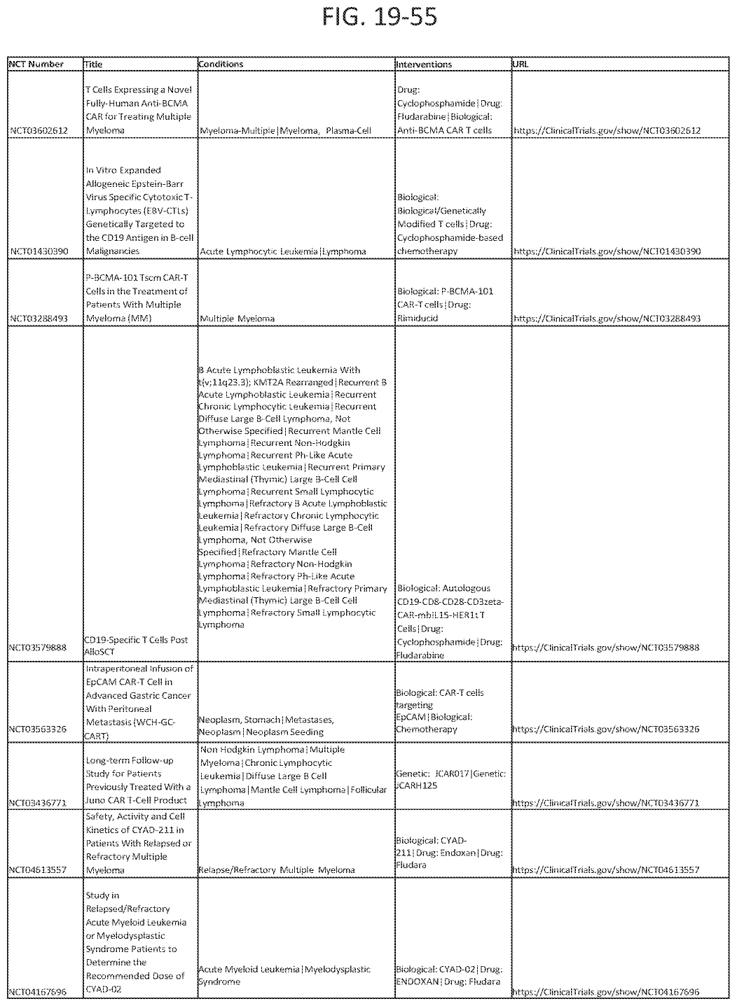

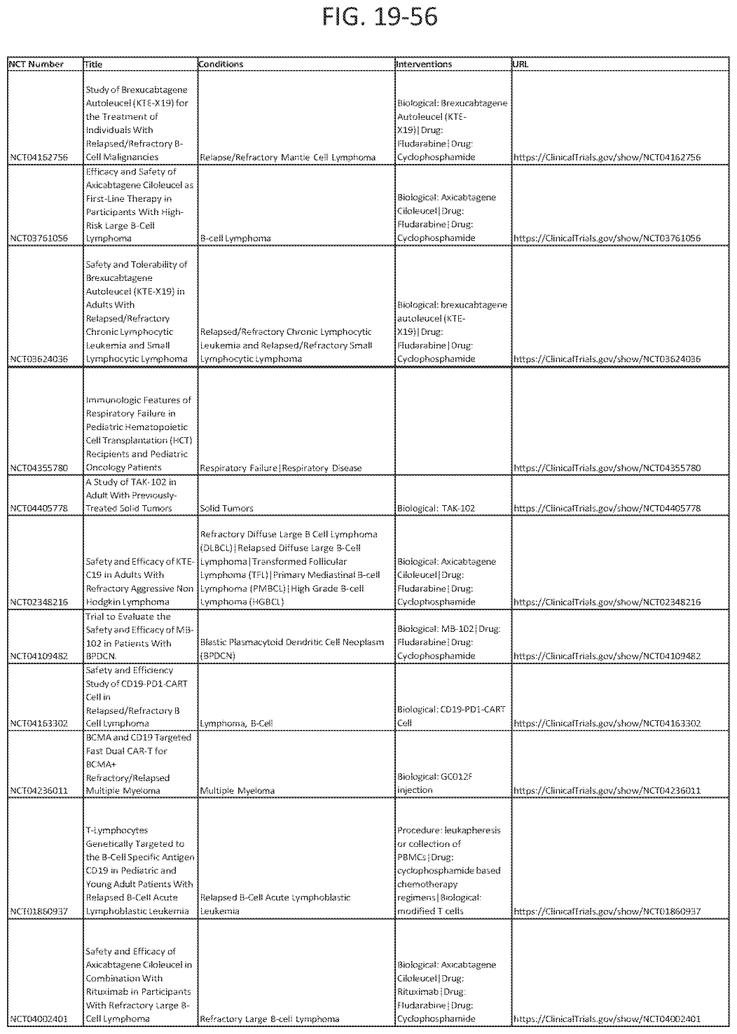

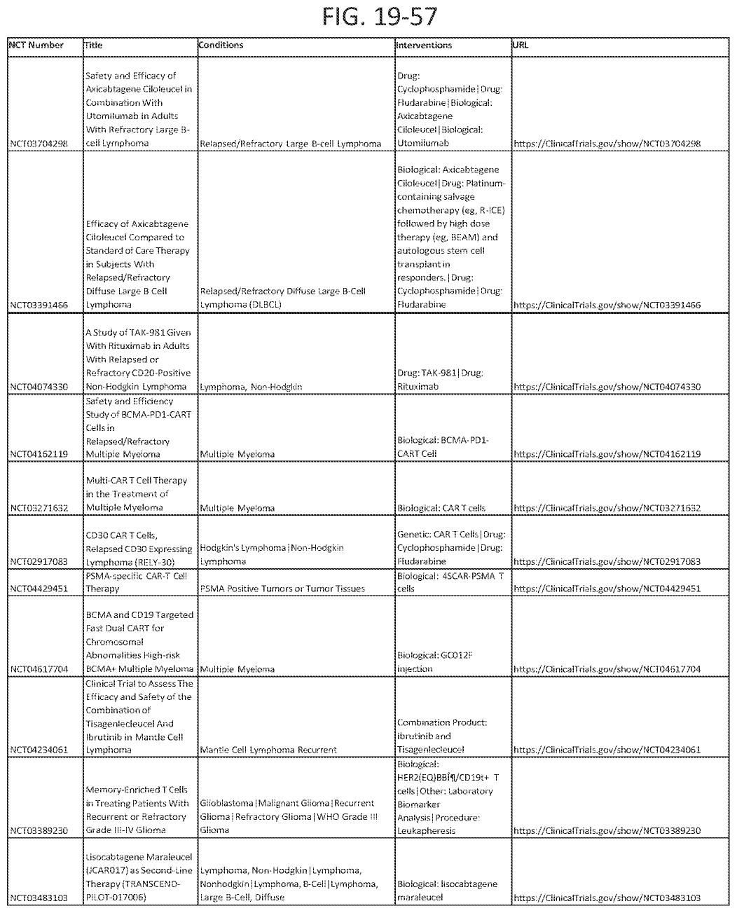

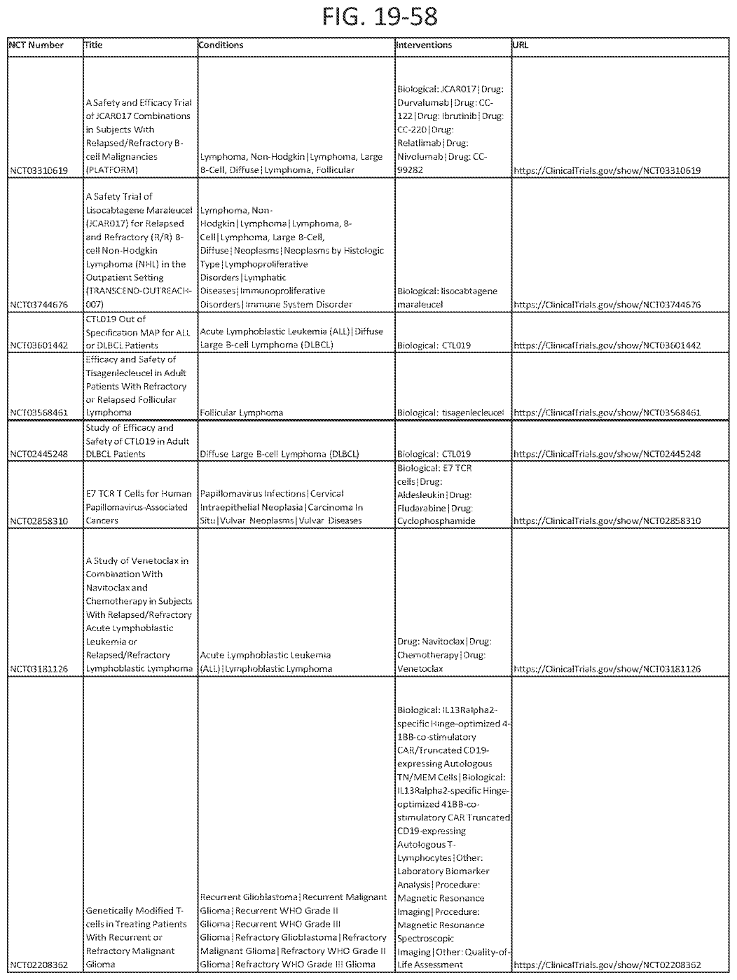

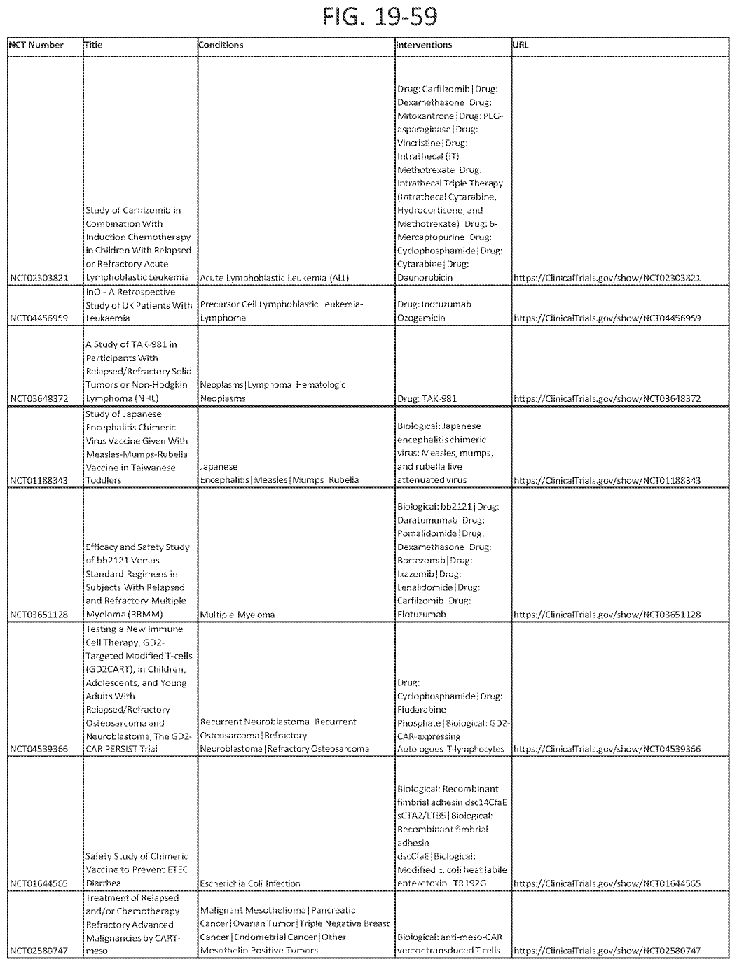

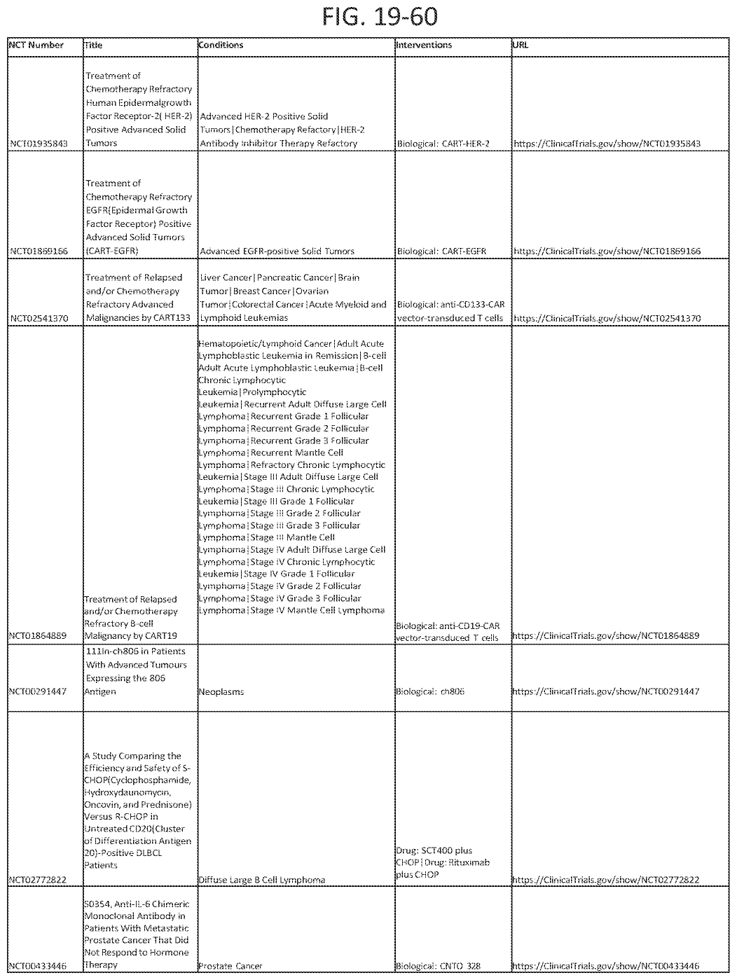

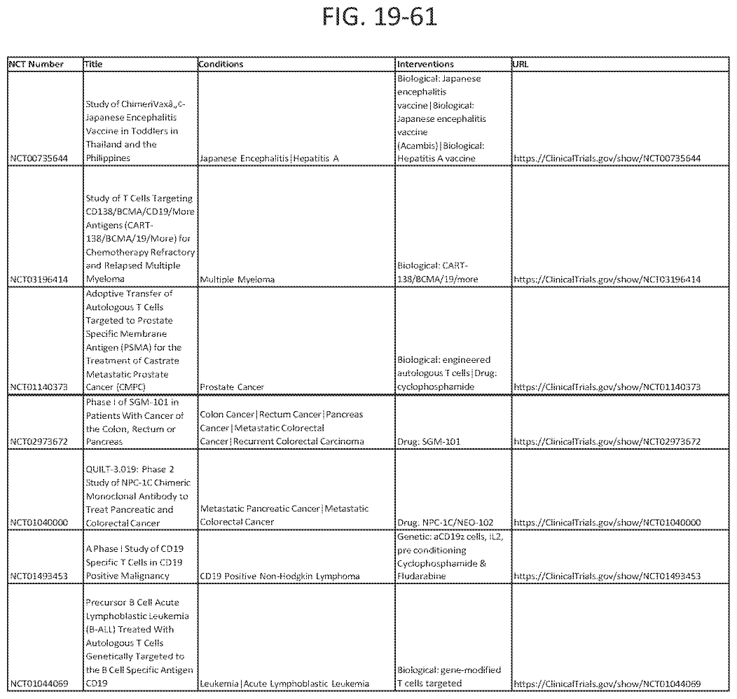

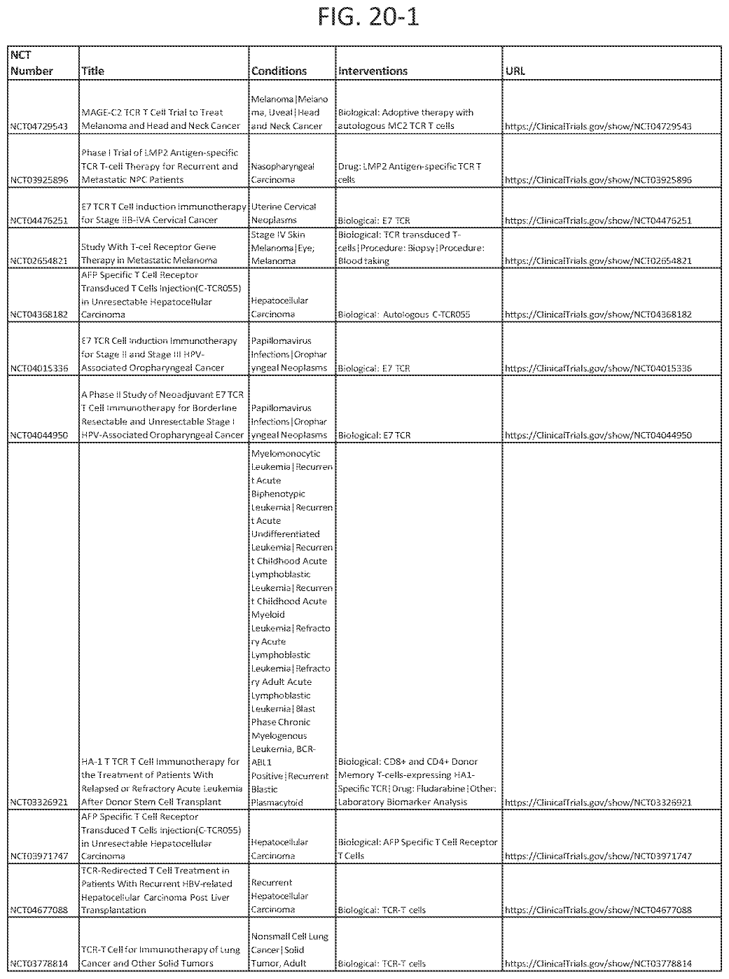

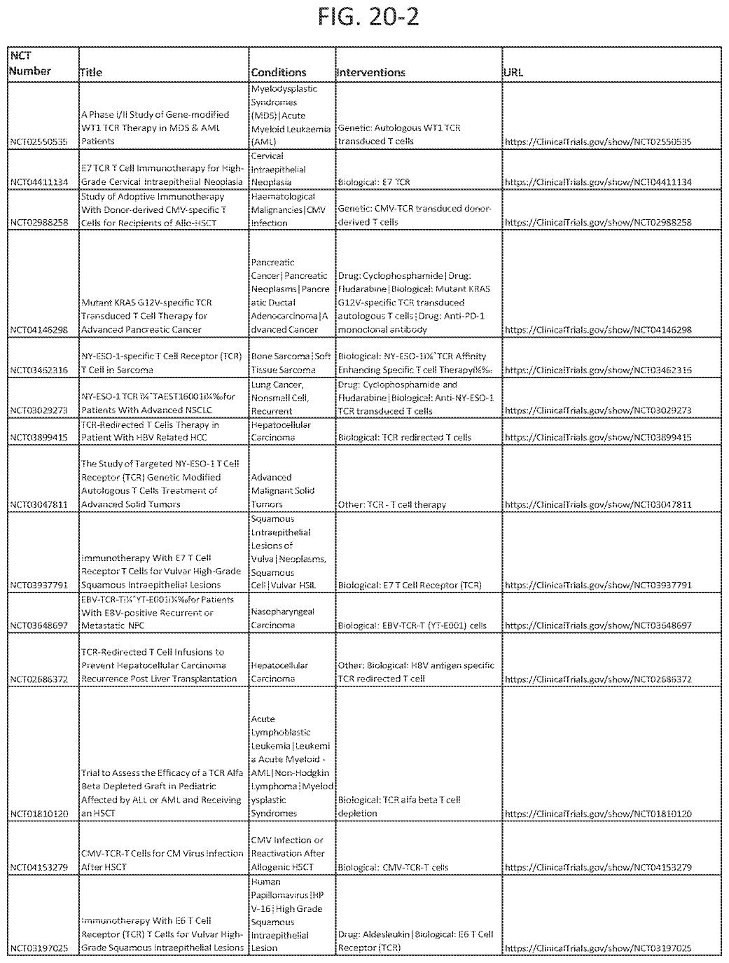

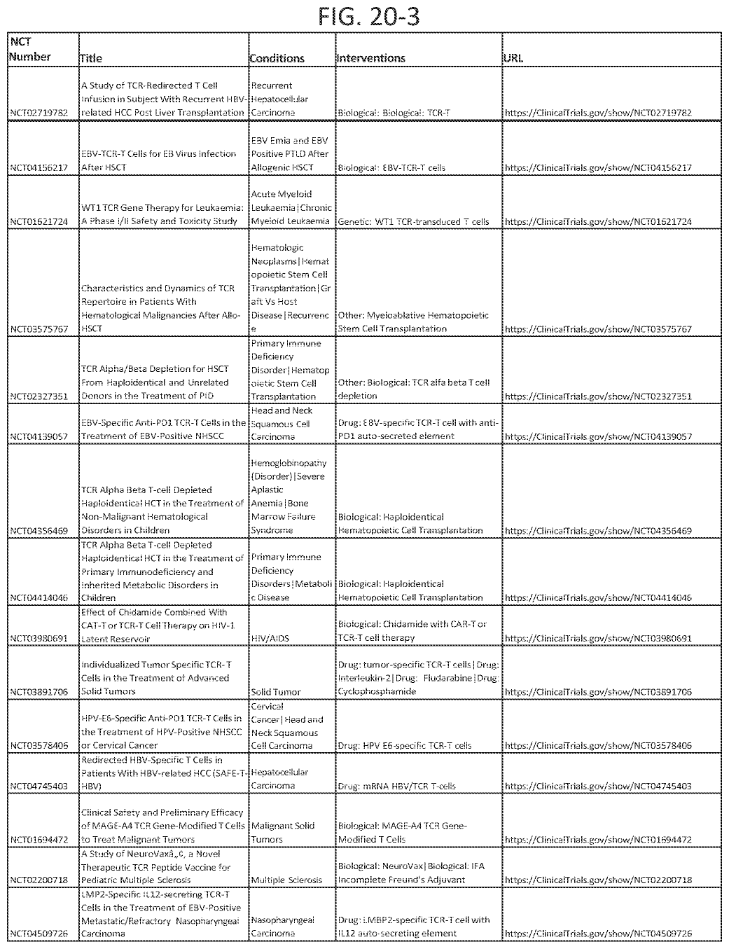

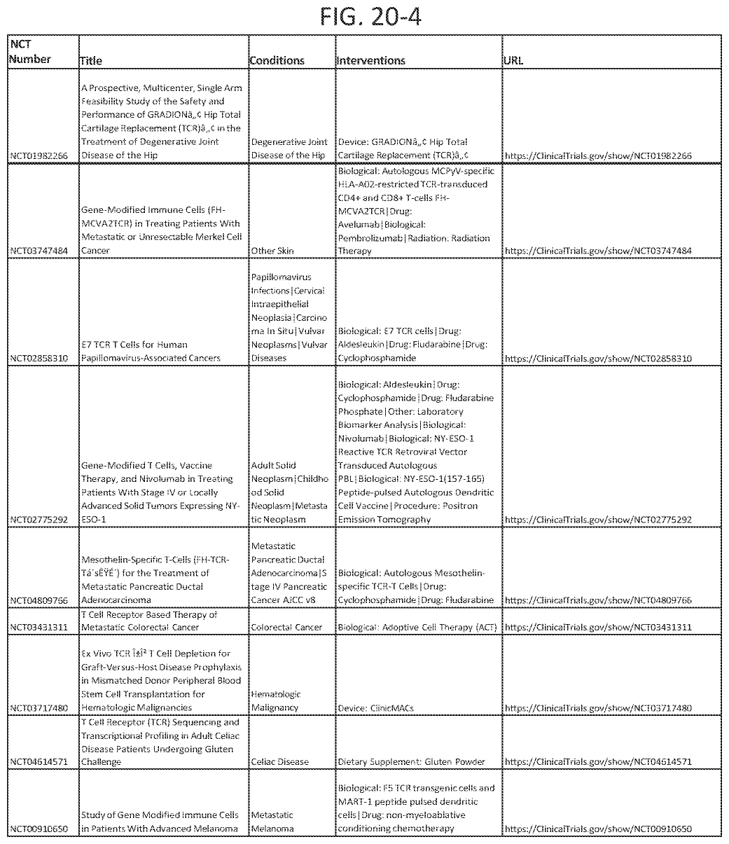

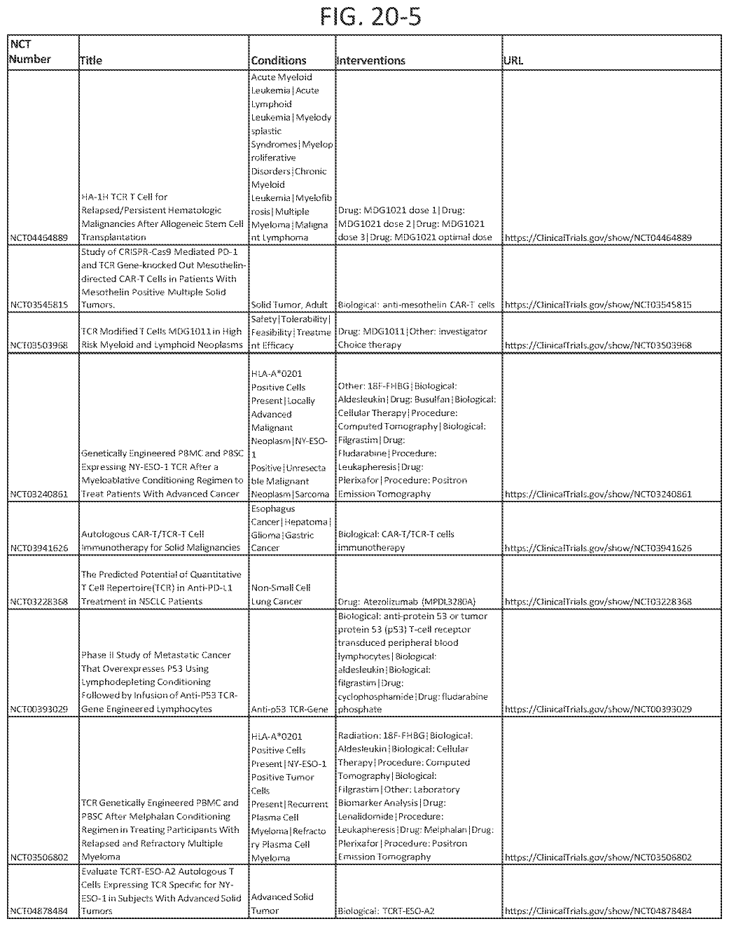

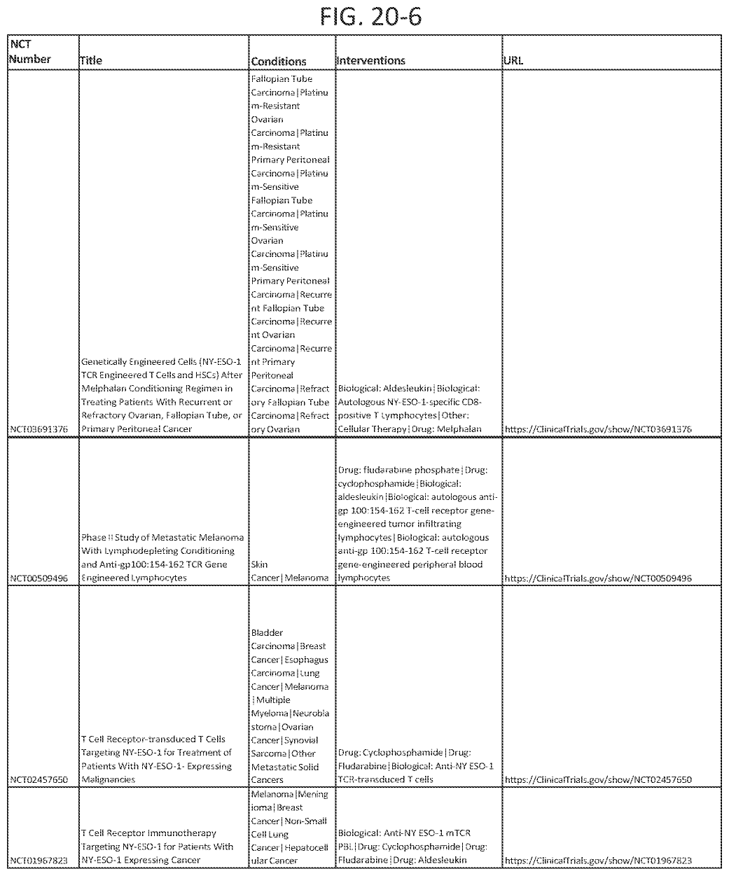

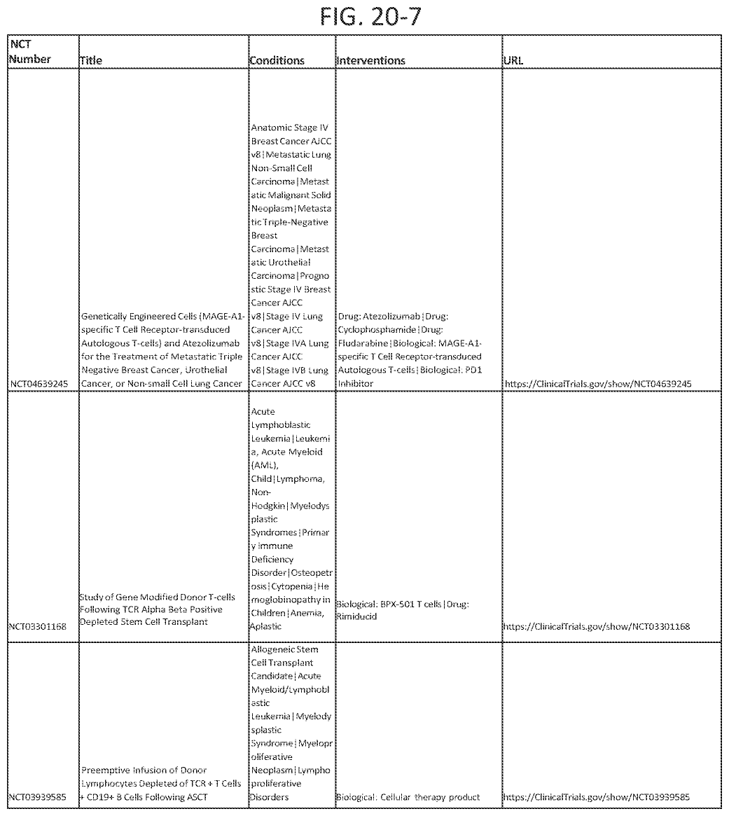

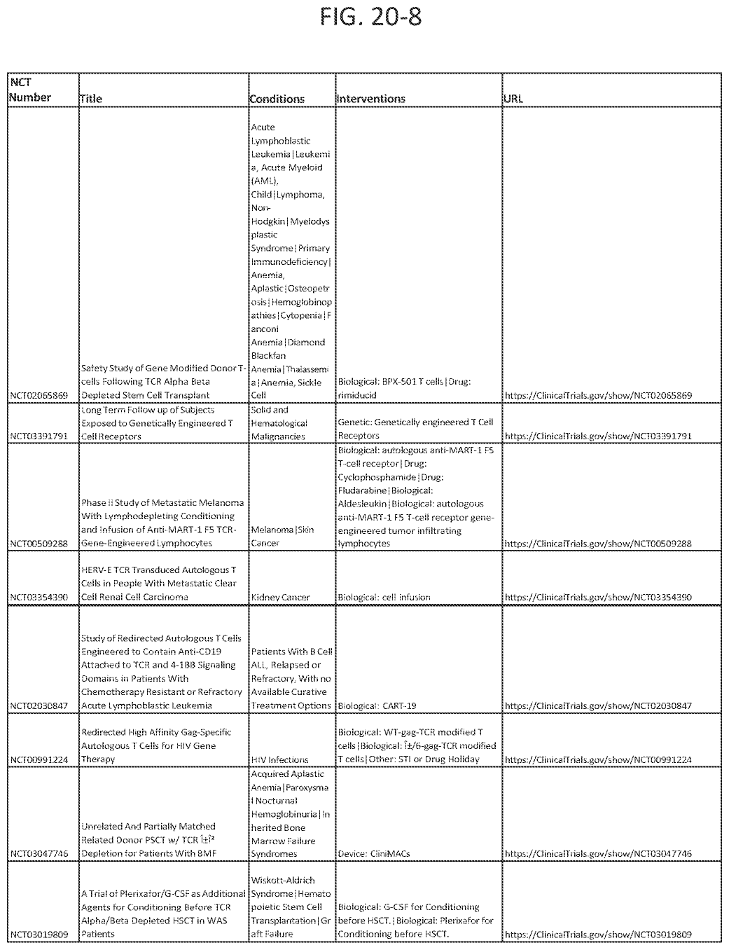

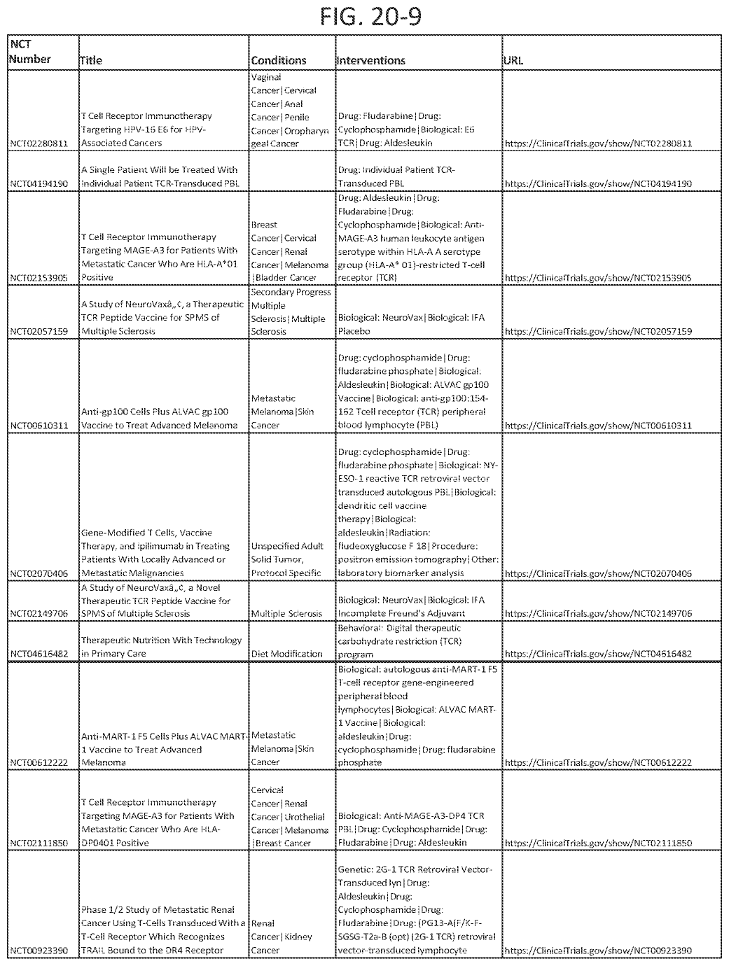

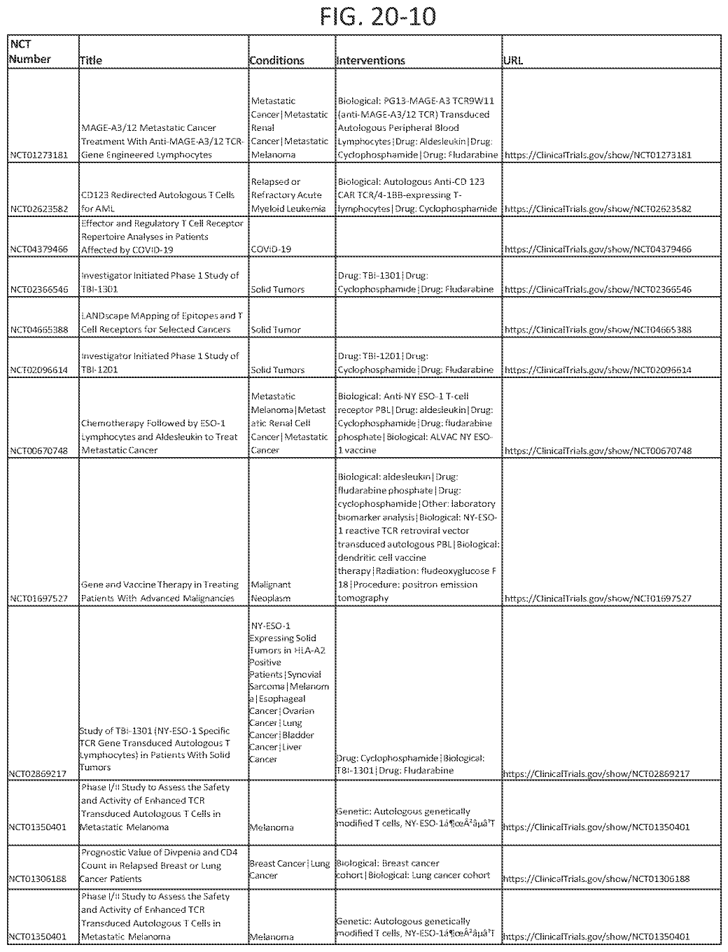

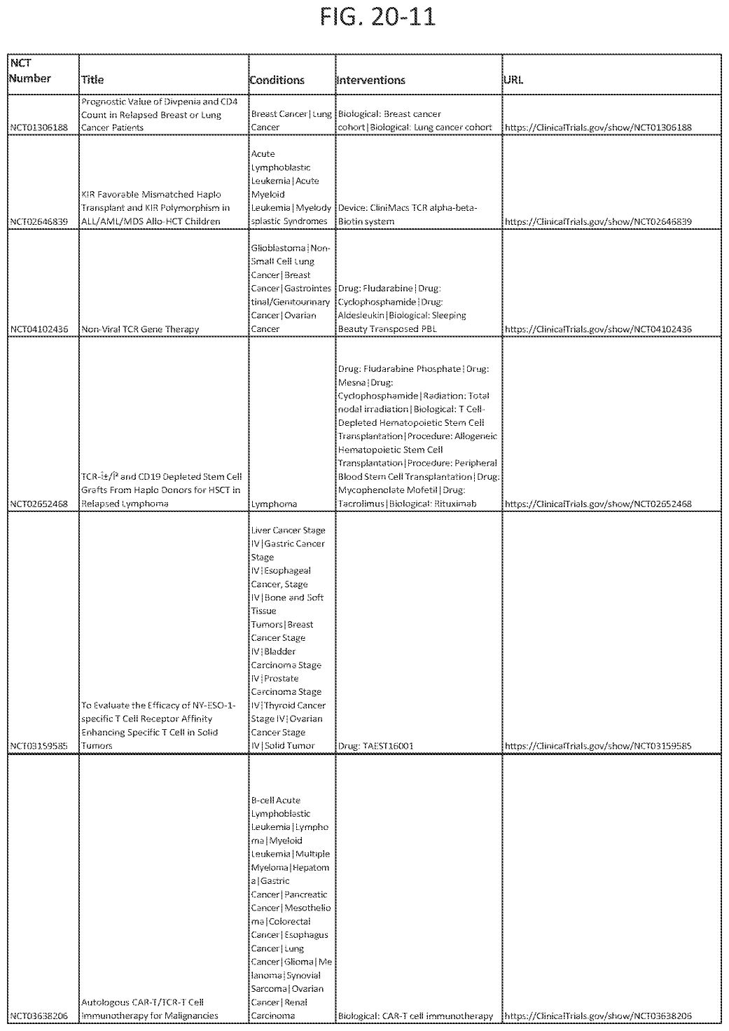

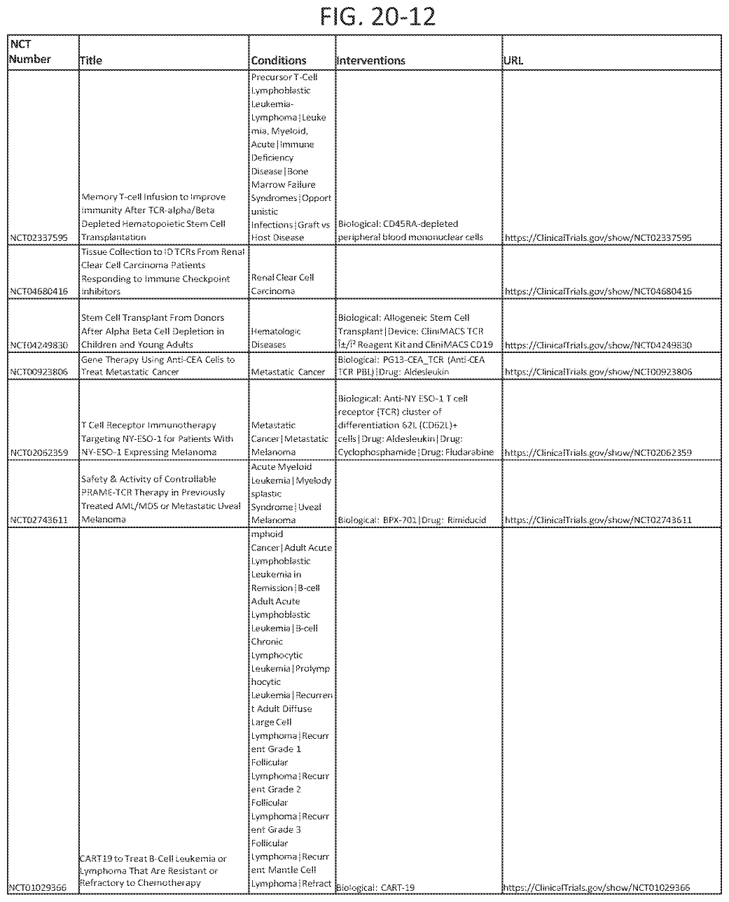

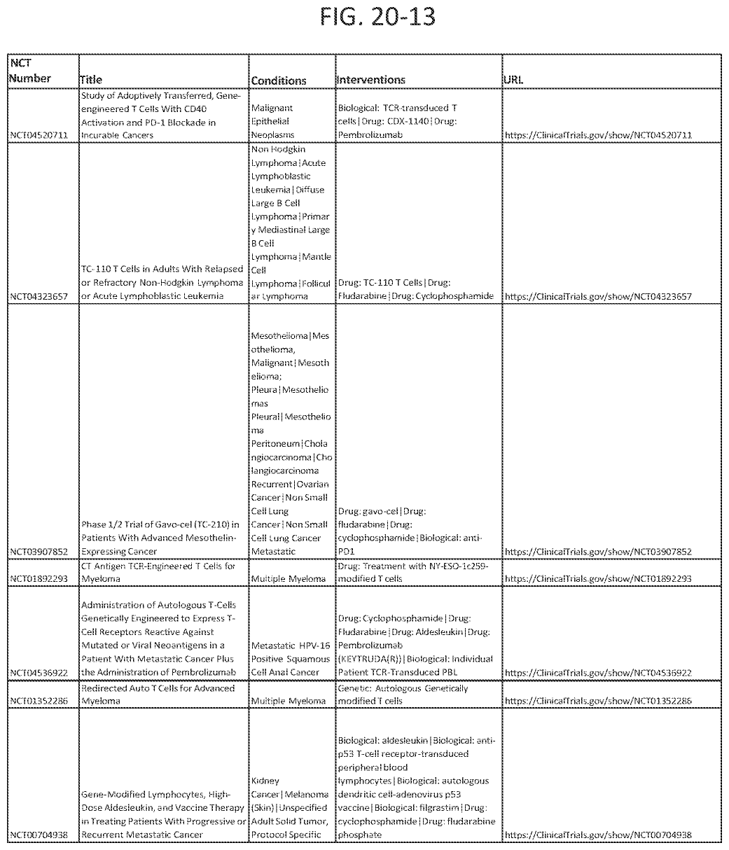

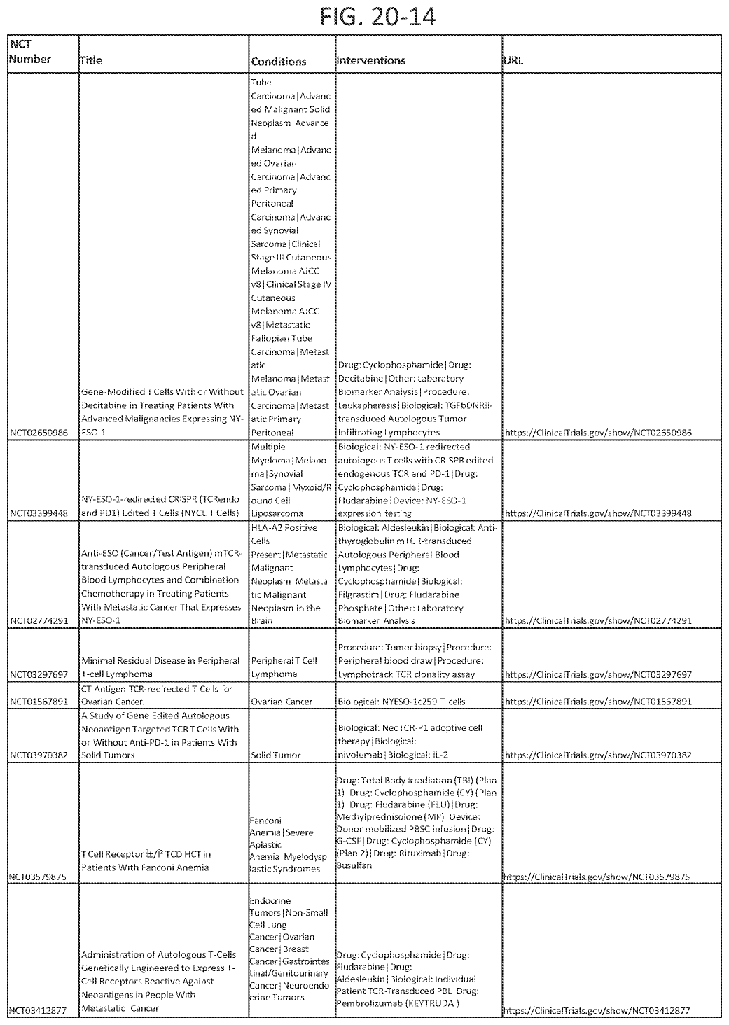

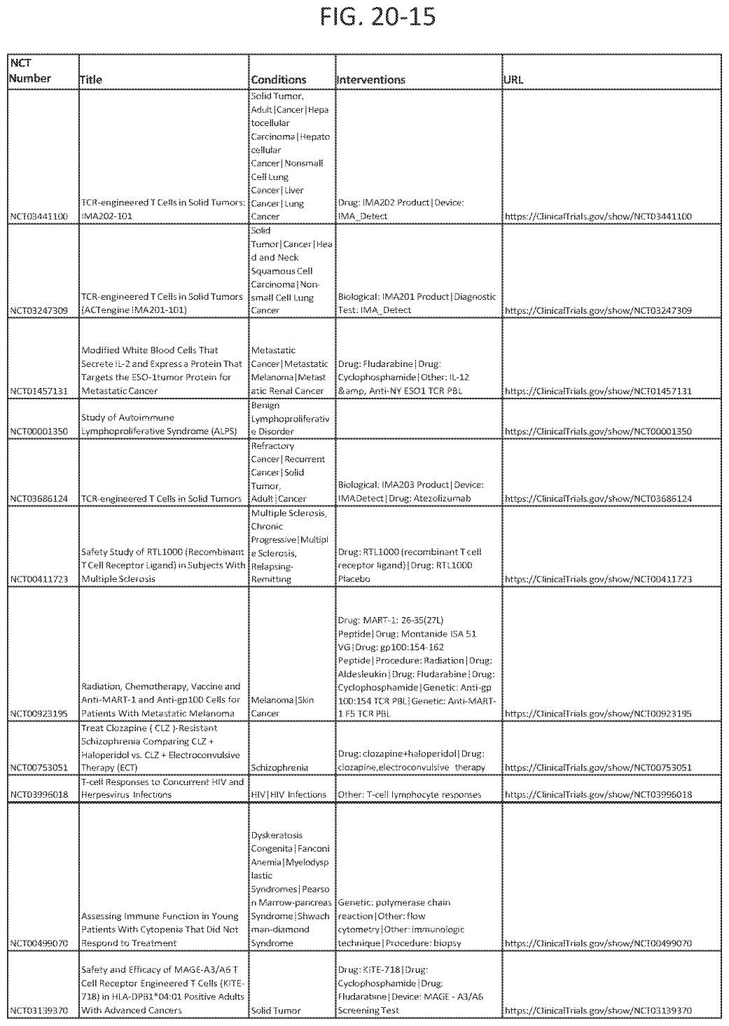

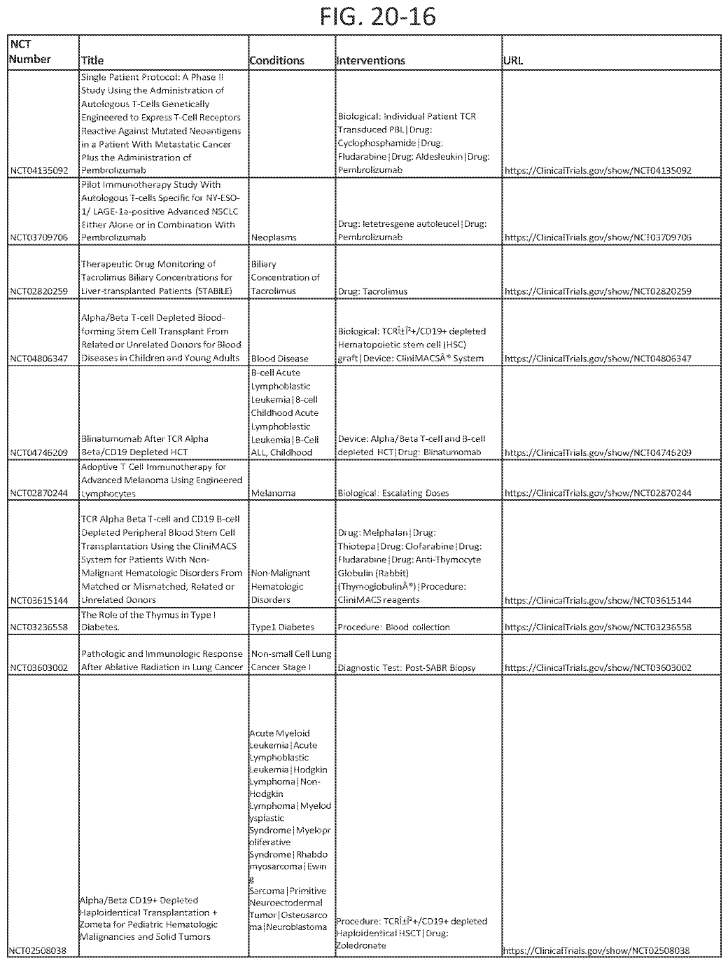

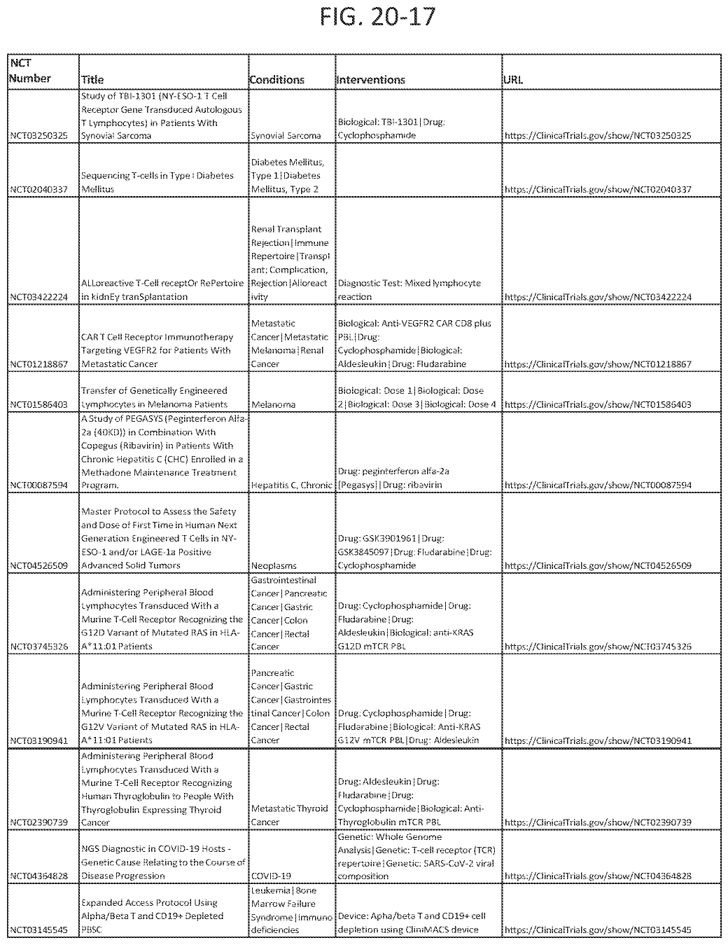

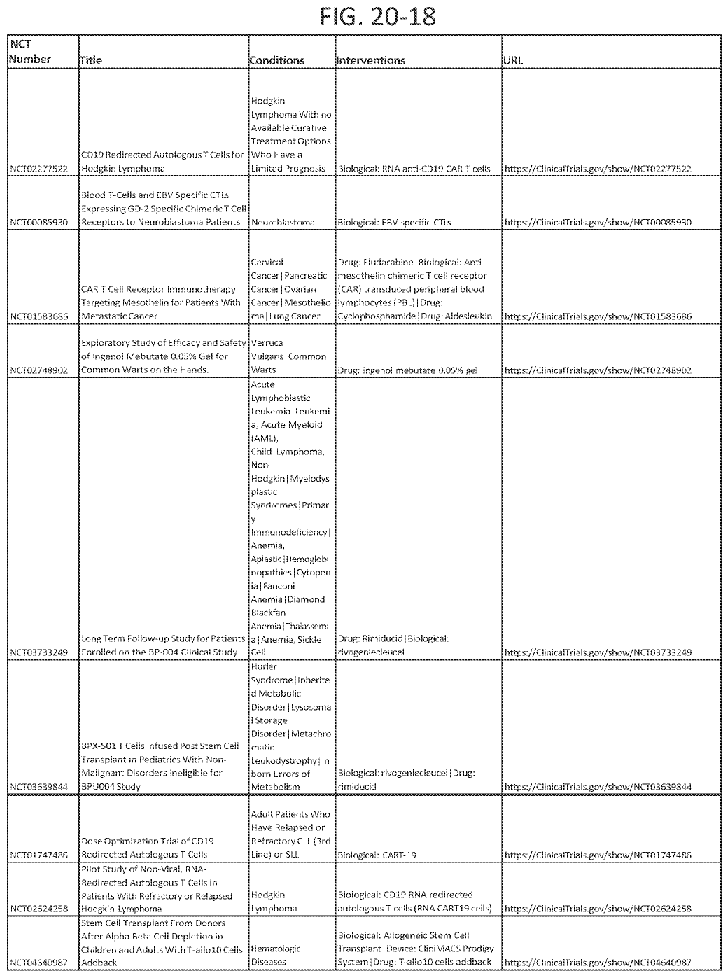

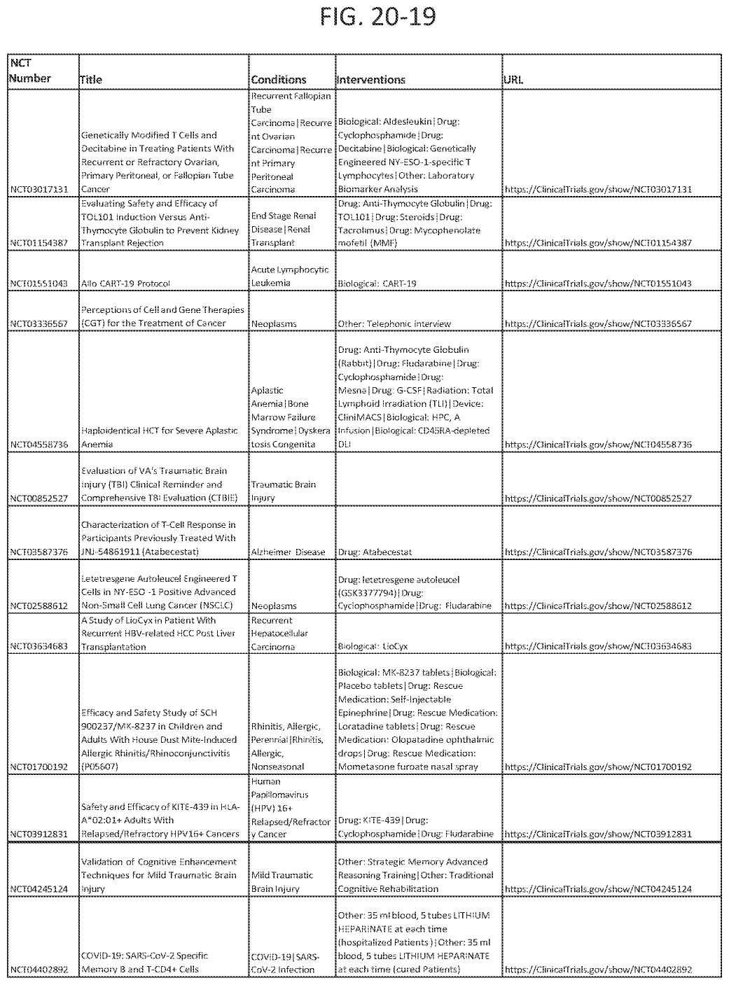

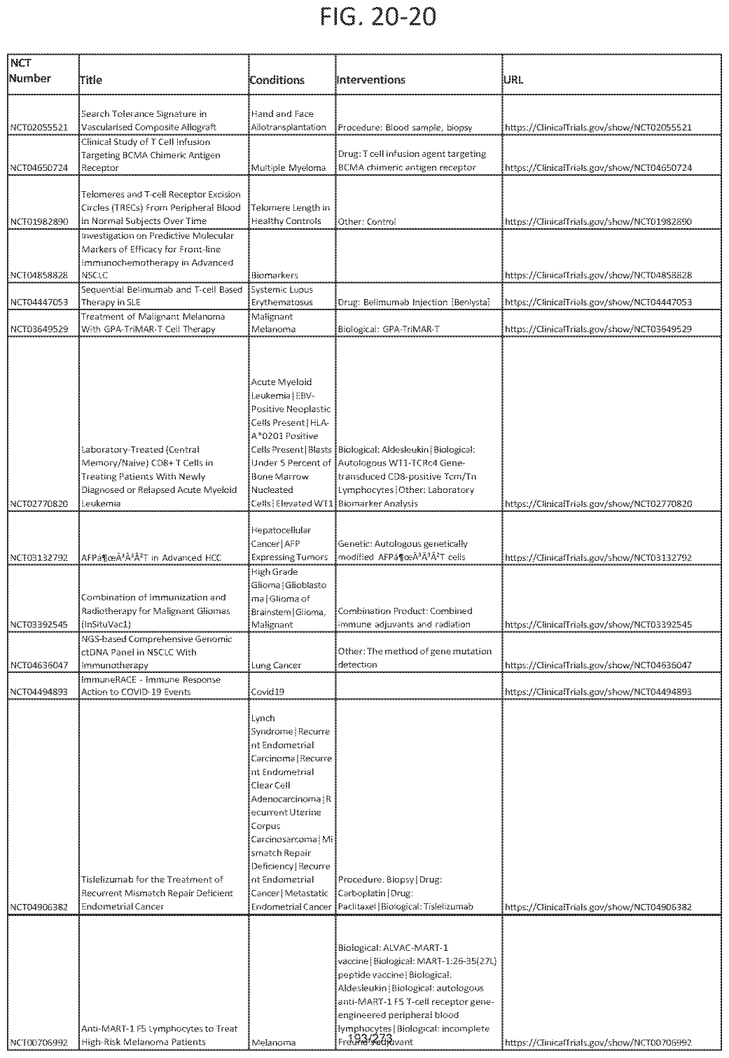

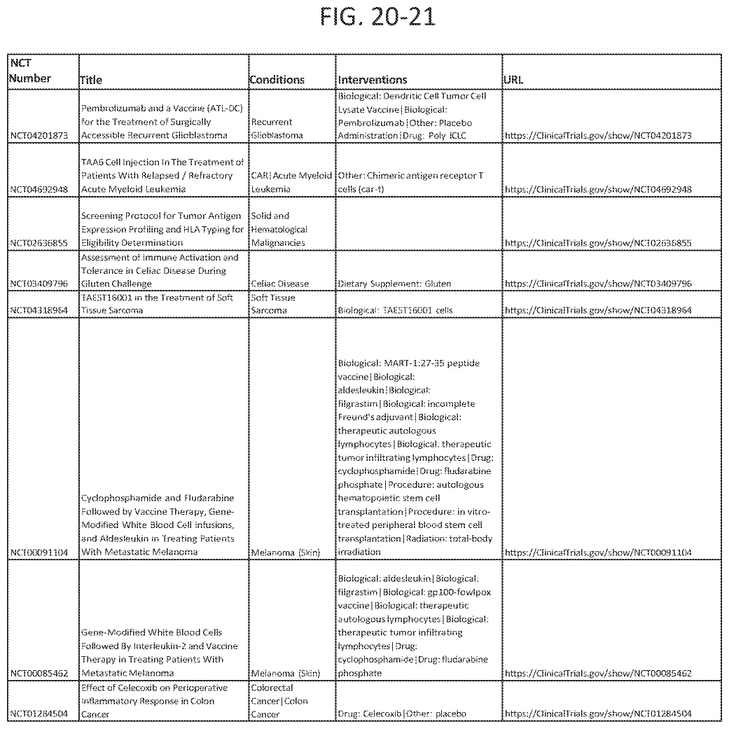

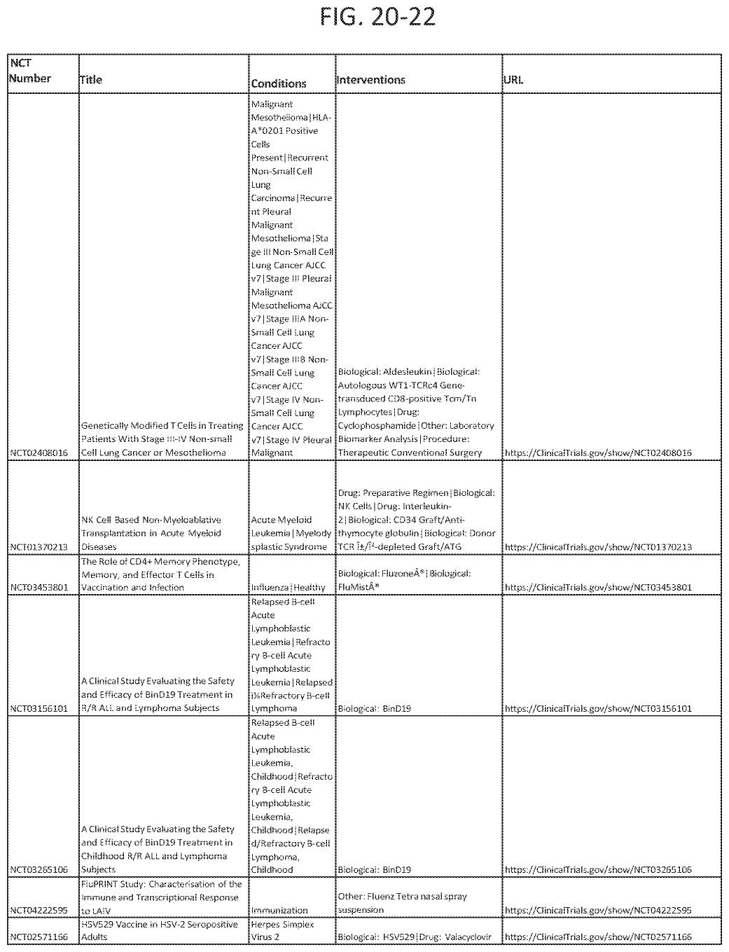

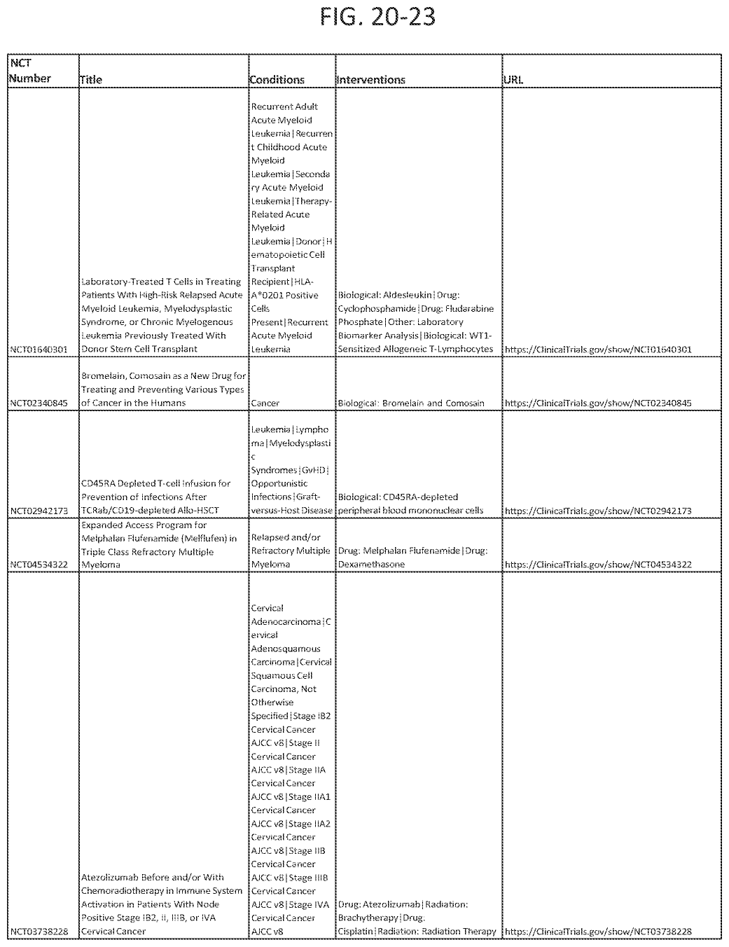

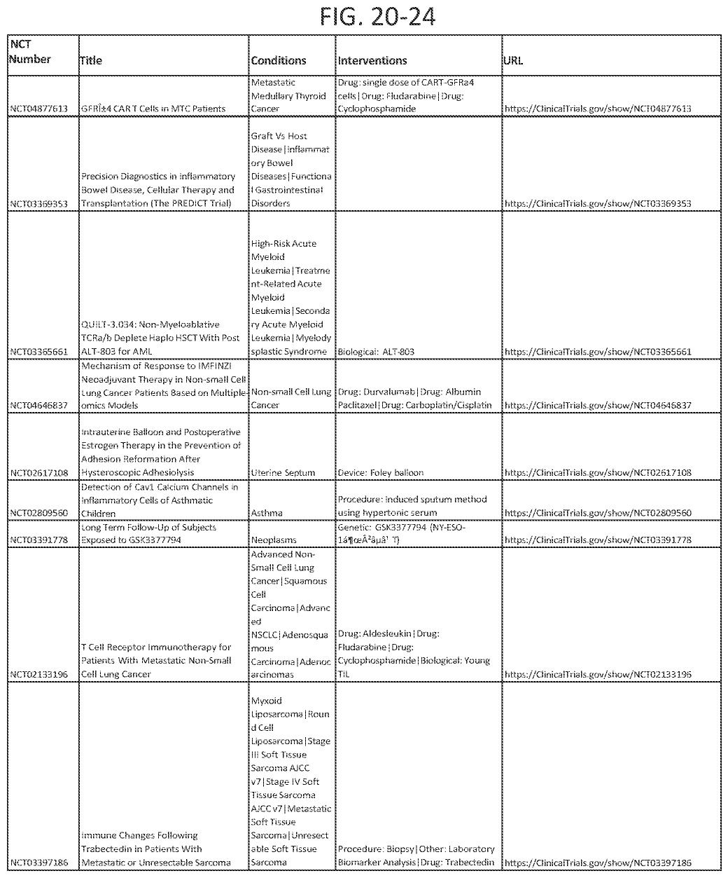

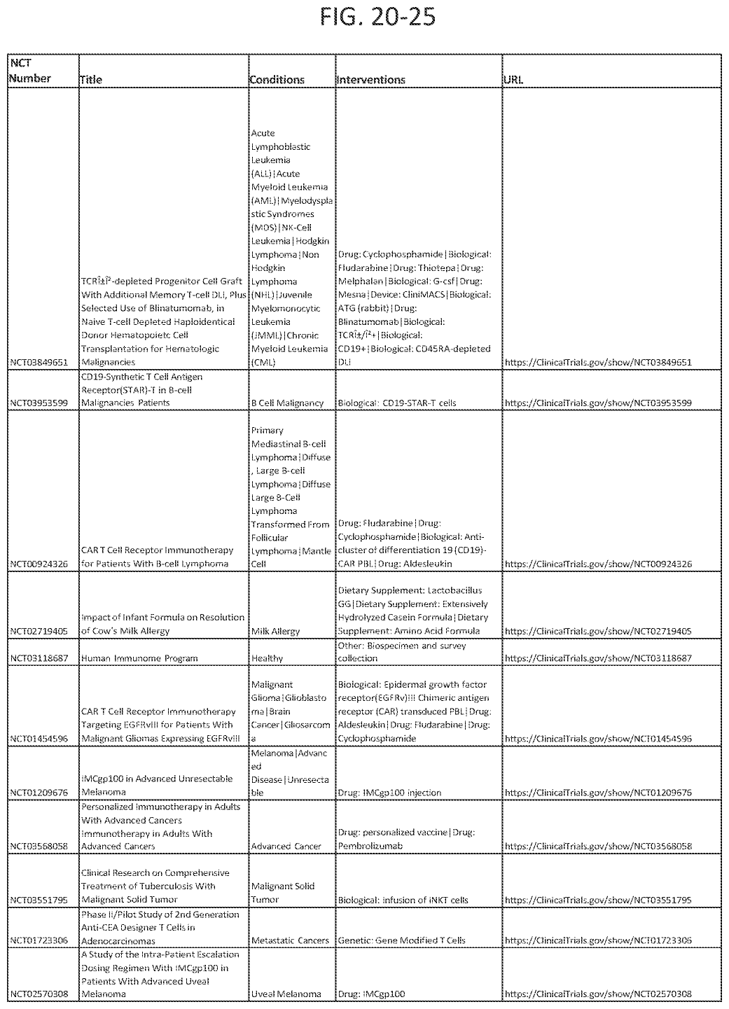

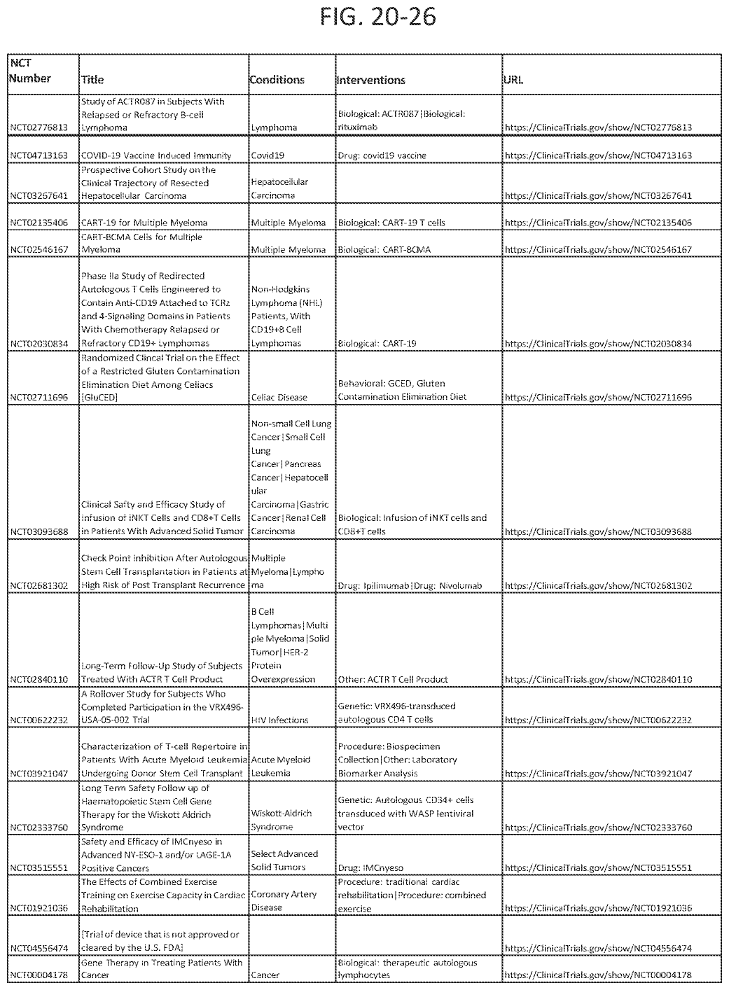

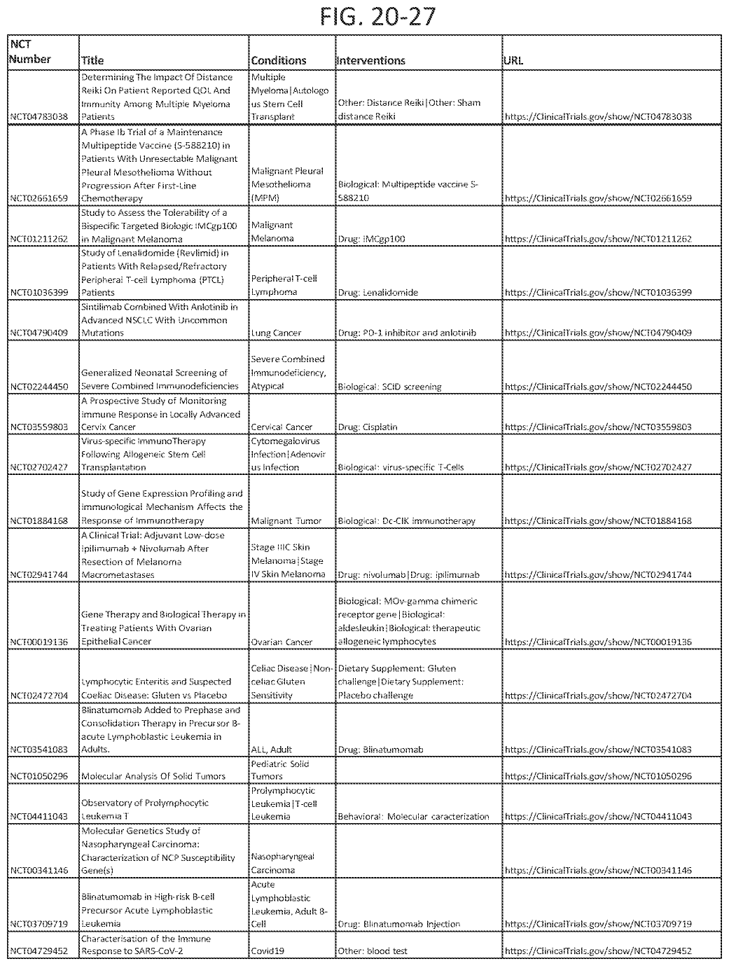

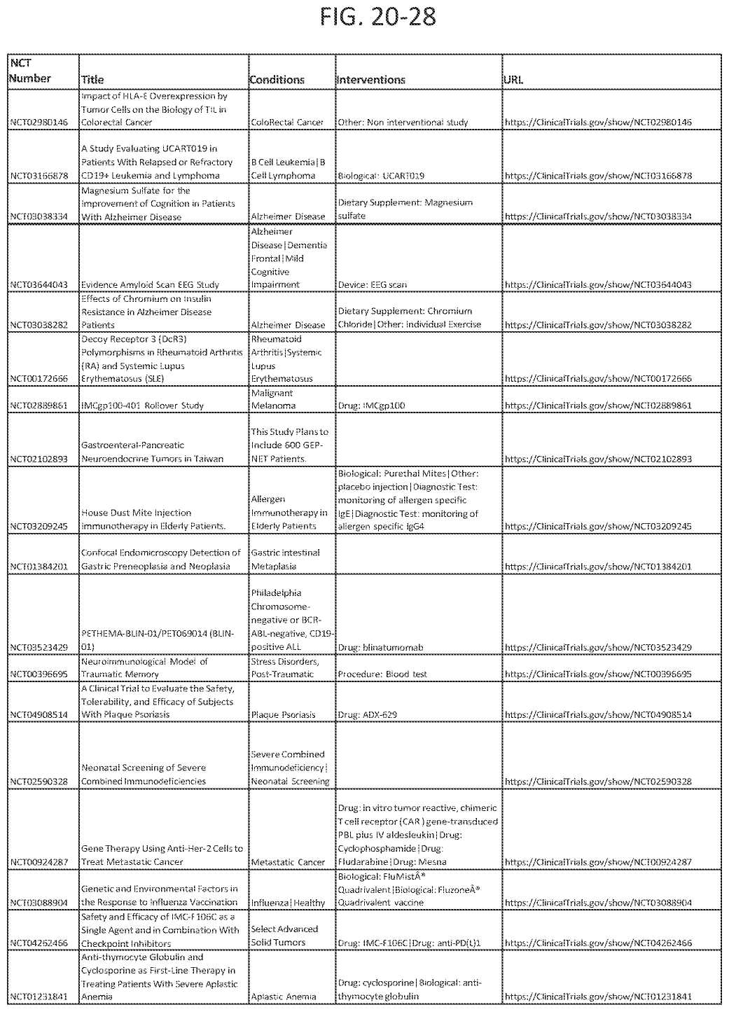

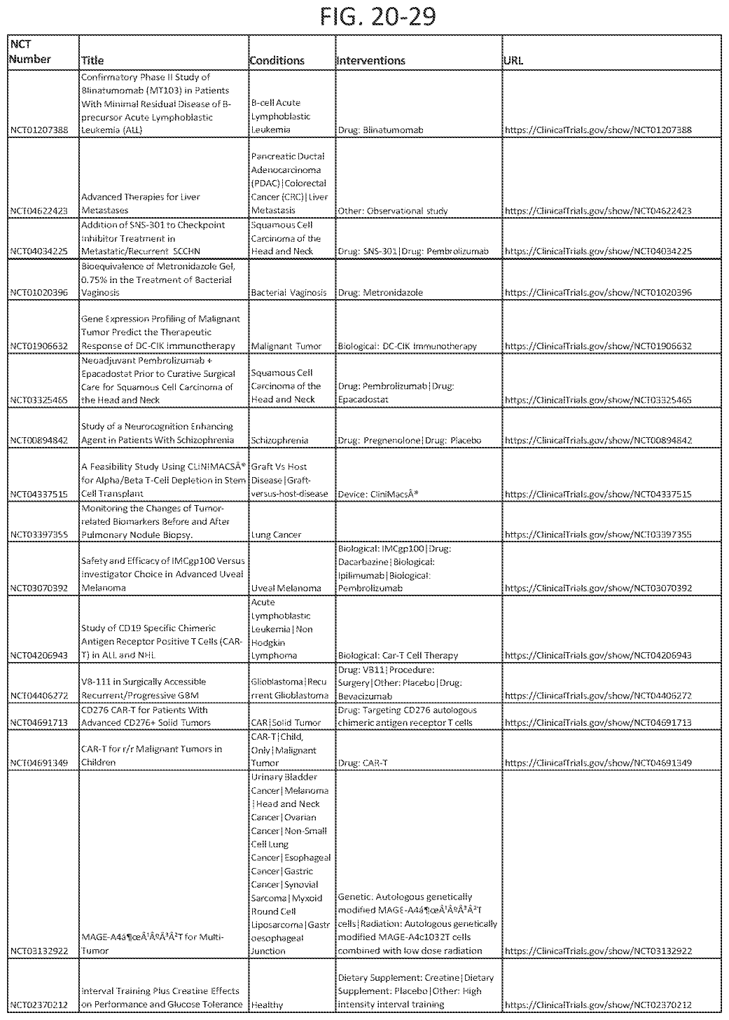

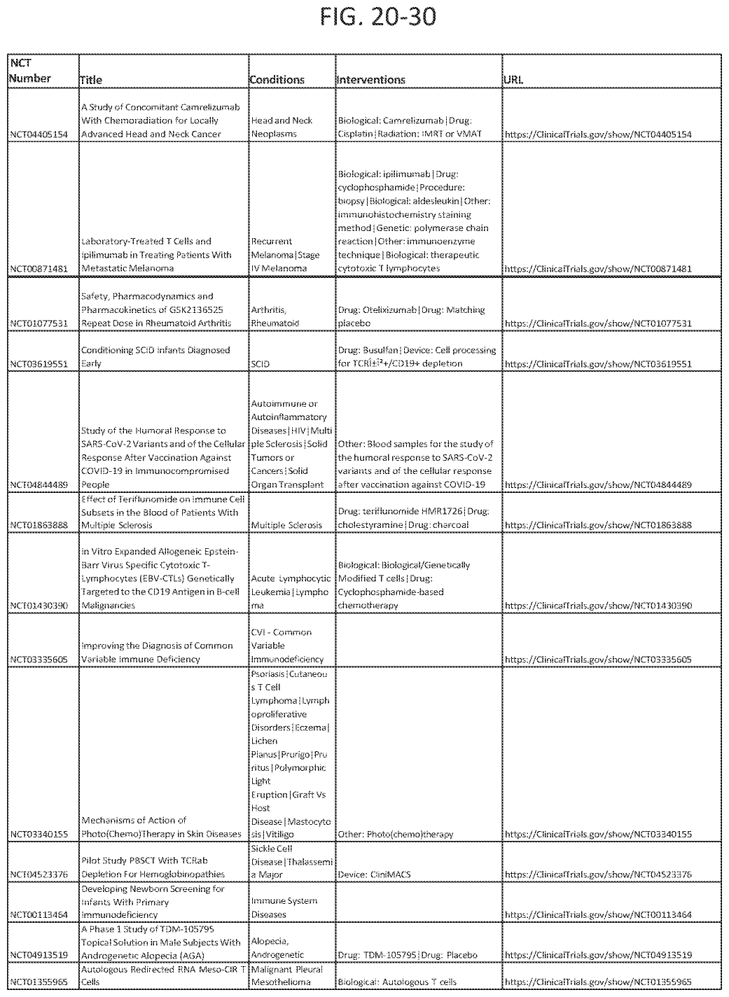

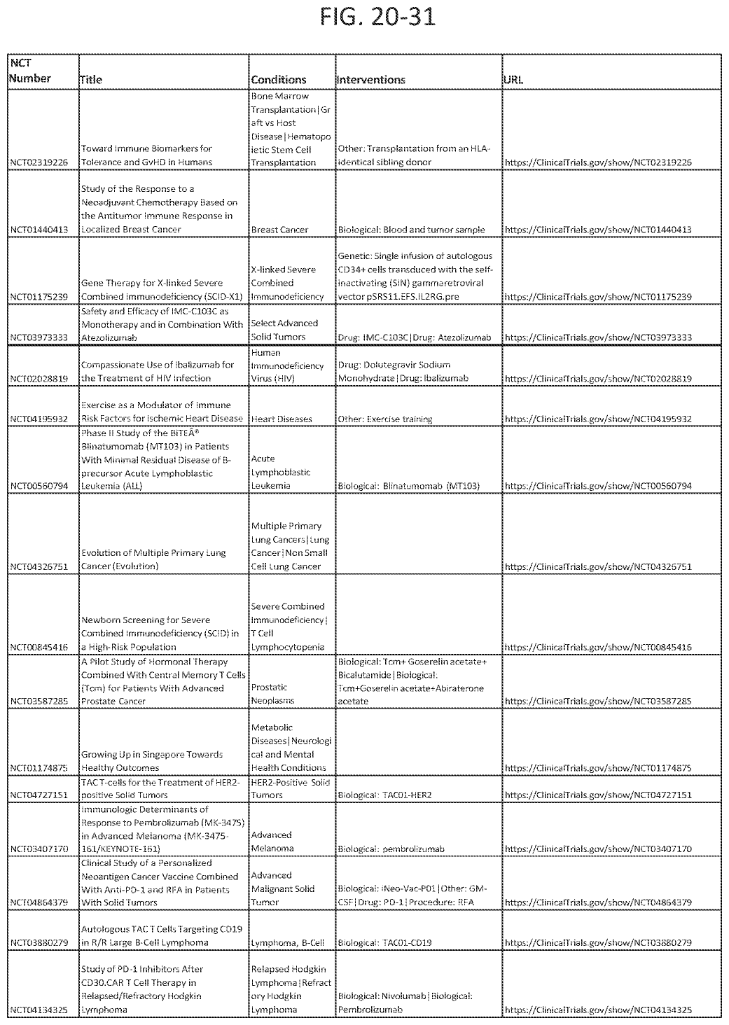

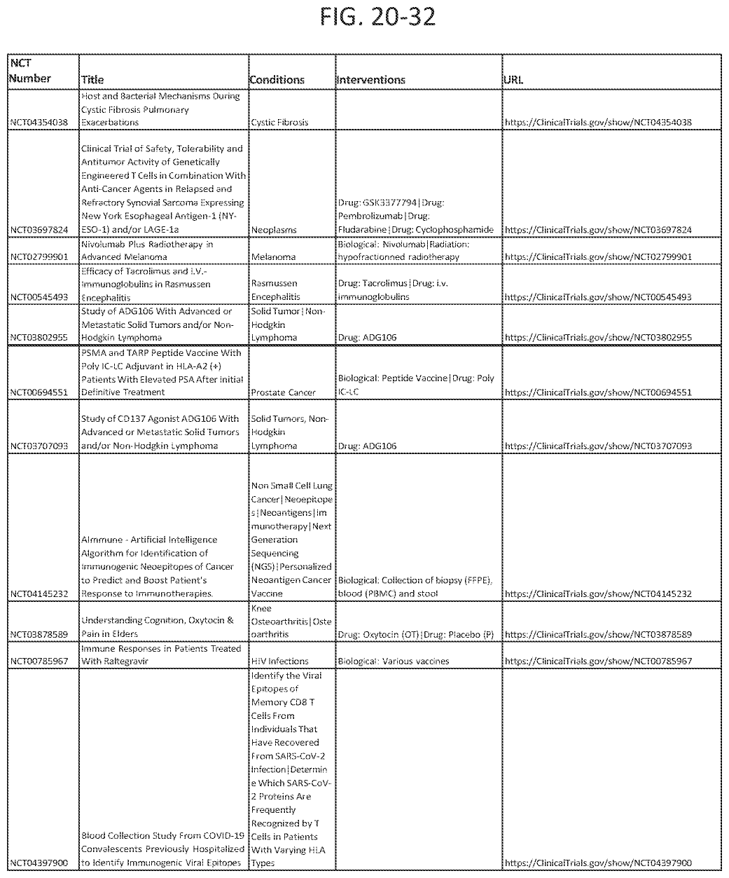

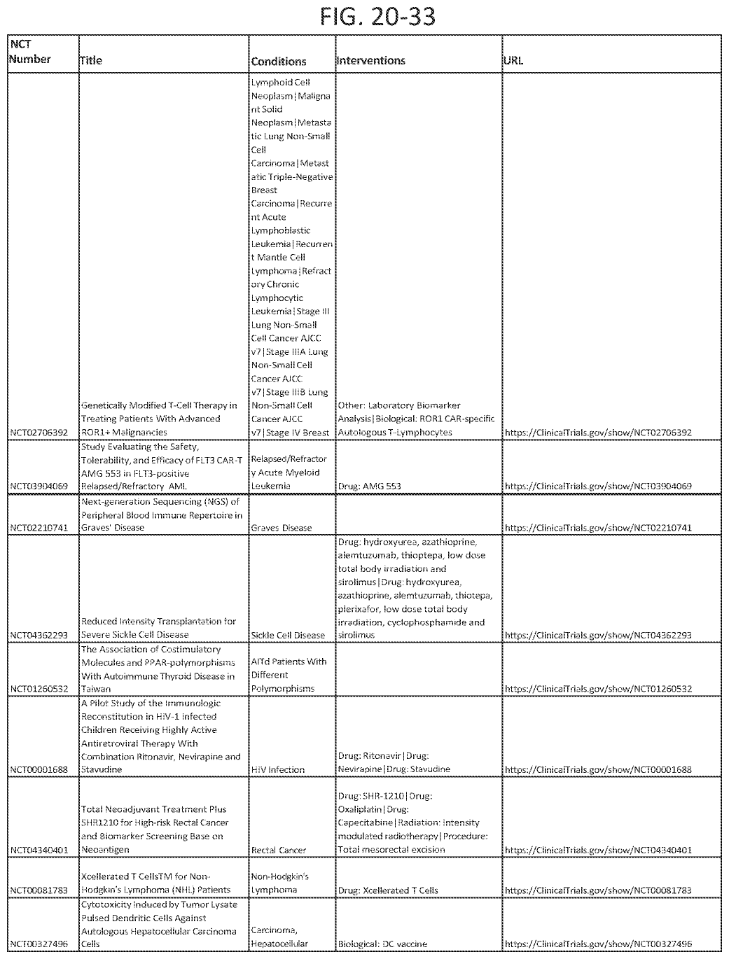

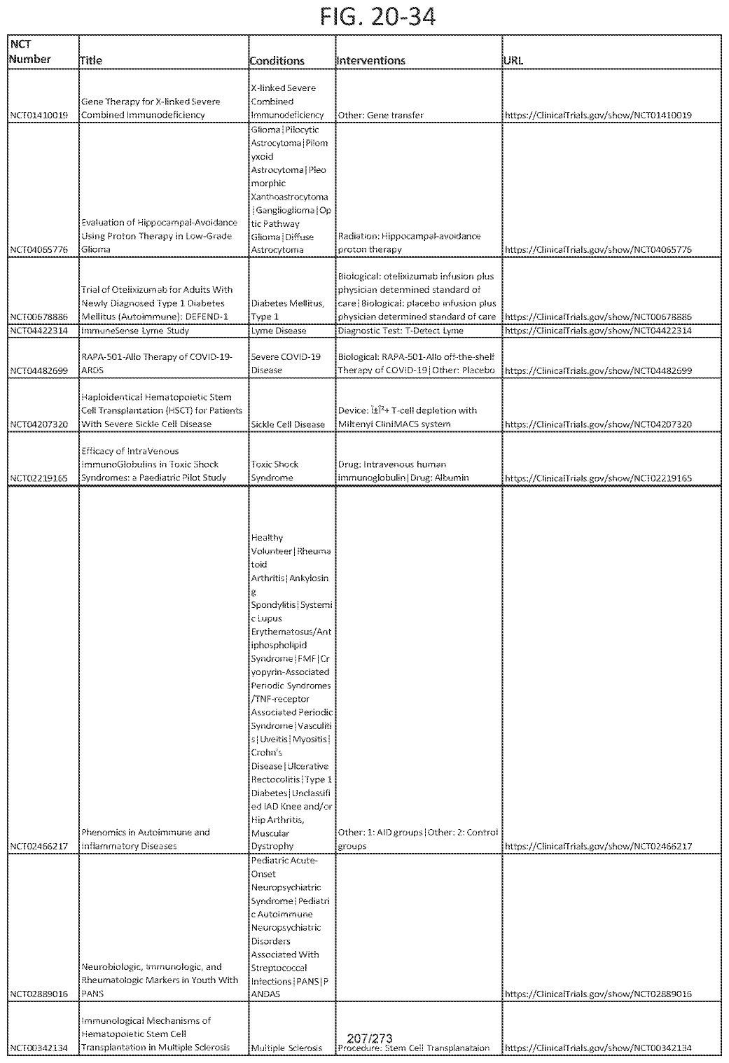

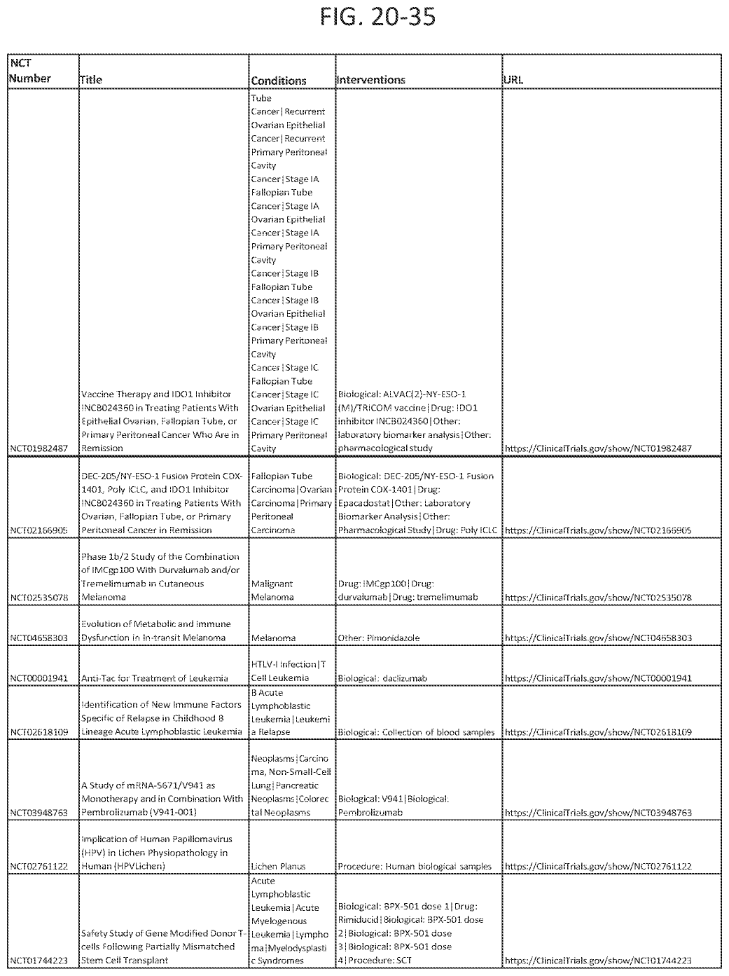

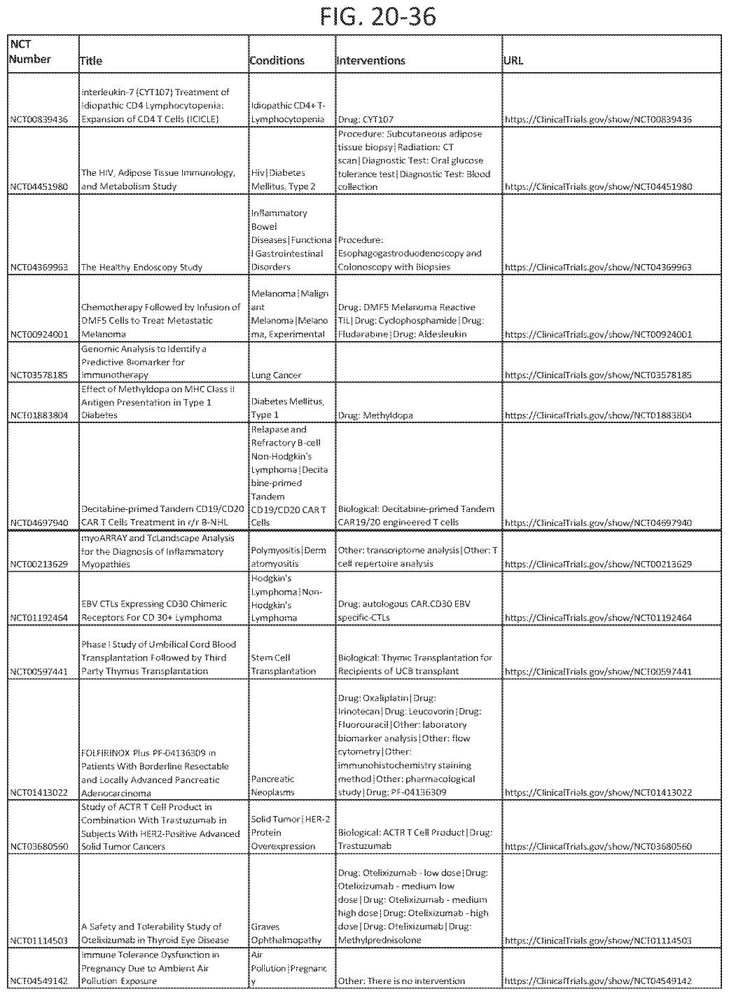

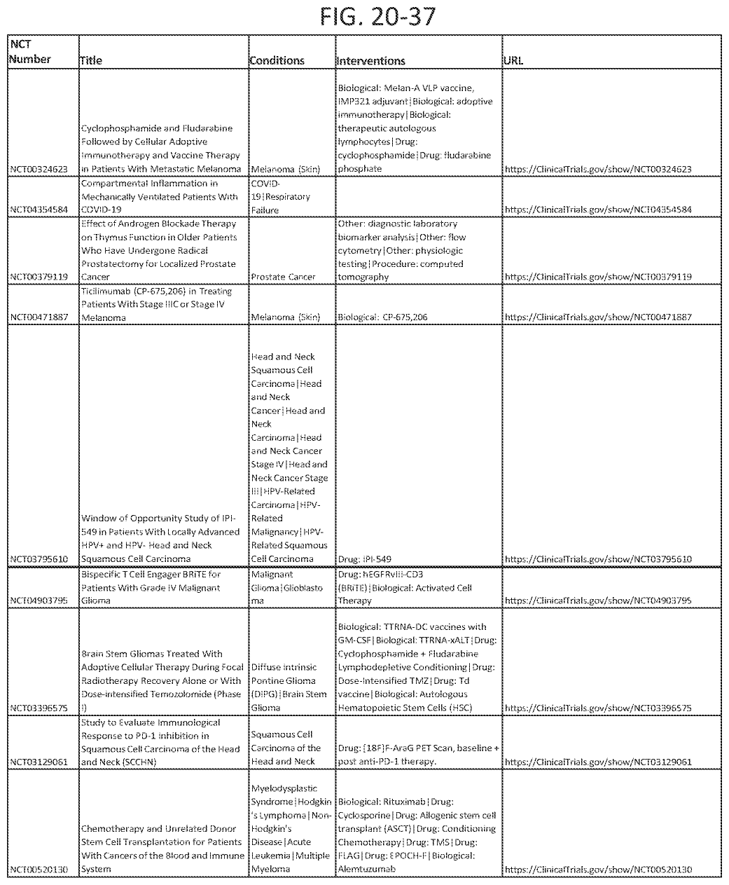

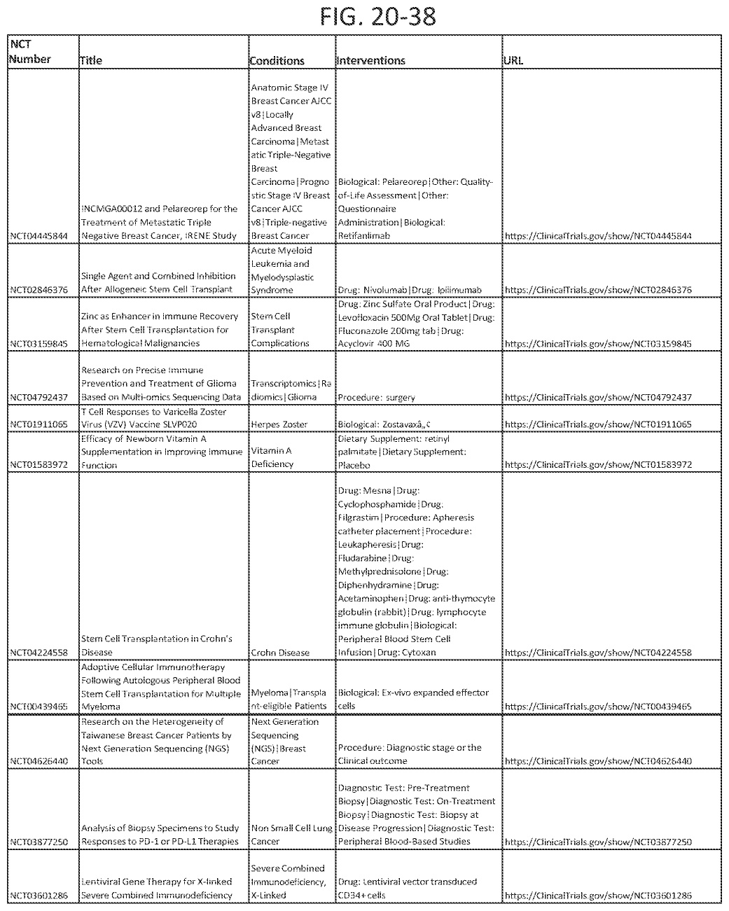

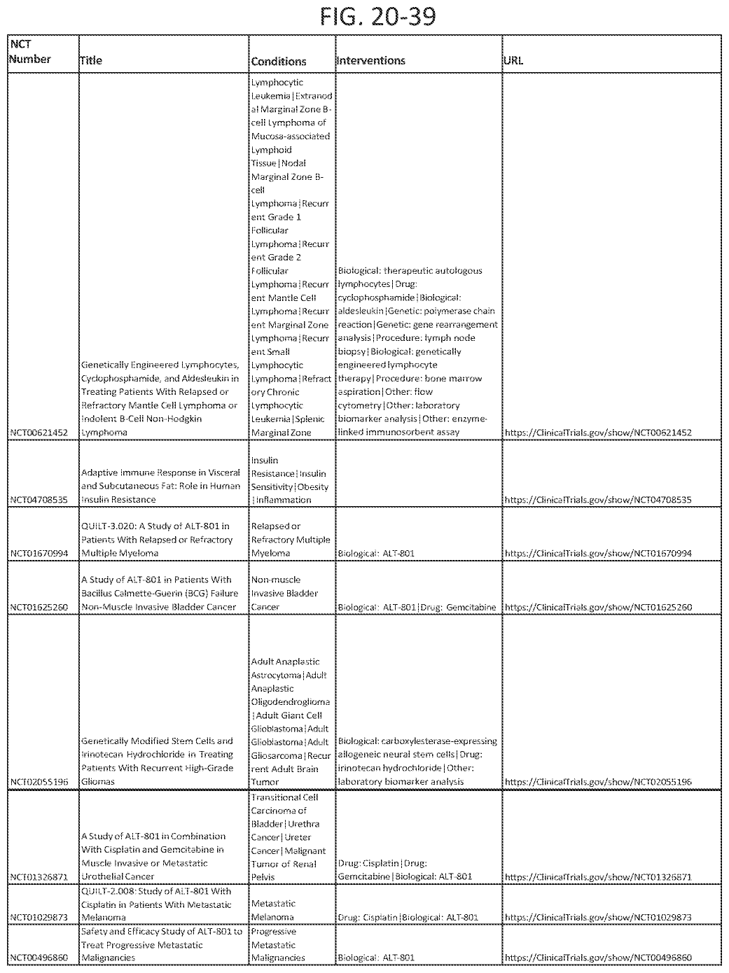

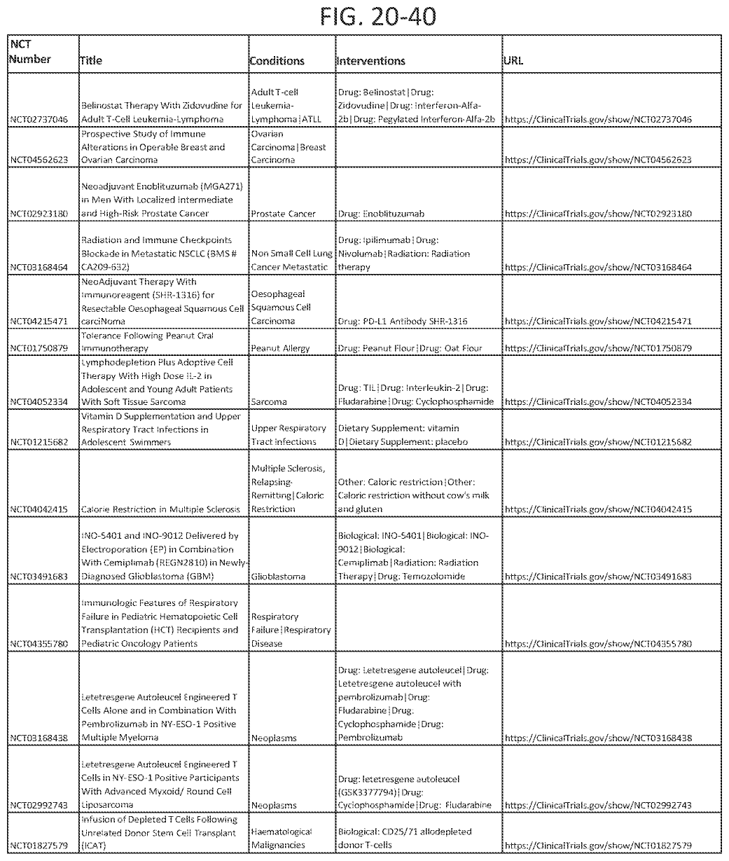

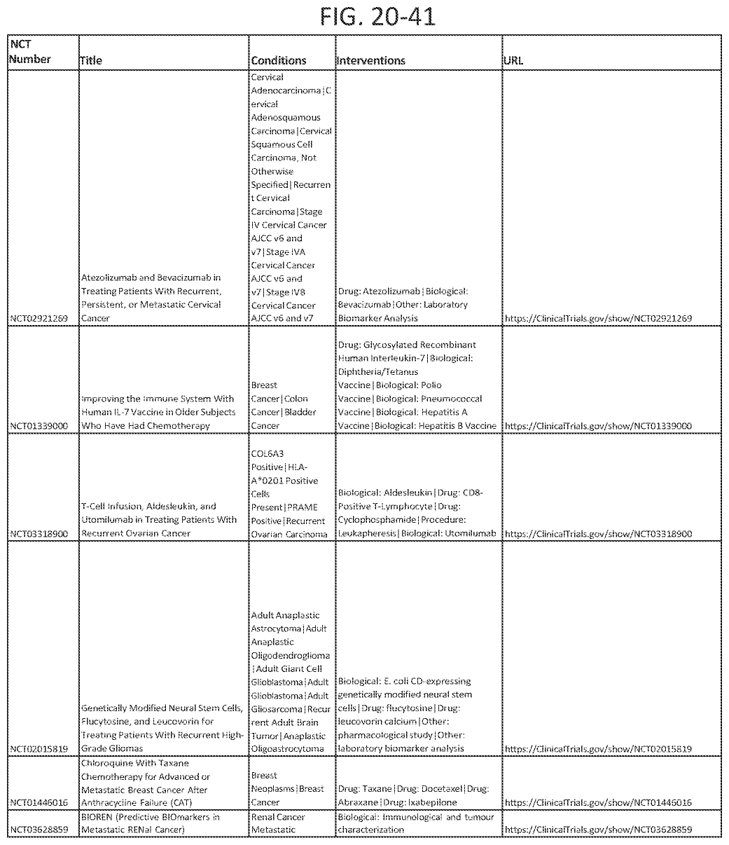

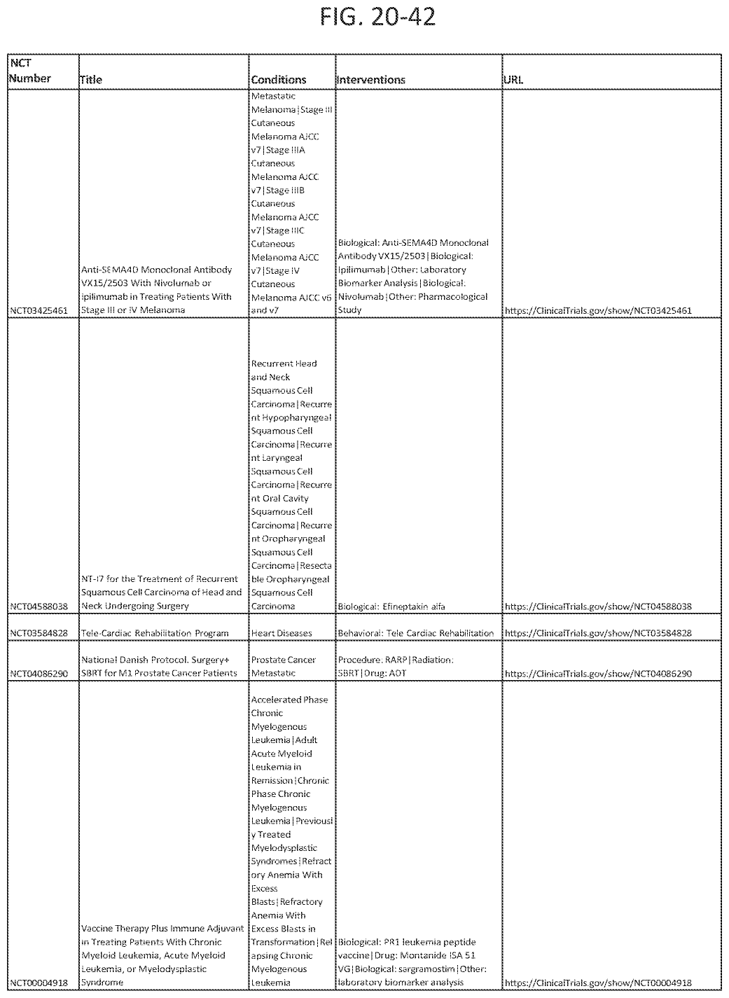

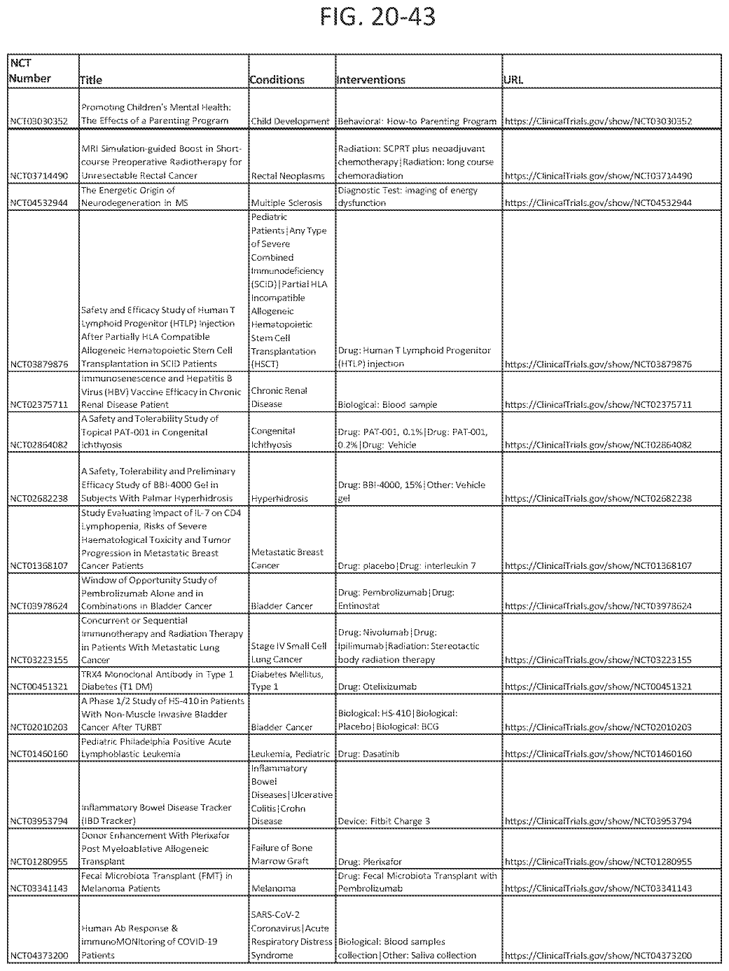

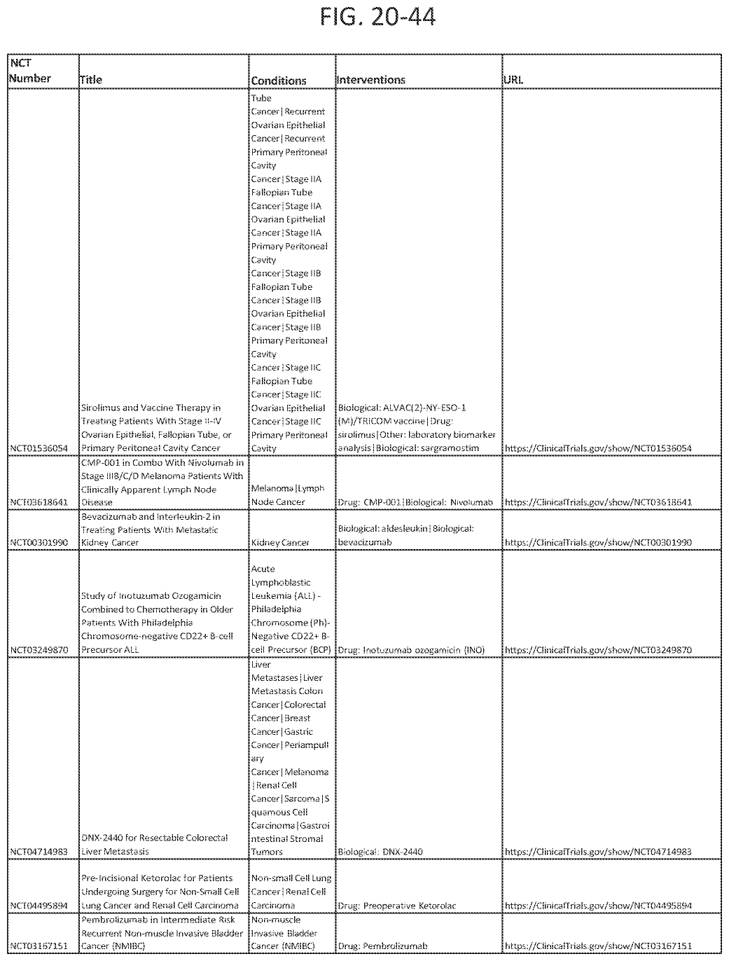

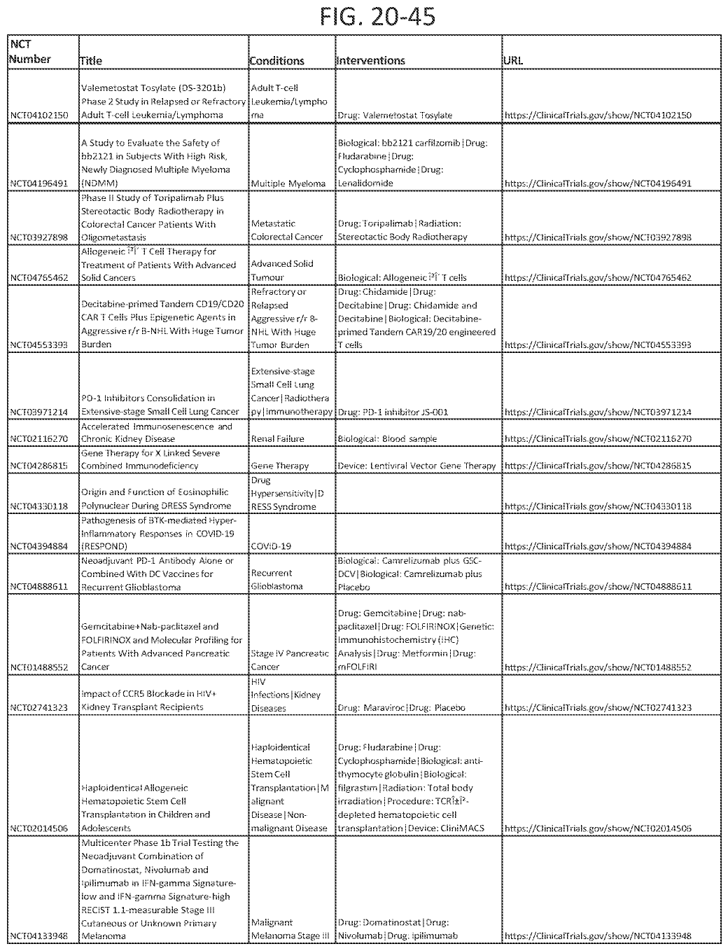

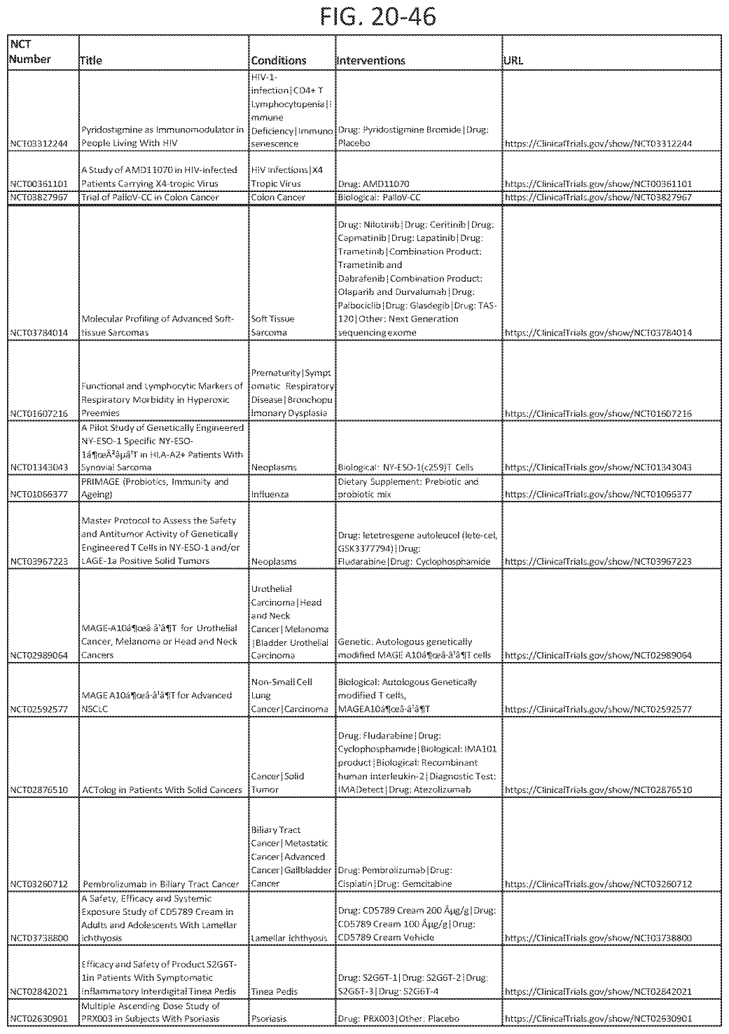

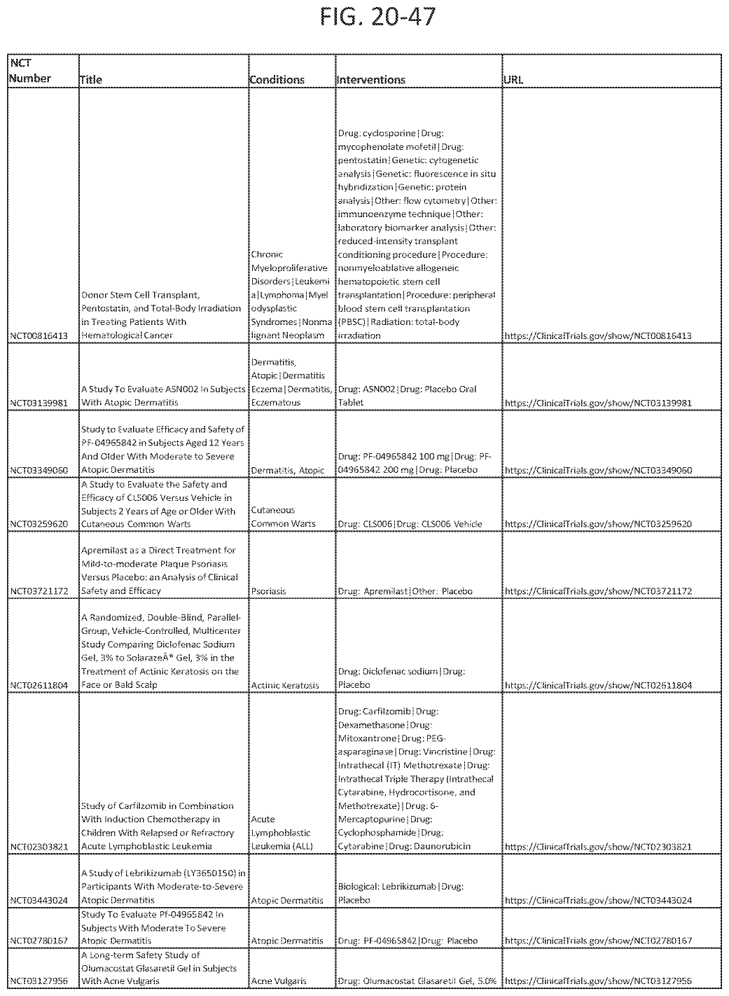

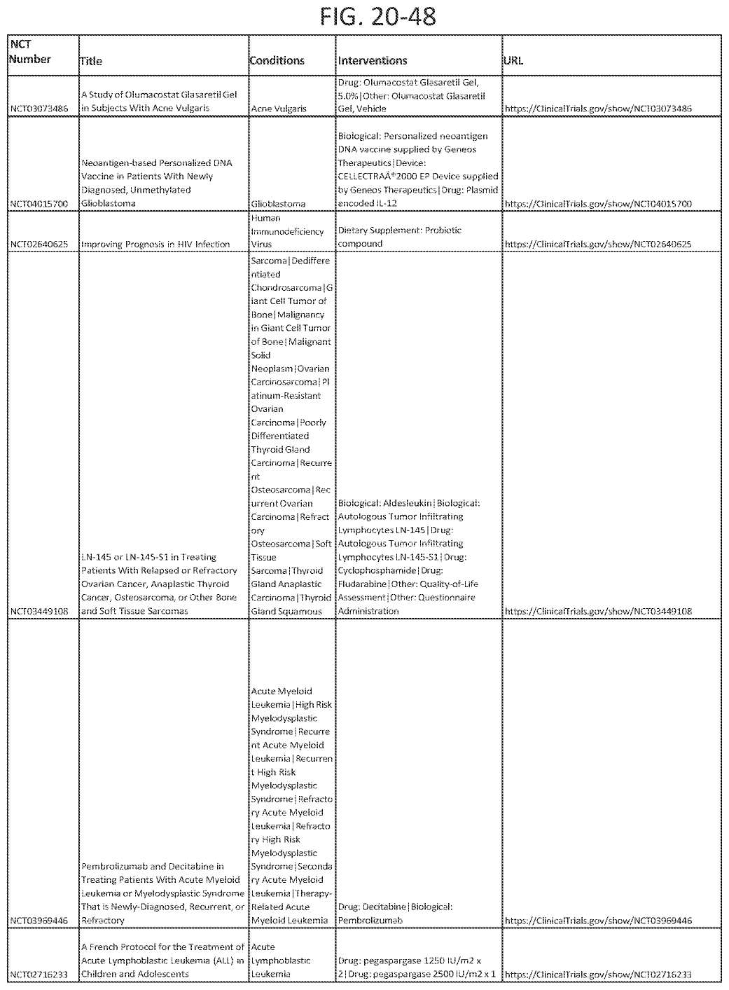

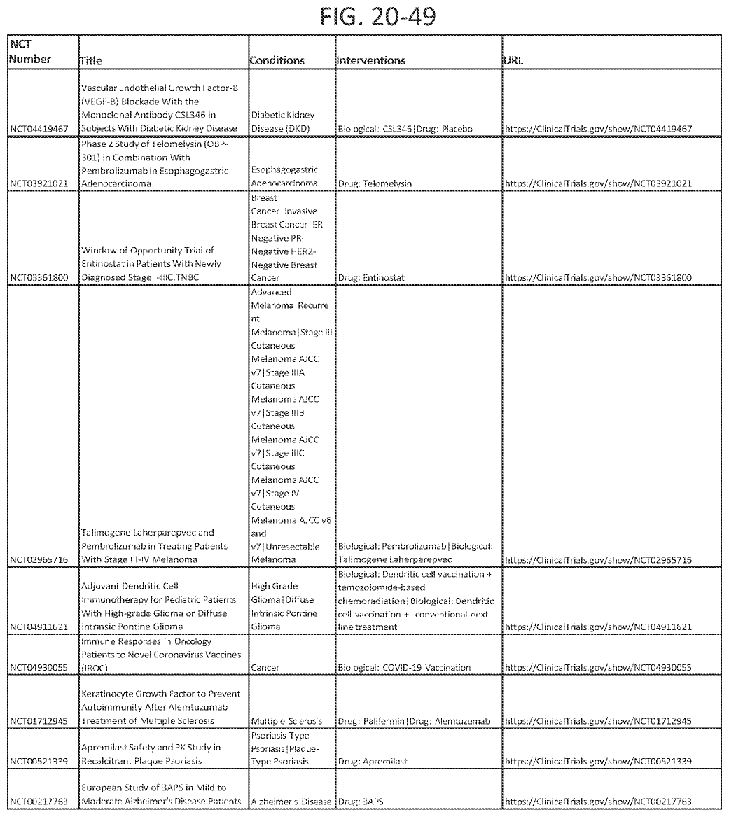

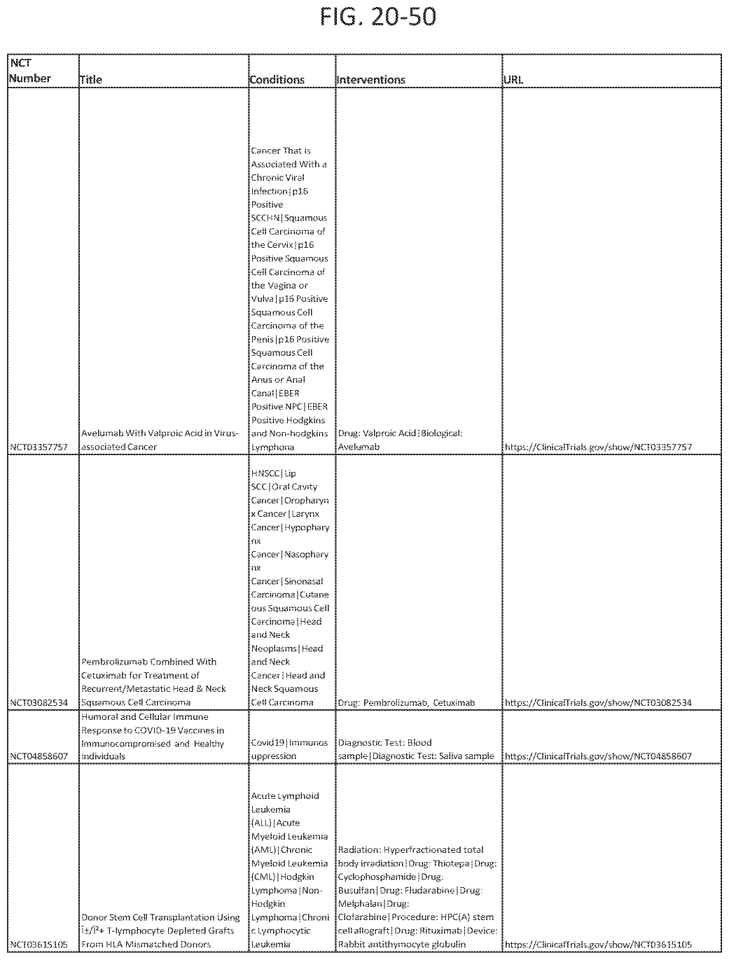

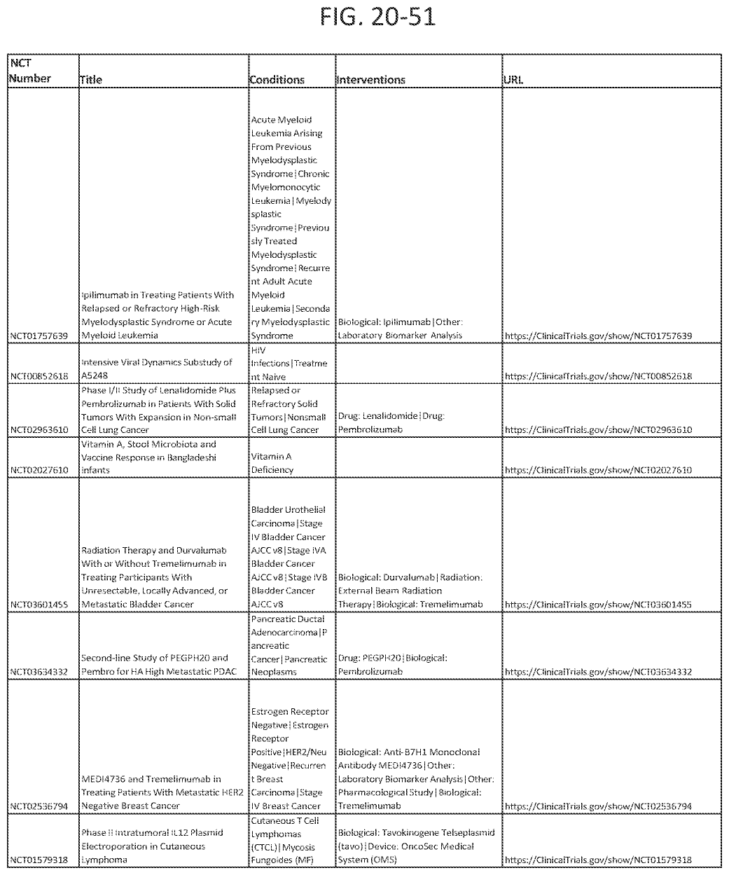

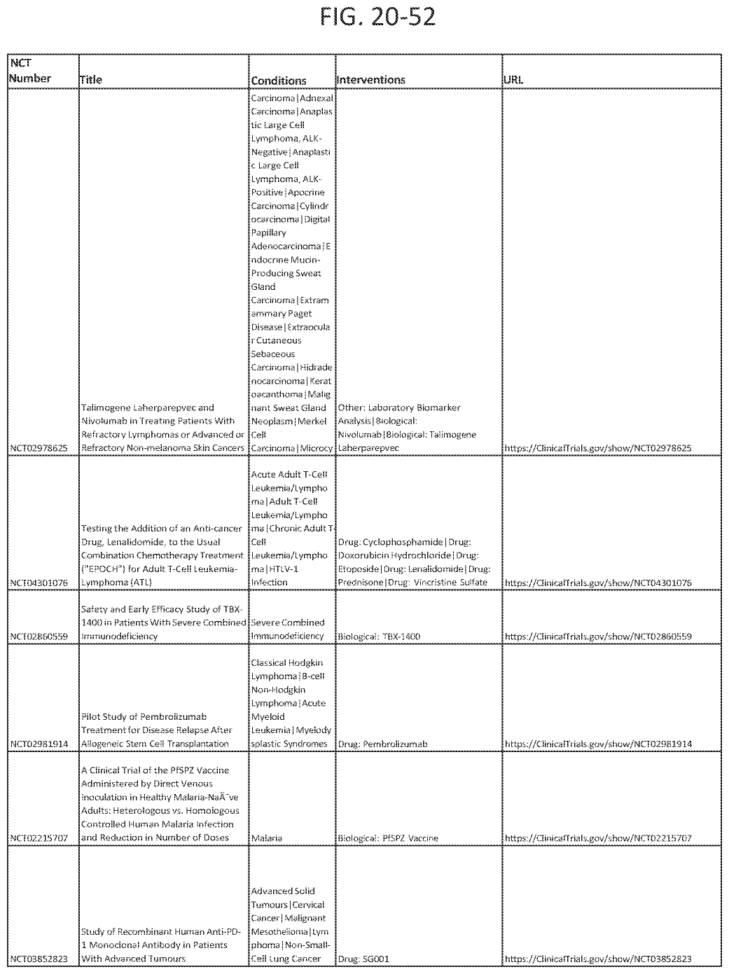

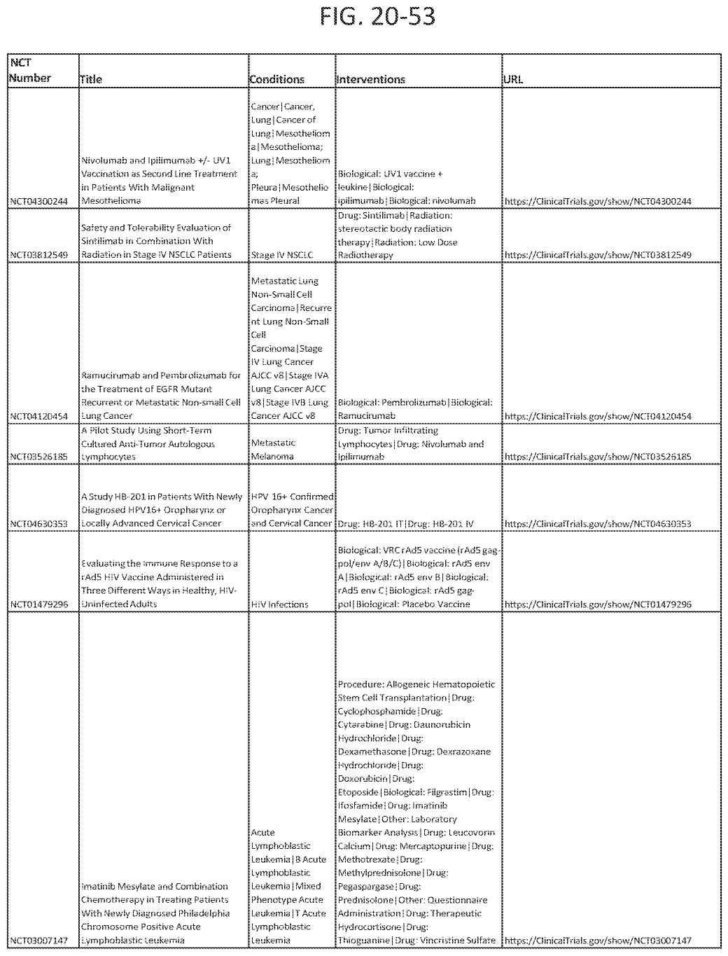

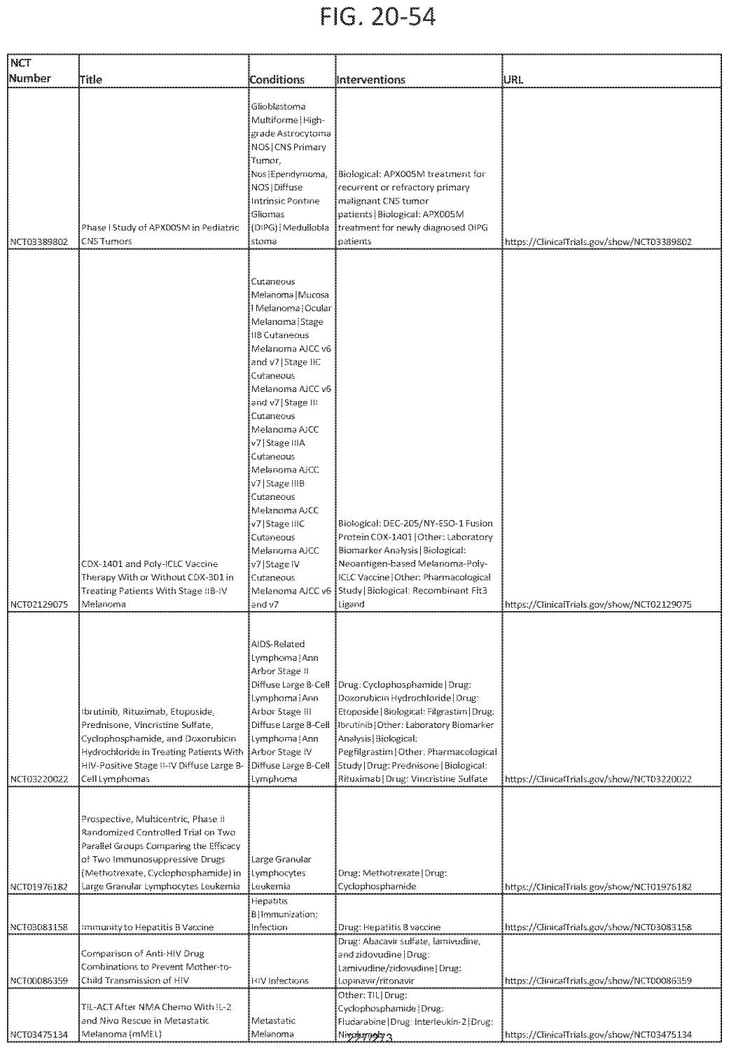

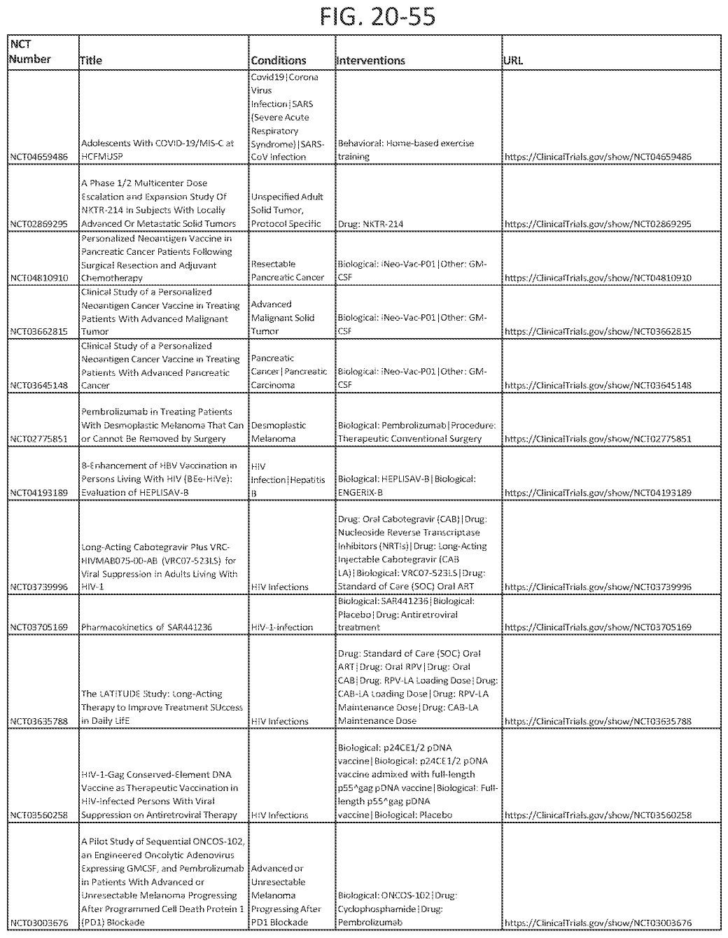

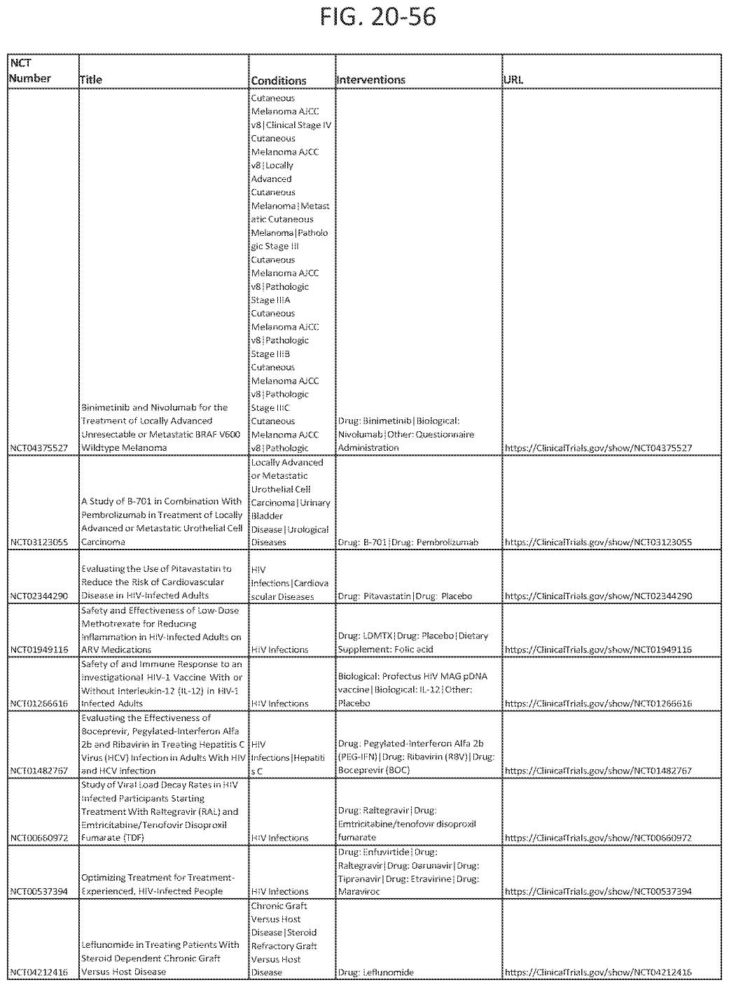

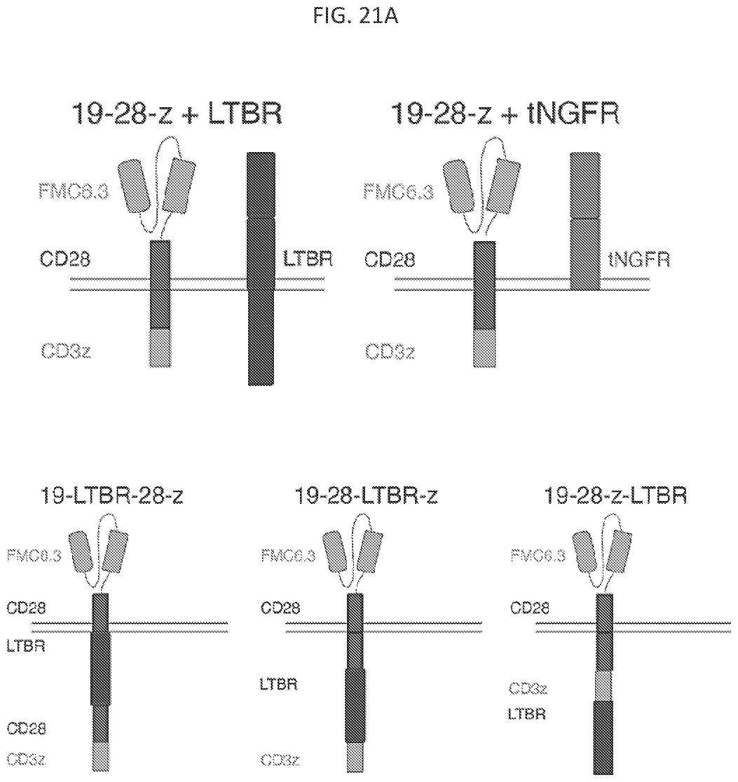

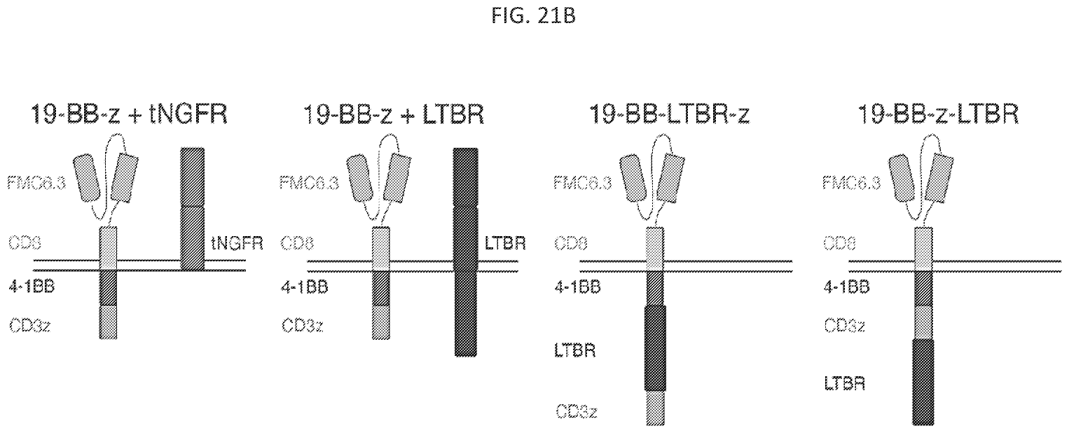

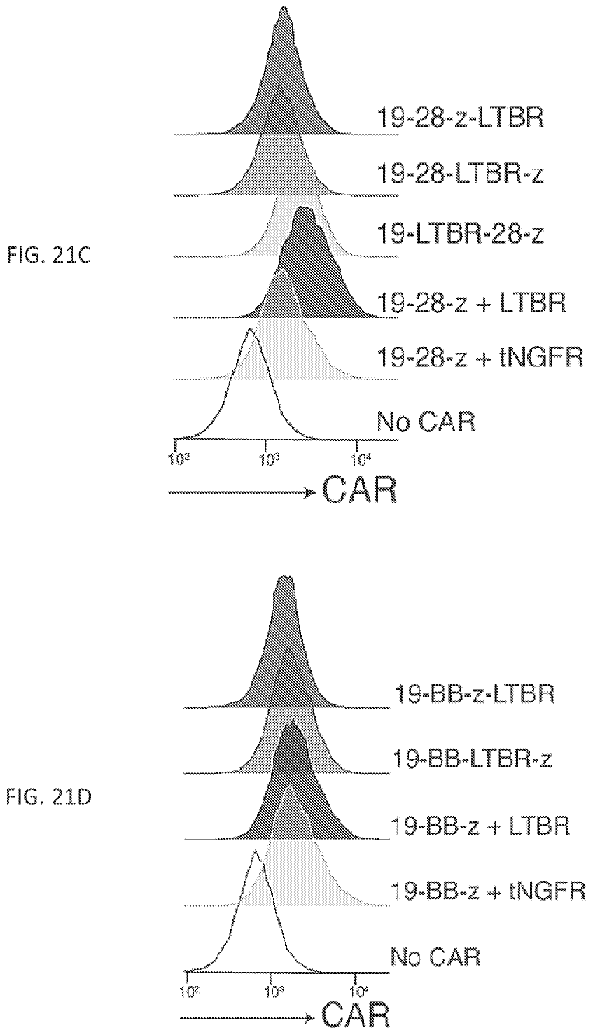

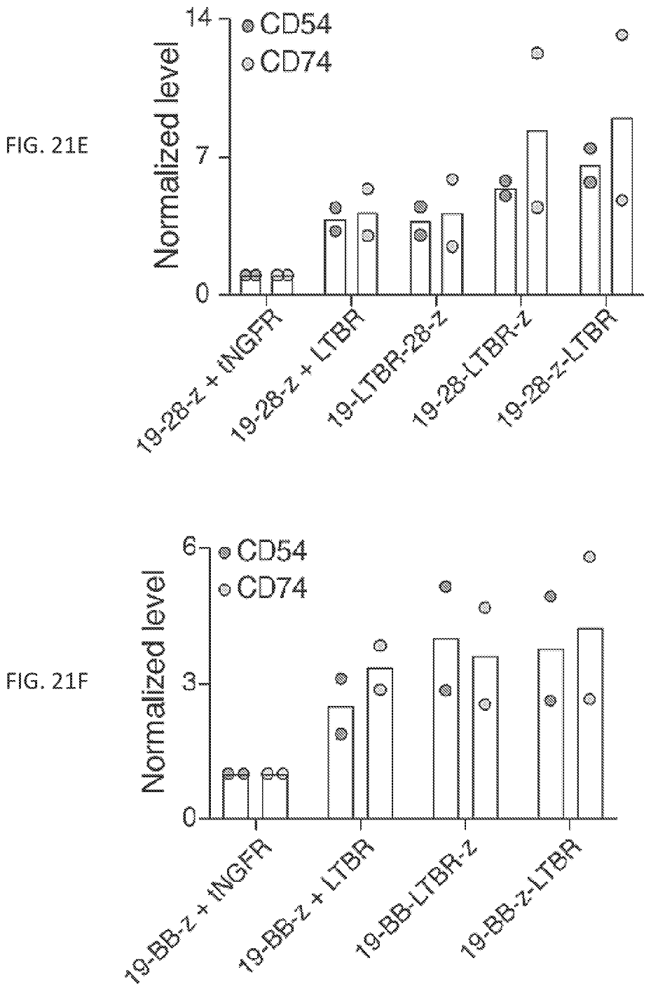

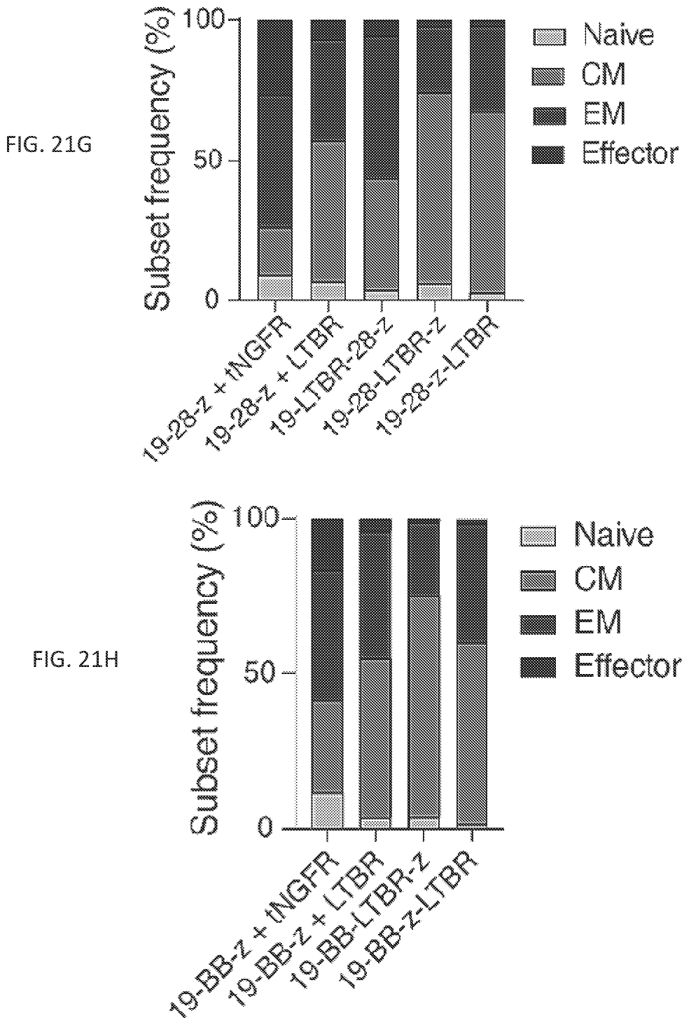

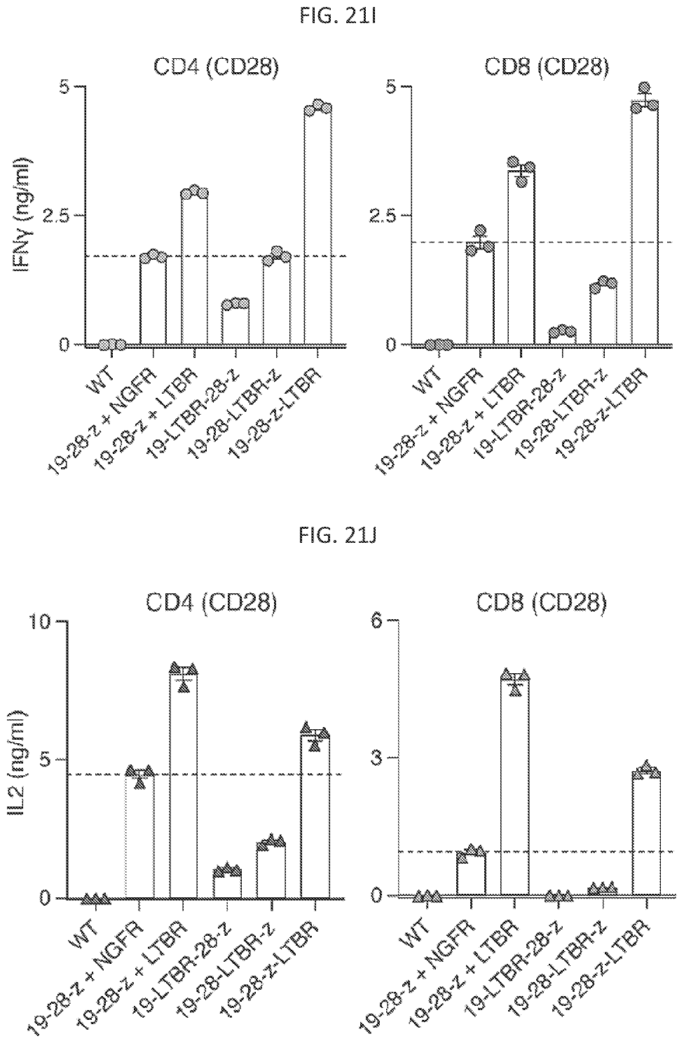

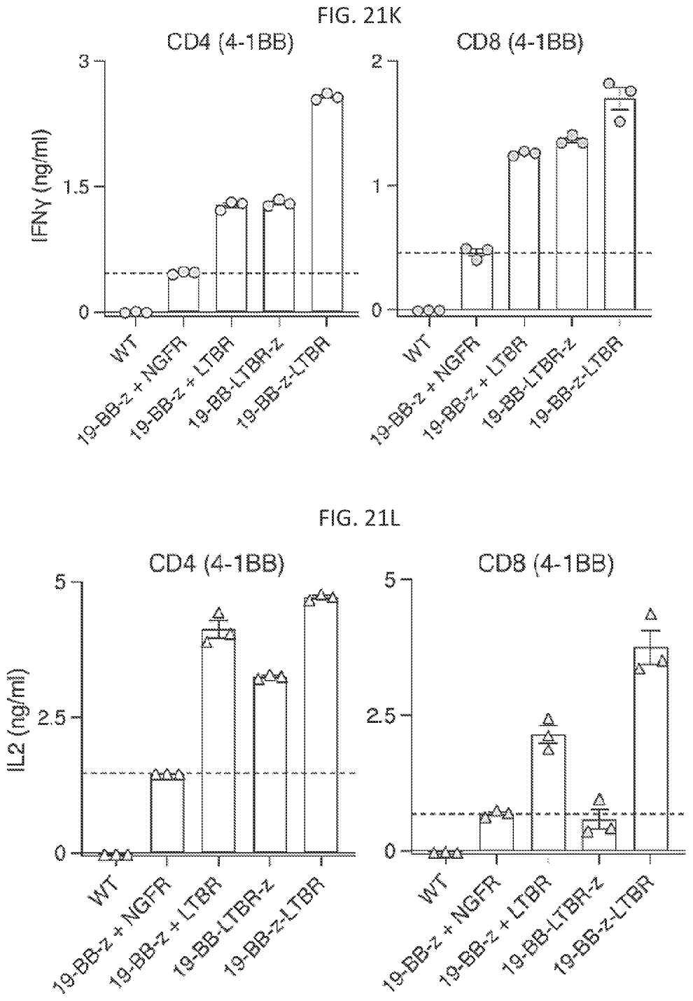

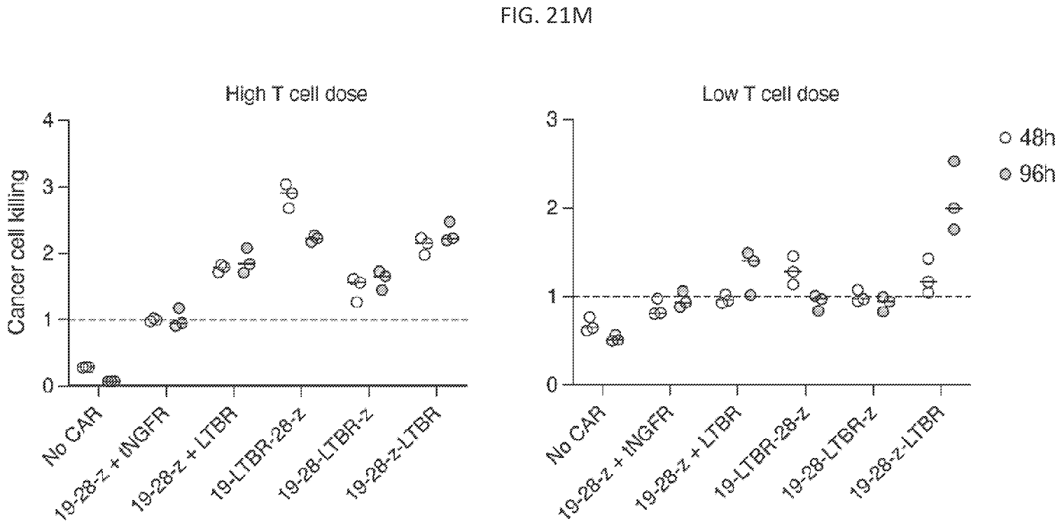

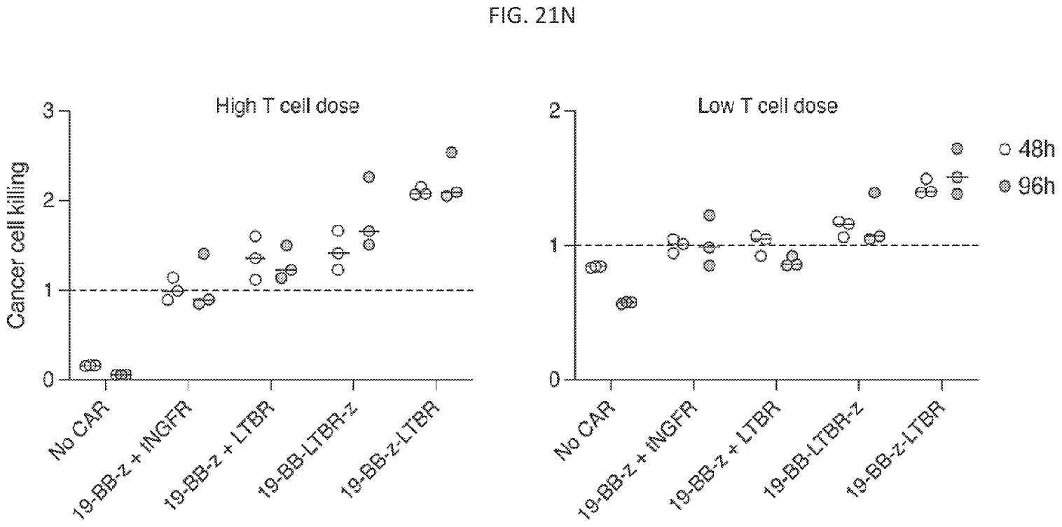

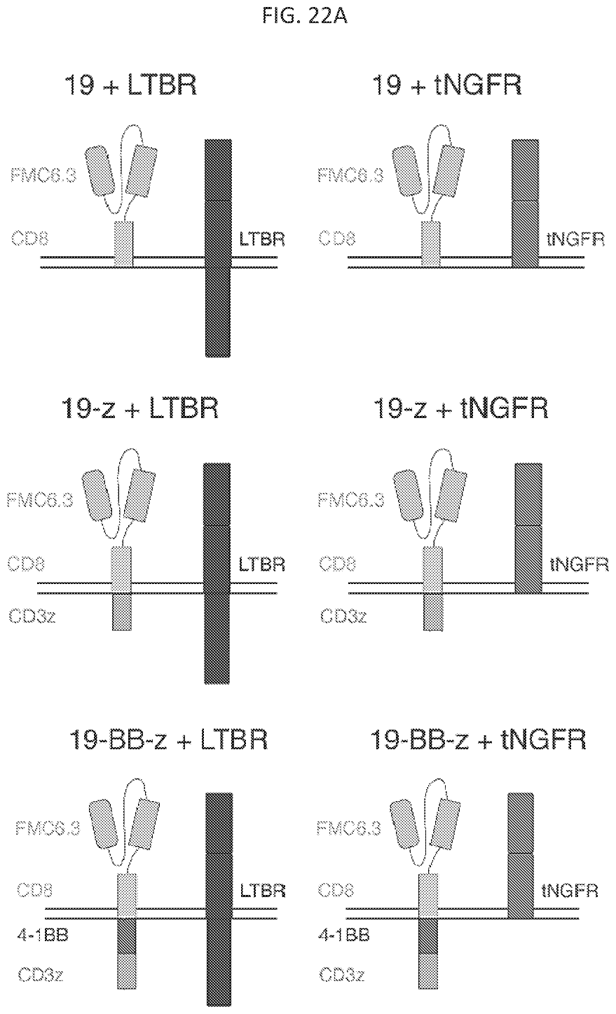

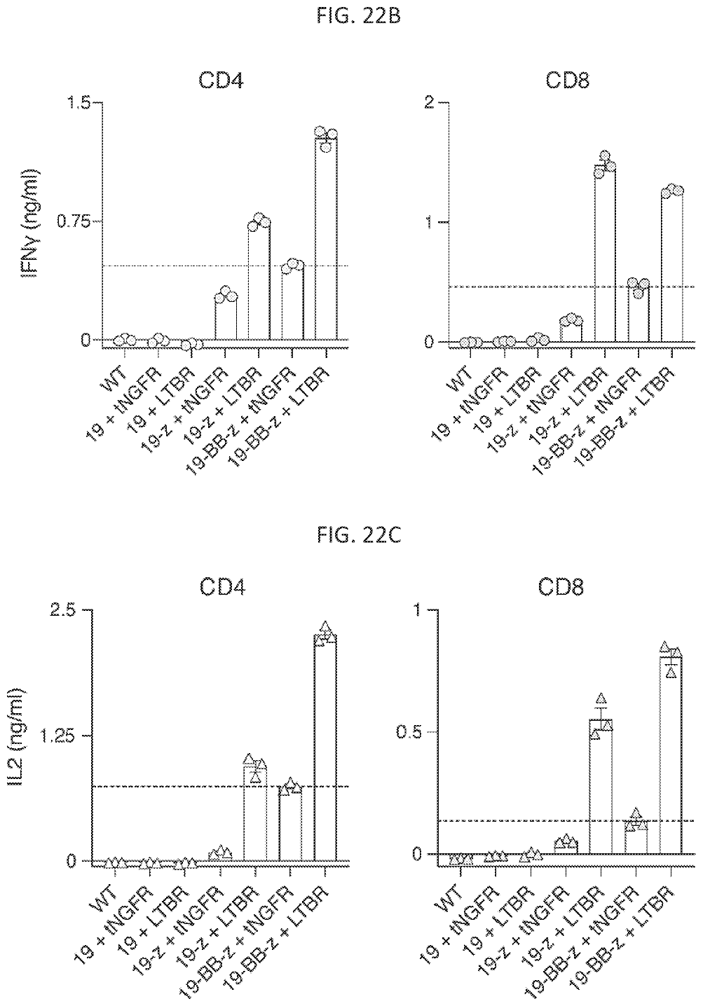

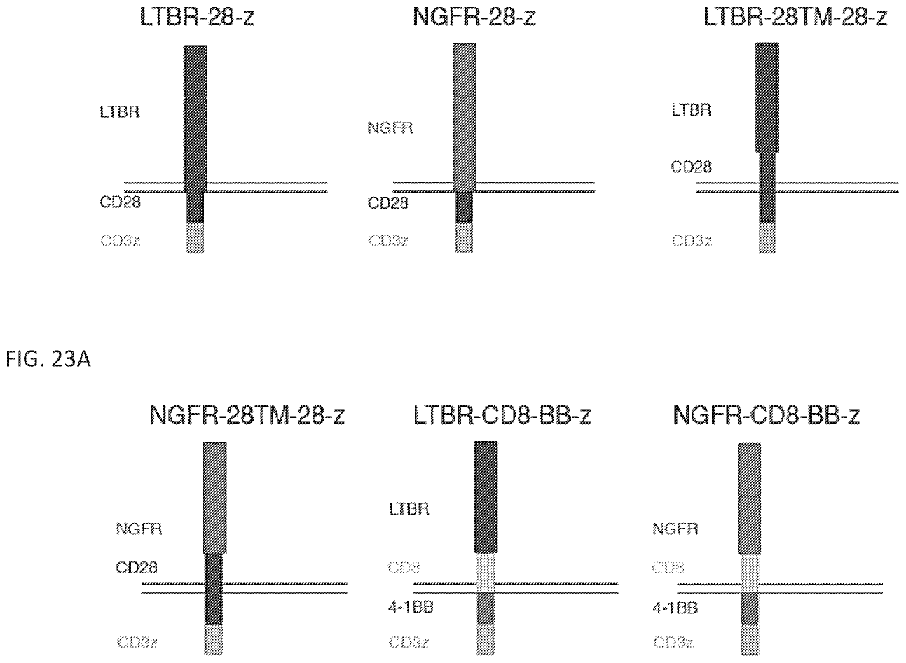

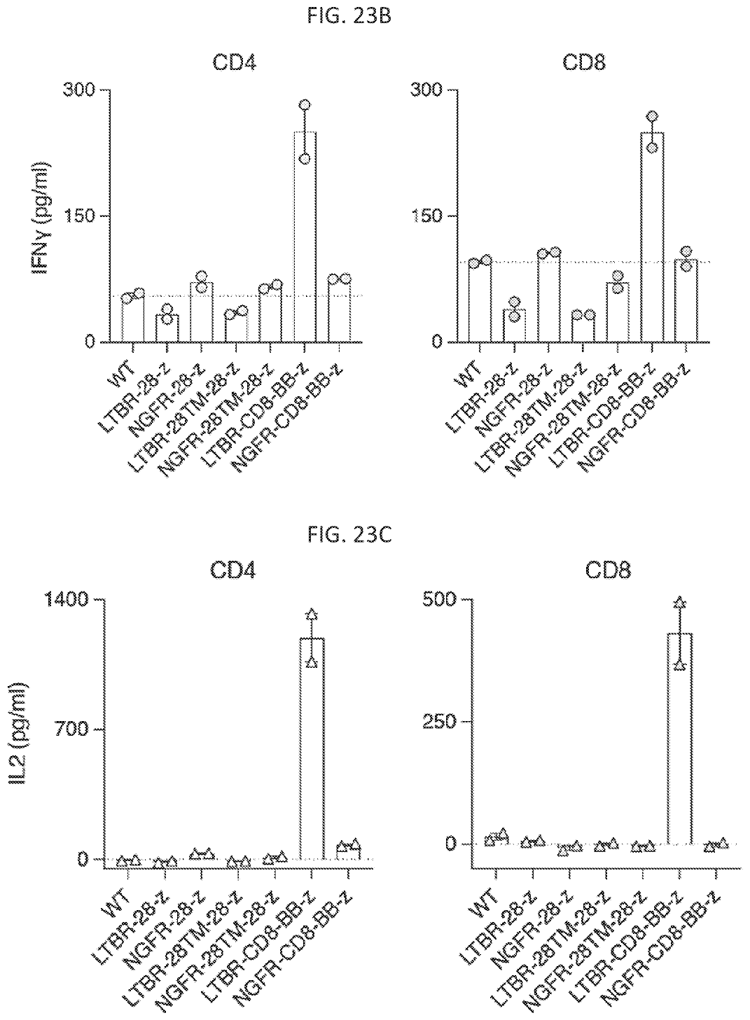

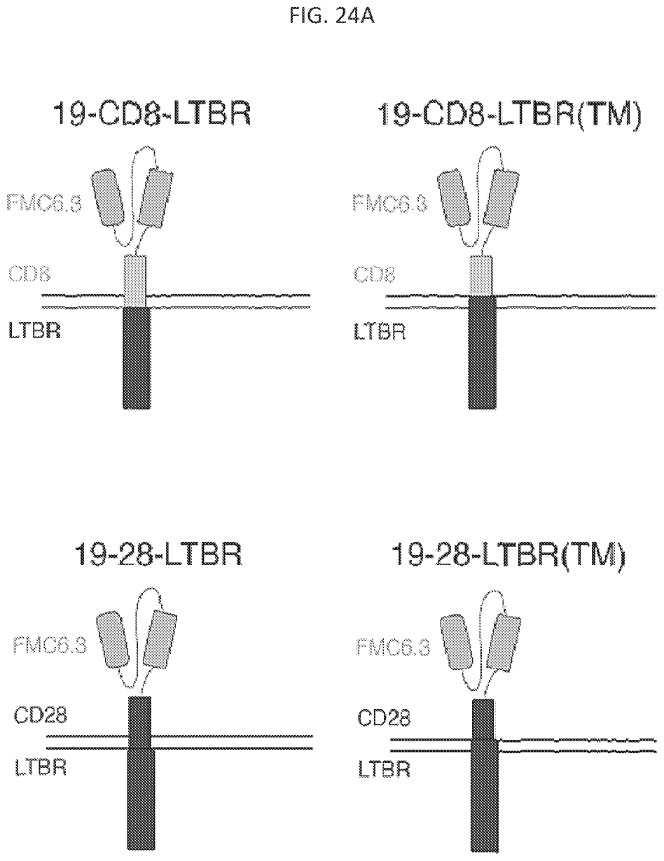

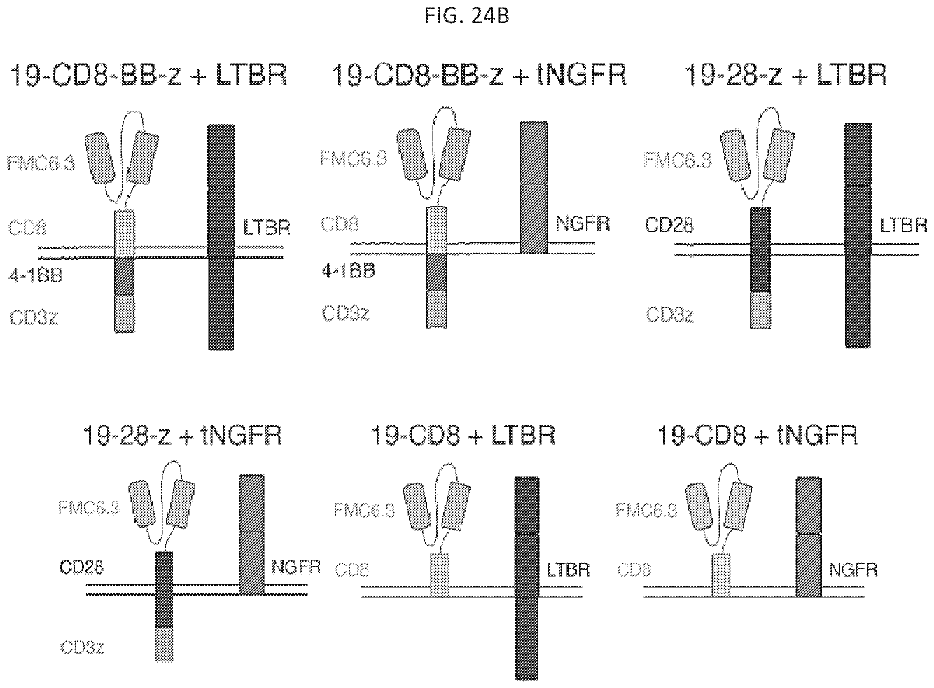

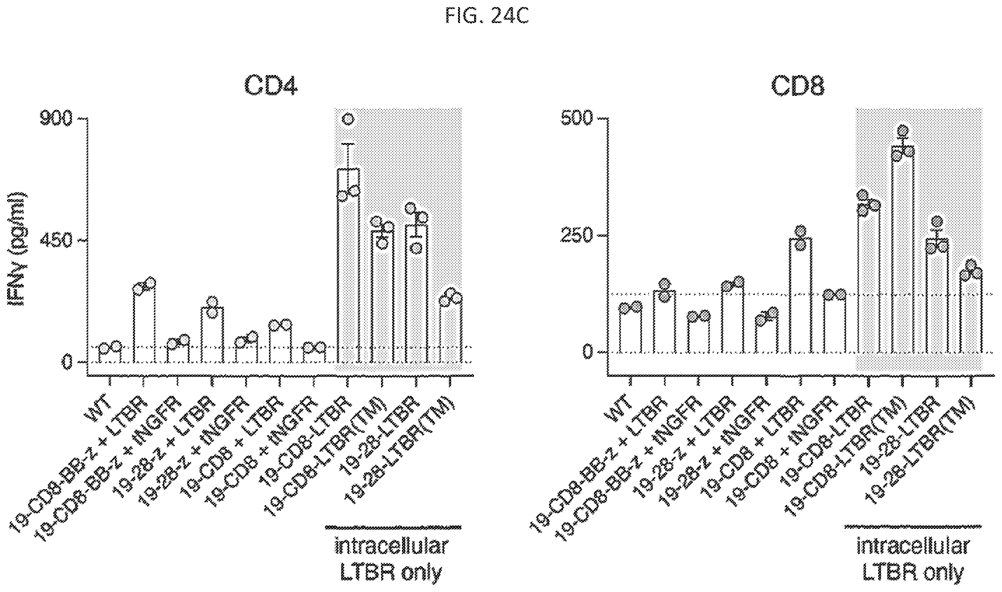

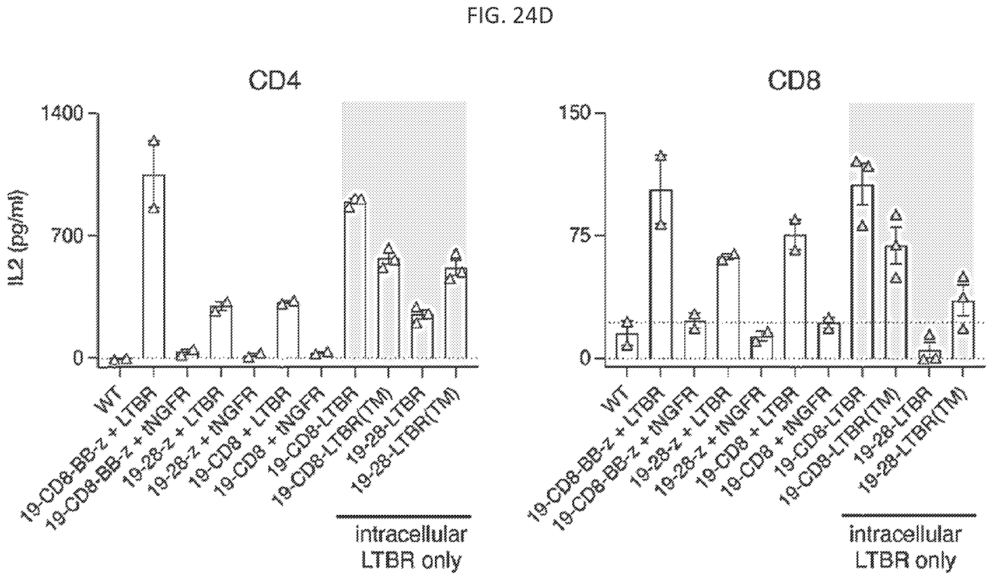

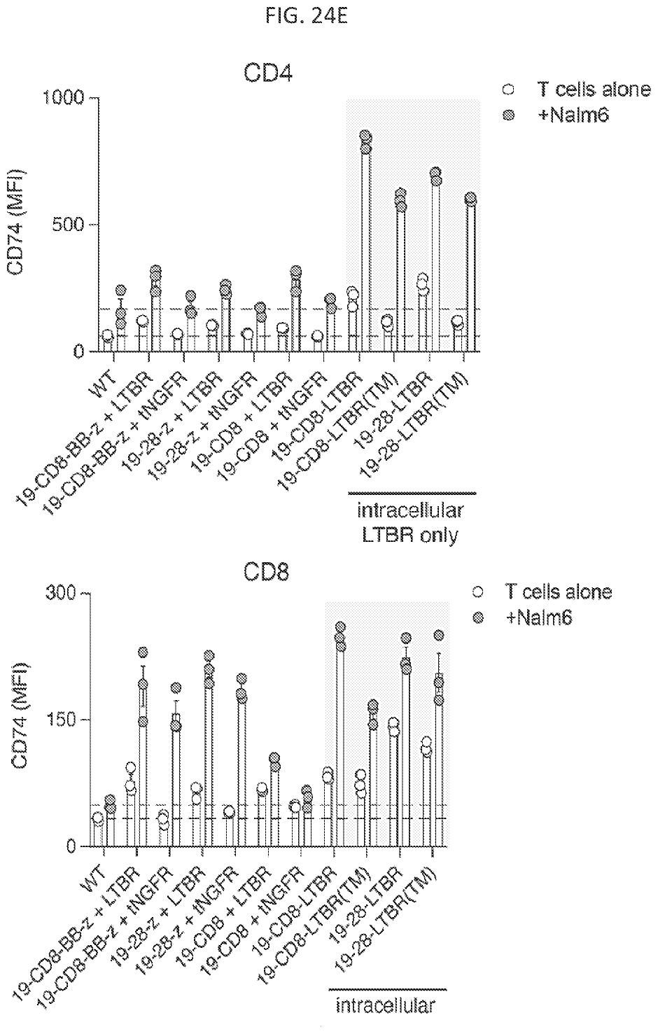

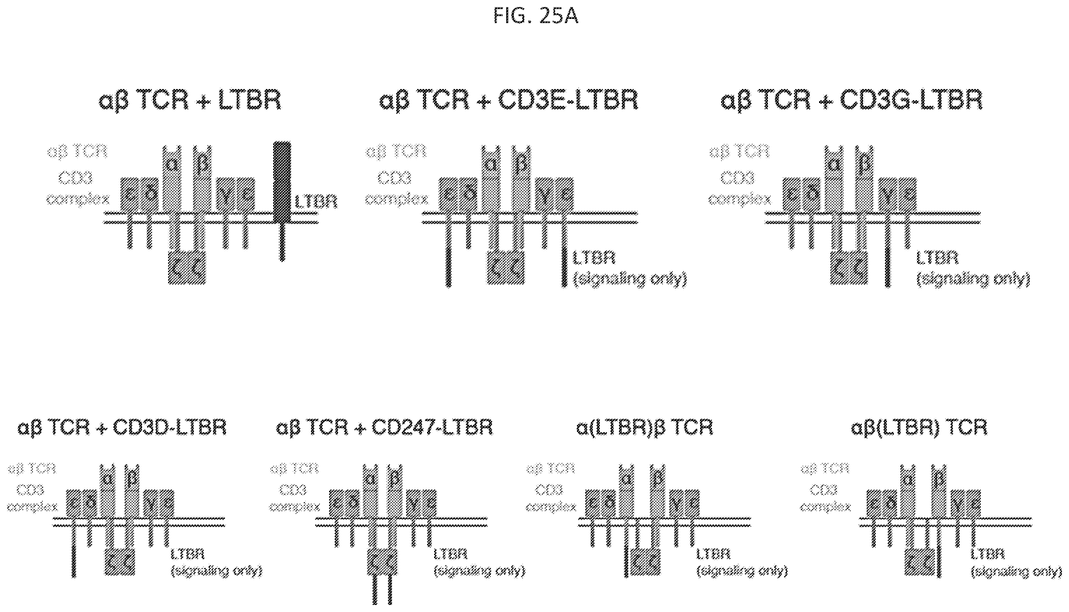

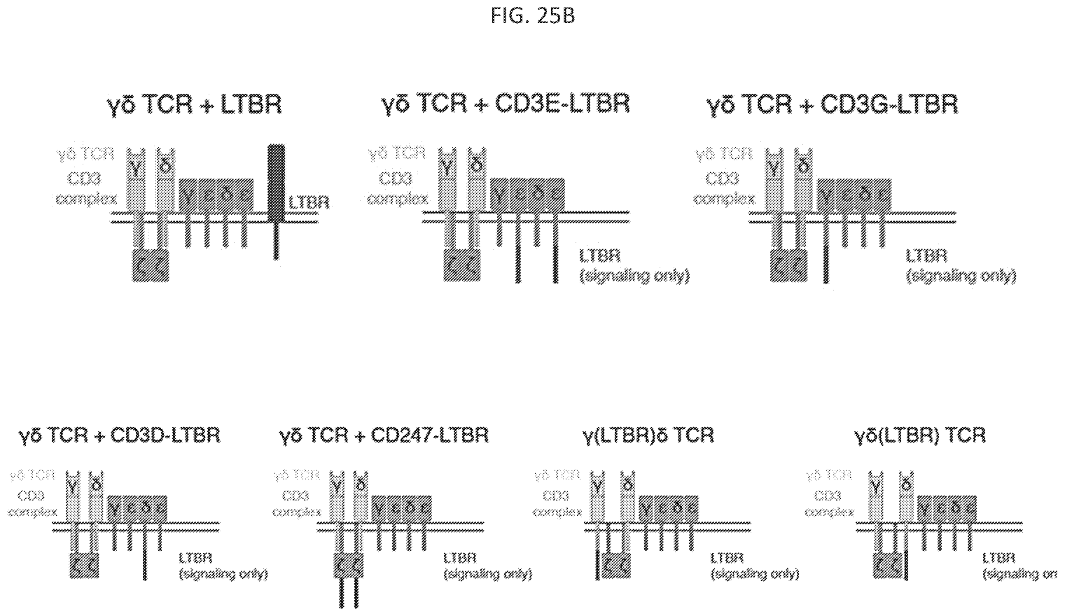

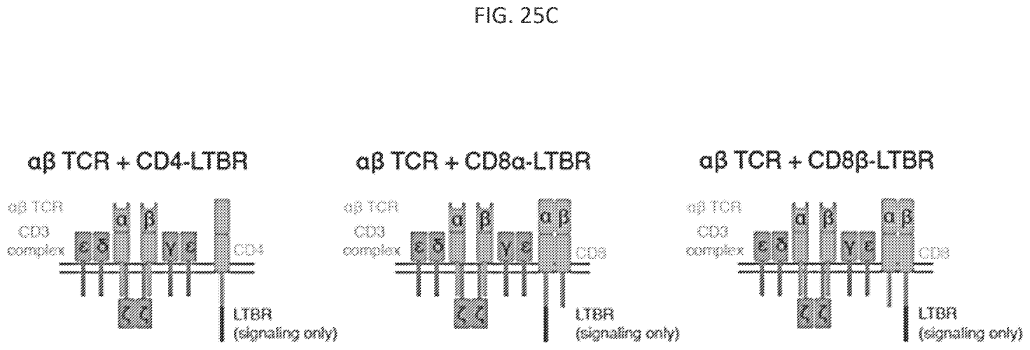

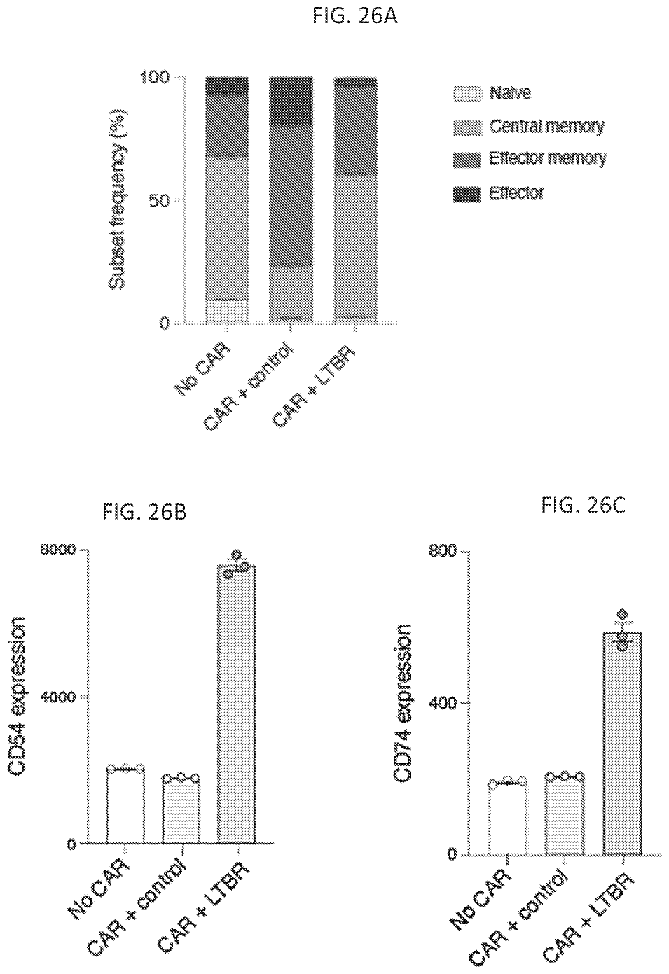

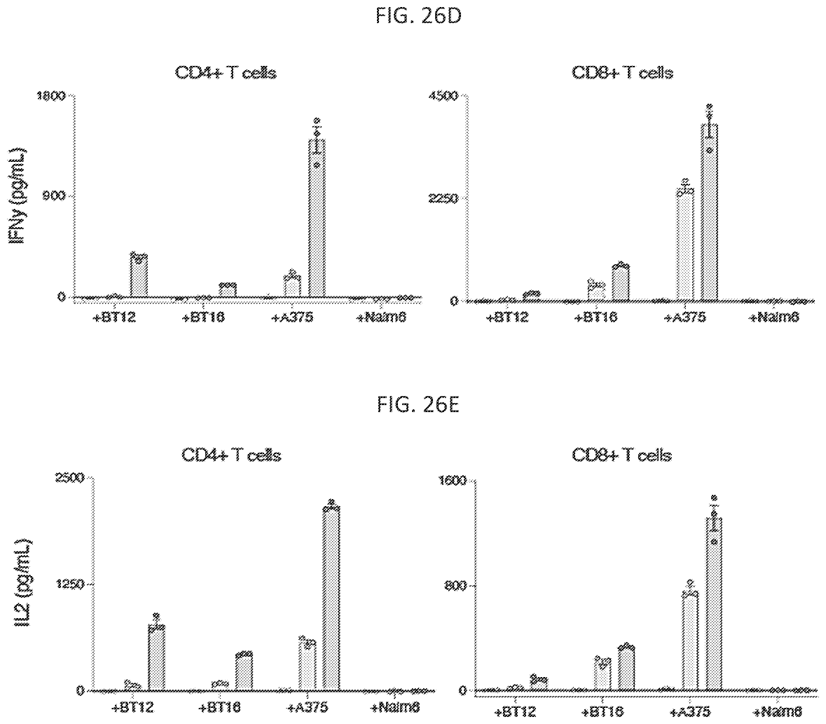

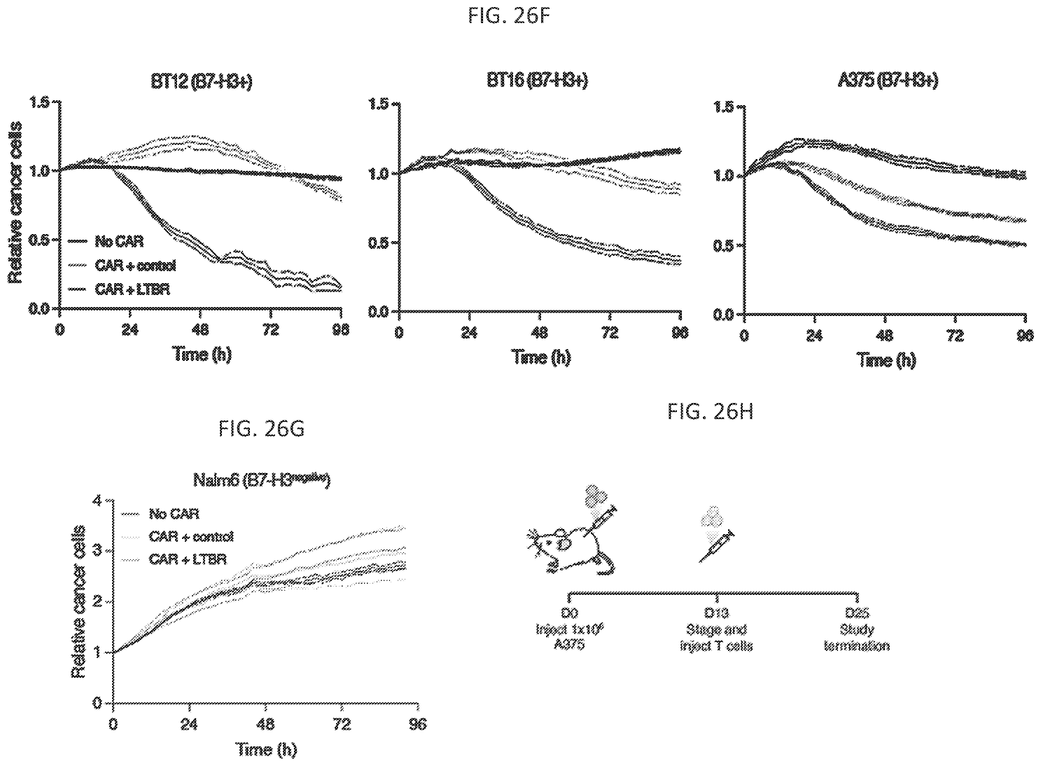

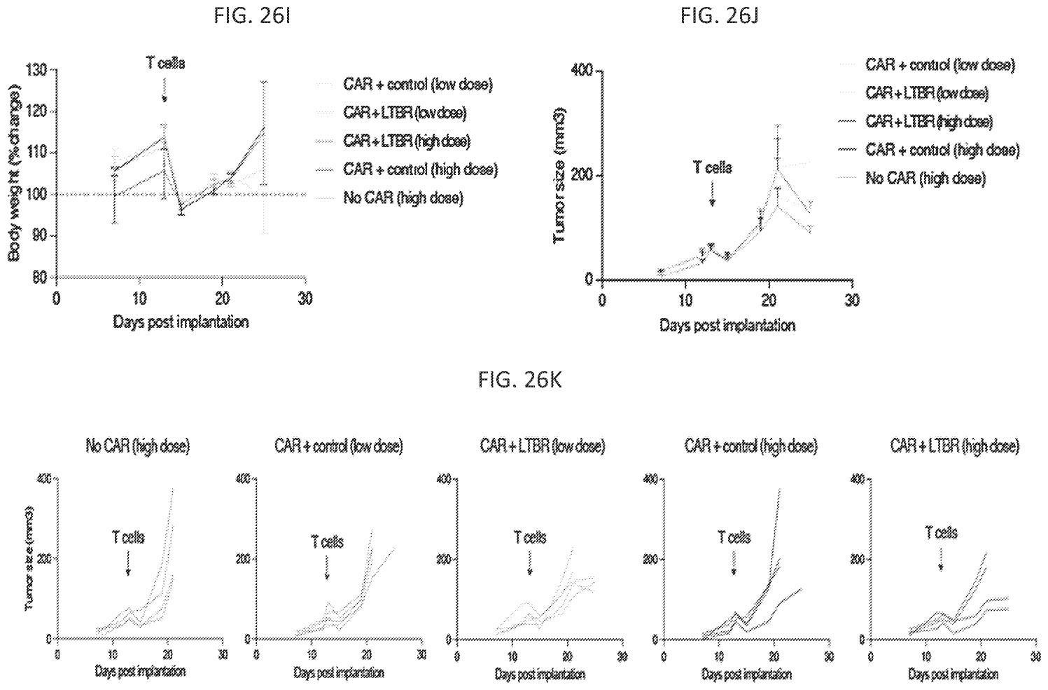

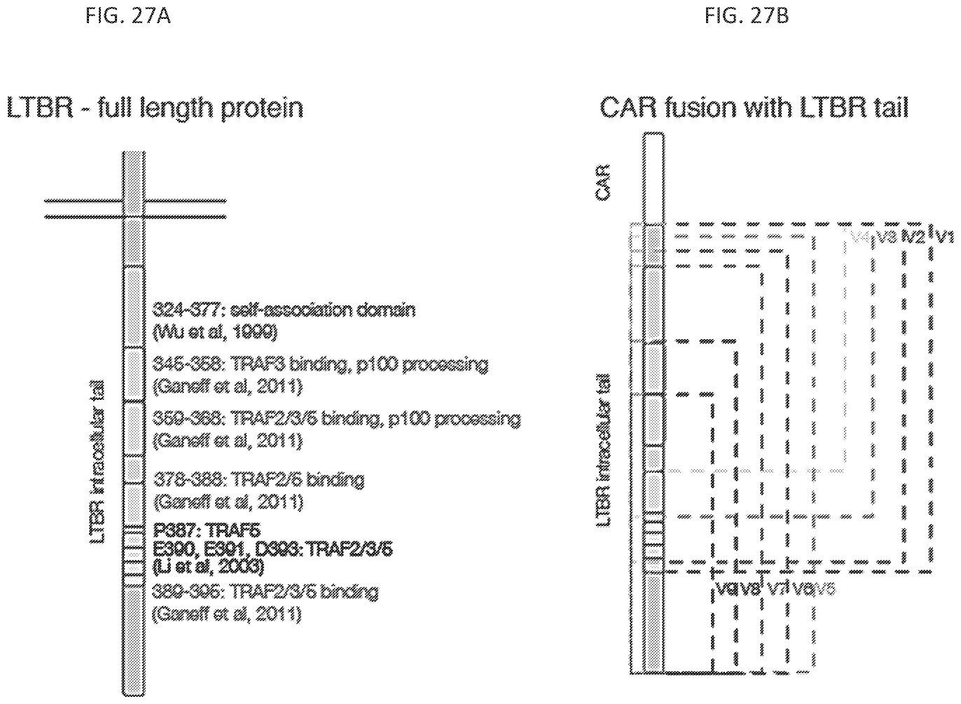

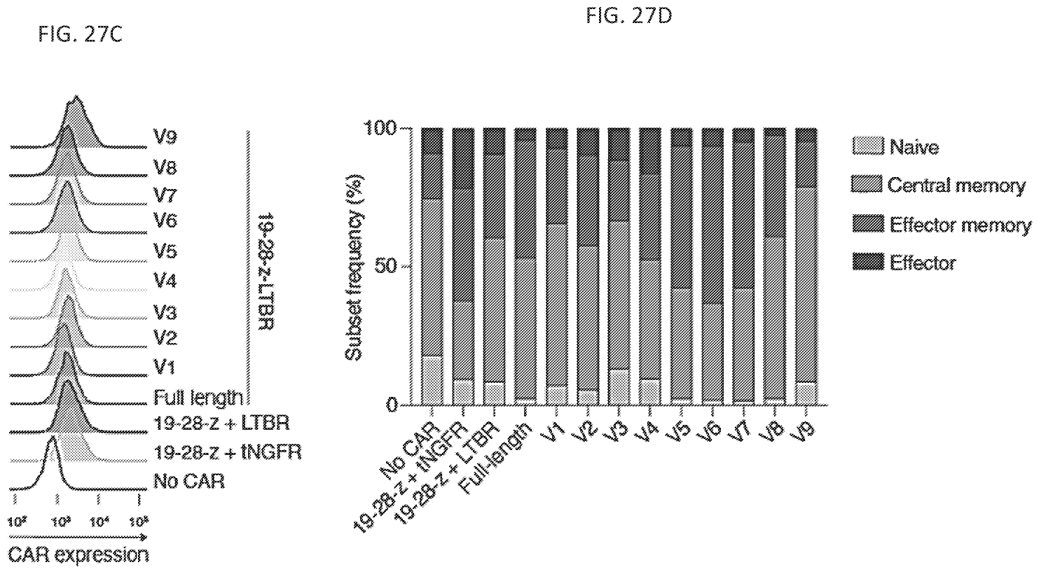

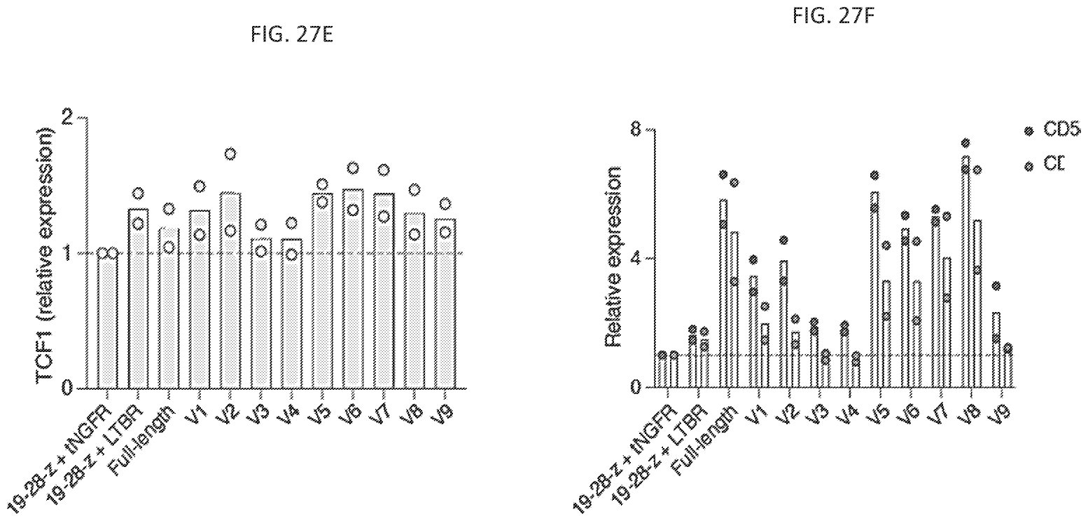

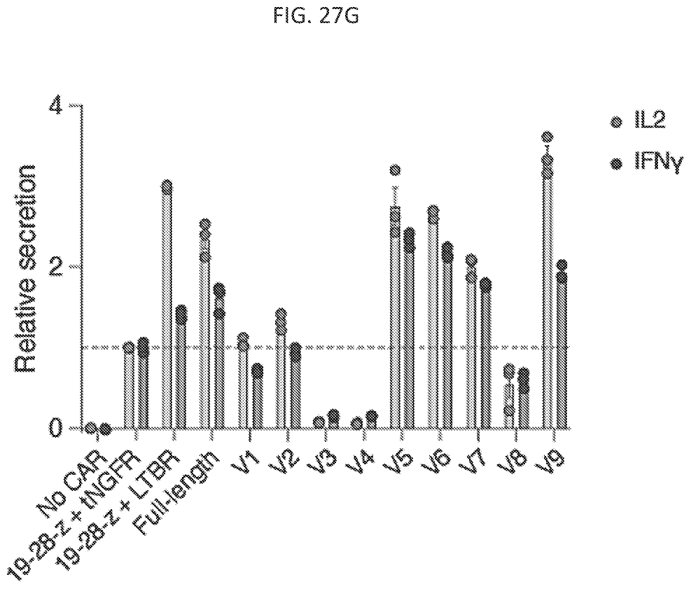

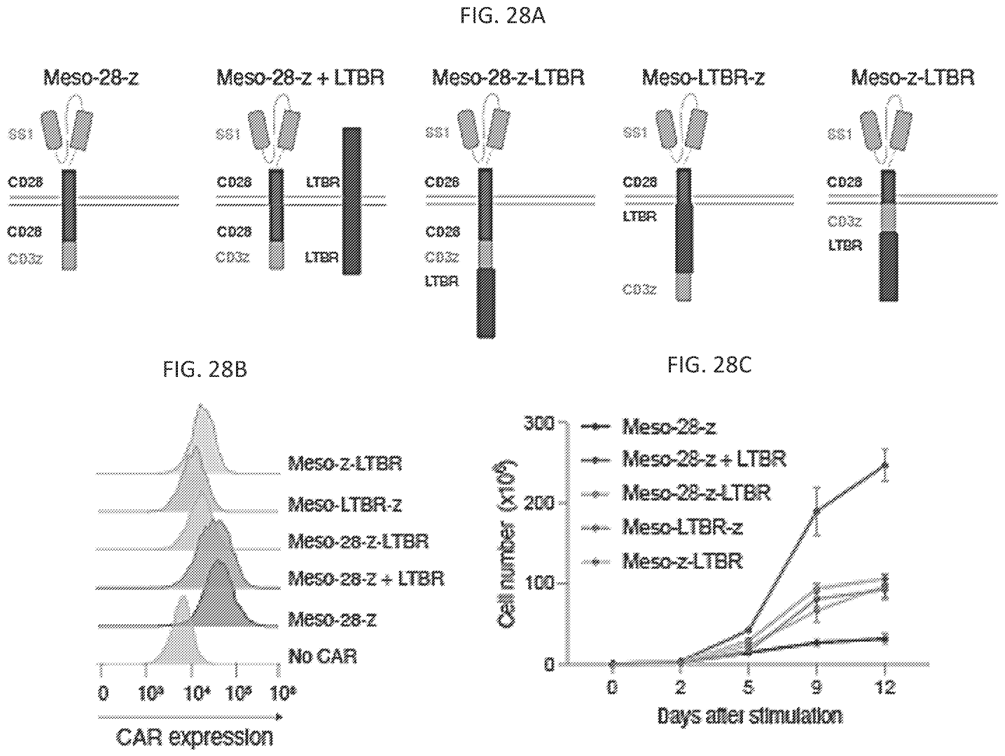

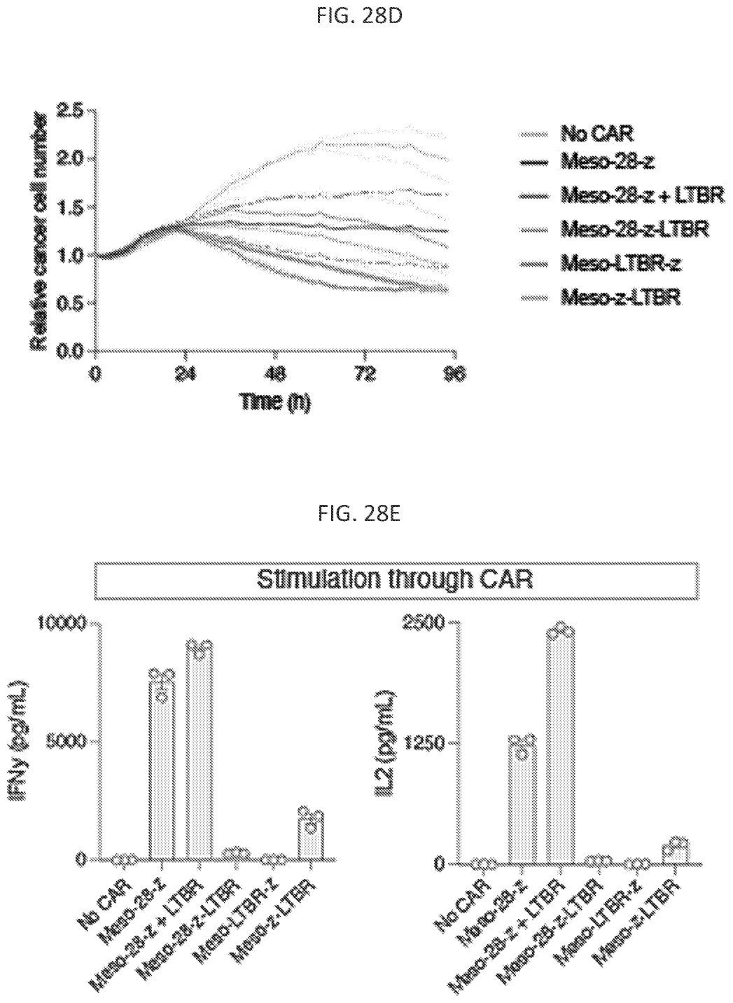

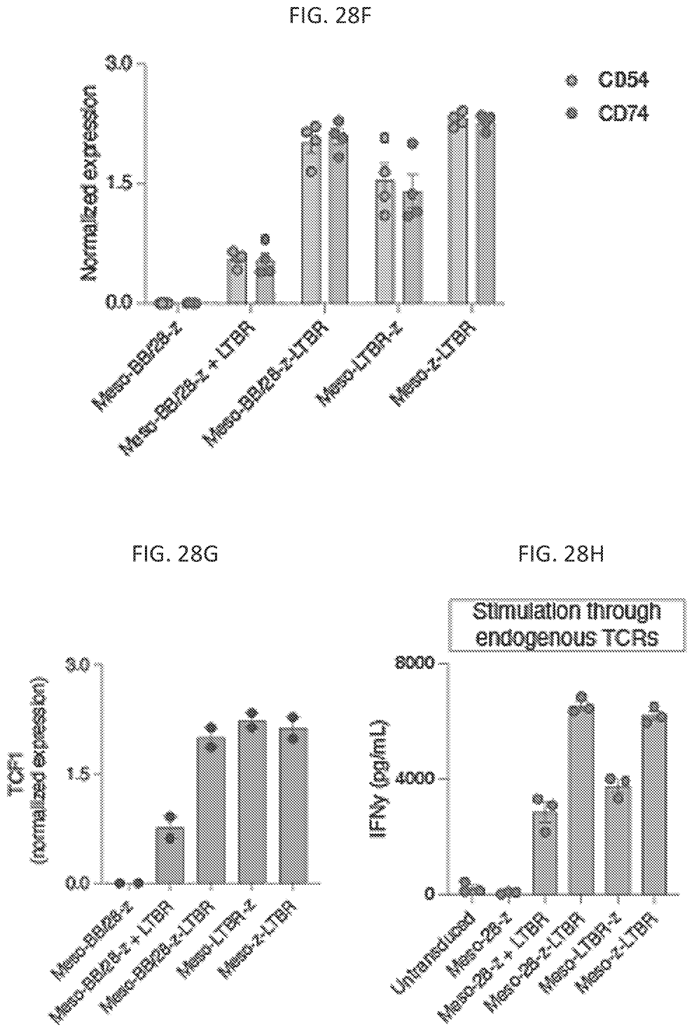

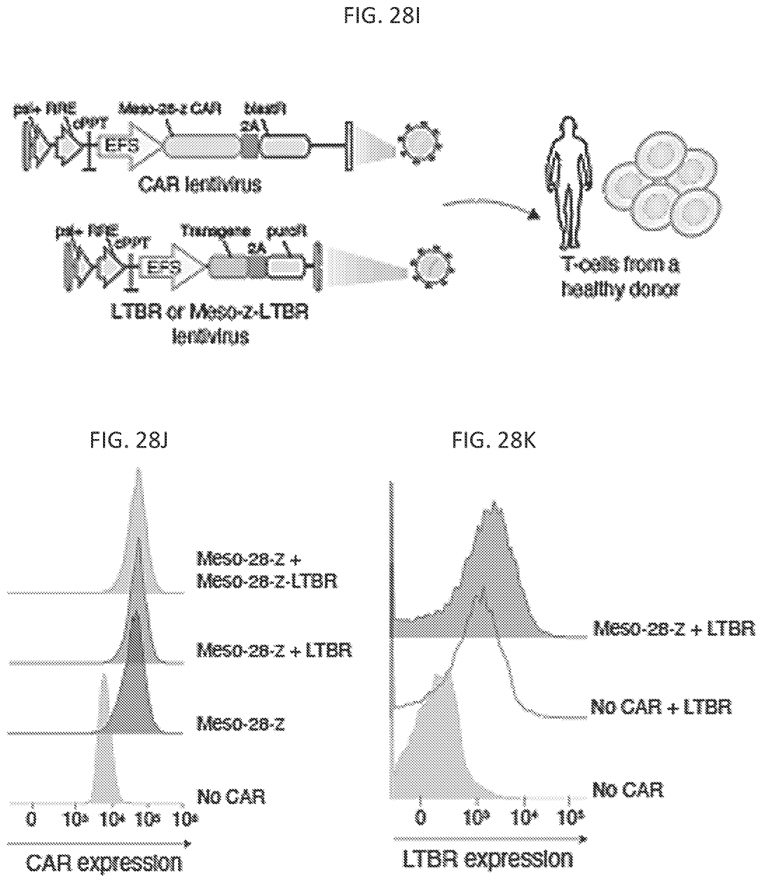

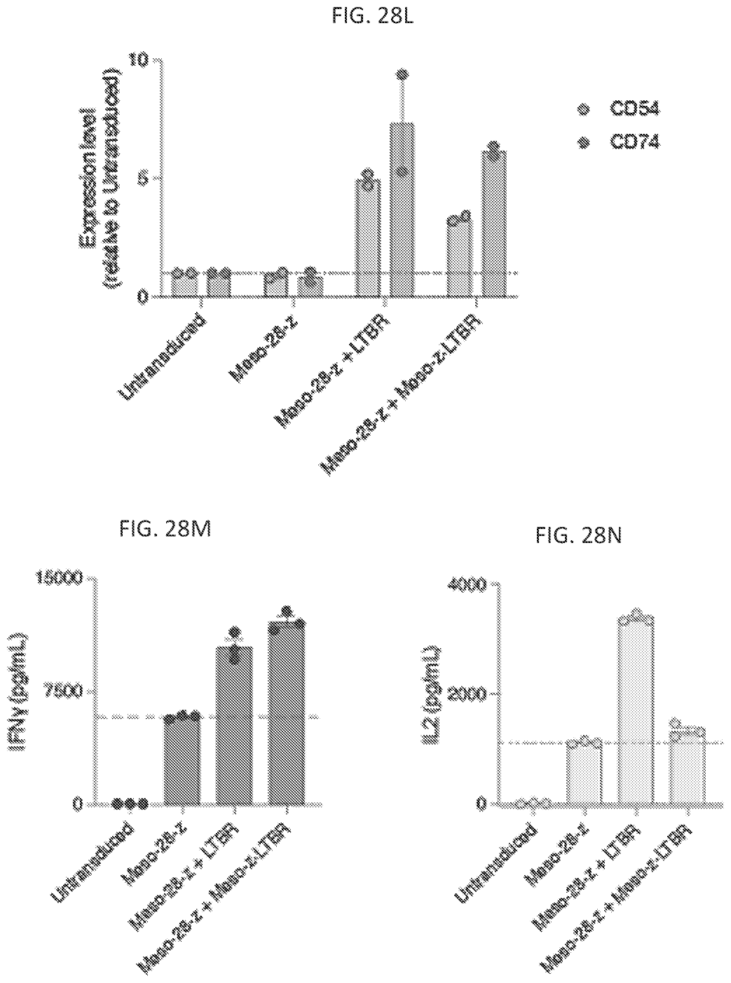

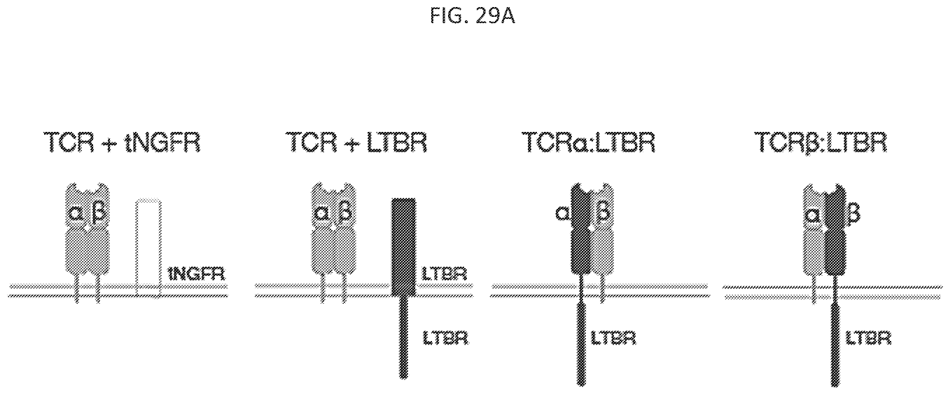

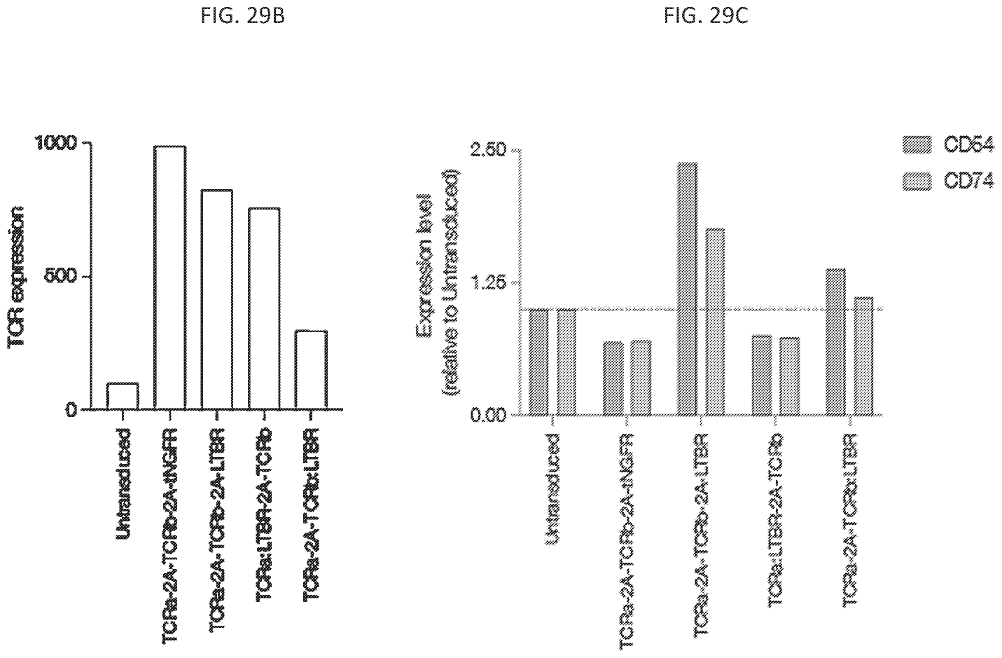

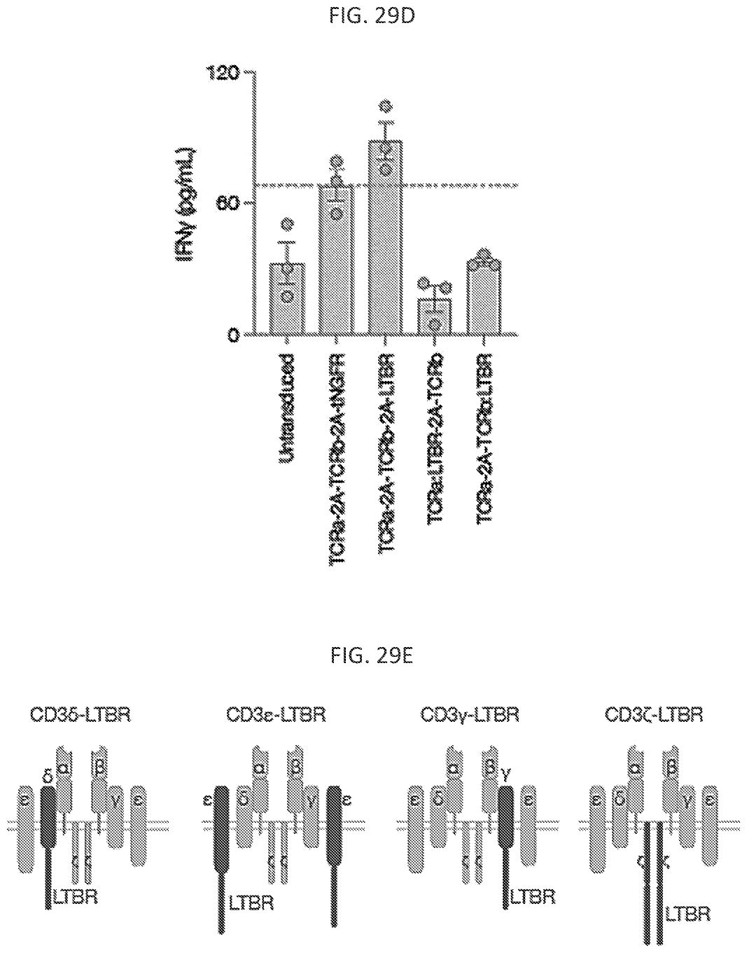

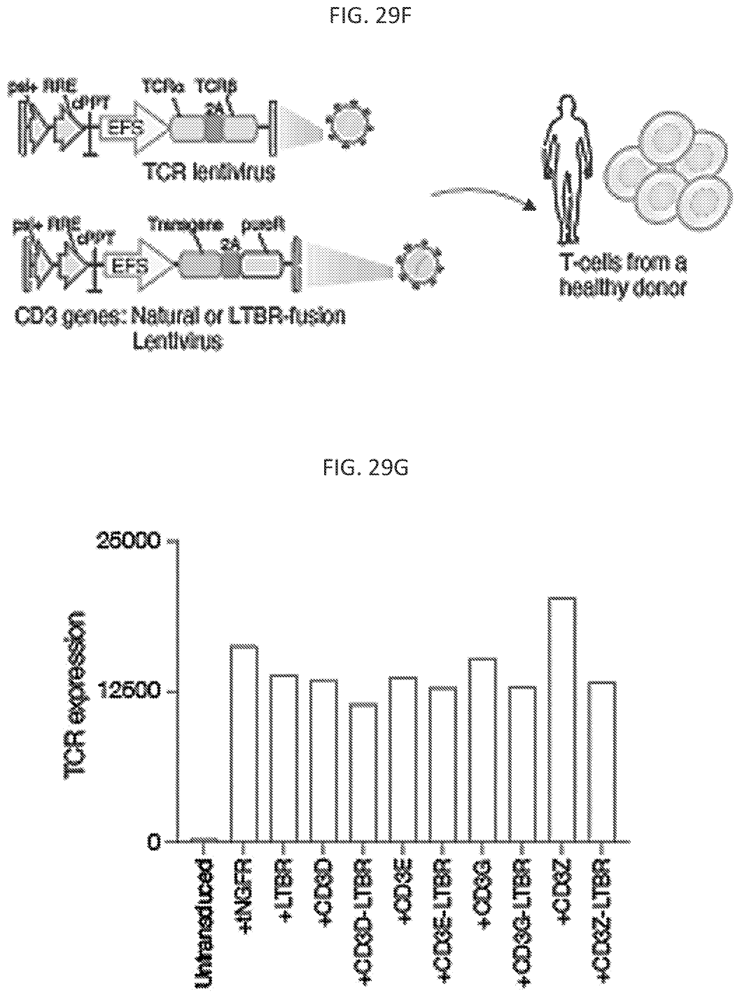

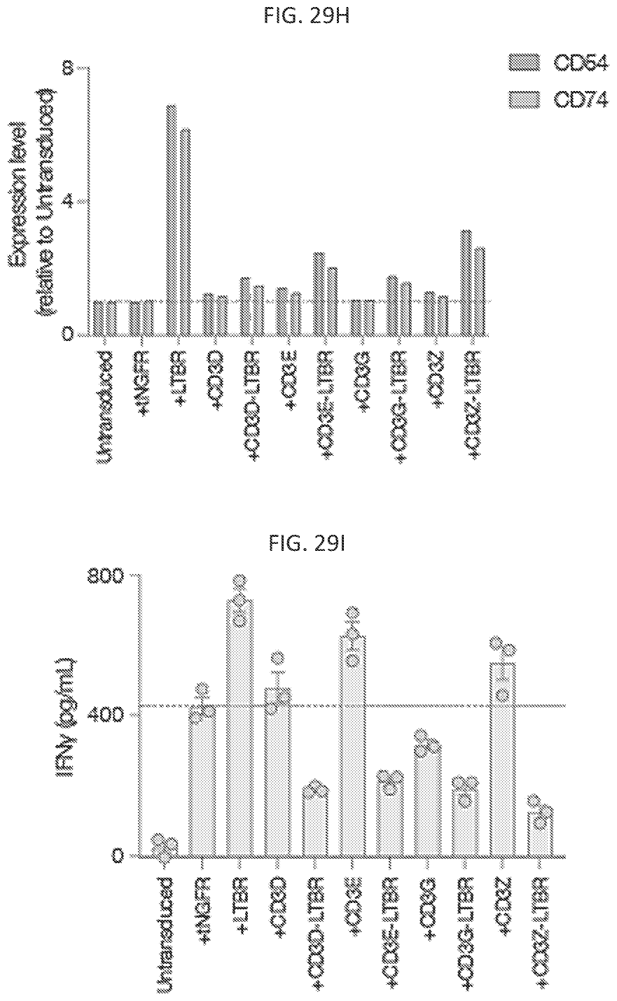

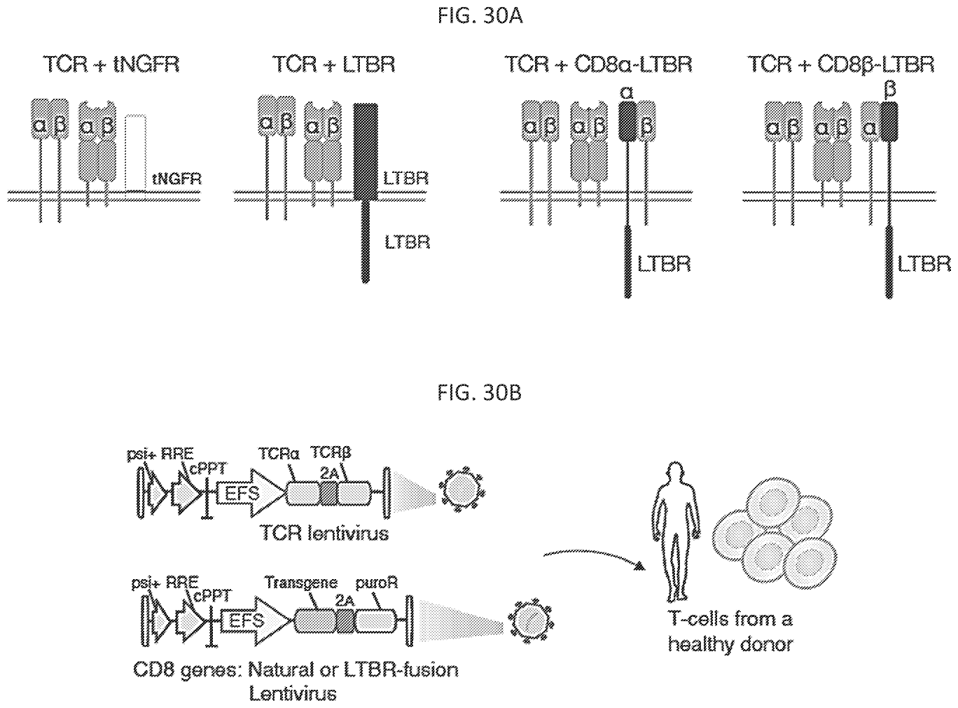

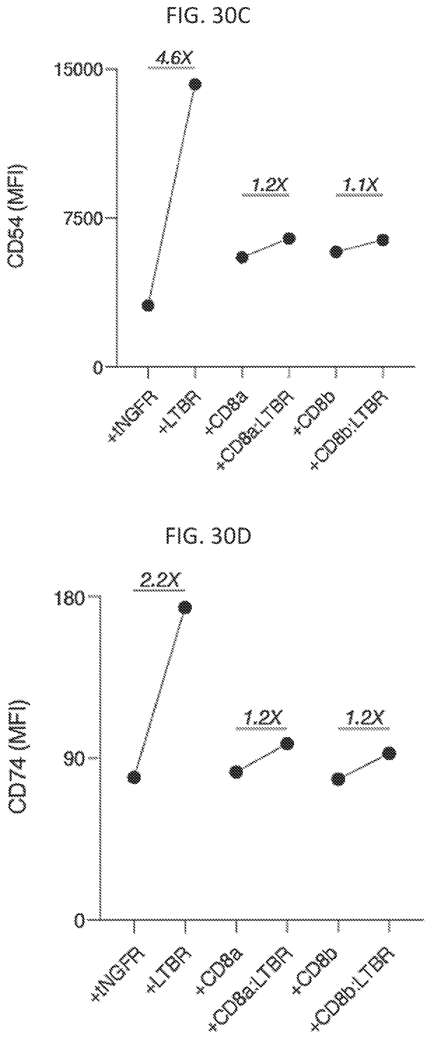

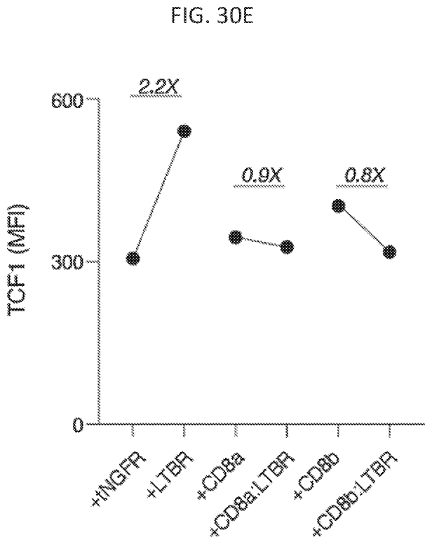

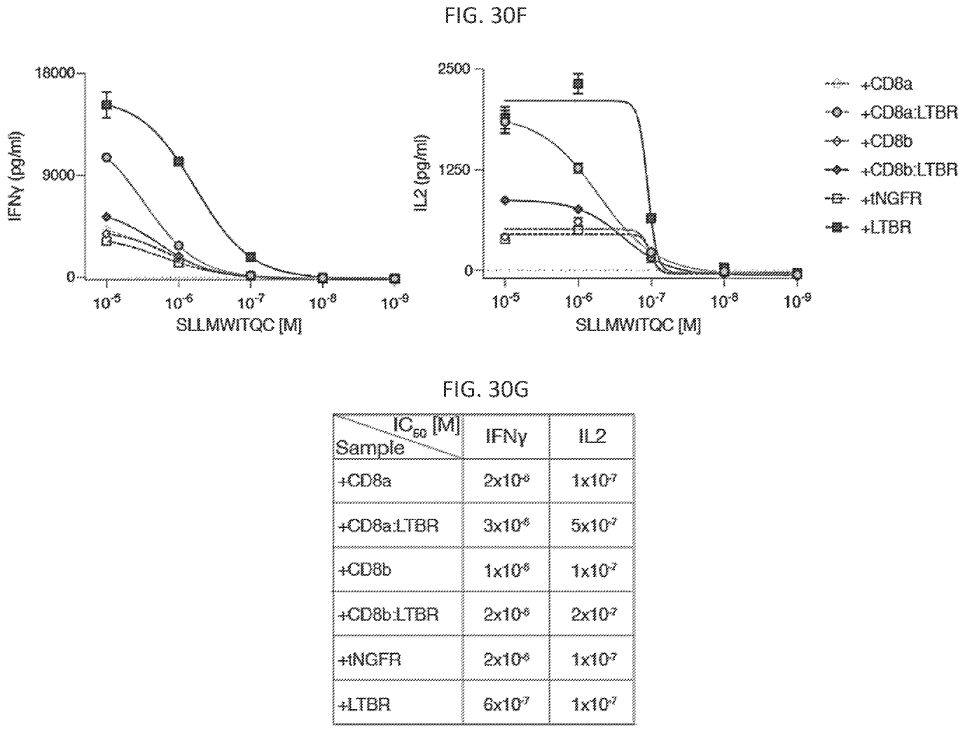

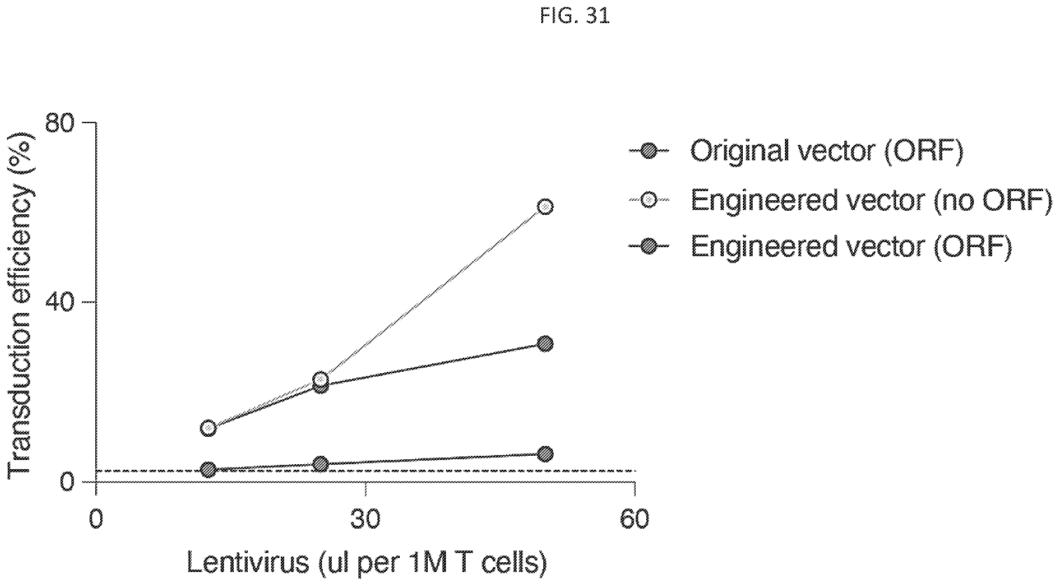

To validate the top-ranked ORFs and understand their effect on other relevant aspects of T cell function, we subcloned 33 ORFs from the library into a vector co-expressing a P2A-linked puromycin resistance gene from the same promoter. We chose a truncated nerve growth factor receptor (tNGFR), Jacking its intracellular domain, as a control that has no effect on T cell phenotype²¹, CD4⁺ and CD8⁺ populations were separately isolated from several screen-independent healthy donors and transduced with individual ORFs (FIG. 2A). Using flow cytometry on representative ORFs, we confirmed that they were stably and uniformly expressed in both subsets of T cells for the duration of the experiment (FIG. 7A, FIG. 7B).

Fourteen days after isolation, we restimulated the cells and measured the relative increase in cell numbers. We found that 16 tested ORFs significantly improved cell proliferation compared with tNGFR, and that proliferation was well correlated between CD4⁺ and CD8⁺ cells (Spearman's r=0.61, P=0.002) (FIG. 2B, FIG. 2C, FIG. 7C-FIG. 7H). Having established that the top ORFs improve T cell proliferation, we next tested whether there is also a change in other T cell phenotypes and functions, such as increased cell cycle entry, expression of the activation markers IL2RA (CD25) and CD40L (CD154), and cytokine secretion. Most of the ORFs tested showed no difference in cycling (FIG. 21, FIG. 2J), but showed higher expression of both CD25 and CD154 in T cells after stimulation (FIG. 2D, FIG. 8A), further corroborating their effect in improving the magnitude of T cell responses.

Finally, we measured the secretion of the cytokines interleukin-2 (IL-2) and IFNγ after restimulation with CD3/CD28 (FIG. 2E, FIG. 8B-FIG. 8E). Although our screen was not designed to identify genes that modulate cytokine secretion, several ORFs could both improve T cell proliferation and boost IL-2 or IFNγ secretion (FIG. 2F). The strongest effect was observed for LTBR, which increased the secretion of both these cytokines in CD4⁺ and CD8⁺ T cells by more than fivefold.

### Single-Cell Analysis of ORF Phenotypes

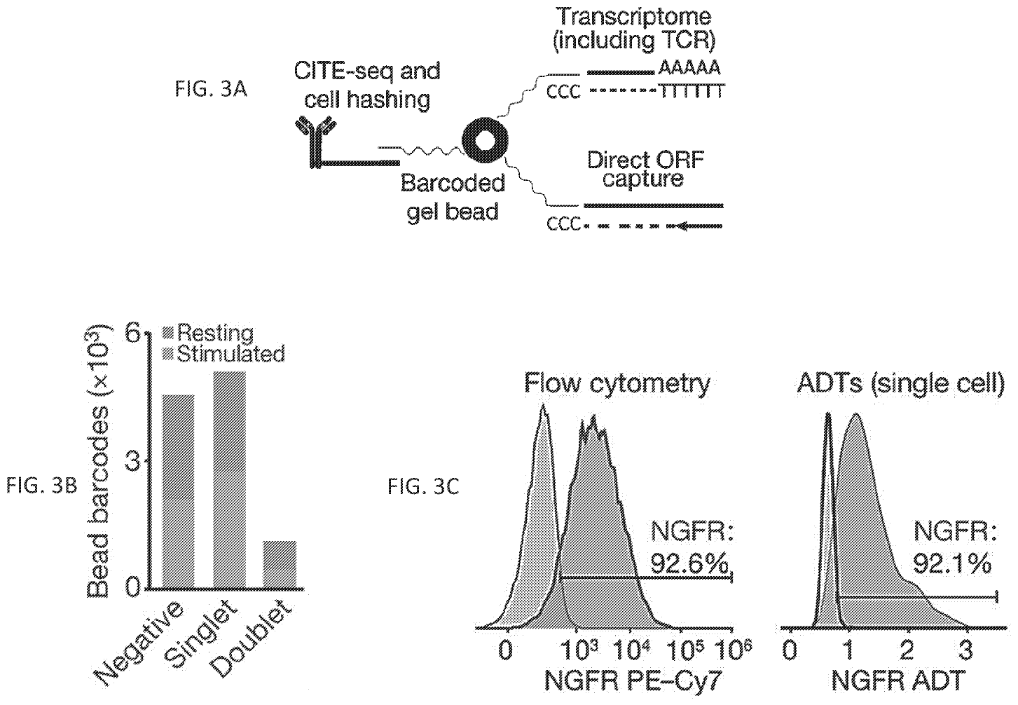

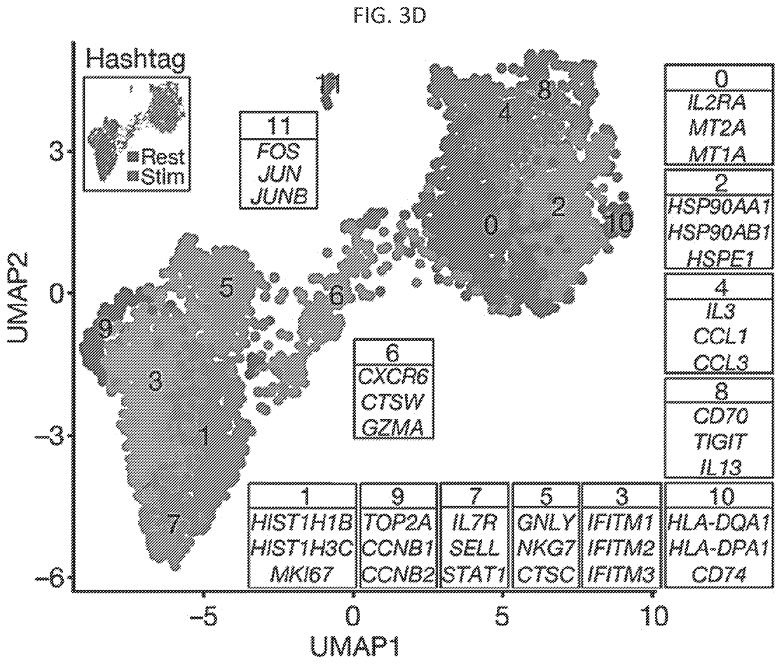

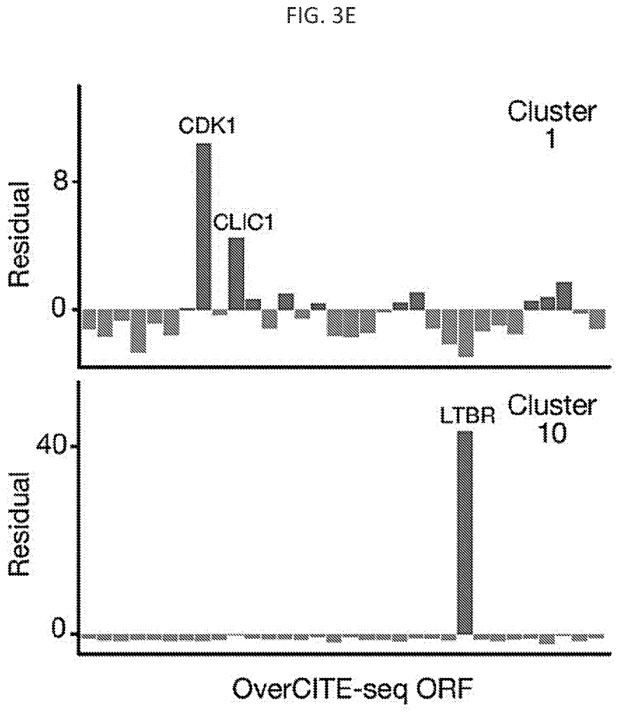

Building on our quantification of how each ORF affects proliferation, activation and cytokine release, we next sought to better understand the underlying mechanisms that drive these changes in cell state. To gain a more comprehensive view of the mechanisms of action of individual ORFs, and to provide a multidimensional characterization of the phenotypic changes they induce, we developed a single-cell sequencing strategy with direct ORF capture. This approach, OverCITE-seq (Overexpression-compatible Cellular Indexing of Transcriptomes and Epitopes by Sequencing) extends previous approaches that we have developed for quantifying surface antigens²² and CRISPR perturbations²³, and allows for high-throughput, single-cell analysis of a pool of T cells with different ORFs. In brief, mRNA from lentivirally integrated ORFs is reverse-transcribed by a primer binding to a constant sequence of the transcript downstream of the ORF and barcoded, along with the cell transcriptome, during template switching. The resulting cDNA pool is then split for separate construction of gene expression and ORF expression libraries (FIG. 3A, FIG. 3B, FIG. 9A).

We optimized and applied OverCITE-seq to a pool of around 30 ORFs transduced into CD8⁺ T cells from a healthy donor. The cell pool was cither left unstimulated (‘resting’) or stimulated with CD3/CD28 antibodies to mimic TCR activation. To gain confidence in how well ORFs are assigned to each single cell, we leveraged the fact that the protein produced by the control gene, tNGFR, is expressed on the cell surface and can thus be captured with a DNA-barcoded antibody²³. The proportion of cells designated as tNGFR positive was consistent when measured by CITE-seq or flow cytometry (FIG. 3C). An analysis of the entire ORF pool showed that single cells assigned with a given ORF had overall the strongest expression of the corresponding gene (FIG. 9B-FIG. 9D), indicating that our ORF capture strategy reliably assigned a genetic perturbation to each single cell.

Unsupervised clustering showed clear separation for stimulated and resting T cells. Within these activation-driven super-clusters we could observe individual clusters associated with a particular cell state or function, such as cell cycle (clusters 1 and 9), macromolecule biosynthesis (cluster 2), type I IFN signaling (cluster 3), cytotoxicity (cluster 6). T cell activation and proliferation (cluster 10), and stress response and apoptosis (cluster 11) (FIG. 3D). Although in many cases several ORFs contributed to a given cluster phenotype (FIG. 9E), we observed a notable enrichment of two ORFs, CDK1 and CLIC1, in cluster 1, characterized by the increased expression of genes that are responsible for chromosome condensation in preparation for cell cycle (FIG. 3E). An even stronger enrichment was observed for cluster 10, which was almost exclusively composed of cells expressing LTBR.

To investigate the mechanisms of genetic perturbations with the strongest transcriptional changes, we looked at the transcriptomic profiles of CD3/CD28-stimulated ORF T cells compared to unstimulated control T cells (FIG. 9F-FIG. 9I). This approach allowed us to identify gene modules that are shared between perturbations or that are perturbation-specific. For example, LTBR and CDK1 showed the strongest enrichment of genes involved in RNA metabolism and cell cycle (CDK4, HSPA8 and BTG3), as well as in the tumor necrosis factor (TNF) signaling pathway (TNFAIP3, TRAF1 and CD70). FOSB appeared to drive an opposite program to LTBR in terms of genes involved in TCR signaling (CD3D, CD3E, LAPTM5 and LAT), cytokine responses (GATA3 and TNERSF4) and the NF-κB pathway (NFκB2, NFKBIA and UBE2N). Finally, we determined that the observed phenotypes were a result of a genetic perturbation rather than an outgrowth of a single clone because virtually every single cell expressed a unique TCR clonotype (FIG. 9J). This result highlights the utility of OverCITE-seq's multimodal capture approach, yielding each T cell's transcriptome, clonotype, cell surface proteome, cell hashing (for treatment or stimulation conditions) and lentiviral ORF identity.

### LTBR Improves Multiple T Cell Functions

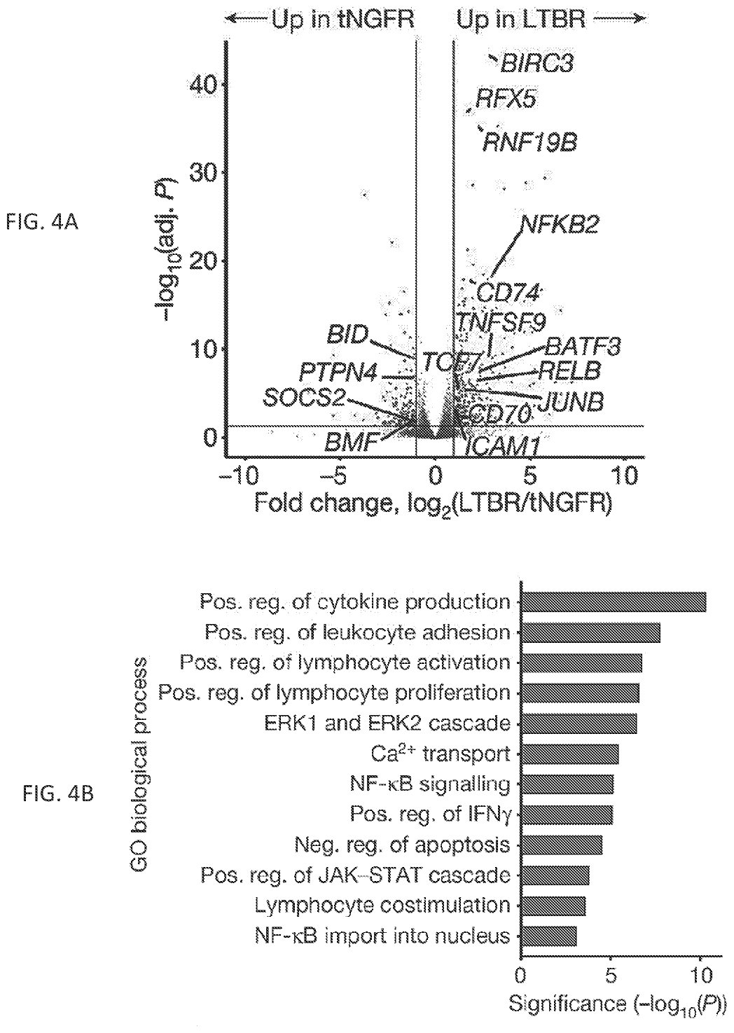

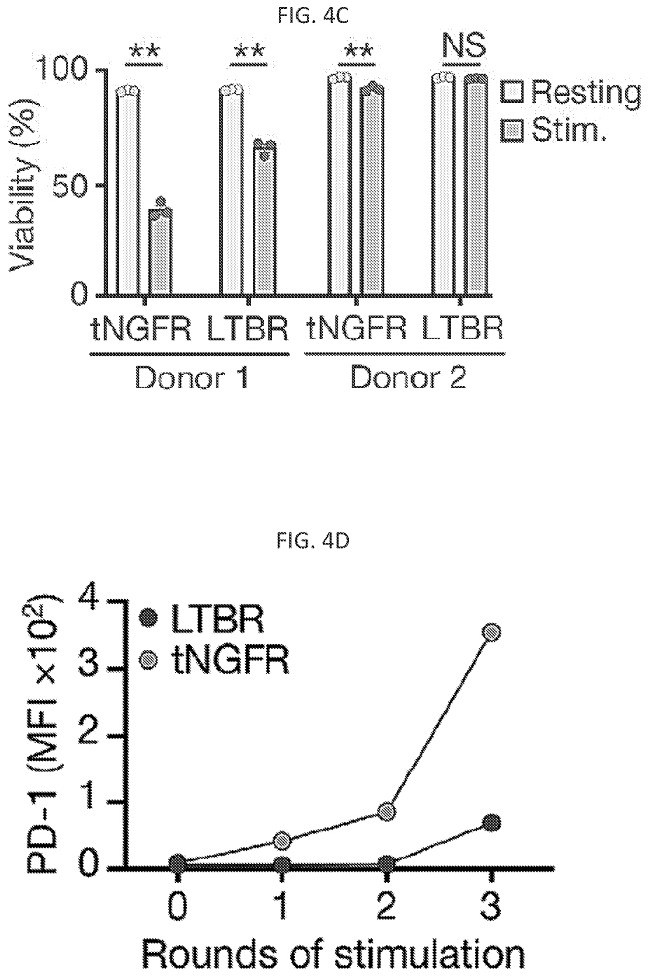

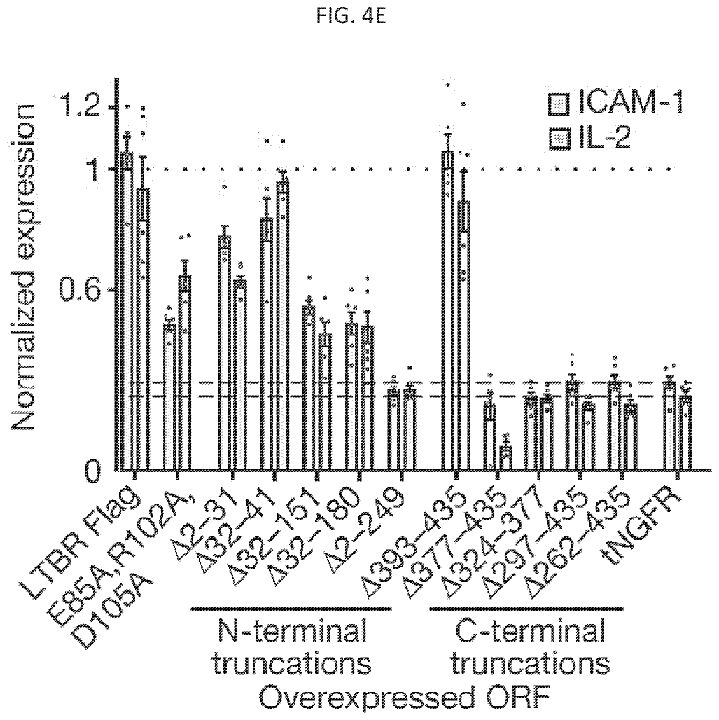

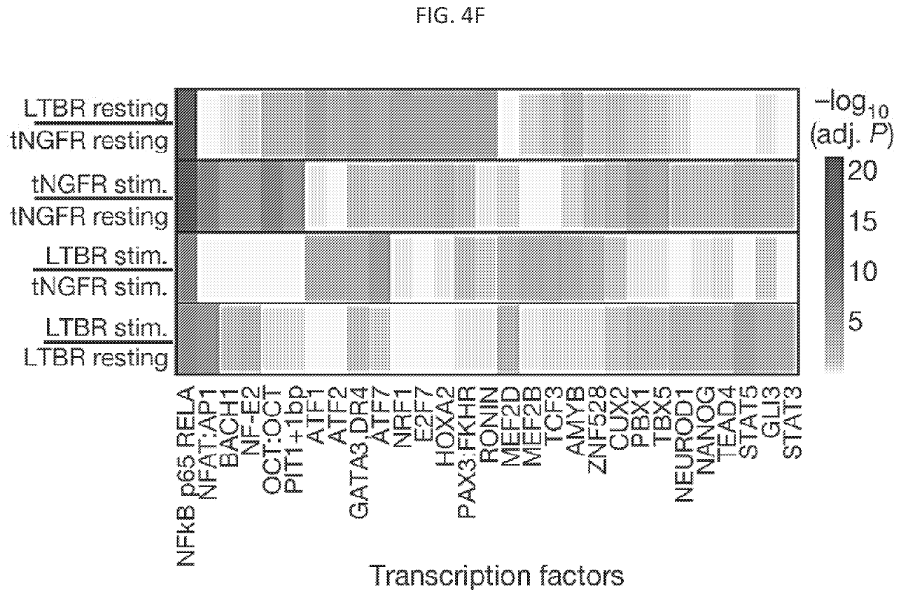

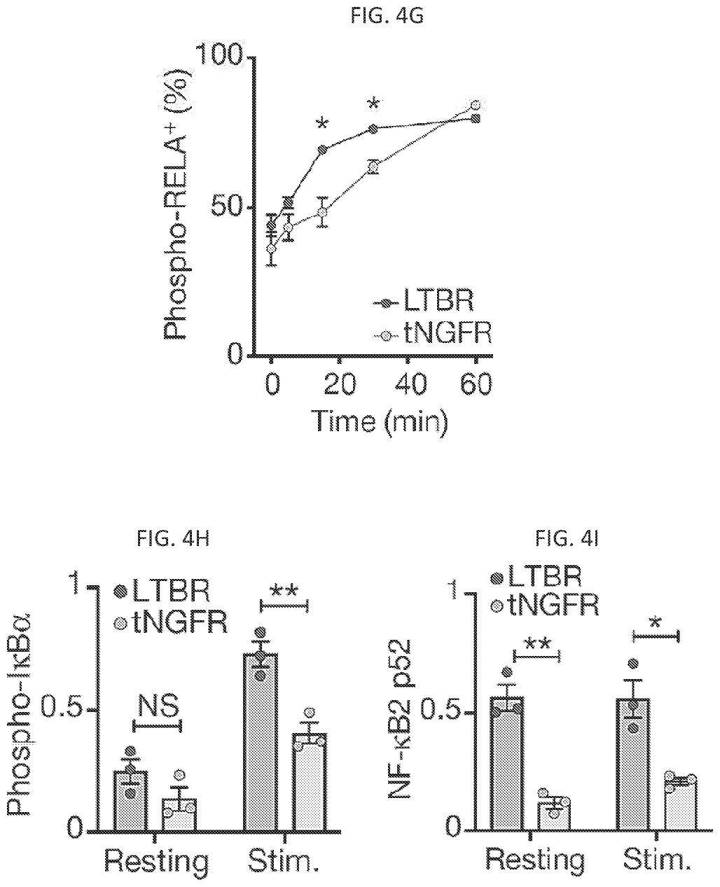

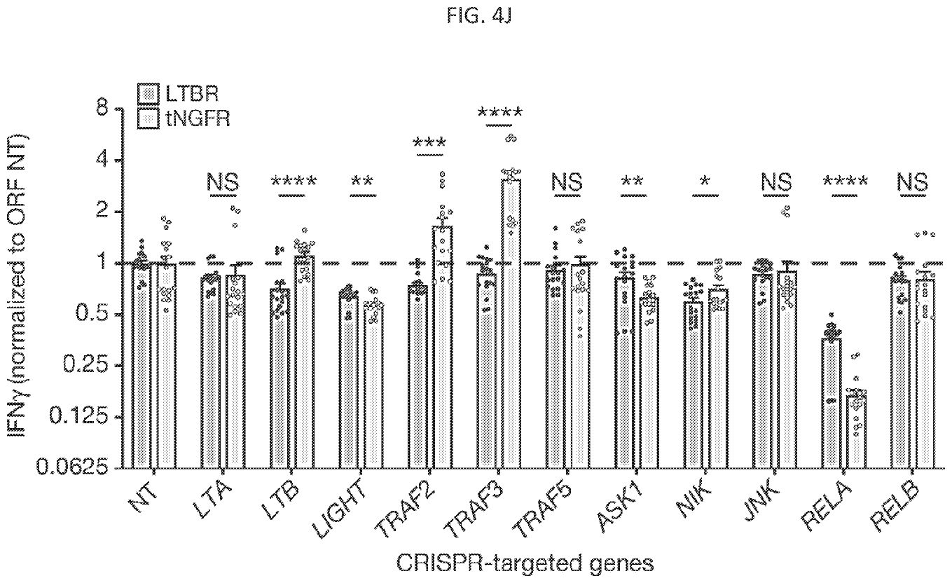

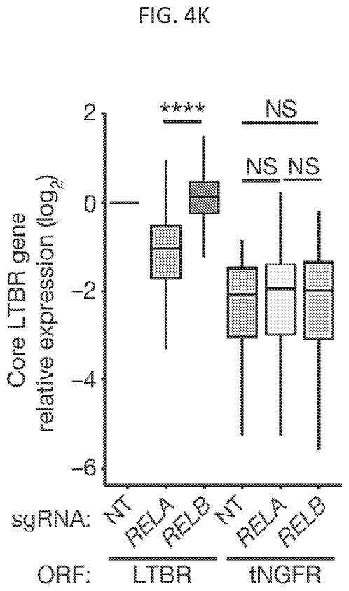

Having identified LTBR as a strong driver of proinflammatory cytokine secretion (FIG. 2E) and profound transcriptional remodeling (FIG. 3D, FIG. 3E), we decided to investigate its mechanisms of action in more detail. LTBR belongs to the tumor necrosis factor receptor superfamily (TNFRSF) and is expressed on a variety of non-immune cell types and on immune cells of myeloid origin, but is absent from lymphocytes (FIG. 10A, FIG. 10B). Using bulk RNA sequencing (RNA-seq), we compared global gene expression between LTBR- and tNGFR-transduced cells, with or without TCR stimulation (FIG. 4A, FIG. 4B, FIG. 10C). In addition to upregulation of MHC-I and II genes (HLA-C, HLA-B, HLA-DPB1, HLA-DPA1 and HLA-DRB6) and transcription factors necessary for MHC-II expression (RFX5 and CHITA), LTBR cells also expressed the MHC-II invariant chain (encoded by CD74). Notably, CD74 has been shown in B cells to activate the pro-survival NF-κB pathway, in particular through upregulation of the anti-apoptotic genes TRAP1 and BIRC3 (both of which are also upregulated in LTBR-overexpressing cells) 24. Similarly, LTBR cells strongly upregulated BATF3, which has been shown to promote the survival of CD8⁺ T cells²⁵. We also observed upregulation of JUNB, a transcription factor involved in IL-2 production²⁶, and TCF7 (encoding TCF1), a key transcription factor responsible for T cell self-renewal²⁷. We confirmed the RNA-seq results at the protein level (FIG. 10D-FIG. 10I). LTBR cells were also more resistant to activation-induced cell death and retained greater functionality after repeated stimulations (FIG. 4C, FIG. 4D, FIG. 10J-FIG. 10M).

LTBR signaling in its endogenous context (in myeloid cells) is triggered either by a heterotrimer of lymphotoxin-α (LTA) and lymphotoxin-B (LTB) or by LIGHT (encoded by the TNFSF14 gene). As LTA, LTB and LIGHT are expressed by activated T cells, we sought to elucidate whether addition of exogenous LTA or LIGHT could modulate the cytokine secretion, differentiation or proliferation of CD3/CD28-stimulated LTBR-overexpressing T cells: however, we found no effect of exogenous ligands on LTBR T cell function (FIG. 11A-FIG. 11E). Thus, although LTBR could potentiate the TCR-driven T cell response, it does not drive activation on its own-which would be a potential safety issue and result in loss of antigen specificity of the engineered T cell response. We also determined that constitutive expression of LTBR is required for maintenance of its phenotype but that there is a substantial lag time between loss of detectable LTBR expression and loss of phenotype (FIG. 11F-FIG. 11I), indicating that transient expression of LTBR may be a safe avenue into a therapeutic application.

Finally, to identify the key domains of the LTBR protein that drive its activity in T cells, we designed a series of point or deletion mutants of LTBR (FIG. 4E, FIG. 11J). In general, we found that the N terminus of LTBR was less sensitive to deletions than the C terminus. Similarly, a partial reduction of the LTBR phenotype was achieved by introducing three alanine point mutations in the key residues for LTA and LTB binding²⁸, or by removal of the signal peptide. Using our C-terminal deletions, we found that a mutant version of LTBR that lacks residues 393-435 showed no difference compared with full-length LTBR, whereas the deletion of residues 377-435 completely abrogated the LTBR phenotype, despite being expressed at a comparable-if not higher-level (FIG. 11K), probably owing to the loss of a binding site for TRAF2, TRAF3 or TRAF529. Moreover, a deletion of the self association domain³⁰ (324-377) also completely abrogated the phenotype.

### LTBR Acts Through Canonical NF-κB in T Cells

LTBR overexpression was shown to induce broad transcriptomic changes in T cells, accompanied by changes in T cell function (FIG. 4A, FIG. 4B). Thus, we sought to determine whether the perturbations in gene expression in LTBR cells were accompanied by epigenetic alterations, leveraging the assay for transposase-accessible chromatin by sequencing (ATAC-seq) (FIG. 12A-FIG. 12G). Comparing the enrichments of specific transcription factor motifs in differentially accessible chromatin regions, we identified NF-κB p65 (RELA) as the most enriched transcription factor in LTBR cells (FIG. 12H, FIG. 12I). Of note, NF-κB p65 and NFAT-AP-1 were the two most enriched transcription factors in open chromatin in stimulated versus resting T cells (both LTBR and tNGFR), in line with their well-established role in T cell activation³¹, but only NF-κB p65 showed strong enrichment in LTBR cells, with and without stimulation (FIG. 4F). This result suggests that LTBR induces a partial T cell activation state but still requires signal 1 (TCR stimulation) for full activation.

We then decided to investigate changes in protein expression and/or phosphorylation of the members of the NF-κB signaling pathway. We observed a more rapid phosphorylation of p65 (RELA) and a strong increase in phosphorylation of an NF-κB inhibitor, IκBα, targeting IκBα for degradation; both of these effects enhance NF-κB activation or transcription (FIG. 4G, FIG. 4H. FIG. 13A-FIG. 13C). In addition to changes in the canonical NF-κB pathway, we also detected an upregulation of key mediators of the non-canonical NF-κB pathway. RELB and NF-κB p52 (FIG. 4I, FIG. 13B, FIG. 13C).

Having established that LTBR activates both the canonical and the non-canonical NF-κB pathways, we sought to determine the molecular basis of this phenomenon by perturbing key genes in the LTBR and NF-κB pathways by co-delivery of LTBR or tNGFR and CRISPR constructs that target II genes involved in the LTBR signaling pathway³² (FIG. 4J, FIG. 13D-FIG. 13O). Knockout of LTB. TRAF2 and NIK (also known as MAP3K14) significantly reduced the secretion of IFNγ from LTBR cells but not (or to a lesser extent) from control (tNGFR) cells, whereas perturbations of LIGHT (also known as TNFSF14), ASK1 (also known as MAP 3KS) and

RELA had a stronger effect on control cells than on LTBR cells. The effect of LTB loss on T cell activation in LTBR cells supports the observation that alanine mutagenesis of key residues involved in LTA or LTB binding (FIG. 4E) partially reduced the LTBR phenotype. Notably, we observed that loss of either TRAF2 or TRAF3 boosted IFNγ secretion in tNGFR cells only, in line with previous findings that T cells from TRAF2, dominant negative mice are hyperresponsive to TCR stimulation³³.

To investigate the potential roles of canonical versus non-canonical NF-κB signaling in LTBR T cells, we decided to analyze the global effects of RELA or RELB loss on the LTBR-driven gene expression profiles. Using bulk RNA-seq on T cells overexpressing LTBR or tNGFR, we discovered that only the loss of RELA significantly downregulated the expression of ‘core’ LTBR genes, whereas loss of RELB had no effect (FIG. 4K, FIG. 13P).

### ORF's Enhance Antigen-Specific Responses

Thus far we have shown that top-ranked genes from the ORF screen improve T cell function using a non-specific, pan-TCR stimulation. We next sought to determine whether a similar improvement could be observed using antigen-specific stimulation (FIG. 5A). To that end, we co-expressed several top-ranked genes with two FDA-approved CARs that target CD19, a B cell marker (FIG. 14A-FIG. 14D). Using LTBR as an example, we demonstrated that ORF expression is achievable with this tricistronic vector (FIG. 14E-FIG. 14I).

Since both CARs use different costimulatory domains, from CD28 or 4-1BB, we wanted to determine whether top-ranked genes that were selected using CD28 co-stimulation could also work in the context of 4-1BB co-stimulation. Nearly all of the top-ranked genes tested, with the exception of AKR1C4, improved upregulation of CD25 and antigen-specific cytokine secretion, with no major differences in the differentiation or exhaustion phenotype (FIG. 5B, FIG. 5C, FIG. 14J-FIG. 14P, FIG. 15A-FIG. 15D).

Although production of IL-2 and IFNγ is crucial for the clonal expansion and antitumor activity of T cells, another vital component of tumor immunosurveillance is direct cytotoxicity. Top-ranked genes had an overall stronger effect on the cytotoxicity of CD28 CAR T cells than 4-1BB CAR T cells (FIG. 5D-FIG. 5F, FIG. 15E, FIG. 15F). Notably, we observed that CAR T cells co-expressing LTBR tended to form large cell clusters; these clusters were typically absent in wells with control cells but are consistent with the overall higher expression of adhesion molecules such as ICAM-1 in LTBR-expressing cells (FIG. 15G). Another important feature of effective antitumor T cells is the ability to maintain functionality despite chronic antigen exposure. In line with our previous findings in the context of LTBR alone (FIG. 4D), CAR T cells expressing LTBR showed a better functionality than matched CAR T cells expressing tNGFR after repeated challenge with target cells (FIG. 5G, FIG. 15H-FIG. 15J).

T cells from healthy donors are relatively easy to engineer and rarely show signs of dysfunction in culture, whereas autologous T cells in patients with cancer are often dysfunctional, showing limited proliferation and effector functions³⁴. To investigate whether top-ranked genes can improve CAR T cell response not only in healthy T cells but also in potentially dysfunctional T cells derived from patients, we transduced CD19 CARs co-expressed with LTBR or a control gene into peripheral blood mononuclear cells (PBMCs) from patients with diffuse large B cell lymphoma. After co-incubation with CD19⁺ target cells, we observed a similar increase in the secretion of IL-2 and IFNγ from LTBR CAR T cells to that seen in healthy donors, indicating that identified ORFs can be successfully used to engineer T cells from patients with lymphoma ex vivo (FIG. 5H, FIG. 15K). Of note, there was no secretion of cytokines in response to CD19-cells, indicating that overexpression of LTBR does not induce a spurious, antigen-independent response.

The screen and subsequent validations were performed in αβ T cells, the predominant subset of T cells in human peripheral blood. Although immunotherapy based on αβ T cells has shown considerable potential in the clinic, γδ T cells present an attractive alternative, owing to their lack of MHC restriction, ability to target broadly expressed stress markers in a cancer-type-agnostic manner and more innate-like characteristics⁵. We therefore sought to determine whether the top genes validated in αβ T cells translated to γδ T cells. After co-incubation with leukemia or pancreatic ductal adenocarcinoma cancer cells, we observed an increase in IL-2 and IFNγ secretion from γδ T cells that were transduced with top-ranked genes (FIG. 5I, FIG. 15L-FIG. 15P). Thus, top-ranked genes from our screen can act on signaling pathways that are conserved between even highly divergent T cell subsets, highlighting their broad applicability for cancer immunotherapy.

### Discussion

In summary, here we developed a genome-scale gain-of-function screen in primary human T cells, in which we examined the effects of nearly 12,000 full-length genes on TCR-driven proliferation in a massively parallel manner. The largest-to our knowledge-previously published gain-of-function study in primary T cells involved 36 constructs, including full-length genes and synthetic receptors³⁵. That approach relied on construct delivery via donor DNA and Cas9-mediated targeted insertion. Although using donor DNA for target gene delivery allows for more flexibility in terms of construct design, especially for engineering synthetic receptors, that method is less scalable and less accessible in terms of cost and complexity than the lentiviral library that we used here. Thus. ORF-based gain-of-function screens are readily applicable to a plethora of T cell phenotypes and settings, and that they offer the opportunity for clinical translation. In fact, all FDA-approved CAR therapies already rely on lentiviral or retroviral integration of a CAR transgene, and therefore an addition of an ORF to this system should pose no major manufacturing or regulatory challenges. The use of ORF-encoding mRNA delivered to CAR T cells before infusion is another translational route, especially if there are safety concerns about the mode of action of a particular ORF.

Gain-of-function screens have the potential to uncover regulators that are tightly controlled, restricted to a specific developmental stage or expressed only in certain circumstances. As shown here, LTBR is canonically absent from cells of lymphoid origin, but, owing to the intact signaling pathway, it can have a synthetic role when introduced to T cells. Although constitutive activation of other TNFRSF members might result in a similar phenotype, one of the features that distinguishes LTBR (and plausibly led to its enrichment, but not that of other TNFRSF members, in the screen) is the formation of an autocrine loop whereby the receptor and its ligands are present in the same cell. It is particularly noteworthy that expression of LTBR boosts IL-2 secretion, as this cytokine is produced exclusively by T cells and not by cell types that endogenously express LTBR. In addition to boosting cytokine secretion, overexpression of LTBR promoted stemness (expression of TCF1) and decreased activation-induced apoptosis, as well as offered a level of protection against phenotypic and functional hallmarks of T cell exhaustion—all of which are features not recapitulated by cell types that endogenously express LTBR. Previous work using overexpression of LTBR in cell lines showed that LTBR has a pro-apoptotic role³⁶, in direct contrast to the phenotype that we observed in primary T cells. Transcript- and protein-level analyses revealed that LTBR drives the constitutive activation of both canonical and non-canonical NF-κB pathways. However, using epigenomic profiling and CRISPR-based functional perturbations we showed that the phenotypic and functional changes resulting from LTBR expression are mediated primarily through activation of the canonical NF-κB pathway, whereas changes in the non-canonical pathway may not be essential for the observed phenotypes-in contrast to the well-established role of non-canonical NF-κB activation in cells that endogenously express LTBR37.

Gene overexpression has been used for pre-clinical enhancement of CAR T cell therapies in numerous studies. For example, armoring CAR T cells with cytokines such as IL-12 or IL-18, which are not typically produced by T cells but are known to improve T cell function when secreted by other cell types, was shown to improve their antitumor activity^{38,39}. Notably, a previous study found that CAR T cell exhaustion can be alleviated by overexpression of e-JUN, a transcription factor identified by RNA-seq as specifically depleted in exhausted cells⁴⁰. Future studies that adapt genome-wide gain-of-function screens to relevant models of immunotherapy will lead to advanced target selection for engineering synthetic cellular therapies that can overcome the immunosuppressive tumor microenvironment and eradicate established cancers.

### Example 3: Improved CAR Solid Tumor Responses

We have shown that LTBR and several other top-ranked genes (ORFs, open reading frames) identified in the screen boost the antitumor response of anti-CD19 CARs in context of a B cell leukemia. Here we tested whether a similar improvement of activity can be seen in conjunction with two clinically-tested anti-mesothelin CARs (using either 4-1BB or CD28 costimulatory domains) in the context of pancreatic cancers. We tested T cells co-expressing a CAR and an ORF against Capan-2, a pancreatic cancer line expressing high levels of mesothelin, the CAR target, and BxPC3, a pancreatic cancer line expressing low levels of mesothelin (FIG. 16A).

Following overnight co-incubation, we determined that all but one ORF tested (that is. AHNAK, BATF, IFNL2, IL 12B, and LTBR) boosted antigen-specific secretion of IFNγ, when used with either CAR (cither 41BB or CD28) against a mesothelin-high cell line Capan-2 (FIG. 16B). In terms of boosting IL-2 secretion, we observed a striking improvement over the negative (tNGFR) control when using LTBR, and to lesser extent AHNAK (FIG. 16C). In terms of reactivity against mesothelin-low line BxPC3, an improvement over the negative control was observed predominantly in T cells overexpressing IL 12B or LTBR (FIG. 16D).

Cytokine secretion is one of the aspects of a productive antitumor response-another one is direct cytotoxicity Therefore, we tested the ability of CAR T cells co-expressing top genes to kill GFP+ Capan2 or BxPC3 cells (FIG. 16E, FIG. 16F). While increased cytotoxicity against mesothelin-high Capan-2 exhibited by CAR T cells overexpressing any of the six top genes tested (including GPD1) was expected given the improvement in cytokine secretion, we also observed increased cytotoxicity against BxPC3 cells. Therefore, we concluded that the top-ranked genes identified in our screen (including but not limited to AHNAK, BATF, GPD1, IFNL2, IL12B, and LTBR) could boost reactivity of diverse CARs (anti-CD19 shown previously, anti-mesothelin shown here) using different costimulatory domains (CD28 or 4-1BB), in different cancer types (including liquid tumors such as B cell leukemia and solid tumors such as pancreatic cancer), and at different target antigen densities (mesothelin-high and mesothelin-low cell lines).

### Example 4: Improved Activity of a TCR in Solid Tumor

T cell therapies can rely on redirecting the cells to a given tumor target using either a CAR or a TCR. The former has the advantage of being able to target tumors in different patients, regardless of their HLA haplotype, while the latter can also target antigens that are intracellular (since epitopes from all cellular proteins are sampled by and displayed on the HLA molecules). Here we used a clinically-tested TCR directed against an epitope from NY-ESO-1, commonly expressed in many cancer histologies, including but not limited to melanoma, multiple myeloma, sarcoma, lung cancer. Due to size restrictions we delivered the TCR and the gene (ORF, open reading frame) on two separate lentiviruses that were used to co-transduce T cells (FIG. 17A). Then, the dual-transduced T cells were selected using puromycin (only T cells transduced with the ORF lentivirus would survive) and using antibody-based selection of NY-ESO-1 TCR positive cells (in presence of dasatinib to prevent T cell activation and thus activation-induced cell death during the selection process).

We then tested the engineered CD8+ T cells against an HLA-A2+NY-ESO-1+ melanoma line A375. Most genes tested increased the secretion one or both of cytokines IFNγ and IL2 (FIG. 17B, FIG. 17C). We also measured direct cytotoxicity against A375 cells and demonstrated that all genes tested showed superior cytotoxicity than the TCR-transduced T cells co-expressing the negative control gene tNGFR (FIG. 17D). Therefore, we concluded that the top-ranked genes identified in our screen (including but not limited to AHNAK, BATF, GPD1, IFNL2, IL 12B, and LTBR) could boost reactivity of T cells engineered with a cancer-specific TCR.

### Example 5: Intracellular Signaling Domain from LTBR Potentiates CAR T Cell Activity

We have demonstrated that LTBR overexpression in T cells, in conjunction with anti-CD19 CARs utilizing either CD28 or 4-1BB costimulatory domains in addition to CD3z signaling domain, increases cytokine (IFNγ and IL-2) secretion and target cell killing (B cell leukemia cell line Nalm6). We have also demonstrated that CAR T cells co-expressing LTBR have an increased expression of core genes that are activated by LTBR, for instance an adhesion molecule ICAM-1 (also known as CD54) and CD74, an invariant part of the MHC-II complex with proposed anti-apoptotic roles. Finally, we have shown that T cells overexpressing LTBR with or without a CAR, show a less differentiated. “younger” phenotype (predominantly central memory [CM], with reduced frequency of terminally differentiated effector cells) which is beneficial in context of cellular therapies.

Here, we inserted the intracellular part of LTBR (https://www.uniprot.org/uniprot/P36941, amino acids 249-435) within the intracellular domains of most commonly used CAR constructs to see if: 1) this insertion does not disrupt the CAR: 2) this insertion bestows similar phenotypic and functional benefit as expressing a full length LTBR as a separate gene in a multicistronic construct, and 3) this insertion boosts CAR activity above that of the CAR and LTBR delivered together but as separate proteins. To that effect we first engineered 3 constructs whereby the intracellular domain of LTBR is inserted into various position in a CAR construct identical to the one used in the FDA-approved axicabragene ciloleucel (here denoted as 19-28-z), specifically: 1) downstream of the CD28 stalk and transmembrane domains but upstream of CD28 signaling and CD3z signaling domains; 2) downstream of the CD28 signaling domain but upstream of CD3z signaling domain; and 3) downstream of both CD28 and CD3z signaling domains (FIG. 21A). These three constructs were then transduced into CD4+ and CD8+ T cells from a healthy donor and compared to a full-length CAR expressed separately with a control gene tNGFR (19-28-z+tNGFR) or LTBR (19-28-z+LTBR). We also engineered two additional constructs whereby the intracellular domain of LTBR is inserted into various position in a CAR construct identical to the one used in the FDA-approved tisagenlecleucel (here denoted as 19-BB-z), specifically: 1) downstream of the 4-1BB signaling domain but upstream of CD3z signaling domain; and 2) downstream of both the 4-1BB and CD3z signaling domains (FIG. 21B). These two constructs were then transduced into CD4+ and CD8+ T cells from a healthy donor and compared to a full-length CAR expressed separately with a control gene tNGFR (19-BB-z+tNGFR) or LTBR (19-BB-z+LTBR).

| SEQ ID | | |
|---|---|---|
| NO | Name | Type |
| 3 | 19-28-z + LTBR protein | AA |
| 4 | 19-28-z + LTBR protein | DNA |
| 5 | 19-28-z + NGFR protein | AA |
| 6 | 19-28-z + NGFR protein | DNA |
| 7 | 19-BB-z + LTBR | AA |
| 8 | 19-BB-z + LTBR | DNA |
| 9 | 19-BB-z + tNGFR | AA |
| 10 | 19-BB-z + tNGFR | DNA |
| 11 | 19-LTBR-28-z protein | AA |
| 12 | 19-LTBR-28-z protein | DNA |
| 13 | 19-28-LTBR-z | AA |
| 14 | 19-28-LTBR-z | DNA |
| 15 | 19-28-z-LTBR | AA |
| 16 | 19-28-z-LTBR | DNA |
| 17 | 19-BB-LTBR-z | AA |
| 18 | 19-BB-LTBR-z | DNA |
| 19 | 19-BB-z-LTBR | AA |
| 20 | 19-BB-z-LTBR | DNA |

We first verified that the surface CAR expression is similar between these constructs, thus indicating that inserting LTBR intracellular domain did not have a profound effect on expression or protein stability of the CAR, and that the observed functional effects were not due to the expression level of the CAR (FIG. 21C, FIG. 21D). Since full-length LTBR induced higher expression of a wide range of genes beneficial for cell therapy, including CD54 and CD74, we tested whether inclusion of the intracellular LTBR domain in the CAR phenocopies the effect of the full length LTBR. In fact, we observed an even higher upregulation of these two markers in cells expressing LTBR as the second or third component in the intracellular CAR domain, namely 19-28-LTBR-z, 19-28-z-LTBR. 19-BB-LTBR-z, and 19-BB-z-LTBR (FIG. 21E, FIG. 21F). This suggests that inclusion of just the signaling domain of LTBR into the CAR may have a more profound effect on T cell state than expressing the whole protein. We also observed an increase in central memory (CM) and decrease in effector phenotype in cells expressing LTBR as the second or third component in the intracellular CAR domain, to a higher extent than in cells expressing full-length LTBR (FIG. 21G, FIG. 21H).

Having established that some of the tested constructs not only phenotypically replicate the effects of full-length LTBR but are in fact superior, we then sought to determine if they also improve antigen-specific response of CAR T cells against CD19+ cancer cells. In terms of cytokine secretion, we observed that the 19-LTBR-28-z construct generally performed worse than the control (“regular” CAR T cells, i.e. 19-28-z+tNGFR) while 19-28-LTBR-z showed similar performance to the control. Most importantly, 19-28-zLTBR outperformed T cells expressing (separately) the CAR and full-length LTBR in terms of IFNg secretion and showed an improvement over control CAR T cells in terms of IL2 secretion (FIG. 21I, FIG. 21J). In cases of CARs utilizing the 4-1BB domain, the construct containing LTBR in the second position (19-BBLTBR-z) showed overall an improvement over the control CAR T cells and similar performance to CAR T-cells co-expressing full-length LTBR-while the construct containing LTBR in the third position outperformed even the CAR+full length LTBR in all cases tested (FIG. 21K, FIG. 21K).

While cytokine secretion (IFNg, IL2) is crucial for T cells to remodel the tumor microenvironment and stimulate T cell proliferation, another key aspect of anticancer activity is direct cytotoxicity, performed predominantly by CD8+ T cells. Therefore, we tested the ability of engineered T cells to kill CD19+ cancer cells. Interestingly, all CD28 LTBR constructs showed an improvement over control CAR at high T cell dose. More importantly, the 19-28-z-LTBR construct was also able to efficiently kill tumor cells at a low T cell dose, where T cells expressing 19-28-z CAR and full-length LTBR provided only a slim benefit over a regular CAR (FIG. 21M). Similarly in cases of 4-1BB CARs: both constructs tested showed an improvement over a regular CAR at high T cell dose but only the 19-BB-z-LTBR construct was more cytotoxic at a low T cell dose (FIG. 2 IN). This is of particular importance for three reasons: 1) clinical efficacy: other than infusing more T cells, there is no possibility to clinically control how many CAR. T cells will encounter tumor cells so they should be able to destroy the cancer cells with as much efficacy as possible, 2) safety: if the bulk of the disease is eliminated rapidly, that reduces the chances of severe adverse events, in particular cytokine release syndrome-especially if this disease debulking can be achieved at a lower T cell number, and 3) manufacturing: due to exhaustion/dysfunction. T cells from some patients fail to expand sufficiently ex vivo prior to re-infusion to achieve the recommended cell dose-if the recommended cell does can be scaled down due to superior efficacy, that may make more patients eligible to receive the therapy.

### Example 6: Intracellular LTBR as an Alternative to CD28 or 4-1BB in 2nd Generation CARs

1st generation CAR constructs contained only the CD3z signaling domain attached to the target recognition modality. These 1st generation receptors were not efficient in vitro and especially in vivo because of lack of a costimulatory signal, thus necessitating the provision of costimulation via inclusion of CD28 or 4-1BB signaling domains in 2nd generation CARs (currently FDA approved). We wanted to assess if the presence of LTBR could improve the response of 1st generation CARs.

Thus, we designed constructs that possessed only the target recognition domain and transmembrane part of a CAR (generation 0)) as well as constructs that also contained the intracellular CD3z signaling domain (1st generation) and compared them to 2nd generation CARs utilizing the 4-1BB costimulatory domain (FIG. 22A). Each generation CAR was co-expressed with either tNGFR (negative control) or full-length LTBR.

| SEQ ID | | |
|---|---|---|
| NO | Name | Type |
| 21 | 19 + tNGFR | AA |
| 22 | 19 + tNGFR | DNA |
| 23 | 19 + LTBR | AA |
| 24 | 19 + LTBR | DNA |
| 25 | 19-z + tNGFR | AA |
| 26 | 19-z + tNGFR | DNA |
| 27 | 19-z + LTBR | AA |
| 28 | 19-z + LTBR | DNA |

When co-incubated with CD19+ leukemia cell line Nalm6, generation 0 CARs showed no response in terms of cytokine (IFNγ, IL2) secretion (FIG. 22B, FIG. 22C). T cells expressing the 1st generation CAR together with tNGFR showed a weaker response than a 2nd generation CAR, as expected. Interestingly, when a 1st generation CAR was co-expressed with LTBR, the response was in all cases stronger compared to a regular 2nd generation CAR (19-BB-z+tNGFR) and in some cases similar to the 2nd generation CAR co-expressed with LTBR. Thus, a CAR containing CD3z and LTBR signaling domain could offer an attractive alternative to conventional 2nd generation CARs (i.e., containing CD28 or 4-1BB derived costimulatory domains). This is further supported by the results described in Example 5.

| SEQ ID | | |
|---|---|---|
| NO | Name | Type |
| 29 | LTBR-28-z | AA |
| 30 | LTBR-28-z | DNA |
| 31 | NGFR-28-z | AA |
| 32 | NGFR-28-z | DNA |
| 33 | LTBR-28TM-28-z | AA |
| 34 | LTBR-28TM-28-z | DNA |
| 35 | NGFR-28TM-28-z | AA |
| 36 | NGFR-28TM-28-z | DNA |

### Example 7: Usage of the Extracellular Domain of LTBR

We have previously shown that LTBR overexpression improves T cell phenotype and function via ligand dependent and independent mechanisms. We wanted to assess whether the extracellular domains of LTBR can provide additional T cell activation via the same mechanisms (ligand binding or tonic signaling) when linked to signaling domains derived from 2nd generation CAR T cells.

To that effect we generated constructs that express the full-length extracellular domain of LTBR (LTBR: https://www.uniprot.org/uniprot/P36941, amino acids 1-248) including the transmembrane part, or utilize the stalk and transmembrane part from FDA-approved CARs-that is, CD28 stalk and transmembrane or CD8 stalk and transmembrane (LTBR: https://www.uniprot.org/uniprot/P36941, amino acids 1-227). In some cases, the extracellular domain of LTBR was linked to CD28 and CD3z or 4-1BB and CD3z signaling domains, as in FDA-approved CARs. In all iterations of the constructs, a matched NGFR fragment was used as a negative control (FIG. 23A).

| SEQ ID | | |
|---|---|---|
| NO | Name | Type |
| 37 | LTBR-CD8-BB-z | AA |
| 38 | LTBR-CD8-BB-z | DNA |
| 39 | NGFR-CD8-BB-Z | AA |
| 40 | NGFR-CD8-BB-z | DNA |
| 41 | 19-CD8-LTBR | AA |
| 42 | 19-CD8-LTBR | DNA |
| 43 | 19-CD8-LTBR(TM) | AA |
| 44 | 19-CD8-LTBR(TM) | DNA |
| 45 | 19-28-LTBR | AA |
| 46 | 19-28-LTBR | DNA |
| 47 | 19-28-LTBR(TM) | AA |
| 48 | 19-28-LTBR(TM) | DNA |

We observed no cytokine (IFNγ, IL2) secretion when the T cells were incubated alone, indicating that ligand availability or tonic signaling mediated by LTBR were not sufficient to activate the cells. However, when the T cells were activated via their T cell receptor (CD3 and CD28 antibodies), we observed a strong increase in secretion of both IFNg and IL2 by T cells expressing a construct that contains the extracellular part of LTBR fused to the CD8 stalk and transmembrane domain, and 4-1BB and CD3z signaling domains (LTBR-CD8-BB-z) (FIG. 23B, FIG. 23C). This result indicates that the extracellular domain of LTBR fused to a CAR in place of or in addition to a targeting moiety (typically derived from an scFv antibody fragment) may be a useful approach to potentiating T cell function.

### Example 8: Usage of the Extracellular Domain of LTBR

We have previously shown that LTBR overexpression improves T cell phenotype and function via ligand dependent and independent mechanisms. We wanted to assess whether the extracellular domains of LTBR can provide additional T cell activation via the same mechanisms (ligand binding or tonic signaling) when linked to signaling domains derived from 2nd generation CAR T cells.

To that effect we generated constructs that express the full-length extracellular domain of LTBR (LTBR: https://www.uniprot.org/uniprot/P3694 1, amino acids 1-248) including the transmembrane part, or utilize the stalk and transmembrane part from FDA-approved CARs-that is, CD28 stalk and transmembrane or CD8 stalk and transmembrane (LTBR: https://www.uniprot.org/uniprot/P36941, amino acids 1-227). In some cases, the extracellular domain of LTBR was linked to CD28 and CD3z or 4-1BB and CD3z signaling domains, as in FDA-approved CARs. In all iterations of the constructs, a matched NGFR fragment was used as a negative control (FIG. 24A).

We observed no cytokine (IFNg, IL2) secretion when the T cells were incubated alone, indicating that ligand availability or tonic signaling mediated by LTBR were not sufficient to activate the cells. However, when the T cells were activated via their T cell receptor (CD3 and CD28 antibodies), we observed a strong increase in secretion of both IFNg and IL2 by T cells expressing a construct that contains the extracellular part of LTBR fused to the CD8 stalk and transmembrane domain, and 4-1BB and CD3z signaling domains (LTBR-CD8-BB-z) (FIG. 24B, FIG. 24C). This result indicates that the extracellular domain of LTBR fused to a CAR in place of or in addition to a targeting moiety (typically derived from an scFv antibody fragment) may be a useful approach to potentiating T cell function.

### Example 9: Modified TCR Complexes

The intracellular signaling domain of LTBR (https://www.uniprot.org/uniprot/P36941, amino acids 249-435*) fused to different components of the TCR complex. FIG. 25A shows the existing design that improved T cell function (i.e. NY-ESO-1 TCR and LTBR expressed separately, see Example 4) as well as fusing LTBR signaling domain to different components of the TCR-CD3 complex, specifically at the C-terminal end of the intracellular domains of CD3epsilon (https://www.uniprot.org/uniprot/P07766), CD3gamma (https://www.uniprot.org/uniprot/P09693). CD3delta (https://www.uniprot.org/uniprot/P04234), and CD247 (also known as CD3zeta, https://www.uniprot.org/uniprot/P20963). The LTBR signaling domain can also be fused to the C-terminal ends of TCR-α or TCR-β chains.

In a similar manner as with using LTBR intracellular domain to modify αβ TCR complex. γδTCR complex can be modified too (FIG. 25B). As shown in FIG. 5I, expression of full-length LTBR boosts γδ T cell functions.

αβ T cells use CD4 to co-receive antigens presented by MHC-II (HLA-DR. HLA-DP, HLA-DQ) or CD8 (composed of CD8α and CD8b chains) to co-receive antigens presented by MHC-I (HLA-A, HLA-B, HLA-C). Thus, intracellular LTBR can be fused to the C-terminal intracellular tail of CD4 or to C-terminal intracellular tails of CD8a or CD8b to improve T cell function (FIG. 25C).

### Example 10: LTBR Co-Delivery Potentiates Antitumor Functions of a B7-H3 Targeting CAR

We have previously shown that co-delivery of LTBR with an anti-CD19 CARs into primary T cells boosts their activity against hematological cancers. See, e.g., Legut et al, Nature, (2022). We have shown similar results with co-delivery of LTBR with an anti-mesothelin CARs to boost T cell activity against a range of solid tumors. We have now expanded this application of LTBR co-delivery to an anti-B7-H3 CAR which is being tested clinically against a range of solid tumors, including but not limited to pediatric cancers, brain tumors, sarcomas and melanoma. See, e.g., Theruvath et al, Nature Medicine (2020).

When LTBR was co-delivered to primary human T cells together with an anti-B7-H3 CAR (MGA271-28-z), we observed (as before in cases of CD19 and mesothelin CARs) that LTBR had a positive impact on CAR T cell phenotype. Specifically. LTBR CAR T cells showed a predominantly central memory phenotype with practically no terminally differentiated effector cells, in contrast with CAR T cells co-expressing a control gene tNGFR (FIG. 26A). LTBR CAR T cells also strongly upregulated markers of LTBR activity, CD54 and CD74 (FIG. 26B, FIG. 26C).

In terms of antitumor efficacy. LTBR CAR T cells, both CD4 and CD8, showed a much stronger response to cancer cells expressing B7-H3 (including two atypical teratoid rhabdoid tumor lines BT12 and BT16, as well as a melanoma line A375) than control CAR T cells while remaining inert to a B7-H3-negative leukemia line Nalm6 (FIG. 26D-FIG. 26G). This improvement of antitumor response was observed across all T cell functions tested, including secretion of key cytokines IFNγ and IL2, as well as direct cell killing.

Finally, we assessed the efficacy and safety of LTBR CAR T cells in a mouse xenograft models. We implanted A375 tumor into the flank of NSG mice and allowed it to grow for two weeks to be reminiscent of advanced solid tumors in human (FIG. 26H). When monitoring body weight, we observed no meaningful body weight loss in mice treated with CAR T cells, with or without LTBR, indicating that LTBR did not result in any overt toxicities (FIG. 26I). When measuring the tumor size, we observed that LTBR CAR T cells resulted in overall lower tumor burden, at both doses tested, than mice treated with control CAR T cells or untransduced T cells, indicating that LTBR improved the CAR T cell efficacy in this model (FIG. 26J, FIG. 26K). Due to rapid progression in this advanced solid tumor model, the study was terminated 12 days after T cell injection, preventing us from monitoring the long-term effect of LTBR T cells on tumor regression and survival.

### Example 11: Identification of the Minimal Intracellular Domain of LTBR that Drives its Function in T Cells

We have previously shown that fusion of the intracellular tail of LTBR to the C-terminus of CD19-targeting CARs, either utilizing CD28 or 4-1BB design (FMC6.3-28-z-LTBR or FMC6.3-BB-z-LTBR) results in CAR T cells that show superior antitumor potency and LTBR-driven phenotype than when the full-length CAR and full-length LTBR are co-expressed as separate molecules. Here we wanted to determine if truncations (either N- or C-terminal) of the LTBR signaling tail could result in a smaller but equally or more potent CAR construct than one incorporating the full-length LTBR signaling tail.

| | LTBR amino acids | Included in |
|---|---|---|
| Construct | (Uniprot: P36941) | SEQ ID NO |
| Full length | 249:435 | 84, 85 |
| V1 | 249:396 | 86, 87 |
| V2 | 249:393 | 88, 89 |
| V3 | 249:387 | 90, 91 |
| V4 | 249:377 | 92, 93 |
| V5 | 262:435 | 94, 95 |
| V6 | 297:435 | 96, 97 |
| V7 | 324:435 | 98, 99 |
| V8 | 345:435 | 100, 101 |
| V9 | 358:435 | 102, 103 |

We began by annotating the LTBR intracellular tail based on the literature to identify key functional domains (FIG. 27A). We then designed a series of 4 N-terminal truncation and 5° C.-terminal truncations of the LTBR tail, for a stepwise removal of unannotated parts (which we presumed not to play any biological role) and extending into key signaling domains that were previously annotated (FIG. 27B). We then fused the resulting truncation mutants to the C-terminus of a CD28-based CD19 CAR and compared them with either a CD28-based CAR incorporating the full-length LTBR signaling tail (19-28-z-LTBR) or regular CAR (19-28-z) co-expressed with LTBR or control gene tNGFR. The sequences are shown in SEQ ID Nos: 84-103. Each sequence includes 19-28-z (SEQ ID NO: 134, 135). LTBR variant as indicated above, P2A, puromycin.

All CAR-LTBR fusions resulted in similar levels of CAR surface expression (FIG. 27C).

In terms of differentiation status of T cells, the LTBR truncation fusions showed a range of phenotypes, with certain variants (e.g., V7) resembling full length LTBR very closely while certain other variants (e.g., V4) more reminiscent of regular CAR T cells not expressing LTBR (FIG. 27D). Similarly, full-length LTBR fusion as well as most truncation mutants, with the exception of V3 and V4, resulted in a stronger expression of TCF1, a key transcription factor governing T cell stemness and self-renewal (FIG. 27E). Another indication of the successful induction of the LTBR program, which involves differential expression of hundreds of genes critical for the immune response (See, e.g., Legut et al, Nature (2022)), is surface expression of CD54 and CD74 markers. While certain truncation variants, such as V3. V4 and V9, failed to upregulate CD54 and CD74, the remaining variants showed similar activity to full-length LTBR fusion (FIG. 27F).

In addition to phenotypic measurements, we also assayed the antigen-specific antitumor response by co-incubating T cells expressing anti-CD19 CARs, with or without LTBR fusion, with CD19+ leukemia cells. To quantify that response, we measured the secretion of key antitumor cytokines IFNγ and IL2 (FIG. 27G). In line with the CD54 and CD74 quantification, we observed a similar boost over “regular” CAR (19-28−2+tNGFR) with VS-V7 variants as with full-length LTBR fusion. V1 and V2 showed no improvement of cytokine secretion over “regular” CAR, despite increased levels of CD54 and CD74, while V3 and V4 showed hardly any cytokine secretion, showing that those designs interfered with the CAR itself. Interestingly, V8 variant resulted in greatest upregulation of CD54 and CD74 but an inferior cytokine secretion while V) showed mild upregulation of CD54 and CD74 but strong boost of antitumor activity. This suggests that phenotypic effects of LTBR (CD54, CD74) can be de-coupled from functional effects (IL2, IFNγ).

### Example 12: LTBR Signaling Tail Fusion to a Mesothelin Targeting CAR

We have previously shown that co-expression of full-length LTBR together with mesothelin-targeting CARs, utilizing both the 4-1BB and CD28 designs, boosts antitumor reactivity of CAR T cells as well as that fusing the intracellular tail of LTBR to CD19-targeting CARs, utilizing both the 4-1BB and CD28 designs, results in superior phenotype and function than co-expression of a regular CD19-targeting CAR and LTBR as two independent molecules.

To extend that observation, we fused the intracellular tail of LTBR to mesothelin-targeting CARs (see FIG. 28A for schematic representations of the CAR-LTBR fusion designs). Specifically, we selected the C-terminus of the meso-28-z CAR for LTBR fusion, based on the optimal LTBR positioning within the CAR construct determined in the CD19 system. We also investigated replacing the CD28 costimulatory domain with LTBR, placing it either upstream or downstream of CD3z, testing whether LTBR can be used instead of CD28 to provide costimulation.

| SEQ ID | | |
|---|---|---|
| NO | Name | Type |
| 104 | Meso-28-z-LTBR-P2A-puroR | DNA |
| 105 | Meso-28-z-LTBR-P2A-puroR | AA |
| 106 | Meso-BB-z-LTBR-P2A-puroR | DNA |
| 107 | Meso-BB-z-LTBR-P2A-puroR | AA |
| 108 | Meso-LTBR-z-P2A-puroR (CD28 stalk | DNA |
| 109 | Meso-LTBR-z-P2A-puroR (CD28 stalk | AA |
| 110 | Meso-LTBR-z-P2A-puroR (CD8 stalk) | DNA |
| 111 | Meso-LTBR-z-P2A-puroR (CD8 stalk) | AA |
| 112 | Meso-z-LTBR-P2A-puroR (CD28 stalk) | DNA |
| 113 | Meso-z-LTBR-P2A-puroR (CD28 stalk) | AA |
| 114 | Meso-z-LTBR-P2A-puroR (CD8 stalk) | DNA |
| 115 | Meso-z-LTBR-P2A-puroR (CD8 stalk) | AA |

When looking at the surface expression of the CAR, we observed, in contrast to our data in the CD19 system, that fusing the LTBR tail in any position within the mesothelin CAR resulted in a markedly decreased surface expression of the CAR (FIG. 28B).

To assess the functional impact of the reduced CAR expression upon fusion with LTBR, we measured key functional outputs of CAR-driven response to mesothelin-positive cancer cells: T cell proliferation, direct cancer cell killing, and secretion of key cytokines. In terms of antigen-driven proliferation, co-expression of the full-length LTBR resulted in the strongest expansion but CAR-LTBR fusions still outperformed the regular, unmodified CAR (FIG. 28C). In terms of cancer cell killing, a CAR design lacking CD28 but containing LTBR tail downstream of CD3z (Meso-z-LTBR) showed similar killing to the regular CAR co-expressed with full-length LTBR (FIG. 28D). Surprisingly, despite better or comparable functional activity of mesothelin CAR-LTBR fusions in terms of proliferation and killing, two out of three constructs tested (Meso-28-2-LTBR and Meso-LTBR-z) showed barely any activity when measuring cytokine secretion (FIG. 28E). Meso-z-LTBR T cells were capable of producing cytokines upon co-incubation with mesothelin+ cancer cells but to lesser extent than regular Meso-28-z CAR T cells.

Given that CAR-specific functions were reduced upon fusion with the LTBR tail, we were wondering about the LTBR-specific functions of those constructs. When looking at key markers of LTBR activity, that is, surface expression of CD54 and CD74 (FIG. 28F), as well as intracellular/nuclear expression of the transcription factor TCF1 (FIG. 28G), we observed that all three CAR-LTBR fusion constructs drive a stronger LTBR program than co-expression of the CAR and full-length LTBR. This phenomenon was observed in both the CD28 and 4-1BB designs. Therefore, we hypothesized that mesothelin CAR-LTBR fusions may be suboptimal to act as a CAR but are superior, non-natural versions of LTBR. To test that further, we stimulated the engineered T cells through their endogenous T cell receptors, as an alternative to providing them with signal I than using CAR-mesothelin binding. Supporting our hypothesis, we observed in that context, where CAR-LTBR fusions were needed only to provide LTBR-like signal 2 costimulation without the need to provide CAR-like signal 1 stimulation, CAR-LTBR fusion T cell functional response was superior to that in T cells expressing CAR and LTBR as two independent molecules (FIG. 28H).

To demonstrate the utility of using CAR-LTBR fusion as a superior LTBR-like molecule to be used in conjunction with an independent antigen receptor, we co-delivered natural LTBR or Meso-z-LTBR together with Meso-28-z CAR T to T cells (FIG. 28I). All constructs tested showed the same CAR expression on the surface (FIG. 28J); also, co-delivery of CAR and LTBR on separate viruses resulted in strong surface expression of LTBR, similar to that in T cells transduced only with the LTBR virus but not the CAR virus-highlighting the potential of LTBR delivery on a separate lentivirus as an alternative to co-expressing from the same lentiviral cassette as an antigen receptor (FIG. 28K).

In this co-delivery setting, CAR+Meso-z-LTBR T cells upregulated LTBR marker genes CD54 and CD74 to the similar extent as CAR+LTBR T cells (FIG. 28L). CAR+Meso-z-LTBR T cells also showed superior antigen-specific response to mesothelin+ cancer cells to that of CAR only T cells and even outperformed CAR+natural LTBR T cells in terms of IFNγ secretion (FIG. 28M, FIG. 28N). Taken together, we have shown utility of Meso-z-LTBR synthetic fusion receptor as an alternative to naturally occurring full length LTBR.

### Example 13: LTBR Signaling Tail Fusion to the Members of the TCR-CD3 Complex

We have previously shown that co-expression of full-length LTBR together with an NY-ESO-1 targeting αβ TCR boosts antitumor reactivity of engineered T cells as well as that fusing the intracellular tail of LTBR to CD19-targeting CARs, utilizing both the 4-1BB and CD28 designs, results in superior phenotype and function than co-expression of a regular CD19-targeting CAR and LTBR as two independent molecules.

To extend this observation, we fused the intracellular tail of LTBR to a or B chains of the TCR heterodimer, or δ, ε, γ or ζ chains of the CD3 complex. CD3 constitutively associates with the TCR and is required for its surface expression and downstream signaling (https://www.pnas.org/doi/10.1073/pnas. 1420936111).

To first test whether LTBR signaling tail can boost TCR activity as a direct fusion, we generated constructs that included LTBR intracellular tail attached to the C-terminus of the short intracellular tail of TCRα or TCRβ (FIG. 29A) and tested them alongside an unmodified TCR co-expressed with either a full length LTBR or an irrelevant gene tNGFR.

| SEQ ID | | |
|---|---|---|
| NO | Name | Type |
| 116 | TCRa:LTBR-P2A-TCRb | DNA |
| 117 | TCRa:LTBR-P2A-TCRb | AA |
| 118 | TCRa-P2A-TCRb:LTBR | DNA |
| 119 | TCRa-P2A-TCRb:LTBR | AA |
| 120 | CD3D:LTBR-P2A-puroR | DNA |
| 121 | CD3D:LTBR-P2A-puroR | AA |
| 122 | CD3E:LTBR-P2A-puroR | DNA |
| 123 | CD3E:LTBR-P2A-puroR | AA |
| 124 | CD3G:LTBR-P2A-puroR | DNA |
| 125 | CD3G:LTBR-P2A-puroR | AA |
| 126 | CD3Z:LTBR-P2A-puroR | DNA |
| 127 | CD3Z:LTBR-P2A-puroR | AA |

We first looked at whether LTBR fusion affects surface expression of the TCR. We noticed that LTBR fusion to TCR-β, but not TCR-α, strongly reduced its surface expression (FIG. 29B). We then examined the key phenotypic hallmarks of LTBR activity, that is surface expression of CD54 and CD74. Only LTBR-TCR-β, but not LTBR-TCR-α, resulted in an increased expression of CD54 and CD74, albeit to a lesser extent than full length LTBR (FIG. 29C). In terms of antigen-specific activity, both LTBR-TCR-α and LTBR-TCR-β fusions were proven to be non-functional, resulting in a similar level of cytokine secretion as untransduced cells upon co-incubation with NY-ESO-1+ melanoma cell line A375 (FIG. 29D). Therefore, we concluded that direct LTBR tail fusion to TCR chains results in disruption of expression and/or function of the TCR.

CD3 molecules are necessary for mediating TCR signaling. Therefore, we engineered fusions of all four CD3 polypeptides with LTBR signaling tail (FIG. 29E) and sequentially transduced the CD3-LTBR fusions, or unmodified CD3 genes, together with the NY-ESO-1 TCR into T cells (FIG. 29F). In terms of TCR surface expression, there were no major differences between different conditions, as expected given that T cells already express all CD3 genes (FIG. 29G). Looking at phenotypic hallmarks of LTBR activity, we observed that CD3-LTBR fusions resulted in an overall higher expression of CD54 and CD74 than matched unmodified CD3 constructs (FIG. 29H). In terms of antigen-specific response upon co-incubation with NY-ESO-1+ melanoma line A375, CD3-LTBR fusions failed to boost the activity of TCR engineered T cells above the control level (FIG. 29I). Interestingly, overexpression of unmodified CD38 or CD3ζ resulted in a small but significant increase in the antigen-specific response, albeit to the lesser extent than overexpression of full length LTBR. Overall, LTBR-CD3 fusions have failed to outperform natural, full length LTBR across phenotypic and functional assays, presumably due to competition with endogenous, highly expressed components of the CD3 complex.

### Example 14: LTBR Signaling Tail Fusion to the Members of the CD8 Complex

We have shown that co-expression of full-length LTBR together with an NY-ESO-1 targeting αβ TCR boosts antitumor reactivity of engineered T cells (WO 2023/279049) as well as that fusing the intracellular tail of LTBR to CD19-targeting CARs, utilizing both the 4-1BB and CD28 designs, results in superior phenotype and function than co-expression of a regular CD19-targeting CAR and LTBR as two independent molecules.

To extend this observation, we fused the intracellular tail of LTBR to CD8α or CD8β molecules (FIG. 30A). CD8α-CD8β heterodimer is critical for productive engagement of a TCR (such as an NY-ESO-1 targeting αβ TCR used here) with a peptide antigen (in this case an NY-ESO-1 derived peptide SLLMWITQC) presented by an MHC-I molecule (in this case HLA-A2).

| SEQ ID | | |
|---|---|---|
| NN | Name | Type |
| 128 | CD8a:LTBR-P2A-puroR | DNA |
| 129 | CD8a:LTBR-P2A-puroR | AA |
| 130 | CD8b:LTBR-P2A-puroR | DNA |
| 131 | CD8b:LTBR-P2A-puroR | AA |

In this experiment, CD8 T cells, expressing an endogenous CD8α-CD8β heterodimer, were sequentially transduced with an NY-ESO-1 targeting TCR as well as a modifier gene to be overexpressed: a control gene tNGFR, a full-length LTBR. CD8 (wild-type or fused with the LTBR signaling tail) or CD8β (wild-type or fused with the LTBR signaling tail) (FIG. 30B).

We first measured the surface expression of markers of LTBR activity, CD54 and CD74, as well as intracellular expression of a key transcription factor TCF1. As expected, NY-ESO-1 TCR-transduced T cells co-expressing LTBR exhibited a marked increase of expression of both surface markers and the transcription factor, compared with the control gene tNGFR (FIG. 30C-FIG. 30E). T cells transduced with the CD8-LTBR fusions showed slight increase in CD54 and CD74 expression, but not TCF1, compared with the wild-type counterparts, indicating that the LTBR signaling was at least partially active in these constructs.

Finally, we tested the impact of the CD8-LTBR fusion on the functional response of engineered T cells. NY-ESO-1 antigen is expressed on a variety of tumor types (Thomas et al, NY-ESO-1 Based Immunotherapy of Cancer Current Perspectives, Front. Immunol., 1 May 2018, Sec. Cancer Immunity and Immunotherapy, Volume 9-2018). To test the sensitivity and magnitude of response of LTBR engineered T cells to this antigen, we pulsed an HLA-A2+ cell line with different concentrations of the immunodominant peptide epitope derived from the NY-ESO-1 protein, SLLMWITQC. T cell response was measured as secretion of two key cytokines, IFNγ and IL2, after 24 h co-incubation of T cells with the peptide-presenting cell line (FIG. 30F, FIG. 30G). As expected, co-expression of full-length LTBR induced a much stronger T cell response to the antigen than a matched control, tNGFR, even at peptide concentrations as low as 10-7 M. LTBR transduced T cells also showed >3-fold increase in sensitivity to the peptide concentration (IC₅₀ for IFNγ secretion of 6×10⁻⁷ M for LTBR, 2×10⁻⁶ M for tNGFR). Importantly, CD8-LTBR fusions also increased the magnitude of response, for both cytokines measured, over the matched controls. Thus, we demonstrated the functionality and utility of the synthetic CD8-LTBR fusions as an alternative to the full-length natural LTBR.

### Example 15: ORF Library Vector Engineering to Improve Transduction Efficiency

We have previously shown that a pooled, barcoded lentiviral library containing >12,000 human genes can be used to discover new modulators of key T cell functions (Sec, e.g., Legut et al, Nature 2022). Here we show that engineering of the lentiviral vector backbone drastically boosts the functional titer of the library virus, improving the scalability of the approach. The improved ORF library vector (SEQ ID NO: 132) showed >5× increase over the original vector in terms of the transduction efficiency, making it possible to reach ˜30% transduction rate (which is desirable for pooled screening) with as little as 50 μl lentivirus (FIG. 31). As expected, the engineered vector backbone without the ORF library showed an even higher transduction efficiency, due to its smaller size.

### REFERENCES

1. Abramson. J. S, et al. Transcend NHL 001: immunotherapy with the CD19-directed CAR T-cell product JCAR017 results in high complete response rates in relapsed or refractory B-cell non-Hodgkin lymphoma. Blood 128, 4192-4192 (2016).
2. Shifrut, E, et al. Genome-wide CRISPR screens in primary human T cells reveal key regulators of immune function. Cell 175, 1958-1971 (2018).
3. Dong, M. B, et al. Systematic immunotherapy target discovery using genome-scale in vivo CRISPR screens in CD8 T cells. Cell 178, 1189-1204 (2019).
4. Chen, Z, et al. In vivo CD8+ T cell CRISPR screening reveals control by Fli1 in infection and cancer. Cell 184, 1262-1280 (2021).
5. Kabelitz, D., Serrano. R., Kouakanou, L., Peters. C. & Kalyan, S. Cancer immunotherapy with γδ T cells: many paths ahead of us. Cell. Mol. Immunol. 17, 925-939 (2020).
6. Munshi, N. C, et al. Idecabtagene vicleucel in relapsed and refractory multiple myeloma. N. Engl. J. Med. 384, 705-716 (2021).
7. Fraietta, J. A, et al. Determinants of response and resistance to CD19 chimeric antigen receptor (CAR) T cell therapy of chronic lymphocytic leukemia. Nat. Med. 24, 563-571 (2018).
8. Singer, M, et al. A distinct gene module for dysfunction uncoupled from activation in tumor-infiltrating T cells. Cell 166, 1500-1511 (2016).
9. Gurusamy. D, et al. Multi-phenotype CRISPR-Cas9 screen identifies p38 kinase as a target for adoptive immunotherapies. Cancer Cell 37, 818-833 (2020).
10. Legut. M . . . . Dolton, G., Mian, A. A., Ottmann, O. G. & Sewell, A. K. CRISPR-mediated TCR replacement generates superior anticancer transgenic T cells. Blood 131, 311-322 (2018).
11. Ren, J, et al. Multiplex genome editing to generate universal CAR T cells resistant to PDI inhibition. Clin. Cancer Res. 23, 2255-2266 (2017).
12. Wagner, D. L, et al. High prevalence of Streptococcus pyogenes Cas9-reactive T cells within the adult human population. Nat. Med. 25, 242-248(2019).
13. Wu. X, et al. Genome-wide binding of the CRISPR endonuclease Cas9 in mammalian cells. Nat. Biotechnol. 32, 670-676 (2014).
14. Sack, L. M, et al. Profound tissue specificity in proliferation control underlies cancer drivers and aneuploidy patterns. Cell 173, 499-514 (2018).
15. Yuan. J, et al. CTLA-4 blockade enhances polyfunctional NY-ESO-1 specific T cell responses in metastatic melanoma patients with clinical benefit. Proc. Natl Acad. Sci. USA 105, 20410-20415 (2008).
16. Sommermeyer, D, et al. Chimeric antigen receptor-modified T cells derived from defined CD8+ and CD4+ subsets confer superior antitumor reactivity in vivo. Leukemia 30. 492-500 (2016).
17. Fischer, A. M., Katayama. C. D., Pagès, G., Pouysségur. J. & Hedrick, S. M. The role of Erk1 and Erk2 in multiple stages of T cell development. Immunity 23, 431-443 (2005).
18. Lipp. A. M, et al. Lck mediates signal transmission from CD59 to the TCR/CD3 pathway in Jurkat T cells. PLOS One 9, e85934 (2014).
19. Ma. X, et al. Interleukin-23 engineering improves CAR T cell function in solid tumors. Nat. Biotechnol. 38, 448-459 (2020).
20. Seo. H, et al. BATF and IRF4 cooperate to counter exhaustion in tumor-infiltrating CAR T cells. Nat. Immunol. 22, 983-995 (2021).
21. Jamali, A, et al. Highly efficient and selective CAR-gene transfer using CD4− and CD8− targeted lentiviral vectors Mol. Ther. Methods Clin. Dev. 13, 371-379 (2019)
22. Stoeckius, M, et al. Simultaneous epitope and transcriptome measurement in single cells Nat. Methods 14, 865-868(2017).
23. Mimitou, E. P, et al. Multiplexed detection of proteins, transcriptomes, clonotypes and CRISPR perturbations in single cells. Nat. Methods 16, 409-412 (2019).
24. Gil-Yarom. N, et al. CD74 is a novel transcription regulator. Proc. Natl Acad. Sci. USA 114. 562-567 (2017).
25. Ataide, M. A, et al. BATF3 programs CD8+ T cell memory. Nat. Immunol. 21, 1397-1407 (2020).
26. Katagiri, T., Kameda, H., Nakano, H. & Yamazaki, S. Regulation of T cell differentiation by the AP-1 transcription factor JunB. Immunol. Med. 44, 197-203 (2021).
27. Zhao, X., Shan. Q. & Xuc. H.-H. TCF1 in T cell immunity: a broadened frontier. Nat. Rev. Immunol, https://doi.org/10.1038/s41577-021-00563-6 (2021).
28. Sudhamsu, J, et al. Dimerization of LTβR by LTα1β2 is necessary and sufficient for signal transduction. Proc. Natl Acad. Sci USA 110, 19896-19901 (2013).
29. Li. C, et al. Structurally distinct recognition motifs in lymphotoxin-receptor and CD40 for tumor necrosis factor receptor-associated factor (TRAF)-mediated signaling. J. Biol. Chem. 278, 50523-50529 (2003).
30. Wu. M.-Y., Wang, P.-Y., Han, S.-H. & Hsieh, S.-L. The cytoplasmic domain of the lymphotoxin-β receptor mediates cell death in HeLa cells. J. Biol. Chem. 274, 11868-11873 (1999).
31. Macian. F. NFAT proteins: key regulators of T-cell development and function. Nat. Rev. Immunol. 5, 472-484 (2005).
32. Dejardin, E et al. The lymphotoxin-p receptor induces different patterns of gene expression via two NF-κB pathways. Immunity 17, 525-535 (2002).
33. Saoulli. K, et al. CD28-independent. TRAF2-dependent costimulation of resting T cells by 4-1BB ligand. J. Exp. Med. 187, 1849-1862 (1998)
34. Thommen, D. S. & Schumacher, T. N. T cell dysfunction in cancer. Cancer Cell 33, 547-562 (2018).
35. Roth, T. L, et al. Pooled knockin targeting for genome engineering of cellular immunotherapies. Cell 181, 728-744 (2020).
36. VanArsdale. T. L, et al. Lymphotoxin-receptor signaling complex: role of tumor necrosis factor receptor-associated factor 3 recruitment in cell death and activation of nuclear factor B. Proc. Natl Acad. Sci. USA 94, 2460-2465 (1997).
37. Yilmaz, Z. B, et al. Quantitative dissection and modeling of the NF-κB p100-p105 module reveals interdependent precursor proteolysis. Cell Rep. 9, 1756-1769 (2014).
38. Hu, B, et al. Augmentation of antitumor immunity by human and mouse CAR T cells secreting IL-18. Cell Rep. 20, 3025-3033 (2017).
39. Yeku, O. O., Purdon, T. J., Koneru, M., Spriggs, D. & Brentjens, R. J. Armored CAR T cells enhance antitumor efficacy and overcome the tumor microenvironment. Sci. Rep. 7, 10541 (2017).
40. Lynn, R. C, et al, c-Jun overexpression in CAR T cells induces exhaustion resistance. Nature 576, 293-300 (2019).
41. Schmiedel, B. J, et al. Impact of genetic polymorphisms on human immune cell gene expression. Cell 175, 1701-1715 (2018).
42. Legut et al, A genome-scale screen for synthetic drivers of T cell proliferation. Nature 603, 728-723 (March 2022).
43. Theruvath et al, Locoregionally administered B7-H3-targeted CAR T cells for treatment of atypical teratoid/rhaboid tumors. Nature Medicine 26, 712-719 (April 2020).

All publications cited in this specification are incorporated herein by reference. U.S. Provisional Patent Application No. 63/320,100 filed Mar. 15, 2022 is incorporated herein by reference. While the invention has been described with reference to particular embodiments, it will be appreciated that modifications can be made without departing from the spirit of the invention. Such modifications are intended to fall within the scope of the appended claims.

What is claimed is:

1. A lymphocyte genetically modified to express a chimeric antigen receptor (CAR), wherein the CAR comprises:
a) an antigen binding domain;
b) a transmembrane domain; and
c) a signaling domain;
wherein at least one domain comprises an LTBR domain.

2. The modified lymphocyte according to claim 1, wherein the LTBR domain is an LTBR intracellular domain, or fragment or variant thereof.

3. The modified lymphocyte according to claim 2, wherein the LTBR intracellular domain comprises amino acids 249 to 435 of SEQ ID NO: 2, or a fragment, deletion, or variant thereof.

4. The modified lymphocyte according to claim 2 or 3, wherein the LTBR intracellular domain has a deletion in at least amino acids 393 to 435.

5. The modified lymphocyte according to any one of claims 1 to 4, wherein the transmembrane domain is an LTBR domain.

6. The modified lymphocyte according to any one of claims 1 to 5, wherein the extracellular domain is an LTBR domain.

7. The modified lymphocyte according to any one of claims 1 to 6, wherein the lymphocyte is a T cell.

8. The modified lymphocyte according to any one of claims 1 to 7, wherein the lymphocyte is an alpha beta T cell or gamma delta T cell, optionally a Vγ9Vδ2 T cell.

9. The modified lymphocyte according to any one of claims 1 to 6, wherein the lymphocyte is an NK cell or NK T cell.

10. The modified lymphocyte according to any one of claims 1 to 9, wherein the LTBR intracellular domain is positioned between the co-stimulatory signaling domain of and the signaling domain of (c).

11. The modified lymphocyte according to any one of claims 1 to 9, wherein the LTBR domain is positioned after the signaling domain of (c).

12. A lymphocyte genetically modified to express a chimeric antigen receptor (CAR), wherein the CAR comprises:
a) an antigen binding domain;
b) a transmembrane domain;
c) a co-stimulatory signaling domain; and
d) a signaling domain;
wherein at least one domain comprises an LTBR domain.

13. The modified lymphocyte according to claim 12, wherein the LTBR domain is an LTBR intracellular domain, or fragment or variant thereof.

14. The modified lymphocyte according to claim 13, wherein the LTBR intracellular domain comprises amino acids 249 to 435 of SEQ ID NO: 2, or a fragment, deletion, or variant thereof.

15. The modified lymphocyte according to claim 13 or 14, wherein the LTBR intracellular domain has a deletion in at least amino acids 393 to 435.

16. The modified lymphocyte according to any one of claims 12 to 15, wherein the transmembrane domain is an LTBR domain.

17. The modified lymphocyte according to any one of claims 12 to 16, wherein the extracellular domain is an LTBR domain.

18. The modified lymphocyte according to any one of claims 12 to 17, wherein the lymphocyte is a T cell.

19. The modified lymphocyte according to any one of claims 12 to 18, wherein the lymphocyte is an alpha beta T or gamma delta T cell, optionally a Vγ9Vδ2 T cell.

20. The modified lymphocyte according to any one of claims 12 to 18, wherein the lymphocyte is an NK cell or NK T cell.

21. The modified lymphocyte according to any one of claims 12 to 20, wherein the LTBR intracellular domain is positioned between the transmembrane domain of (b) and the co-stimulatory signaling domain of (c).

22. The modified lymphocyte according to any one of claims 1 to 20, wherein the LTBR intracellular domain is positioned between the co-stimulatory signaling domain of (c) and the signaling domain of (d).

23. The modified lymphocyte according to any one of claims 1 to 20, wherein the LTBR domain is positioned after the signaling domain of (c).

24. The modified lymphocyte according to any one of claims 1 to 23, wherein the CAR is selected from Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kyrmriah®), or one of those found in FIG. 19, modified to include an LTBR domain.

25. A nucleic acid molecule comprising a sequence that encodes a chimeric antigen receptor (CAR), wherein the CAR comprises:
a) an antigen binding domain;
b) a transmembrane domain; and
c) a signaling domain;
wherein at least one domain comprises an LTBR domain.

26. The nucleic acid molecule according to claim 25, wherein the LTBR domain is an LTBR intracellular domain, or fragment or variant thereof.

27. The nucleic acid molecule according to claim 26, wherein the LTBR intracellular domain comprises amino acids 249 to 435 of SEQ ID NO: 2, or a fragment, deletion, or variant thereof.

28. The nucleic acid molecule according to claim 26 or 27, wherein the LTBR intracellular domain has a deletion in at least amino acids 393 to 435.

29. The nucleic acid molecule according to any one of claims 25 to 28, wherein the transmembrane domain is an LTBR domain.

30. The nucleic acid molecule according to any one of claims 25 to 29, wherein the extracellular domain is an LTBR domain.

31. The nucleic acid molecule according to any one of claims 25 to 30, wherein the LTBR intracellular domain is positioned between the co-stimulatory signaling domain of and the signaling domain of (c).

32. The nucleic acid molecule according to any one of claims 25 to 30, wherein the LTBR domain is positioned after the signaling domain of (c).

33. A nucleic acid molecule comprising a sequence that encodes a chimeric antigen receptor (CAR), wherein the CAR comprises:
a. an antigen binding domain;
b. a transmembrane domain;
c. a co-stimulatory signaling domain; and
d. a signaling domain;
wherein at least one domain comprises an LTBR domain.

34. The nucleic acid molecule according to claim 33, wherein the LTBR domain is an LTBR intracellular domain, or fragment or variant thereof.

35. The nucleic acid molecule according to claim 34, wherein the LTBR intracellular domain comprises amino acids 249 to 435 of SEQ ID NO: 2, or a fragment, deletion, or variant thereof.

36. The nucleic acid molecule according to claim 34 or 35, wherein the LTBR intracellular domain has a deletion in at least amino acids 393 to 435.

37. The nucleic acid molecule according to any one of claims 33 to 36, wherein the transmembrane domain is an LTBR domain.

38. The nucleic acid molecule according to any one of claims 33 to 37, wherein the extracellular domain is an LTBR domain.

39. The nucleic acid molecule according to any one of claims 33 to 38, wherein the LTBR intracellular domain is positioned between the transmembrane domain of (b) and the co-stimulatory signaling domain of (c).

40. The nucleic acid molecule according to any one of claims 33 to 39, wherein the LTBR intracellular domain is positioned between the co-stimulatory signaling domain of (c) and the signaling domain of (d).

41. The nucleic acid molecule according to any one of claims 33 to 40, wherein the LTBR domain is positioned after the signaling domain of (c).

42. The nucleic acid molecule according to any one of claims 25 to 41, wherein the CAR is selected from Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kymriah®), or one of those found in FIG. 19, modified to include an LTBR domain.

43. An expression cassette comprising the nucleic acid molecule according to any one of claims 25 to 42.

44. The expression cassette according to claim 43, further comprising an expression control sequence.

45. The expression cassette according to claim 44, wherein the expression control sequence comprises a promoter.

46. The expression cassette according to claim 45, wherein the promoter is a constitutive promoter or an inducible promoter.

47. A modified lymphocyte comprising the nucleic acid molecule according to any one of claims 25 to 42 or the expression cassette according to any one of claims 43 to 46.

48. A method of producing a modified lymphocyte comprising introducing the nucleic acid molecule according to any one of claims 25 to 42 or the expression cassette according to any one of claims 43 to 46 into the lymphocyte.

49. A method of treating cancer in a subject in need thereof, the method comprising administering a composition comprising the modified lymphocyte according to any one of claims 1 to 24 or 47 to the subject.

50. The method according to claim 49, wherein the subject has lymphoma, optionally B cell lymphoma, follicular lymphoma, and mantle cell lymphoma.

51. The method according to claim 49, wherein the subject has a solid tumor cancer.

52. The method according to claim 49, wherein the subject has leukemia.

53. The method according to claim 49, wherein the subject has multiple myeloma.

54. The method according to claim 49, wherein the subject bas a virally-driven cancer.

55. The method according to claim 54, wherein the subject has HPV.

56. The method according to claim 54, wherein the subject has a cancer selected from Burkitt's lymphoma, liver cancer, Kaposi's sarcoma, cervical cancer, head cancer, neck cancer, anal cancer, pancreatic cancer, melanoma, oral cancer, pharyngeal cancer, penile cancer, adult T-cell lymphoma, and merkel cell carcinoma.

57. A method of treating a viral disease in a subject in need thereof, the method comprising administering a composition comprising the modified lymphocyte according to any one of claim 1 to 24 or 47 to the subject.

58. The method according to claim 57, wherein the disease is HIV or HPV.

59. A method of treating an autoimmune in a subject in need thereof, the method comprising administering a composition comprising the modified lymphocyte according to any one of claims 1 to 24 or 47 to the subject.

60. The method according to claim 29, wherein the disease is an autoimmune disorder.

61. A method of increasing proliferation, or T cell effector function including cytokine production and/or secretion, the method comprising administering a composition comprising the modified lymphocyte according to any one of claim 1 to 24 or 47 to the subject.

62. The method according to claim 61, wherein the T cell is obtained from a human prior to treating the T cell to overexpress LTBR, and the treated T cell is reintroduced into a human.

63. A fusion protein comprising an LTBR domain and at least one domain from a second protein, that is not LTBR.

64. The fusion protein according to claim 63, wherein the second protein is CD4.

65. The fusion protein according to claim 63, wherein the second protein is CD8A or CD8B.

66. The fusion protein according to claim 63, wherein the second protein is CD3E.

67. The fusion protein according to claim 63, wherein the second protein is CD3D.

68. The fusion protein according to claim 63, wherein the second protein is CD3G.

69. The fusion protein according to claim 63, wherein the second protein is CD3Z.

70. The fusion protein according to any one of claims 63-69, wherein the LTBR domain is an LTBR intracellular domain, or fragment or variant thereof.

71. The fusion protein according to claim 70, wherein the LTBR intracellular domain comprises amino acids 249 to 435 of SEQ ID NO: 2, or a fragment, deletion, or variant thereof.

72. The fusion protein according to claim 70 or 71, wherein the LTBR intracellular domain has a deletion in at least amino acids 393 to 435.

73. A nucleic acid molecule comprising a sequence that encodes the fusion protein according to any one of claims 63 to 72.

74. A host cell comprising the nucleic acid molecule according to claim 73.

75. The host cell according to claim 74, which is a lymphocyte.

76. The host cell according to claim 75, which is a T cell.

77. A lymphocyte genetically modified to express a T cell receptor (TCR), wherein the TCR comprises an alpha chain fused to an LTBR intracellular domain.

78. A lymphocyte genetically modified to express a T cell receptor (TCR), wherein the TCR comprises a beta chain fused to an LTBR intracellular domain.

79. A lymphocyte genetically modified to express a T cell receptor (TCR), wherein the TCR comprises a gamma chain fused to an LTBR intracellular domain.

80. A lymphocyte genetically modified to express a T cell receptor (TCR), wherein the TCR comprises a delta chain fused to an LTBR intracellular domain.

81. The modified lymphocyte according to any one of claims 77 to 80, wherein the LTBR domain is an LTBR intracellular domain, or fragment or variant thereof.

82. The modified lymphocyte according to claim 81, wherein the LTBR intracellular domain comprises amino acids 249 to 435 of SEQ ID NO: 2, or a fragment, deletion, or variant thereof.

83. The modified lymphocyte according to claim 81 or 82, wherein the LTBR intracellular domain has a deletion in at least amino acids 393 to 435.

84. The modified lymphocyte according to any one of claims 77 to 83, wherein the lymphocyte is a T cell.

85. The modified lymphocyte according to any one of claims 77 to 84, wherein the lymphocyte is an alpha beta T cell or gamma delta T cell, a Vγ9Vδ2 T cell.

86. The modified lymphocyte according to any one of claims 77 to 83, wherein the lymphocyte is an NK cell or NK T cell.

87. The modified lymphocyte according to any one of claims 77 to 86, wherein the TCR is tebentafusp-tebn (Kimmtrak®) or one of those found in FIG. 20, modified to include an LTBR domain.

88. A nucleic acid molecule comprising a sequence that encodes a chimeric antigen receptor (CAR), wherein the CAR comprises:
a. an antigen binding domain;
b. a transmembrane domain; and
c. a signaling domain;
wherein at least one domain comprises an LTBR domain.

89. The nucleic acid molecule according to claim 88, wherein the LTBR domain is an LTBR intracellular domain, or fragment or variant thereof.

90. The nucleic acid molecule according to claim 89, wherein the LTBR intracellular domain comprises amino acids 249 to 435 of SEQ ID NO: 2, or a fragment, deletion, or variant thereof.

91. The nucleic acid molecule according to claim 88 or 89, wherein the LTBR intracellular domain has a deletion in at least amino acids 393 to 435.

92. An expression cassette comprising the nucleic acid molecule according to any one of claims 88 to 91.

93. The expression cassette according to claim 92, further comprising an expression control sequence.

94. The expression cassette according to claim 93, wherein the expression control sequence comprises a promoter.

95. The expression cassette according to claim 94, wherein the promoter is a constitutive promoter or an inducible promoter.

96. A modified lymphocyte comprising the nucleic acid molecule according to any one of claims 88 to 91 or the expression cassette according to any one of claims 92 to 95.

97. A method of producing a modified lymphocyte comprising introducing the nucleic acid molecule according to any one of claims 88 to 91 or the expression cassette according to any one of claims 92 to 95 into the lymphocyte.

98. A method of treating cancer in a subject in need thereof, the method comprising administering a composition comprising the modified lymphocyte according to any one of claim 77 to 87 or 96 to the subject.

99. The method according to claim 98, wherein the subject has lymphoma, optionally B cell lymphoma, follicular lymphoma, and mantle cell lymphoma.

100. The method according to claim 99, wherein the subject has a solid tumor cancer.

101. The method according to claim 98, wherein the subject bas leukemia.

102. The method according to claim 98, wherein the subject has multiple mycloma.

103. The method according to claim 98, wherein the subject has multiple uveal melanoma.

104. The method according to claim 98, wherein the subject has a virally-driven cancer.

105. The method according to claim 98, wherein the subject has HPV.

106. The method according to claim 104, wherein the subject has a cancer selected from Burkitt's lymphoma, liver cancer, Kaposi's sarcoma, cervical cancer, head cancer, neck cancer, anal cancer, oral cancer, pharyngeal cancer, penile cancer, adult T-cell lymphoma, and merkel cell carcinoma.

107. A method of increasing proliferation, or T cell effector function including cytokine production and/or secretion, the method comprising administering a composition comprising the modified lymphocyte according to any one of claim 77 to 87 or 96 to the subject.

108. The method according to claim 107, wherein the T cell is obtained from a human prior to modifying the lymphocyte, and the modified lymphocyte is reintroduced into a human.

109. The nucleic acid molecule according to XX, wherein the CAR targets CD19, mesothelin, ROR1, B7-H3, IL13Rα2, GD2, Her2, Glypican 3, CD7, NY-ESO-1, CD30, MAGE-A1, LMP2, PD1, KRAS G12V, CD20, CD22, CD171, CD123, CD38, CD10, BAFFR, PSMA, or mucin.

110. A fusion protein comprising:
a. an antigen binding domain:
b. LTBR:
c. and at least one domain from a second protein that is not LTBR.

111. The fusion protein according to claim 110, wherein the second protein is CD4.

112. The fusion protein according to claim 110, wherein the second protein is CD8A or CD8B.

113. The fusion protein according to claim 110, wherein the second protein is CD3E.

114. The fusion protein according to claim 110, wherein the second protein is CD3D.

115. The fusion protein according to claim 110, wherein the second protein is CD3G.

116. The fusion protein according to claim 110, wherein the second protein is CD3Z.

117. The fusion protein according to any one of claims 110-116, wherein the LTBR domain is an LTBR intracellular domain, or fragment or variant thereof.

118. A nucleic acid molecule comprising a sequence that encodes the fusion protein according to any one of claims 110 to 117.

119. A modified lymphocyte comprising the nucleic acid molecule according to claim 118.

120. A modified lymphocyte comprising the nucleic acid molecule according to claim 118 and further comprising a nucleic acid molecule that encodes a CAR.

121. The modified lymphocyte according to claim 120, wherein the CAR and the antigen binding domain both target mesothelin.

122. A lymphocyte genetically modified to express a T cell receptor (TCR) and a CD8 alpha chain fused to an LTBR intracellular domain.

123. A lymphocyte genetically modified to express a T cell receptor (TCR) and a CD8 beta chain fused to an LTBR intracellular domain.

124. The modified lymphocyte according to claim 122 or 123, wherein the LTBR domain is an LTBR intracellular domain, or fragment or variant thereof.

125. The modified lymphocyte according to claim 124, wherein the LTBR intracellular domain comprises amino acids 249 to 435 of SEQ ID NO: 2, or a fragment, deletion, or variant thereof.

126. The modified lymphocyte according to claim 124 or 125, wherein the LTBR intracellular domain has a deletion in at least amino acids 393 to 435.

127. The modified lymphocyte according to any one of claims 122 to 126, wherein the lymphocyte is a T cell.

128. The modified lymphocyte according to any one of claims 122 to 127, wherein the lymphocyte is an alpha beta T cell or gamma delta T cell, a Vγ9Vδ2 T cell.

129. The modified lymphocyte according to any one of claims 122 to 126, wherein the lymphocyte is an NK cell or NK T cell.

130. The modified lymphocyte according to any one of claims 122 to 129, wherein the TCR is tcbentafusp-tcbn (Kimmtrak®) or one of those found in FIG. 20.

131. An engineered lentiviral vector comprising the sequence of SEQ ID NO: 132, or a sequence sharing at least 90% identity with SEQ ID NO: 132, optionally with an open reading frame (ORF) for a gene of interest and/or a barcode inserted within the sequence.

132. The engineered vector of claim 131, wherein the ORF and/or barcode are inserted after nucleotide 3291 of SEQ ID NO: 132.
